(19) United States
(12) Patent Application Publication
(10) Pub. No.: US 2025/0195673 A1
(43) Pub. Date: Jun. 19, 2025

(54) COMPOSITIONS AND METHODS FOR SELECTIVE DEPLETION OF TARGET MOLECULES

(71) Applicants: Fred Hutchinson Cancer Center, Seattle, WA (US); Blaze Bioscience, Inc., Seattle, WA (US)

(72) Inventors: Zachary CROOK, Bothell, WA (US); James OLSON, Seattle, WA (US); Roland K. STRONG, Seattle, WA (US); Natalie Winblade NAIRN, Seattle, WA (US); Stephen TAPSCOTT, Seattle, WA (US); Kenneth GRABSTEIN, Mercer Island, WA (US)

(21) Appl. No.: 18/037,923

(22) PCT Filed: Nov. 29, 2021
(86) PCT No.: PCT/US2021/061035

§ 371 Date: May 19, 2023

Related U.S. Application Data
(60) Provisional application No. 63/119,195, filed on Nov. 30, 2020.

(51) Int. Cl. A61K 47/64; C07K 14/00; C07K 16/28

(52) U.S. Cl. CPC A61K 47/64; C07K 14/001; C07K 16/2863; C07K 2317/569; C07K 2317/92

(57) ABSTRACT

Described herein are compositions and methods for selective depletion of target molecules using a recyclable CDP-receptor-binding mediated complex to elicit endocytosis and cellular degradation of the target. Exemplary compositions containing a peptide, such as a CDP peptide, that bind a transferrin receptor can be linked to a peptide that binds a target molecule. Such compositions can be used to selectively recruit the target molecule to endosomes via transferrin receptor-mediated endocytosis of the composition and the bound target molecule. Once inside the endosome, the acidic pH can lead to release of the target molecule from the composition due to pH-dependent binding of the composition for the target molecule, and the transferrin receptor portion is recycled back to the cell surface for “reloading”. The target molecule can then be trafficked into lysosomes wherein it is degraded.

### CROSS-REFERENCE

The present application claims the benefit of U.S. Provisional Application No. 63/119,195, entitled “COMPOSITIONS AND METHODS FOR SELECTIVE DEPLETION OF TARGET MOLECULES,” filed on Nov. 30, 2020, which application is herein incorporated by reference in its entirety for all purposes.

### SEQUENCE LISTING

The instant application contains a Sequence Listing which has been submitted electronically in ASCII format and is hereby incorporated by reference in its entirety. Said ASCII copy, created on Nov. 24, 2021, is named 108406-702531_SL.txt and is 665,995 bytes in size.

### BACKGROUND

Accumulation or over-expression of soluble and cell surface proteins is indicated in a variety of human diseases, ranging from neurodegenerative diseases to cancer. Furthermore, numerous diseases are associated with mutations in soluble or cell surface proteins resulting in constitutive activity, resistance to treatment, or dominant negative activity. However, many of these proteins have been deemed “undruggable,” “difficult to drug,” or “yet to be drugged” targets due to challenges in targeting them with small molecule therapeutics. For example, in the neurodegenerative Alzheimer's disease, the amyloid protein which accumulates to form plaques in the brain as a marked aspect of the disease lacks therapeutic agents that target the protein despite its critical role in neurodegeneration. There is a need for compositions and methods to target and selectively deplete soluble and cell surface proteins associated with disease.

### SUMMARY

In various aspects, the present disclosure provides a peptide complex comprising: a cellular receptor-binding peptide; and a target-binding peptide complexed with the cellular receptor-binding peptide, wherein (i) the target-binding peptide is engineered to have an affinity for a target that is lower in an endosome than in an extracellular environment, (ii) the cellular receptor-binding peptide is engineered to have an affinity for a cellular receptor is lower in an endosome than in an extracellular environment, or both (i) and (ii).

In some aspects, the affinity of the target-binding peptide for the target, the affinity of the cellular receptor binding peptide for the cellular receptor, or both is pH dependent. In some aspects, the affinity of the target-binding peptide for the target, the affinity of the cellular receptor-binding peptide for the cellular receptor, or both is ionic strength dependent.

In various aspects, the present disclosure provides a peptide complex comprising: a cellular receptor binding peptide; and a target-binding peptide complexed with the cellular receptor-binding peptide, wherein (i) an affinity of the target-binding peptide for a target is pH dependent, (ii) an affinity of the cellular receptor-binding peptide for a cellular receptor is pH dependent, or both (i) and (ii).

In some aspects, the cellular receptor-binding peptide is a transferrin receptor-binding peptide or a PD-L1-binding peptide. In some aspects, the cellular receptor-binding peptide is a transferrin receptor-binding peptide. In some aspects, the cellular receptor-binding peptide is a PD-L1-binding peptide. In some aspects, the cellular receptor is a transferrin receptor or PD-L1. In some aspects, the cellular receptor is a transferrin receptor. In some aspects, the cellular receptor is PD-L1.

In some aspects, the cellular receptor-binding peptide binds to the cellular receptor at a pH of from pH 4.5 to pH 7.4, from pH 5.5 to pH 7.4, or from pH 6.5 to pH 7.4. In some aspects, the cellular receptor-binding peptide is capable of binding the cellular receptor with a dissociation constant (KD) of no more than 100 nM, no more than 20 nM, no more than 10 nM, no more than 5 nM, no more than 2 nM, no more than 1 nM, no more than 0.5 nM, no more than 0.4 nM, no more than 0.3 nM, no more than 0.2 nM, or no more than 0.1 nM at pH 7.4. In some aspects, the cellular receptor-binding peptide is capable of binding the cellular receptor with a dissociation constant (KD) of no more than 100 nM, no more than 20 nM, no more than 10 nM, no more than 5 nM, no more than 2 nM, no more than 1 nM, no more than 0.5 nM, no more than 0.4 nM, no more than 0.3 nM, no more than 0.2 nM, or no more than 0.1 nM at pH 5.5. In some aspects, the affinity of the cellular receptor for the cellular receptor is pH-independent. In some aspects, the affinity of the cellular receptor-binding peptide for the cellular receptor at pH 7.4 and at pH 5.5 differs by no more than 2-fold, no more than 5-fold, no more than 10-fold, no more than 15-fold, no more than 20-fold, no more than 25-fold, no more than 30-fold, no more than 40-fold, or no more than 50-fold.

In some aspects, the affinity of the cellular receptor-binding peptide for the cellular receptor is pH dependent. In some aspects, the affinity of the cellular receptor-binding peptide for the cellular receptor decreases as pH decreases. In some aspects, the affinity of the cellular receptor-binding peptide for the cellular receptor is higher at pH 7.4 than at pH 5.5.

In some aspects, the affinity of the target-binding peptide for the target is pH dependent. In some aspects, the affinity of the target-binding peptide for the target decreases as pH decreases. In some aspects, the affinity of the target-binding peptide for the target is higher at a higher pH than at a lower pH. In some aspects, the higher pH is pH 7.4, pH 7.2, pH 7.0, or pH 6.8. In some aspects, the lower pH is pH 6.5, pH 6.0, pH 5.5, pH 5.0, or pH 4.5. In some aspects, the affinity of the target-binding peptide for the target is higher at pH 7.4 than at pH 6.0. In some aspects, the affinity of the target-binding peptide for the target is higher at pH 7.4 than at pH 5.5. In some aspects, the target-binding peptide is capable of binding the target molecule with a dissociation constant (KD) of no more than 100 nM, no more than 20 nM, no more than 10 nM, no more than 5 nM, no more than 2 nM, no more than 1 nM, no more than 0.5 nM, no more than 0.4 nM, no more than 0.3 nM, no more than 0.2 nM, no more than 1 nM, or no more than 0.1 nM at pH 7.4. In some aspects, the target-binding peptide is capable of binding the target molecule with a dissociation constant (KD) of no less than 1 nM, no less than 2 nM, no less than 5 nM, no less than 10 nM, no less than 20 nM, no less than 50 nM, no less than 100 nM, no less than 200 nM, or no less than 500 nM at pH 5.5. In some aspects, the affinity of the target-binding peptide for the target at pH 7.4 is at least 2-fold, at least 3-fold, at least 4-fold, at least 5-fold, at least 6-fold, at least 7-fold, at least 8-fold, at least 9-fold, at least 10-fold, at least 15-fold, or at least 20-fold greater than the affinity of the target binding peptide for the target at pH 5.5. In some aspects, the target-binding peptide comprises one or more histidine amino acid residues. In some aspects, the affinity of the target-binding peptide for the target decreases as ionic strength increases. In some aspects, the target-binding peptide comprises one or more polar or charged amino acid residues capable of forming polar or charge-charge interactions with the target molecule.

In some aspects, the cellular receptor-binding peptide is conjugated to the target binding peptide. In some aspects, the cellular receptor-binding peptide and the target binding peptide form a single polypeptide chain. In some aspects, the peptide complex comprises a dimer dimerized via a dimerization domain. In some aspects, the dimerization domain comprises an Fc domain. In some aspects, the dimer is a homodimer dimerized via a homodimerization domain. In some aspects, the homodimerization domain comprises a sequence that has at least 80%, at least 90%, at least 92%, at least 93%, at least 94%, at least 95%, at least 96%, at least 97%, at least 98%, or at least 99% sequence identity with any one of SEQ ID NO: 245-SEQ ID NO: 259. In some aspects, the dimer is a heterodimer dimerized via a first heterodimerization domain and a second heterodimerization domain. In some aspects, the first heterodimerization domain comprises a sequence that has at least 80%, at least 90%, at least 92%, at least 93%, at least 94%, at least 95%, at least 96%, at least 97%, at least 98%, or at least 99% sequence identity with any one of SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, or SEQ ID NO: 286. In some aspects, the second heterodimerization domain comprises a sequence that has at least 80%, at least 90%, at least 92%, at least 93%, at least 94%, at least 95%, at least 96%, at least 97%, at least 98%, or at least 99% sequence identity with any one of SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, or SEQ ID NO: 287.

In some aspects, the target-binding peptide is linked to the dimerization domain via a peptide linker. In some aspects, the cellular receptor-binding peptide is linked to the dimerization domain via a peptide linker. In some aspects, the cellular receptor-binding peptide is linked to the target binding peptide via a peptide linker. In some aspects, the peptide linker has a length of from 1 to 50 amino acid residues, from 2 to 40 amino acid residues, from 3 to 20 amino acid residues, or from 3 to 10 amino acid residues. In some aspects, the peptide linker comprises glycine and serine amino acids. In some aspects, the peptide linker has a persistence length of no more than 6 Å, no more than 8 Å, no more than 10 Å, no more than 12 Å, no more than 15 Å, no more than 20 Å, no more than 25 Å, no more than 30 Å, no more than 40 Å, or no more than 50 Å. In some aspects, the peptide linker is derived from an immunoglobulin peptide. In some aspects, the peptide linker is derived from a double-knot toxin peptide. In some aspects, the peptide linker comprises a sequence of any one of SEQ ID NO: 129-SEQ ID NO: 141, SEQ ID NO: 195-SEQ ID NO: 218, SEQ ID NO: 223-SEQ ID NO: 227, or SEQ ID NO: 391.

In some aspects, the cellular receptor-binding peptide, the target-binding peptide, or both comprises a miniprotein, a nanobody, an antibody, an antibody fragment, an scFv, a DARPin, or an affibody. In some aspects, the antibody comprises an IgG, or wherein the antibody fragment comprises a Fab, a F(ab)2, an scFv, or an (scFv)2. In some aspects, the miniprotein comprises a cystine-dense peptide, an affitin, an adnectin, an avimer, a Kunitz domain, a nanofittin, a fynomer, a bicyclic peptide, a beta-hairpin, or a stapled peptide. In some aspects, the cellular receptor-binding peptide comprises at least one disulfide bond, at least two disulfide bonds, at least three disulfide bonds, or at least four disulfide bonds.

In some aspects, the target-binding peptide comprises at least one disulfide bond, at least two disulfide bonds, at least three disulfide bonds, or at least four disulfide bonds. In some aspects, the cellular receptor-binding peptide comprises at least six cysteine residues. In some aspects, the at least six cysteine residues are positioned at amino acid positions 4, 8, 18, 32, 42, and 46 of the cellular receptor-binding peptide. In some aspects, the at least six cysteine residues form at least three disulfide bonds.

In some aspects, the cellular receptor-binding peptide comprises a sequence of any one of SEQ ID NO: 148-SEQ ID NO: 177. In some aspects, the cellular receptor-binding peptide comprises a sequence that has at least 80%, at least 90%, at least 92%, at least 93%, at least 94%, at least 95%, at least 96%, at least 97%, at least 98%, or at least 99% sequence identity with any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64, or at least 80%, at least 90%, at least 92%, at least 93%, at least 94%, at least 95%, at least 96%, at least 97%, at least 98%, or at least 99% sequence identity with a fragment of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. In some aspects, the cellular receptor-binding peptide comprises a sequence that has at least 80%, at least 90%, at least 92%, at least 93%, at least 94%, at least 95%, at least 96%, at least 97%, at least 98%, or at least 99% sequence identity with SEQ ID NO: 96, or at least 80%, at least 90%, at least 92%, at least 93%, at least 94%, at least 95%, at least 96%, at least 97%, at least 98%, or at least 99% sequence identity with a fragment of SEQ ID NO: 96. In some aspects, the cellular receptor-binding peptide comprises a sequence of SEQ ID NO: 96. In some aspects, the cellular receptor-binding peptide comprises a sequence of any one of SEQ ID NO: 392-SEQ ID NO: 399. In some aspects, the cellular receptor-binding peptide comprises a sequence that has at least 80%, at least 90%, at least 92%, at least 93%, at least 94%, at least 95%, at least 96%, at least 97%, at least 98%, or at least 99% sequence identity with any one of SEQ ID NO: 187, SEQ ID NO: 233-SEQ ID NO: 239, SEQ ID NO: 400-SEQ ID NO: 456, or SEQ ID NO: 241, or at least 80%, at least 90%, at least 92%, at least 93%, at least 94%, at least 95%, at least 96%, at least 97%, at least 98%, or at least 99% sequence identity with a fragment of any one of SEQ ID NO: 187, SEQ ID NO: 233-SEQ ID NO: 239, SEQ ID NO: 400-SEQ ID NO: 456, or SEQ ID NO: 241. In some aspects, the cellular receptor-binding peptide comprises a sequence that has at least 80%, at least 90%, at least 92%, at least 93%, at least 94%, at least 95%, at least 96%, at least 97%, at least 98%, or at least 99% sequence identity with SEQ ID NO: 187, SEQ ID NO: 235, SEQ ID NO: 236, SEQ ID NO: 238, SEQ ID NO: 239, SEQ ID NO: 400, or SEQ ID NO: 401 or at least 80%, at least 90%, at least 92%, at least 93%, at least 94%, at least 95%, at least 96%, at least 97%, at least 98%, or at least 99% sequence identity with a fragment of SEQ ID NO: 187, SEQ ID NO: 235, SEQ ID NO: 236, SEQ ID NO: 238, SEQ ID NO: 239, SEQ ID NO: 400, or SEQ ID NO: 401. In some aspects, the cellular receptor-binding peptide comprises a sequence of SEQ ID NO: 187, SEQ ID NO: 235, SEQ ID NO: 236, SEQ ID NO: 238, SEQ ID NO: 239, SEQ ID NO: 400, or SEQ ID NO: 401. In some aspects, the fragment comprises at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, or at least 50 amino acid residues.

In some aspects, the cellular receptor-binding peptide comprises one or more histidine residues at a cellular receptor-binding interface. In some aspects, the target-binding peptide comprises one or more histidine residues at a target-binding interface. In some aspects, the target-binding peptide is a PD-L1-binding peptide, an EGFR-binding peptide, or a TNFα-binding peptide. In some aspects, the PD-L1-binding peptide comprises a sequence that has at least 80%, at least 90%, at least 92%, at least 93%, at least 94%, at least 95%, at least 96%, at least 97%, at least 98%, or at least 99% sequence identity with any one of SEQ ID NO: 233, SEQ ID NO: 234, SEQ ID NO: 187, SEQ ID NO: 235-SEQ ID NO: 239, SEQ ID NO: 400-SEQ ID NO: 456, or SEQ ID NO: 240. In some aspects, the EGFR-binding peptide binds EGFR variant III or tyrosine kinase inhibitor-resistant EGFR. In some aspects, the EGFR-binding peptide comprises a sequence that has at least 80%, at least 90%, at least 92%, at least 93%, at least 94%, at least 95%, at least 96%, at least 97%, at least 98%, or at least 99% sequence identity with SEQ ID NO: 243, SEQ ID NO: 244, SEQ ID NO: 219, or SEQ ID NO: 242. In some aspects, the EGFR-binding peptide comprises a sequence of SEQ ID NO: 242. In some aspects, the EGFR-binding peptide comprises a sequence of SEQ ID NO: 243.

In some aspects, the target is a cell surface molecule, a growth factor receptor, secreted peptide, a secreted protein, a circulated molecule, a cell signaling molecule, an extracellular matrix macromolecule, a neurotransmitter, a cytokine, a growth factor, a tumor associated antigen, a tumor specific antigen or a hormone, a checkpoint inhibitor, an immune checkpoint inhibitor, an inhibitory immune receptor, a ligand of an inhibitory immune receptor, a macrophage surface protein, a lipopolysaccharide, an antibody, an inhibitory immune receptor, a tumor associated antigen, a tumor specific antigen, or an autoantibody. In some aspects, the target is collagen, elastin, a microfibrillar protein, a proteoglycan, CD200R, CD300a, CD300f, CEACAM1, FcgRiib, ILT-2, ILT-3, ILT-4, ILT-5, LAIR-1, PECAM-1, PILR-alpha, SIRL-1, and SIRP-alpha, CLEC4A, Ly49Q, MIC, CD3, CD47, CD28, CD137, CD89, CD14, CD16, CD29, CD44, CD71, CD73, CD90, CD105, CD166, CD27, CD39, CD24, CD25, CD74, CD40L, MUC1, MUC16, MUC2, MUC5AC, MUC4, OX40, 4-1BB, HLA-G, LAG3, Tim3, TIGIT, GITR, TCR, TNF-α, EGFR, EGFRvIII, TKI-resistant EGFR, HER2, ERBB3, PDGFR, FGF, VEGF, VEGFR, IGFR1, CTLA4, STRO1, complement factor C4, complement factor C1q, complement factor C1s, complement factor C1r, complement factor C3, complement factor C3a, complement factor C3b, complement factor C5, complement factor C5a, TGFβ, PCSK9, P2Y6, HER3, RANK, tau, amyloid ß, huntingtin, α-synuclein, glucocerebrosidase, α-glucosidase, IL-1, IL-1R, IL-1α, IL-1β, IL-2, IL-2R, IL-4, IL-5, IL-6, IL-6R, IL-10, IL-10R, IL-17, IL-23, IL-12, p40, a member of the B7 family, c-Met, SIGLEC, MCP-1, an MHC, an MHC I, an MHC II, PD-1, or PD-L1. In some aspects, the target is PD-L1, EGFR, or TNFα.

In some aspects, the peptide complex comprises a sequence that has at least 80%, at least 90%, at least 92%, at least 93%, at least 94%, at least 95%, at least 96%, at least 97%, at least 98%, or at least 99% sequence identity with any one of SEQ ID NO: 288-SEQ ID NO: 313 or SEQ ID NO: 315-SEQ ID NO: 346; or at least 80%, at least 90%, at least 92%, at least 93%, at least 94%, at least 95%, at least 96%, at least 97%, at least 98%, or at least 99% sequence identity with any one of SEQ ID NO: 347, SEQ ID NO: 348, SEQ ID NO: 351, SEQ ID NO: 352, SEQ ID NO: 355, SEQ ID NO: 356, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 371, SEQ ID NO: 373, SEQ ID NO: 376, SEQ ID NO: 378, SEQ ID NO: 382, SEQ ID NO: 384, SEQ ID NO: 387, or SEQ ID NO: 389. In some aspects, the peptide complex comprises a sequence of: SEQ ID NO: 288, SEQ ID NO: 289, SEQ ID NO: 307, SEQ ID NO: 313, SEQ ID NO: 327, SEQ ID NO: 328, SEQ ID NO: 332, SEQ ID NO: 333, SEQ ID NO: 337, SEQ ID NO: 338, SEQ ID NO: 342, or SEQ ID NO: 343; SEQ ID NO: 292, SEQ ID NO: 293, SEQ ID NO: 310, SEQ ID NO: 315, or SEQ ID NO: 316 heterodimerized with SEQ ID NO: 302, SEQ ID NO: 305, SEQ ID NO: 339, SEQ ID NO: 340, SEQ ID NO: 344, or SEQ ID NO: 345; SEQ ID NO: 296 heterodimerized with SEQ ID NO: 302, SEQ ID NO: 339, or SEQ ID NO: 344; SEQ ID NO: 298; SEQ ID NO: 299 heterodimerized with SEQ ID NO: 301; SEQ ID NO: 331 or SEQ ID NO: 336 heterodimerized with SEQ ID NO: 330 or SEQ ID NO: 335; or SEQ ID NO: 292, SEQ ID NO: 315, or SEQ ID NO: 316 heterodimerized with SEQ ID NO: 329, SEQ ID NO: 330, SEQ ID NO: 334, or SEQ ID NO: 335. In some aspects, the peptide complex comprises a sequence of: SEQ ID NO: 290, SEQ ID NO: 291, SEQ ID NO: 308, SEQ ID NO: 317, SEQ ID NO: 318, SEQ ID NO: 322, or SEQ ID NO: 323; SEQ ID NO: 292, SEQ ID NO: 294, SEQ ID NO: 315, SEQ ID NO: 316, heterodimerized with SEQ ID NO: 304, SEQ ID NO: 306, SEQ ID NO: 319, SEQ ID NO: 320, SEQ ID NO: 321, SEQ ID NO: 324, or SEQ ID NO: 325; SEQ ID NO: 295 or SEQ ID NO: 297, heterodimerized with SEQ ID NO: 304, SEQ ID NO: 319, SEQ ID NO: 321, or SEQ ID NO: 324; SEQ ID NO: 298 or SEQ ID NO: 300, heterodimerized with SEQ ID NO: 303; or SEQ ID NO: 326, heterodimerized with SEQ ID NO: 306, SEQ ID NO: 311, SEQ ID NO: 320 or SEQ ID NO: 325.

In some aspects, an off rate of the cellular receptor-binding peptide from the cellular receptor is slower than a recycling rate of the cellular receptor. In some aspects, an off rate of the cellular receptor-binding peptide from the cellular receptor is no faster than 1 minute, no faster than 2 minutes, no faster than 3 minutes, no faster than 4 minutes, no faster than 5 minutes, no faster than 7 minutes, no faster than 10 minutes, no faster than 15 minutes, or no faster than 20 minutes. In some aspects, the peptide complex is capable of being endocytosed via receptor-mediated endocytosis. In some aspects, the receptor-mediated endocytosis is transferrin receptor-mediated endocytosis. In some aspects, the cellular receptor-binding peptide remains bound to the cellular receptor inside an endocytic vesicle. In some aspects, the peptide complex is recycled when the cellular receptor-binding peptide is bound to the cellular receptor and the cellular receptor is recycled. In some aspects, the target is released or dissociated from the target-binding peptide when the peptide complex is endocytosed via receptor-mediated endocytosis.

In some aspects, the target is an extracellular protein, a circulating protein, or a soluble protein. In some aspects, the target is a cell surface protein. In some aspects, the target is a transmembrane protein. In some aspects, the peptide complex further comprises a second target-binding peptide. In some aspects, the second target-binding peptide binds a second target. In some aspects, the target and the second target form a dimer when bound to the target-binding peptide and the second target binding peptide. In some aspects, dimerization of the target and the second target increases a rate of endocytosis of the target and the second target. In some aspects, the second target is the same as the target.

In some aspects, the peptide complex further comprises a half-life modifying agent coupled to the cellular receptor-binding peptide, the target-binding peptide, or both. In some aspects, the half-life modifying agent is a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some aspects, the molecule that binds to albumin is a serum albumin-binding peptide. In some aspects, the serum albumin-binding peptide comprises a sequence of any one of SEQ ID NO: 178, SEQ ID NO: 179, or SEQ ID NO: 193. In some aspects, the cellular receptor-binding peptide, the target-binding peptide, or both is recombinantly expressed.

In some aspects, the target-binding peptide is configured to dissociate from the target at pH 6.5, pH 6.0, pH 5.5, pH 5.0, or pH 4.5. In some aspects, the cellular receptor-binding peptide is configured to dissociate from the cellular receptor at pH 6.5, pH 6.0, pH 5.5, pH 5.0, or pH 4.5.

In various aspects, the present disclosure provides a method of selectively depleting a target molecule, the method comprising: contacting a peptide complex comprising a cellular receptor-binding peptide a target-binding peptide complexed with the cellular receptor-binding peptide to a cell expressing a cellular receptor; binding the target-binding peptide to the target molecule under extracellular conditions; binding the cellular receptor-binding peptide to the cellular receptor under extracellular conditions; endocytosing the peptide complex, the target molecule, and the cellular receptor; unbinding the target-binding peptide from the target molecule, the cellular-receptor-binding peptide from the cellular receptor, or both under endosomal conditions; and degrading the target molecule, thereby depleting the target molecule.

In various aspects, the present disclosure provides a method of selectively depleting a target molecule, the method comprising: contacting a peptide complex as described herein to a cell expressing a cellular receptor; binding the target-binding peptide to the target molecule under extracellular conditions; binding the cellular receptor-binding peptide to the cellular receptor under extracellular conditions; endocytosing the peptide complex, the target molecule, and the cellular receptor into an endocytic or lysosomal compartment; releasing the target-binding peptide from the target molecule, the cellular-receptor-binding peptide from the cellular receptor, or both under endosomal conditions; and degrading the target molecule, thereby depleting the target molecule.

In some aspects, the method further comprises recycling the peptide complex and the cellular receptor. In some aspects, the cellular receptor is a transferrin receptor or PD-L1 and the cellular receptor-binding peptide is a transferrin receptor-binding peptide or a PD-L1-binding peptide. In some aspects, the cellular receptor-binding peptide is a transferrin receptor-binding peptide and the cellular receptor is a transferrin receptor. In some aspects, the cellular receptor-binding peptide is a PD-L1-binding peptide and the cellular receptor is PD-L1. In some aspects, the endocytosing comprises receptor-mediated endocytosis. In some aspects, the cellular receptor-binding peptide remains bound to the cellular receptor in the endocytic or lysosomal compartment. In some aspects, the target molecule is degraded in the endocytic or lysosomal compartment. In some aspects, the receptor-mediated endocytosis is transferrin receptor-mediated endocytosis.

In some aspects, the target molecule is an extracellular protein, a circulating protein, or a soluble protein. In some aspects, the target molecule is a cell surface protein. In some aspects, the target molecule is a transmembrane protein. In some aspects, the method comprises penetrating a cellular layer comprising a blood brain barrier (BBB) with the peptide complex. In some aspects, the target molecule is degraded in the central nervous system. In some aspects, the cell expresses the cellular receptor.

In some aspects, the method comprises binding the cellular receptor-binding peptide to the cellular receptor with a dissociation constant (KD) of no more than 50 μM, no more than 5 μM, no more than 500 nM, no more than 100 nM, no more than 40 nM, no more than 30 nM, no more than 20 nM, no more than 10 nM, no more than 5 nM, no more than 2 nM, no more than 1 nM, no more than 0.5 nM, no more than 0.4 nM, no more than 0.3 nM, no more than 0.2 nM, or no more than 0.1 nM under the extracellular conditions. In some aspects, the method comprises binding the cellular receptor-binding peptide to the cellular receptor with a dissociation constant (KD) of no more than 50 μM, no more than 5 μM, no more than 500 nM, no more than 100 nM, no more than 40 nM, no more than 30 nM, no more than 20 nM, no more than 10 nM, no more than 5 nM, no more than 2 nM, no more than 1 nM, no more than 0.5 nM, no more than 0.4 nM, no more than 0.3 nM, no more than 0.2 nM, or no more than 0.1 nM under the endosomal conditions.

In some aspects, the target-binding peptide remains bound to the target molecule in the endocytic compartment. In some aspects, the method comprises binding the target-binding peptide to the target molecule with a dissociation constant (KD) of no more than 50 μM, no more than 5 μM, no more than 500 nM, no more than 100 nM, no more than 40 nM, no more than 30 nM, no more than 20 nM, no more than 10 nM, no more than 5 nM, no more than 2 nM, no more than 1 nM, no more than 0.5 nM, no more than 0.4 nM, no more than 0.3 nM, no more than 0.2 nM, or no more than 0.1 nM under the extracellular conditions. In some aspects, the method comprises binding the target-binding peptide to the target molecule with a dissociation constant (KD) of no less than 1 nM, no less than 2 nM, no less than 5 nM, no less than 10 nM, no less than 20 nM, no less than 50 nM, no less than 100 nM, no less than 200 nM, or no less than 500 nM under the endosomal conditions. In some aspects, the method comprises binding the cellular receptor-binding peptide to the cellular receptor with an affinity that differs by no more than 2-fold, no more than 5-fold, no more than 10-fold, no more than 15-fold, no more than 20-fold, no more than 25-fold, no more than 30-fold, no more than 40-fold, or no more than 50-fold under the extracellular conditions as compared to the endosomal conditions. In some aspects, the method comprises forming one or more polar or charge-charge interactions between the target-binding peptide and the target molecule.

In some aspects, the cellular receptor binding peptide comprises a sequence that has at least 80%, at least 90%, at least 92%, at least 93%, at least 94%, at least 95%, at least 96%, at least 97%, at least 98%, or at least 99% sequence identity with any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. In some aspects, the cellular receptor binding peptide comprises a sequence that has at least 80%, at least 90%, at least 92%, at least 93%, at least 94%, at least 95%, at least 96%, at least 97%, at least 98%, or at least 99% sequence identity with SEQ ID NO: 96. In some aspects, the cellular receptor-binding peptide comprises a sequence of SEQ ID NO: 96. In some aspects, the cellular receptor-binding peptide comprises a sequence of any one of SEQ ID NO: 392-SEQ ID NO: 399. In some aspects, the cellular receptor-binding peptide comprises a sequence that has at least 80%, at least 90%, at least 92%, at least 93%, at least 94%, at least 95%, at least 96%, at least 97%, at least 98%, or at least 99% sequence identity with any one of SEQ ID NO: 187, SEQ ID NO: 233-SEQ ID NO: 239, SEQ ID NO: 400-SEQ ID NO: 456, or SEQ ID NO: 241, or at least 80%, at least 90%, at least 92%, at least 93%, at least 94%, at least 95%, at least 96%, at least 97%, at least 98%, or at least 99% sequence identity with a fragment of any one of SEQ ID NO: 187, SEQ ID NO: 233-SEQ ID NO: 239, SEQ ID NO: 400-SEQ ID NO: 456, or SEQ ID NO: 241. In some aspects, the cellular receptor-binding peptide comprises a sequence that has at least 80%, at least 90%, at least 92%, at least 93%, at least 94%, at least 95%, at least 96%, at least 97%, at least 98%, or at least 99% sequence identity with SEQ ID NO: 187, SEQ ID NO: 235, SEQ ID NO: 238, or SEQ ID NO: 239 or at least 80%, at least 90%, at least 92%, at least 93%, at least 94%, at least 95%, at least 96%, at least 97%, at least 98%, or at least 99% sequence identity with a fragment of SEQ ID NO: 187, SEQ ID NO: 235, SEQ ID NO: 238, or SEQ ID NO: 239. In some aspects, the cellular receptor-binding peptide comprises a sequence of SEQ ID NO: 187, SEQ ID NO: 235, SEQ ID NO: 238, or SEQ ID NO: 239.

In some aspects, the method further comprises binding a second target molecule with a second target-binding peptide. In some aspects, the target molecule and the second target molecule dimerize when bound to the target-binding peptide and the second target-binding peptide. In some aspects, the method comprises increasing a rate of endocytosis of the target molecule and the second target molecule upon dimerization of the target molecule and the second target molecule. In some aspects, the second target molecule is degraded upon endocytosis of the target molecule and the second target molecule. In some aspects, the second target molecule is the same as the target molecule.

In various aspects, the present disclosure provides a method of treating a disease or condition in a subject, the method comprising: administering to the subject a peptide complex comprising a cellular receptor-binding peptide a target-binding peptide complexed with the cellular receptor-binding peptide; binding the target-binding peptide under extracellular conditions to a target molecule associated with the disease or condition on a cell of the subject expressing the target molecule and a cellular receptor; binding the cellular receptor-binding peptide under extracellular conditions to the cellular receptor on the cell of the subject; endocytosing the peptide complex, the target molecule, and the cellular receptor; unbinding the target-binding peptide from the target molecule, the cellular-receptor-binding peptide from the cellular receptor, or both under endosomal conditions; and degrading the target molecule, thereby treating the disease or condition.

In various aspects, the present disclosure provides a method of treating a disease or condition in a subject, the method comprising: administering to the subject a peptide complex as described herein; binding the target-binding peptide under extracellular conditions to a target molecule associated with the disease or condition on a cell of the subject expressing the target molecule and a cellular receptor; binding the cellular receptor-binding peptide under extracellular conditions to the cellular receptor on the cell of the subject; endocytosing the peptide complex, the target molecule, and the cellular receptor; unbinding the target-binding peptide from the target molecule, the cellular-receptor-binding peptide from the cellular receptor, or both under endosomal conditions; and degrading the target molecule, thereby treating the disease or condition.

In some aspects, the target molecule is a cell surface molecule, a growth factor receptor, secreted peptide, a secreted protein, a circulated molecule, a cell signaling molecule, an extracellular matrix macromolecule, a neurotransmitter, a cytokine, a growth factor, a tumor associated antigen, a tumor specific antigen or a hormone, a checkpoint inhibitor, an immune checkpoint inhibitor, an inhibitory immune receptor, a ligand of an inhibitory immune receptor, a macrophage surface protein, a lipopolysaccharide, an antibody, an inhibitory immune receptor, a tumor associated antigen, a tumor specific antigen, or an autoantibody. In some aspects, the target molecule is collagen, elastin, a microfibrillar protein, a proteoglycan, CD200R, CD300a, CD300f, CEACAM1, FcgRiib, ILT-2, ILT-3, ILT-4, ILT-5, LAIR-1, PECAM-1, PILR-alpha, SIRL-1, and SIRP-alpha, CLEC4A, Ly49Q, MIC, CD3, CD47, CD28, CD137, CD89, CD14, CD16, CD29, CD44, CD71, CD73, CD90, CD105, CD166, CD27, CD39, CD24, CD25, CD74, CD40L, MUC1, MUC16, MUC2, MUC5AC, MUC4, OX40, 4-1BB, HLA-G, LAG3, Tim3, TIGIT, GITR, TCR, TNF-α, EGFR, EGFRvIII, TKI-resistant EGFR, HER2, ERBB3, PDGFR, FGF, VEGF, VEGFR, IGFR1, CTLA4, STRO1, complement factor C4, complement factor C1q, complement factor C1s, complement factor C1r, complement factor C3, complement factor C3a, complement factor C3b, complement factor C5, complement factor C5a, TGFβ, PCSK9, P2Y6, HER3, RANK, tau, amyloid ß, huntingtin, α-synuclein, glucocerebrosidase, α-glucosidase, IL-1, IL-1R, IL-1α, IL-1β, IL-2, IL-2R, IL-4, IL-5, IL-6, IL-6R, IL-10, IL-10R, IL-17, IL-23, IL-12, p40, a member of the B7 family, c-Met, SIGLEC, MCP-1, an MHC, an MHC I, an MHC II, PD-1, or PD-L1. In some aspects, the target molecule is a receptor tyrosine kinase. In some aspects, the receptor tyrosine kinase is EGF receptor, ErbB, Insulin receptor, PDGF receptor, VEGF receptor, FGF receptor, CCK receptor, NGF receptor, HGF receptor, Eph receptor, AXL receptor, TIE receptor, RYK receptor, DDR receptor, RET receptor, ROS receptor, LTK receptor, ROR receptor, MuSK receptor, or LMR receptor. In some aspects, the target molecule is a pathogen or a pathogen surface molecule.

In some aspects, the disease or condition is a cancer, a neurodegenerative disease, a lysosomal storage disease, an inflammatory disease, an autoimmune disease, a neuroinflammatory disease, an immune disease, or pain. In some aspects, the cancer is breast cancer, liver cancer, colon cancer, brain cancer, leukemia, lymphoma, non-Hodgkin lymphoma, myeloma, blood-cell-derived cancer, lung cancer, sarcoma, stomach cancer, a gastrointestinal cancer, glioblastoma, head and neck cancer, non-small-cell lung cancer, squamous non-small cell lung cancer, pancreatic cancer, ovarian cancer, blood cancer, skin cancer, liver cancer, kidney cancer, or colorectal cancer. In some aspects, the cancer is TKI-resistant, cetuximab-resistant, necitumumab-resistant, or panitumumab-resistant. In some aspects, the cancer is an advanced cancer, a metastatic cancer, a metastatic cancer in the central nervous system, metastatic breast cancer, metastatic skin cancer, a refractory cancer, a KRAS wild type cancer, a KRAS mutant cancer, or an exon20 mutant non-small-cell lung cancer. In some aspects, the target molecule is HER2, EGFR, FGFR-1, PD-L1, VEGF, PD-1, CD38, GD2, SLAMF7, CTLA-4, CCR4, CD20, PDGFRα, VEGFR2, CD33, CD30, CD22, CD79B, Nectin-4, or TROP2. In some aspects, the target molecule is EGFR or PD-L1. In some aspects, the method further comprises administering an additional therapy to the subject. In some aspects, the additional therapy comprises radiation, chemotherapy, platinum therapy, or anti-metabolic therapy. In some aspects, the additional therapy comprises fluorouracil, FOLFIRI, irinotecan, FOLFOX, gemcitabine, or cisplatin.

In some aspects, the neurodegenerative disease is Alzheimer's disease, amyotrophic lateral sclerosis, Friedreich's ataxia, Huntington's disease, Parkinson's disease, or spinal muscular atrophy. In some aspects, the target molecule is tau, amyloid ß, huntingtin, or α-synuclein. In some aspects, the lysosomal storage disease is Gaucher's Disease or Pompe Disease. In some aspects, the target molecule is glucocerebrosidase or α-glucosidase. In some aspects, the inflammatory disease is rheumatoid arthritis, psoriasis, multiple sclerosis, glomerulonephritis, lupus, inflammatory bowel disease, ulcerative colitis, Crohn's disease, cutaneous vasculitis, neuroinflammatory disease, inflammation-associated neurodegeneration, Alzheimer's disease, stroke, traumatic brain injury, Sjogren's disease, or cystic fibrosis. In some aspects, the target molecule is apolipoprotein E4, TNF-α, IL-1, IL-6, IL-7, IL-12, or IL-23. In some aspects, the target molecule is TNF-α. In some aspects, the cell is a cancer cell, an immune cell, a central nervous system cell, a neuronal cell, a T cell, a B cell, a macrophage, a monocyte, a neutrophil, a dendritic cell, a mast cell, a basophil, or an eosinophil.

In some aspects, the method further comprises forming a ternary complex between the selective depletion complex, the target molecule, and the cellular receptor. In some aspects, formation of the ternary complex increases recycling or turnover of the cellular receptor, the target molecule, or both. In some aspects, formation of the ternary complex increases binding of the target molecule to the cellular receptor.

### INCORPORATION BY REFERENCE

All publications, patents, and patent applications cited in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1A-FIG. 1G illustrate a Coomassie stained gel of human soluble transferrin receptor (hTfR) ectodomain protein and flow cytometry plots showing successive enrichment of cells that bind to hTfR ectodomain from a pooled, highly diverse peptide library.

FIG. 1A illustrates a Coomassie stained gel of transferrin receptor (TfR) protein showing successful purification of TfR.

FIG. 1B illustrates a flow cytometry plot of cells displaying candidate TfR-binding peptides after one flow sort. Cells were sorted based on ability to bind to TfR labeled with a fluorescent streptavidin. Data points in the upper right region represent cells expressing a candidate peptide, quantified by GFP fluorescence, that bind TfR, quantified by fluorescence of the fluorescent TfR-streptavidin.

FIG. 1C illustrates a negative control flow cytometry plot of cells displaying candidate TfR-binding peptides after one flow sort. Cells were stained based on ability to bind to a control protein labeled with a fluorescent streptavidin. Data points in the upper right region represent cells expressing a candidate peptide, quantified by GFP fluorescence, that bind to the negative control protein, quantified by fluorescence of the fluorescent control protein-streptavidin.

FIG. 1D illustrates a flow cytometry plot of cells displaying candidate TfR-binding peptides after a second flow sort, following the first cell sort illustrated in FIG. 1B. Cells were sorted based on ability to bind to TfR labeled with a fluorescent streptavidin. Data points in the upper right region represent cells expressing a candidate peptide, quantified by GFP fluorescence, that bind TfR, quantified by fluorescence of the fluorescent TfR-streptavidin.

FIG. 1E illustrates a negative control flow cytometry plot of cells displaying candidate TfR-binding peptides after a second flow sort, following the first cell sort illustrated in FIG. 1B and FIG. 1C. Cells were stained based on ability to bind to a control protein labeled with a fluorescent streptavidin. Data points in the upper right region represent cells expressing a candidate peptide, quantified by GFP fluorescence, that bind to the negative control protein, quantified by fluorescence of the fluorescent control protein-streptavidin.

FIG. 1F illustrates a flow cytometry plot of cells displaying candidate TfR-binding peptides after a third flow sort, following the second cell sort illustrated in FIG. 1D. Cells were sorted based on ability to bind to TfR labeled with a fluorescent streptavidin. Data points in the upper right region represent cells expressing a candidate peptide, quantified by GFP fluorescence, that bind TfR, quantified by fluorescence of the fluorescent TfR-streptavidin. The box indicates cells expressing peptides that bind to TfR.

FIG. 1G illustrates a negative control flow cytometry plot of cells displaying candidate TfR-binding peptides after a third flow sort, following the second cell sort illustrated in FIG. 1D and FIG. 1E. Cells were stained based on ability to bind to a control protein labeled with a fluorescent streptavidin. Data points in the upper right region represent cells expressing a candidate peptide, quantified by GFP fluorescence, that bind to the negative control protein, quantified by fluorescence of the fluorescent control protein-streptavidin. The box indicates cells expressing peptides that bind to the negative control protein.

FIG. 2A-FIG. 2D illustrate flow cytometry of cells displaying a single clonal TfR-binding peptide and screened for binding to either TfR or a negative control protein to confirm binding of the TfR-binding peptide identified in FIG. 1A-FIG. 1G to TfR. Flow cytometry was performed using TfR or the control protein labeled with either streptavidin or an anti-His antibody to verify that binding was not dependent on the streptavidin label.

FIG. 2A illustrates a negative control flow cytometry plot of cells expressing a TfR-binding peptide of SEQ ID NO: 1 (x-axis, GFP) screened for binding to a negative control protein labeled (y-axis, stained with a fluorescent anti-His antibody).

FIG. 2B illustrates a flow cytometry plot of cells expressing a TfR-binding peptide of SEQ ID NO: 1 (x-axis, GFP) screened for binding to TfR (y-axis, stained with a fluorescent anti-His antibody). The box indicates cells that express the TfR-binding peptide and bind to TfR.

FIG. 2C illustrates a negative control flow cytometry plot of cells expressing a TfR-binding peptide of SEQ ID NO: 1 (x-axis, GFP) screened for binding to a negative control protein labeled (y-axis, stained with a fluorescent streptavidin).

FIG. 2D illustrates a flow cytometry plot of cells expressing a TfR-binding peptide of SEQ ID NO: 1 (x-axis, GFP) screened for binding to TfR (y-axis, stained with a fluorescent streptavidin). The box indicates cells that express the TfR-binding peptide and bind to TfR.

FIG. 3A and FIG. 3B illustrate TfR-binding for peptide variants arising from permuting enriched variants from site-saturation mutagenesis (SSM). Each graph represents a round of completed SSM and each shaded bar within the applicable graph indicates the number of mutations in the specific variant peptide denoted under the bar as compared to the respective reference peptide sequence with which the round of SSM was started (SEQ ID NO: 1 in FIG. 3A, or SEQ ID NO: 2 in FIG. 3B). The data show the relative binding affinity of the identified peptides to TfR, representing the last step of SSM employed showing the next generation molecules.

FIG. 3A illustrates the level of hTfR binding for variants comprising sequences of SEQ ID NO: 3-SEQ ID NO: 23, derived from a site-saturation mutagenesis (SSM) for affinity maturation of the peptide having a sequence of SEQ ID NO: 1.

FIG. 3B illustrates the level of hTfR binding for peptide variants having sequences of SEQ ID NO: 24-SEQ ID NO: 28 and SEQ ID NO: 30-SEQ ID NO: 32, derived from a site-saturation mutagenesis (SSM) for affinity maturation of the starting peptide having a sequence of SEQ ID NO: 2.

FIG. 4 illustrates surface plasmon resonance (SPR) curves showing binding of TfR-binding peptide variants with different affinities to TfR. Dissociation kinetics were quantified for each peptide variant. The surface plasmon resonance (SPR) trace over time is shown using 300 nM of each of SEQ ID NO: 1, SEQ ID NO: 2, SEQ ID NO: 4, and SEQ ID NO: 32 to hTfR. SEQ ID NO: 32 show the strongest binding to TfR, as evaluated by SPR. Data was normalized to the maximum response of each trace.

FIG. 5 illustrates a surface plasmon resonance (SPR) trace showing hTfR-binding for varying concentrations of the peptide having a sequence of SEQ ID NO: 2. Based on this data, the dissociation constant (K_{D}) of the peptide of SEQ ID NO: 2 was determined to be 8.7 nM.

FIG. 6 illustrates a surface plasmon resonance (SPR) trace showing hTfR-binding for varying concentrations of the peptide having a sequence of SEQ ID NO: 4. Based on this data, the dissociation constant (K_{D}) of the peptide of SEQ ID NO: 4 was determined to be 14.8 nM.

FIG. 7 illustrates binding and single cycle kinetics data of SEQ ID NO: 32 binding to captured biotinylated hTfR by surface plasmon resonance (SPR). 5 concentrations of a peptide having a sequence of SEQ ID NO: 32 (0.037 nM, 0.11 nM, 0.33 nM, 1 nM, 3 nM) were injected over 2 densities of captured biotinylated (Bt)-hTfR and analyzed globally. Analysis parameters were held constant for high and low density runs, and data from both channels was included in the same analysis. Based on this data, the dissociation constant (K_{D}) of the peptide of SEQ ID NO: 32 was determined to be 216 μM, the association rate (kₐ) was determined to be 8.55×10⁶ M⁻¹s⁻¹, and the dissociation rate (k_{d}) was determined to be 1.85×10⁻³ s⁻¹.

FIG. 8 illustrates binding and single cycle kinetics data of SEQ ID NO: 30 binding to captured biotinylated hTfR by SPR. 5 concentrations of a peptide having a sequence of SEQ ID NO: 30 (0.037 nM, 0.11 nM, 0.33 nM, 1 nM, 3 nM) were injected over 2 densities of captured Bt-hTfR and analyzed globally. Analysis parameters were held constant for high and low density runs, and data from both channels was included in the same analysis. Based on this data, the dissociation constant (K_{D}) of the peptide of SEQ ID NO: 30 was determined to be 486 μM, the association rate (kₐ) was determined to be 8.57×10⁶ M⁻¹s⁻¹, and the dissociation rate (k_{d}) was determined to be 4.16×10⁻³ s⁻¹.

FIG. 9A-FIG. 9C illustrate the purification and testing of a soluble transferrin receptor (TfR) ectodomain to assess whether it will bind to transferrin.

FIG. 9A illustrates a surface plasmon resonance (SPR) trace of holo or apo transferrin (Tf) binding to the purified TfR ectodomain. The data shows that holo Tf binds the TfR ectodomain, but apo Tf does not, as shown by the increase in response (RU) over time for the holo Tf, but not the apo Tf. This data validates that the soluble TfR used in the screen for TfR-Binding CDP peptides comprises the endogenous protein structure of TfR on the surface of the cell providing data that the binders have utility for receptor mediated endocytosis.

FIG. 9B illustrates a schematic of a vector display scaffold and target engagement used to screen for and optimize peptide binding properties. The surface display vector (SDGF) encoding a GFP-tagged construct of the binder (e.g., SEQ ID NO: 1, SEQ ID NO: 2, or SEQ ID NO: 32) is expressed on the cell surface. A target protein (e.g., TfR) labeled with a fluorescent dye (“Co-Stain”) bind to the surface-expressed binder. Fluorescence intensity of the co-stain is used as a measure of peptide affinity for the target since cells expressing a peptide with a high affinity for the target protein will recruit more co-stained target than cells expressing a peptide with lower affinity for the target protein.

FIG. 9C illustrates flow cytometry to verify specificity of TfR binding for Machupo virus glycoprotein, a known TfR binding target, as measured by the amount of Alexa Fluor 647-TfR (co-stain in FIG. 9B) bound. Cells transfected with Machupo virus glycoprotein (SDGF-MaCV) are tested with a combination of biotinylated TfR and Alexa Fluor 647-labeled streptavidin (Strep-647), SDGF-MaCV cells and Alexa Fluor 647-labeled elastase, or SDGF-elafin cells and TfR+Strep-647. The elastase and elafin cells conjugates fail to bind cells. These results showed that the soluble TfR used in the peptide screens comprises the endogenous protein structure and demonstrated both the specificity of TfR binding to its endogenous ligand, and the utility of SDGF as a means to identify novel TfR binding partners.

FIG. 10A-FIG. 10C show data using flow cytometry to identify the binding of a TfR-binding cystine-dense peptide (CDP, SEQ ID NO: 32) fused with GFP to TfR labeled with streptavidin-AlexaFluor647 (strep-647) under pH conditions representing the physiologic extracellular environment (pH 7.4) or the endosomal environment (pH 5.5).

FIG. 10A illustrates flow cytometry results in a binding assay to measure binding of a TfR-binding cystine-dense peptide (CDP) (SEQ ID NO: 32) to TfR at pH 7.4, representing the physiologic extracellular environment. Cells expressing SEQ ID NO: 32 were stained with 10 nM of TfR and 10 nM Strep-647 at pH 7.4. The box indicates the “slice” gate used in the quantitation shown in FIG. 10C.

FIG. 10B illustrates flow cytometry results in a binding assay to measure binding of a TfR-binding CDP (SEQ ID NO: 32) to TfR at pH 5.5. Cells expressing SEQ ID NO: 32 were stained with 10 nM of TfR and 10 nM Strep-647 at pH 5.5, representing the endosomal environment. The box indicates the “slice” gate used in the quantitation shown in FIG. 10C.

FIG. 10C illustrates a comparison of the labeling efficiency of the TfR-binding peptide at pH 7.4 measured in FIG. 10A and the labeling efficiency at pH 5.5 measured in FIG. 10B. The results show that the binding of the TfR-binding cystine-dense peptide (CDP, SEQ ID NO: 32) is robust and comparable both at physiologic extracellular and endosomal conditions.

FIG. 11A schematically illustrates a workflow for developing compositions for selective depletion of a target molecule. Target-binding peptides are identified by staining an expression library containing target-binding peptide candidates with labeled target molecule. Target-binding peptides from the library are distinguished by accumulation of signal from bound target molecules. Optionally, identified target-binding peptides are selected and further matured for binding, for example using point mutation screens. The identified target-binding peptides are modified for pH-dependent binding, for example by performing histidine point mutation scans as illustrated in FIG. 11D. The resulting pH-dependent target-binding peptides are linked (e.g., as fusion peptides) to a recycler (e.g., a TfR-binding peptide), to form a selective depletion complex.

FIG. 11B schematically illustrates in vitro validation of the ability of the selective depletion complex to deplete the target, such as from the cell surface or the media.

FIG. 11C schematically illustrates phenotypic screening of selective depletion complexes. The selective depletion complexes can be validated by testing target depletion in cells expressing the selective depletion complexes. Complexes can be further tested in healthy cells and in transformed cell lines to measure disease-specific functionalities of the selective depletion complexes. Specificity of the complexes can be measured by testing for changes in a target-specific cellular function, such as cancer-specific growth inhibition upon depletion of an apoptosis inhibitor.

FIG. 11D illustrates an example of a histidine substitution scan to introduce pH-dependent binding affinity into a target-binding peptide. A histidine substitution scan of a PD-L1-binding CDP (SEQ ID NO: 187) is shown. The peptide sequence is provided above and to the side, and each black box represents a first and second site in which His could be substituted. Those falling along the diagonal from the top-left to the bottom-right represent single His substitutions. A peptide library containing the identified histidine-containing peptides may be generated and screened, for example using the workflow shown in FIG. 11A.

FIG. 12A schematically illustrates a method for selectively depleting a soluble target molecule using a composition comprising a target-binding peptide with pH-dependent binding and a TfR-binding peptide, such as a TfR-binding peptide with pH-independent binding. The composition binds to TfR and to the soluble target molecule and is endocytosed via transferrin receptor-mediated endocytosis. The target molecule is released upon acidification of the endocytic compartment and some or all of the target molecule is degraded in a lysosomal compartment. The TfR and the composition are recycled to the cell surface.

FIG. 12B schematically illustrates a method for selectively depleting a surface target molecule using a composition comprising a target-binding peptide with pH-dependent binding and a TfR-binding peptide, such as a TfR-binding peptide with pH-independent binding. The composition binds to TfR and to the surface target molecule and is endocytosed via transferrin receptor-mediated endocytosis. The target molecule is released upon acidification of the endocytic compartment and some or all of the target molecule is degraded in a lysosomal compartment. The TfR and the composition are recycled to the cell surface.

FIG. 13A and FIG. 13B illustrate the production and purity of peptides fused to a serum albumin-binding peptide (SA21).

FIG. 13A shows production and purity of a TfR-binding peptide fused to a serum albumin-binding peptide (SA21) corresponding to SEQ ID NO: 181. The peptide of SEQ ID NO: 181 was produced as a siderocalin (SCN, SEQ ID NO: 147) fusion, and then cleaved from SCN by TEV. Purity was verified by SDS-PAGE (left) and RP-HPLC (right) under DTT reducing (“R”) or non-reducing (“NR”) conditions. SDS-PAGE was also run on the uncleaved (“U”) siderocalin-CDP fusion peptide. This data indicates that SEQ ID NO: 181 fused to SCN was successfully produced and then cleaved by TEV cleavage, to yield the free CDP fusion of SEQ ID NO: 181.

FIG. 13B shows production and purity of a peptide fused to SA21 corresponding to SEQ ID NO: 182. The peptide of SEQ ID NO: 182 was produced as a SCN fusion, and then cleaved from SCN by TEV. Purity was verified by SDS-PAGE (left) and RP-HPLC (right) under DTT reducing (“R”) or non-reducing (“NR”) conditions. SDS-PAGE was also run on the uncleaved (“U”) siderocalin-CDP fusion peptide. This data indicates that SEQ ID NO: 182 fused to SCN was successfully produced and then cleaved by TEV cleavage, to yield the free CDP fusion of SEQ ID NO: 182.

FIG. 14A schematically illustrates a CDP-CDP dimer containing a target-binding CDP linked to a TfR-binding CDP via a double-knot toxin (DkTx) peptide linker (SEQ ID NO: 139, KKYKPYVPVTTN).

FIG. 14B schematically illustrates a CDP-CDP dimer containing a target-binding CDP linked to a TfR-binding CDP via a poly-GlySer linker (SEQ ID NO: 138, GGGSGGGSGGGS).

FIG. 14C schematically illustrates a CDP-CDP dimer containing a target-binding CDP linked to a TfR-binding CDP via a human IgG linker with a Cys-to-Ser mutation at position 5 (SEQ ID NO: 140, EPKSSDKTHT).

FIG. 15 schematically illustrates a TfR-binding peptide non-covalently linked to a target-binding peptide via an Fc bispecific dimer.

FIG. 16A schematically illustrates a TfR-binding peptide and target-binding peptide fusion containing an albumin binding protein (e.g., SEQ ID NO: 192) in between the target-binding peptide and the TfR-binding peptide and separated by peptide linkers (e.g., any one of SEQ ID NO: 129-SEQ ID NO: 141 or SEQ ID NO: 195-SEQ ID NO: 218).

FIG. 16B schematically illustrates a TfR-binding peptide and target-binding peptide fusion containing an albumin binding protein (e.g., SEQ ID NO: 192) fused to the target-binding peptide by a peptide linker (e.g., any one of SEQ ID NO: 129-SEQ ID NO: 141 or SEQ ID NO: 195-SEQ ID NO: 218).

FIG. 16C schematically illustrates a TfR-binding peptide and target-binding peptide fusion containing an albumin binding protein (e.g., SEQ ID NO: 192) fused to the TfR-binding peptide by a peptide linker (e.g., any one of SEQ ID NO: 129-SEQ ID NO: 141 or SEQ ID NO: 195-SEQ ID NO: 218).

FIG. 17A illustrates SDS-PAGE gels of expressed and TEV-cleaved CDP-CDP dimers containing a TfR-binding peptide (SEQ ID NO: 2) fused to an ion channel inhibitory CDP (Z1E-AnTx, Z1P-AnTx, EWSS-ShK, HsTx, Pro-Vm24, or Vm24) via either a DkTx linker (SEQ ID NO: 139) or a GS3 linker (SEQ ID NO: 141 or SEQ ID NO: 195-SEQ ID NO: 218). The expression product after TEV cleavage contained SCN-CDP dimer, SCN, and CDP dimer. The band for the dimer present on each gel is denoted with a rectangle. This demonstrates that the CDP dimers were successfully expressed and cleaved from SCN. Each gel contained, from left to right, a molecular weight latter (“L”), the peptide sample under non-reducing conditions (“NR”), and the peptide sample under reducing conditions (“R”).

FIG. 17B illustrates SDS-PAGE (left), RP-HPLC (center), and channel inhibition assays (right) for a TfR-binding peptide (SEQ ID NO: 32, top), a Vm24 ion channel inhibitory peptide (middle), and a CDP-CDP dimer containing the TfR-binding peptide fused to the Vm25 ion channel inhibitory peptide (bottom). “Folded” indicates the sample was analyzed under non-reducing conditions and “unfolded” indicates the sample was analyzed under reducing conditions. This data indicates that a target-binding CDP (here, an ion channel inhibiting CDP) can be dimerized with a TfR-binding peptide (such as SEQ ID NO:32), can be expressed, folded, and purified, and that the target-binding CDP can maintain its target binding function while in the dimer with the TfR-binding CDP (function shown here is ion channel inhibition).

FIG. 18A-FIG. 18D shows flow staining data illustrating that TfR-binding peptides are cross-reactive with murine TfR (mTfR) in cell surface binding assays. 293F cells expressing either human or mouse TfR from their surface were stained with soluble TfR-binding peptides that were directly labeled with AlexaFluor 647 dye. This shows that TfR-binding peptides bind both human (hTfR, SEQ ID NO: 190) and murine TfR.

FIG. 18A illustrates the species specificity of the TfR used in these experiments, in this case human TfR. Data is displayed as two topographical density maps and indicates flow cytometry data of transferrin stained with Anti-hTfR (CD71) antibody. The upper density map, oriented diagonally from lower left to upper right, depicts 293ST+SDGF-hTfR. The lower density map, oriented horizontally, depicts 293ST+SDGF-mTfR. The y-axis shows hTfR+Streptavidin from 0 to 10⁷, in increments of 10 on a log scale. The x-axis shows GFP from 0 to 10⁶, in increments of 10 on a log scale.

FIG. 18B illustrates the species specificity of the TfR used in these experiments, in this case murine TfR. Data is displayed as two topographical density maps and indicates flow cytometry data of transferrin stained with Anti-mTfR (CD71) antibody. The upper density map, oriented diagonally from lower left to upper right, depicts 293ST+SDGF-mTfR. The lower density map, having three lobes, depicts 293ST+SDGF-hTfR. The y-axis shows hTfR+Streptavidin from 10⁴ to 10⁷, in increments of 10 on a log scale. The x-axis shows GFP from 0 to 10⁶, in increments of 10 on a log scale.

FIG. 18C illustrates quantification of binding of the peptide having a sequence of SEQ ID NO: 1, the peptide having a sequence of SEQ ID NO: 2, the peptide having a sequence of SEQ ID NO: 30, and the peptide having a sequence of SEQ ID NO: 32 to human TfR. Data is displayed as four topographical density maps and indicates flow cytometry data using 293ST cells+SDGF-hTFR. Three density maps appear nearly superimposed and are oriented above a fourth density map. The lower density map is oriented horizontally and depicts SEQ ID NO: 1 (1^{st} gen). The upper three density maps are oriented diagonally from lower left to upper right. The density map slightly above the other two corresponds to SEQ ID NO: 32 (3^{rd} gen). The density map slightly below the other two corresponds to SEQ ID NO: 2 (2^{nd} gen). The third density map corresponds to SEQ ID NO: 30 (3^{rd} gen). This data illustrates that the peptide having a sequence of SEQ ID NO: 1, the peptide having a sequence of SEQ ID NO: 2, the peptide having a sequence of SEQ ID NO: 30, and the peptide having a sequence of SEQ ID NO: 32 bind human TfR, while the peptide having a sequence of SEQ ID NO: 1 has weaker binding relative to the other three peptides tested. The y-axis shows hTfR+Streptavidin from 0 to 10⁷, in increments of 10 on a log scale. The x-axis shows GFP from 0 to 10⁶, in increments of 10 on a log scale.

FIG. 18D illustrates quantification of binding of the peptide having a sequence of SEQ ID NO: 1, the peptide having a sequence of SEQ ID NO: 2, the peptide having a sequence of SEQ ID NO: 30, and the peptide having a sequence of SEQ ID NO: 32 to murine TfR. Data is displayed as four topographical density maps and indicates flow cytometry data using 293ST cells+SDGF-mTFR. Three density maps appear nearly superimposed and are oriented above a fourth density map. The lower density map is oriented horizontally and depicts SEQ ID NO: 1 (1^{st} gen). The upper three density maps are oriented diagonally from lower left to upper right. The density map slightly above the other two corresponds to SEQ ID NO: 32 (3^{rd} gen). The density map slightly below the other two corresponds to SEQ ID NO: 2 (2^{nd} gen). The third density map corresponds to SEQ ID NO: 30 (3^{rd} gen). This data illustrates that the peptide having a sequence of SEQ ID NO: 2, the peptide having a sequence of SEQ ID NO: 30, and the peptide having a sequence of SEQ ID NO: 32 bind murine TfR, whereas the peptide having a sequence of SEQ ID NO: 1 did not demonstrate binding to mTfR under the conditions tested. The y-axis shows hTfR+Streptavidin from 0 to 10⁷, in increments of 10 on a log scale. The x-axis shows GFP from 0 to 10⁶, in increments of 10 on a log scale.

FIG. 19A and FIG. 19B illustrate CDP-NT peptide complexes which induce an IP₁ response downstream of the neurotensin receptor (NTSR) both in CRE-Luciferase (CRE-Luc) mice and in mammalian cells.

FIG. 19A illustrates the relevant pathways influencing CRE-driven luciferase in the CRE-Luc mice. PLC denotes phospholipase C. AC denotes adenylyl cyclase. CaMK denotes calmodulin-dependent protein kinase. CREB denotes the cAMP response element binding protein. PKA denotes protein kinase A. PDE denotes cAMP phosphodiesterase. FS denotes forskolin. Rol denotes rolipram. GPCR denotes a G-protein-coupled receptor.

FIG. 19B shows FRET data illustrating in vitro neurotensin (NT) receptor engagement showing IP₁ accumulation only in response to NT or NT peptide complexes in HEK-293 cells expressing NTSR1. IP₁ is measured using an assay kit (CisBio 62IPAPEB) with a readout of FRET ratio. N=3 wells for all except vehicle, which had N=36. Horizontal bar indicates sample mean. mTF=murine transferrin. Baseline HEK293=mean assay value for HEK293 cells (N=36 wells) that do not express NTSR1, included as a reference.

FIG. 20A schematically illustrates mechanisms of resistance to tyrosine kinase inhibitors (TKIs) or anti-EGFR antibody therapies (e.g., cetuximab) in EGFR-driven cancer cells. EGFR-driven cancer cells with normal EGFR (panel 1) are sensitive to both anti-EGFR antibodies and tyrosine kinase inhibitors, resulting in reduced downstream KRAS and MEK signaling in response to either treatment (indicated by gray dashed arrows). Mutations in EGFR that prevent TKI binding (panel 2) are resistant to TKIs, showing little or no change in downstream signaling in response to TKI treatment (indicated by solid black arrows); TKI-resistant EGFR-driven cancer cells may still be sensitive to anti-EGFR antibodies. Heterodimerization with and cross-activation by other related growth factor receptors (e.g., HER2, ERBB3, or MET) render EGFR-driven cancer cells in which the dimerization partner is overexpressed (panel 3) insensitive to one or both of anti-EGFR antibodies and TKIs. EGFR-driven cancer cells in which EGFR is constitutively active (panel 4), such as EGFR variant III (EGFRvIII), are insensitive to anti-EGFR antibodies that prevent dimerization-driven activation of EGFR; cells with constitutively active EGFR may still be sensitive to TKIs.

FIG. 20B schematically illustrates use of selective depletion complexes (SDCs) to overcome resistance mechanisms in EGFR-driven cancer cells. This shows that SDCs can be effective against EGFR-driven cancer, including those cancers or cancer cells with normal EGFR as well as those cancers or cancer cells with resistance to TKI or EGFR antibody therapy. EGFR-driven cancer cells with Normal EGFR (panel 1) in an EGFR-driven cancer cell is effectively depleted by an SDC, resulting in reduced downstream KRAS and MEK signaling (indicated by gray dashed arrows) in response to SDC treatment. Mutated EGFR that prevent TKI binding (panel 2) is effectively depleted by an SDC, resulting in reduced downstream KRAS and MEK signaling in response to SDC treatment. EGFR heterodimerized with and cross-activated by an overexpressed growth factor receptor (e.g., HER2, ERBB3, or MET, panel 3) is effectively depleted by an SDC, resulting in reduced downstream KRAS and MEK signaling in response to SDC treatment. Depletion of the heterodimerized EGFR also has the potential to deplete the heterodimerization partner (e.g., HER2, ERBB3, or MET, panel 3). Constitutively active EGFR (panel 4), such as EGFRvIII, is effectively depleted by an SDC, resulting in reduced downstream KRAS and MEK signaling in response to SDC treatment.

FIG. 21 shows flow sorting data illustrating enrichment of peptides with pH-dependent binding to PD-L1. This data shows that pH-dependent binding peptides can be generated through flow sorting. A histidine-doped library based on a PD-L1-binding peptide (SEQ ID NO: 187), prepared as described in FIG. 11D, was screened for peptides that exhibited stronger PD-L1 binding at neutral pH (7.4) and weaker binding at acidic pH (5.5). The input library was initially screened for high PD-L1 binding at pH 7.4. The second and third rounds of screening (“Sort 1” and “Sort 2,” respectively) were performed at pH 5.5 to mimic endosomal pH, enriching for poor PD-L1 binding at this pH. The final round of screening (“Sort 3”) was performed at pH 7.4. Differential binding at pH 7.4 and pH 5.5 was observed following screening (“Sort 4”). The areas encompassed by the 5-sided polygon in each graph denotes the population that was selected during sorting. Darker topographical density maps indicate staining with PD-L1 under pH 7.4 conditions and lighter topographical density maps indicate staining with PD-L1 under pH 5.5 conditions.

FIG. 22 shows binding data at pH 7.4 (left bars) and at pH 5.5 (right bars) for pH-dependent PD-L1-binding peptide variants identified in FIG. 21. Variants of SEQ ID NO: 187 with E2H, M13H, and K16H substitutions, individually and in combination, were screened for pH-dependent binding to PD-L1. Peptide variants containing substitutions at E2H (SEQ ID NO: 234), M13H (SEQ ID NO: 235), K16H (SEQ ID NO: 236), E2H and M13H (SEQ ID NO: 237), E2H and K16H (SEQ ID NO: 233), M13H and K16H (SEQ ID NO: 238), or E2H, M13H, and K16H (SEQ ID NO: 239) exhibited varying degrees of pH-dependent binding to PD-L1. “UTF” indicates untransfected cells (negative control). The parent peptide (SEQ ID NO: 187) exhibited some degree of pH-dependent binding to PD-L1. Some variants of SEQ ID NO: 187 exhibited more pH-dependence in PD-L1 binding than the parent, while some variants of SEQ ID NO: 187 exhibited less pH-dependence in PD-L1 binding than the parent. The peptide of SEQ ID NO: 234 was shown to have a high difference in binding at pH 7.4 versus pH 5.5, demonstrating higher binding at pH 7.4 than at pH 5.5. The peptide of SEQ ID NO: 233 (black arrow) is shown to have a particularly high difference in binding at pH 7.4 versus pH 5.5, also demonstrating higher binding at pH 7.4 than at pH 5.5. This data illustrates the generation of peptides that bind PD-L1 at higher levels at pH 7.4 and at lower levels at pH 5.5.

FIG. 23A schematically illustrates the domain configuration of selective depletion complexes, such as those utilized in assays shown in FIG. 23B and FIG. 23C. Selective depletion complexes contained, from N-terminus to C-terminus, a target-binding peptide, a first peptide linker (GGGGS×4, SEQ ID NO: 224), an albumin binding peptide (SEQ ID NO: 227), a second peptide linker (GGGGS×4, SEQ ID NO: 224), and a TfR-binding peptide.

FIG. 23B shows an SDS-PAGE gel of two purified selective depletion complexes arranged as illustrated in FIG. 23A, and two negative controls complexes where the TfR-binding peptide is replaced with a peptide that does not bind TfR. Peptide 1 (SEQ ID NO: 367) contained a target-binding peptide that binds EGFR (SEQ ID NO: 244) and a peptide that does not significantly bind TfR corresponding to SEQ ID NO: 232. Peptide 2 (SEQ ID NO: 328) contained a target-binding peptide that binds EGFR (SEQ ID NO: 244) and a high affinity TfR-binding peptide corresponding to SEQ ID NO: 96. Peptide 3 (SEQ ID NO: 357) contained a target-binding peptide that binds PD-L1 (SEQ ID NO: 187) and a peptide that does not significantly bind TfR corresponding to SEQ ID NO: 232. Peptide 4 (SEQ ID NO: 356) contained a target-binding peptide that binds PD-L1 (SEQ ID NO: 187) and a high affinity TfR-binding peptide corresponding to SEQ ID NO: 96. This data indicates the production and purity of these peptides.

FIG. 23C shows ternary complex formation of the four peptide complexes shown in FIG. 23B with cells expressing EGFR (left) or PD-L1 (right). Cells were stained with fluorescently labeled TfR to detect ternary complex formation between a target protein expressed on the cell surface, the peptide complex, and TfR. Peptide 2 (SEQ ID NO: 328), which contained an EGFR-binding peptide and a high affinity TfR-binding peptide, formed ternary complexes with EGFR-expressing cells but not with PD-L1-expressing cells. Peptide 4 (SEQ ID NO: 356), which contained a PD-L1-binding peptide and a high affinity TfR-binding peptide, formed ternary complexes with PD-L1-expressing cells but not with EGFR-expressing cells. Peptides 1 and 3, which did not contain high affinity TfR-binding peptides, did not form ternary complexes. This data indicates that peptides complexes containing a target-binding peptide and a TfR-binding peptide can form ternary complexes on a cell surface with the target and with TfR.

FIG. 24A schematically illustrates ternary complex formation between a selective depletion complex (SDC, containing a target-binding peptide, a receptor-binding peptide, and a His tag (SEQ ID NO: 228)), a target protein expressed on a cell surface, and a transferrin receptor expressed on a cell surface.

FIG. 24B shows binding data for peptide complexes with (+) or without (−) a target-binding peptide that binds PD-L1 (SEQ ID NO: 187, “PDL1”) and with or without a receptor-binding peptide that binds TfR (SEQ ID NO: 96, “TfR”) to cells that express TfR with or without expressing PD-L1 (“PDL1”). All peptide complexes contained a His tag (SEQ ID NO: 228). The 1^{st} bar corresponds to PBS negative control, no peptide complex. The 2^{nd} and 3^{rd} bars were measured using a peptide complex of SEQ ID NO: 357. The 4^{th} and 5^{th} bars were measured using a peptide complex of SEQ ID NO: 356 capable of binding both PD-L1 and TfR. A peptide complex that contains both a PD-L1 binding peptide and a TfR-binding peptide can be a selective depletion complex (SDC). Binding was measured using a fluorescent anti-His antibody that bound to the His-tag on the peptide complexes. High levels of binding were observed using an SDC that binds both PD-L1 and TfR on cells that are expressing both PD-L1 and TfR. This data shows that when a cell is expressing both the target and the receptor, an SDC that containing binding peptides to both the target and the receptor will bind to that cell at high levels (5^{th} bar). The data also shows that a peptide complex that binds TfR will bind to a cell that is expressing TfR (4^{th} bar), even though adding a surface target binder increases SDC binding (5^{th} bar), presumably due to cooperative binding. Cooperative binding could possibly also be achieved by using an SDC with two TfR-binding peptides.

FIG. 25A schematically illustrates examples of monovalent selective depletion complexes containing a single target-binding moiety (EGFR-binding nanobodies or PD-L1-binding CDPs in this example) and a single receptor-binding moiety (TfR-binding CDPs or scFvs in this example). These can be arranged in a single protein, where both moieties are separated by a linker, or as a dimeric complex where one monomer contains a TfR-binding moiety, and another contains a target-binding moiety. Active catalytic molecules are those for which the TfR-binding moiety binds in a pH-independent fashion and the target-binding moiety binds in a pH-dependent fashion. Active non-catalytic molecules are those for which the TfR-binding moiety binds in a pH-dependent fashion and the target-binding moiety binds in a pH-independent fashion. Either active catalytic or active non-catalytic molecules would be expected to cause selective depletion of their target; non-catalytic molecules would travel with the target down the endosomal degradation pathway, while catalytic molecules would follow TfR back to the cell surface to bind another target. Representative control molecules are those where both TfR-binding and target-binding moieties bind in a pH-independent fashion but would not be expected to cause a selective depletion of their target either as effectively or to the same degree as the active catalytic or active non-catalytic molecules, or would not cause selective depletion of the target at all or in a significant manner. Other controls can be used to assess TfR-dependency of the active catalytic or active non-catalytic molecules, and could include comparatively measuring a depletion of a molecule that does not bind TfR, which control would not be expected to cause a selective depletion of their target either as effectively or to the same degree as the active catalytic or active non-catalytic molecules, or would not cause selective depletion of the target at all or in a significant manner.

FIG. 25B schematically illustrates examples of selective depletion complexes with differing valence for TfR- and/or target-binding. The figure illustrates Fc fusions where the TfR-binding moiety (a pH-independent TfR-binding CDP in this case) may be present once in the molecule (monovalent) or twice in the molecule (bivalent), and the target-binding moiety (a pH-dependent EGFR-binding nanobody in this case) may be present once in the molecule (monovalent) or twice in the molecule (bivalent). Fc fusions in which the two monomers are not identical can be assembled via knob-in-holes (KIH) dimerization. Multivalent selective depletion complexes can also be expressed as a single polypeptide chain (not shown).

FIG. 26A shows a co-crystal structure of a high-affinity PD-L1-binding CDP (SEQ ID NO: 187, cartoon) binding to or docked with PD-L1 (surface, with lighter shading denoting oxygen and darker shading denoting nitrogen).

FIG. 26B shows relative binding enrichment, shown as absolute value of average SSM enrichment, of PD-L1-binding CDP variants containing amino acid substitutions in resolved (R) residues or unresolved (UR) residues, as seen in the co-crystal structure of FIG. 26A. Substitutions at resolved residues had a greater impact, either positive or negative, on binding than substitutions at unresolved residues (**: P=0.0055), showing that resolved played a greater role in interactions with PD-L1 than unresolved residues.

FIG. 26C shows an overlay of PD-1 (mesh) with SEQ ID NO: 187 (cartoon) at the binding interface with PD-L1 (surface, with lighter shading denoting oxygen and darker shading denoting nitrogen). The PD-1 binding site overlaps with SEQ ID NO: 187, showing that SEQ ID NO: 187 would be expected to compete with PD-1 for binding to PD-L1.

FIG. 26D shows a zoomed in view of the SEQ ID NO: 187 PD-L1 co-crystal structure of FIG. 26A from two different angles. Residues of SEQ ID NO: 187 that interact with PD-L1, including K5, V9, W12, M13, K16, V39, F40, L43, and D44, are shown as sticks. Residues of PD-L1 that interact with SEQ ID NO: 187, including Y56, Q66, R113, M115, A121, and Y123, are also labeled.

FIG. 26E shows isolated side chains of select residues in SEQ ID NO: 187 (gray) at the PD-L1-binding interface relative the parent CDP (black, minimally clashing rotamers). Labeled residues of SEQ ID NO: 187, including M13, V39, F40, and L43, correspond to substitutions relative to parent CDP that improved binding to PD-L1.

FIG. 26F shows a zoomed in view of the binding interface between SEQ ID NO: 187 (cartoon) and PD-L1 (surface). The PD-L1 surface is color-coded for human (Hs) versus murine (Mm) homology, wherein white corresponds to identical residues, darker shading corresponds to similar residues, and lighter shading corresponds to dissimilar residues. These differences in the binding interface between human and murine PD-L1 are consistent with the lack of murine PD-L1 cross-reactivity seen with SEQ ID NO: 187.

FIG. 26G shows a co-crystal structure of SEQ ID NO: 187 and PD-L1 in which SEQ ID NO: 187 is illustrated as a wire diagram with side chains of interest shown with thick sticks (top). PD-1 binding to PD-L1 is shown at bottom for comparison.

### DETAILED DESCRIPTION

Described herein are compositions and methods for selective depletion of a target molecule using cellular endocytic pathways (e.g., transferrin receptor-mediated endocytosis). Extracellular, soluble, and cell-surface proteins mediate signaling between cells and organs, including growth, cell death, inflammation, metabolism, and more. Such proteins are regularly cycled through production, use, and degradation, and their degradation is typically within the endosomal-lysosomal pathway. In this pathway, endocytic vesicles containing material taken up from extracellular space as well as embedded membrane proteins become acidified and fuse with or enter lysosomes containing enzymes that degrade such proteins. Selective removal of certain cell surface or soluble proteins, either from circulation or disease-associated tissues, via selective delivery to the lysosome can be used to treat disease conditions, including diseases resulting from over-expression or accumulation of soluble or cell surface proteins or diseases associated with mutations (e.g., mutations causing constitutive activity, resistance to treatment, or dominant negative activity) in soluble or surface proteins. Alternatively or in addition, the selective depletion complexes described herein can be used to deliver an administered therapeutic drug to an endosomal or lysosomal compartment, for example to treat lysosomal storage diseases like Gaucher's Disease (deficiency of glucocerebrosidase) or Pompe Disease (deficiency of α-glucosidase). A therapeutic molecule (e.g., a lysosomal enzyme for an enzyme replacement therapy) can be administered with a selective depletion complex comprising a target-binding peptide that binds the therapeutic molecule, thereby delivering the therapeutic molecule to the endosome or lysosome. In some embodiments, a selective depletion construct can function as a selective delivery complex and facilitate delivery of active enzymes to an endosome or lysosome. For example, a lysosomal enzyme can be delivered using a selective depletion complex and can retain enzymatic activity in the endosome or lysosome. Administration of a lysosomal enzyme in combination with a selective depletion complex comprising a target-binding peptide that binds the lysosomal enzyme can increase the therapeutic response per dose of enzyme administered relative to administration of the lysosomal enzyme alone. For either selective depletion of target proteins or delivery of lysosomal proteins, lysosomal delivery could be accomplished by taking advantage of existing protein uptake and recycling mechanisms, and engineering of pH-dependent binding domains into target-binding molecules.

A unique example of an endocytic pathway that can be used for selective depletion of target molecules is via transferrin receptor (TfR) internalization and trafficking, which is normally used for transferrin recycling via transferrin receptor (TfR) for iron delivery to cells and tissues. Transferrin is known as a serum chaperone for iron ions destined for redox sensitive intracellular enzymes. Iron-loaded transferrin (holo-transferrin) delivers iron to cells via specific binding to TfR, which is then trafficked to endosomes, where the pH is reduced by native proton pumps. Under acidic conditions, transferrin loses its iron binding affinity, releasing iron inside the cell, but maintains its TfR-binding affinity. The TfR:transferrin complex is natively recycled back to the cell surface, exposing transferrin to neutral pH conditions. Transferrin unbound by iron (apo-transferrin) no longer has TfR affinity under neutral pH conditions at the cell surface, and is released back into circulation to pick up more iron, and repeat the process, in what is essentially a catalytic process for iron delivery to cells.

The compositions and methods of this disclosure exploit the transferrin receptor endocytic and recycling pathways to deliver target molecules (e.g., soluble or cell surface proteins) to endocytic vesicles for lysosomal degradation. The compositions and methods of this disclosure can be used to selectively degrade specific target receptor or soluble proteins that are over-expressed in disease via this pathway. As a result of lysosomal degradation of the target receptors or soluble proteins, the compositions and methods described effectively reduce, diminish, eliminate or deplete the target receptors from the cell surface or soluble proteins in circulation, which has many applications in medicine as described herein. Selective depletion complexes of the present disclosure comprising a TfR-binding peptide (e.g., a TfR-binding cystine-dense peptide) coupled to a target-binding peptide (e.g., a target-binding cystine-dense peptide, a target-binding antibody, a target-binding nanobody, a target-binding antibody fragment, or other targeting agent) can recruit a target molecule to the TfR by binding to both the TfR (via the TfR-binding peptide) and to the target (via the target-binding peptide). Upon endocytosis, the TfR can carry the selective depletion complex and the target molecule into the endocytic vesicle. In some embodiments, the TfR-binding peptide of the selective depletion complex can have high affinity for TfR at extracellular pH (about pH 7.4) to endosomal pH (about pH 5.5), inclusive. The TfR-binding peptide can maintain its affinity for TfR upon internalization and as the endosomal compartment acidifies. The target-binding peptide of the selective depletion complex can have higher affinity for the target molecule at extracellular pH and lower affinity for the target molecule at a lower endosomal pH. Inside the endocytic vesicle, the selective depletion complex can remain bound to TfR and release the target molecule upon acidification of the endosome. Once the target is released, the selective depletion complex can remain bound to TfR while TfR is recycled to the cell surface to be reloaded with another target molecule, and the target molecule can remain in the endosome where it is delivered to a lysosome and degraded. In some embodiments, the TfR-binding peptide of the selective depletion complex can have higher affinity for TfR at extracellular pH and lower affinity for the target molecule at a lower endosomal pH. Inside the endocytic vesicle, the selective depletion complex can release from TfR upon acidification of the endosome.

The methods of the present disclosure can comprise contacting a cell (e.g., a cell expressing TfR) with a selective depletion complex (e.g., a molecule comprising a TfR-binding peptide and a target-binding peptide). The selective depletion complex can recruit target molecules into endocytic vesicles via transferrin receptor-mediated (TfR-mediated) endocytosis.

The target molecule can be released in the endocytic vesicle where it is delivered to the lysosome and degraded. The selective depletion complex can remain bound to the TfR and can remain bound to TfR as TfR is recycled to the cell surface. Such methods can be used to deplete a target molecule, such as a molecule associated with a disease or a condition. For example, the methods of the present disclosure can be used to selectively deplete a soluble protein or a cell surface protein that is over-expressed, contains a disease-associated mutation (e.g., a mutation causing constitutive activity, resistance to treatment, or dominant negative activity), or accumulates in a disease or a condition.

In some embodiments, the presently described selective depletion complex can comprise peptide conjugates, peptide complexes, peptide constructs, fusion peptides, or fusion molecules such as linked by chemical conjugation of any molecule type, such as small molecules, peptides, or proteins, or by recombinant fusions of peptides or proteins, respectively (e.g., a peptide construct or a peptide complex). The terms “fusion peptide” and “peptide fusion” are used interchangeably herein. In some embodiments, the peptide constructs or peptide complexes can be produced biologically or synthetically. Thus, in some cases, a selective depletion complex can comprise a TfR-binding peptide domain linked to another molecule or group of molecules such as small molecules, peptides, or proteins or other macromolecules such as nanoparticles.

In some embodiments, the presently described selective depletion complexes can be peptide complexes comprising one or more TfR-binding peptides as described herein conjugated to, linked to, or fused to one or more target-binding peptides, one or more active agents (e.g., therapeutic agents, detectable agents, or combinations thereof), or combinations thereof. Selective depletion complexes as described herein can include chemical conjugates and recombinant fusion molecules. In some cases, a chemical conjugate can comprise a TfR-binding peptide as described herein that is chemically conjugated to or linked to another peptide (e.g., a target-binding peptide), a molecule, an agent, or a combination thereof. Molecules can include small molecules, peptides, polypeptides, proteins, or other macromolecules (e.g., nanoparticles) and polymers (e.g., nucleic acids, polylysine, or polyethylene glycol). In some cases, a TfR-binding peptide of the present disclosure is conjugated to another peptide or a molecule via a linker. Linker moieties can include cleavable (e.g., pH sensitive or enzyme-labile linkers) or stable linkers. In some embodiments, a peptide complex is a fusion molecule (e.g., a fusion peptide or fusion protein) that can be recombinantly expressed, and wherein the fusion molecule can comprise one or more TfR-binding peptides fused to one or more other molecules peptides, polypeptides, proteins, or other macromolecules that can be recombinantly expressed.

The selective depletion complexes of this disclosure (e.g., complexes comprising a TfR-binding peptide and a target-binding peptide) can have a therapeutic effect at a lower dose or a longer lasting therapeutic effect as compared to lysosomal delivery molecules that are degraded and not recycled to the cell surface. Rather than being degraded in the lysosome, the selective depletion complexes of this disclosure can be recycled back to the cell surface to “reload” with the target, meaning that the potential for one selective depletion complex of this disclosure can drive the degradation of multiple target molecules with a potentially catalytic effect. A lysosomal delivery molecule that is not recycled to the cell surface can itself be degraded or can accumulate in the lysosome without being re-used or “reloaded”. The selective depletion complexes of this disclosure (e.g., complexes comprising a TfR-binding peptide and a target-binding peptide) can have a wider therapeutic window (i.e., the dosage above which a therapeutic pharmacodynamic response is observed but below which toxicity is observed) as compared to lysosomal delivery molecules that are not recycled to the cell surface. The therapeutic window of a drug (e.g., a selective depletion complex of the present disclosure) is the dose range at which the drug is effective without having unacceptable toxic effects. The selective depletion complexes of this disclosure (e.g., complexes comprising a TfR-binding peptide and a target-binding peptide) can be used with less risk of toxicity. The selective depletion complexes of this disclosure (e.g., complexes comprising a TfR-binding peptide and a target-binding peptide) can be used at lower molar dosage than alternative therapies (e.g., lysosomal delivery molecules) that are not recycled to the cell surface. Because of the selectivity and re-usable nature of the selective depletion complexes of this disclosure in the cell, as therapeutic agents they are advantageously not depleted as rapidly as non-recyclable delivery compositions targeted to lysosomes which are depleted as they are used. Moreover, because of the selectivity and recycling aspect of the selective depletion complexes of this disclosure, as therapeutic agents they are advantageously less toxic than non-selective therapeutic agents. This is particularly advantageous for applications in cancer, where therapeutic agents can be non-selective and highly toxic and exhibit detrimental side effects on normal cells, organs and tissues, or require lower than effective therapeutic doses less able to reduce, cure, ablate disease.

The selective depletion complexes of this disclosure (e.g., complexes comprising a TfR-binding peptide and a target-binding peptide) can have less immunogenicity than an alternative therapy (e.g., a lysosomal delivery molecule) that contains sugars, glycans, polymers containing sugar-like molecules, or other derivatives. A selective depletion complex of this disclosure can have less immunogenicity than an alternative therapy (e.g., a lysosomal delivery molecule) that targets the mannose-6-phosphate receptor or the asialoglycoprotein receptor. A selective depletion complex of this disclosure can be manufactured by a single recombinant expression and can have improved manufacturing yield, purity, cost, or manufacturing time than a molecule that has multiple synthetic steps to generate a ligand for mannose-6-phosphate receptor or the asialoglycoprotein receptor. A selective depletion complex of this disclosure can have a greater therapeutic effect or a lower therapeutic dose due to the ability to design the linker for maximal ability to bind for the TfR and the target at the same time, including of the target is bound in the cell surface. The TfR-binding peptides, TfR-binding peptide conjugates, or TfR-binding fusion peptides of this disclosure can have fewer epitopes to trigger an adaptive immune response, resulting in reduced immunogenicity as compared to TfR-binding antibody-based therapeutics. The TfR-binding peptides, TfR-binding peptide conjugates, or TfR-binding fusion peptides of this disclosure can exhibit more facile and less disruptive incorporation of active agents into protein fusion complexes as compared to TfR-binding antibody-based therapeutics. The TfR-binding peptides, TfR-binding peptide conjugates, or TfR-binding fusion peptides of this disclosure can have a smaller surface area, resulting in lower risk for off-target binding, as compared to TfR-binding antibody-based therapeutics. The TfR-binding peptides, TfR-binding peptide conjugates, or TfR-binding fusion peptides of this disclosure can be formulated at a higher molar concentration than TfR-binding antibody-based therapeutics due to their lower molecule weight, lower hydrodynamic radius, or lower molar solution viscosity.

In some embodiments, the TfR-binding peptides, TfR-binding peptide conjugates, or TfR-binding fusion peptides of this disclosure exhibit lower on-target toxicity than an anti-TfR antibody or other therapeutic agents when administered to a subject at the same molar dose or at a similarly effective dose. In some embodiments, the TfR-binding peptides, TfR-binding peptide conjugates, or TfR-binding fusion peptides exhibit lower off-target toxicity than an antibody or other therapeutic agent when administered to a subject at the same molar dose or a similarly effective dose. For example, the TfR-binding peptides, TfR-binding peptide conjugates, or TfR-binding fusion peptides of this disclosure can be administered to a subject at about 1-fold, 2-fold, 3-fold, 4-fold, 5-fold, 10-fold, 15-fold, 20-fold, 25-fold, 30-fold, 40-fold, 50-fold, 60-fold, 70-fold, 80-fold, 90-fold, 100-fold higher molar dose than an antibody while providing similar or lower observed toxicity. In some embodiments, the TfR-binding peptides, TfR-binding peptide conjugates, or TfR-binding fusion peptides of this disclosure exhibit higher efficacy than an anti-TfR antibody or other therapeutic agent when administered to a subject at the same dose by weight as the anti-TfR antibody or other therapeutic agent. The TfR-binding peptides of the present disclosure, when fused to a half-life extending moiety (e.g., Fc, SA21, PEG), can be delivered at even lower doses while preserving activity and efficacy and, thus, is far superior to administering an anti-TfR antibody or other therapeutic agent.

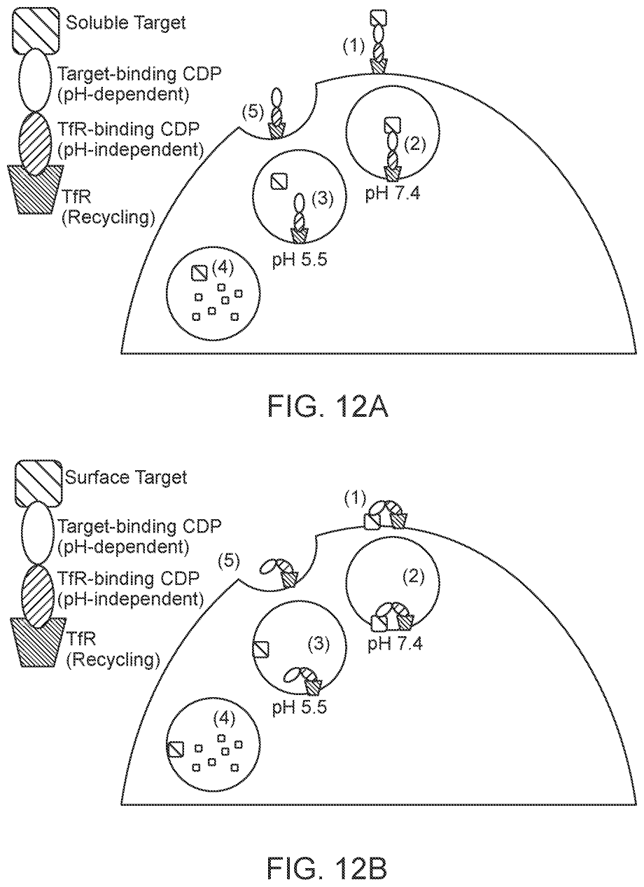

In some embodiments, the present disclosure provides peptides (e.g., CDPs, knotted peptides, or hitchins), chemical conjugates (e.g., comprising one or more TfR-binding peptides and one or more active agents), or recombinantly expressed fusion molecules (e.g., comprising one or more TfR-binding peptides and one or more active agents) that bind to TfR. The TfR-binding peptides can be cystine-dense peptides (CDPs). The terms “peptides”, “miniproteins”, “proteins”, “CDPs”, “TfR-binding peptides,” “TfR-binding CDPs,” “TfR-binding peptides,” and “engineered TfR-binding peptides” are used interchangeably herein. The binding of peptides described in the present disclosure to TfR can facilitate transcytosis of the selective depletion complex, peptide, peptide complex or peptide construct (e.g., fusion protein, or peptide conjugated to, linked to, or fused to an agent) across a cell barrier (e.g., the BBB). The binding of peptides described in the present disclosure to TfR can facilitate endocytosis of the selective depletion complex, peptide, or peptide complex in any cell that expresses TfR, or in cell that express TfR at higher levels, including some cancer cells, hepatic cells, spleen cells, and bone marrow cells. Also disclosed herein is the use of a mammalian surface display screening platform to screen a diverse library of CDPs and identify CDPs that specifically bind to human TfR. Such identified peptides can be modified to improve binding to TfR and used in selective depletion complexes as the peptide or peptide complex that binds TfR and is recycled to the cell surface (e.g., the pH-independent TFR-binding CDP as shown in FIG. 12A and FIG. 12B). Also disclosed herein is the use of a mammalian surface display screening platform to screen a diverse library of CDPs and identify CDPs that specifically bind to a target that is desired to be degraded. Such identified peptides can be optimized for binding to a selected target and used in selective depletion complexes as the peptide or peptide complex that binds such selected target and is released in the endosome for degradation within the cell (e.g., the pH-dependent target-binding CDP as shown in FIG. 12A and FIG. 12B). Further affinity maturation can be subsequently implemented to produce an allelic series of TfR-binding CDPs or target-binding CDPs as appropriate with varying affinities. In some embodiments, TfR-binding CDPs or target-binding CDPs are identified and binding can be determined by crystallography or other methods. Peptides of the present disclosure can have cross-reactivity across species. For example, the peptides disclosed herein, in some cases, bind to human and murine TfR. Peptides disclosed herein can accumulate in the CNS and can penetrated the BBB via engagement of the TfR, following intravenous administration. Disclosed herein are TfR-binding CDPs for use as therapeutic delivery agents in oncology, autoimmune disease, acute and chronic neurodegeneration, and pain management. Delivery of active or pharmaceutical agents via TfR-binding CDP can be advantageous over conventional anti-TfR antibodies due to simpler manufacturing (peptides can be made via biologic or synthetic means), improved stability, improved therapeutic window, and smaller size (less potential for steric hindrance of cargo activity). Thus, the methods and compositions of the present disclosure can provide a solution to the problem of effectively transporting cargo molecules (e.g., therapeutic and/or diagnostic small molecules, peptides or proteins) into the CNS (e.g., the brain). For example, the peptides of the present disclosure aid in drug delivery to tumors located in the brain.

In some embodiments of the present disclosure, a diverse library of CDPs, knotted peptides, hitchins, or peptides derived from knotted peptides or hitchins can be used in combination with a mammalian surface display screening platform is used to identify peptides that specifically bind to human TfR desired for recycling or to a target desired for degradation. (See e.g., Crook et al. (2017) Mammalian display screening of diverse cystine-dense peptides for difficult to drug targets. Nat Commun 8:2244). In some embodiments, a diverse library of CDPs, knotted peptides, hitchins, or peptides derived from knotted peptides or hitchins is mutagenized from endogenous peptide sequences to provide novel peptide sequences. Once TfR-binding or target-binding peptides have been identified, affinity maturation (e.g., site-saturation mutagenesis) can be performed to produce an allelic series of binders with varying (e.g., improved) affinities for TfR or a target. These techniques can be used in combination with various other analytical methods (e.g., crystallography or spectroscopy) in order to determine the nature of peptide-receptor interaction (e.g., critical amino acid residues for receptor binding etc.). In some cases, the peptides of the present disclosure are developed to bind human TfR.

In some embodiments, the engineered peptides of the present disclosure (e.g., histidine-containing or histidine-enriched target-binding peptides) can have a high target binding affinity at physiologic extracellular pH (e.g., a pH from about pH 7.2 to about pH 7.5, a pH of from about pH 6.5 to about 7.5, or a pH of from about pH 6.5 to about pH 6.9) but a significantly reduced binding affinity at lower pH levels such as endosomal pH of about 6.5, about 6.0, or about 5.5. Extracellular pH can be, for example pH 7.4. Extracellular pH can also be lower, including in the tumor microenvironment, such as pH 7.2, 7.0, or 6.8. In some embodiments, for example in a tumor environment, extracellular pH can be from about pH 6.5 to about pH 6.9. Upon endocytosis, the endosome undergoes a decrease in pH. Endosomal pH can decrease by the action of proton pumps or by merging with other vesicles with lower pH. The pH can decrease to 7.0, and then to 6.5, and then to 6.0, and then to 5.5 or lower. Some endosomes are called early endosomes and can have a pH around 6.5. Some of these endosomes become recycling endosomes. Some endosomes are called late endosomes and can have a pH around 5.5. Some endosomes become or merge with lysosomes, where the pH can be 4.5. Enzymes and other factors in the lysosome can cause degradation of the contents of the lysosome. In some embodiments, the target-binding peptides release in the endosome at about pH 7.3, pH 7.2, pH 7.1, pH 7.0, pH 6.9, pH 6.8, pH 6.7, pH 6.6, pH 6.5, pH 6.4, pH 6.3, pH 6.2, pH 6.1, pH 6.0, pH 5.9, pH 5.8, pH 5.7, pH 5.6, pH 5.5, pH 5.4, pH 5.3, pH 5.2, pH 5.1, pH 5.0, pH 4.9, pH 4.8, pH 4.7, pH 4.6, pH 4.5, or lower. In some embodiments, the target-binding peptide may release at any point during the endosomal maturation process upon a decrease in pH following endocytosis. In some cases, histidine scans and comparative binding experiments can be performed to develop and screen for such peptides. In some embodiments, an amino acid residue in a peptide of the present disclosure is substituted with a different amino acid residue to alter a pH-dependent binding affinity to the target or to TfR. The amino acid substitution can increase a binding affinity at low pH, increase a binding affinity at high pH, decrease a binding affinity at low pH, decrease a binding affinity at high pH, or a combination thereof. For example, a peptide that has high affinity to TfR and used in selective depletion complexes as the peptide or peptide complex that binds TfR for recycling to the cell surface can be a pH-independent TfR-binding peptide (e.g., a pH-independent TfR-binding CDP) such that it is not released in the endosome. In some embodiments, the TfR-binding peptide can remain bound to TfR as the ionic strength of the endosomal compartment increases upon acidification of the endosome. In some embodiments the TfR-binding peptides are stable at endosomal pH, and do not release in the endosome for example under acidic conditions, such as pH 6.9, pH 6.8, pH 6.7, pH 6.6, pH 6.5, pH 6.4, pH 6.3, pH6.2, pH 6.1, pH 6.0, pH 5.9, pH 5.8, pH 5.7, pH 5.6, pH 5.5, pH 5.4, pH 5.3, pH 5.2, pH 5.1, pH 5.0, pH 4.9, pH 4.8, pH 4.7, pH 4.6, pH 4.5, or lower. Conversely, a peptide that has high affinity for binding to a selected target and used in selective depletion complexes as the peptide or peptide complex that binds such selected target and is released in the endosome for degradation within the cell can be a pH-dependent target-binding CDP such that it is released in the endosome. In some embodiments, a target-binding peptide can release the target as the ionic strength of the endosomal compartment increases upon acidification of the endosome. In some embodiments the target-binding peptides are less stable at endosomal pH, and release wholly or in part in the endosome for example under acidic conditions, such as pH 7.3, pH 7.2, pH 7.1, pH 7.0, pH 6.9, pH 6.8, pH 6.7, pH 6.6, pH 6.5, pH 6.4, pH 6.3, pH 6.2, pH 6.1, pH 6.0, pH 5.9, pH 5.8, pH 5.7, pH 5.6, pH 5.5, pH 5.4, pH 5.3, pH 5.2, pH 5.1, pH 5.0, pH 4.9, pH 4.8, pH 4.7, pH 4.6, pH 4.5, or lower. In some cases, the TfR-binding peptides of the present disclosure can be optimized for improved intra-vesicular (e.g., intra-endosomal) function while retaining high TfR binding capabilities. Exemplary TfR-binding peptides of the present disclosure are shown in TABLE 1 with amino acid sequences set forth in SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64.

Described herein are, in some embodiments, peptides and peptide complexes and methods of screening for peptides and peptide complexes that bind to a protein or molecule of interest, such as TfR, or bind to a target molecule for depletion, or both. Compared to wild type or endogenous molecules such as transferrin, the methods and compositions as described herein can provide peptides with improved TfR-binding capabilities, or peptides that exhibit improved transport capabilities across the BBB, or any combination thereof. In some cases, the presently described peptides efficiently transport cargo molecules (e.g., target-binding molecules) across endothelial cell layers (e.g., the BBB) or epithelial layers. In some embodiments, the TfR-binding peptides of the present disclosure bind to a TfR and promote vesicular transcytosis. In some cases, the TfR-binding peptides of the present disclosure bind to a cell that overexpress a TfR (e.g., a cancer cell) and promotes uptake of the peptide by the cell. In some aspects a TfR binding peptide or peptide complexes as described herein promotes vesicular transcytosis and uptake by a TfR-overexpressing cell such as a cancer, or a combination thereof. In some cases, the TfR-binding peptides of the present disclosure facilitate TfR-mediated endocytosis of a selective depletion complex and a target molecule.

The TfR-binding peptides of the present disclosure can bind TfR of different species including human, monkey, mouse, and rat TfR. In some cases, variations or mutations in any of the amino acid residues of a TfR-binding peptide can influence cross-reactivity. In some cases, variations or mutations in any of the amino acid residues of a TfR-binding peptide that interact with the bindings site of TfR can influence cross-reactivity.

Described herein are peptides, including, but not limited to, designed or engineered peptides, recombinant peptides, and cystine-dense peptides (CDPs)/small disulfide-knotted peptides (e.g., knotted peptides, hitchins, and peptides derived therefrom), that can be large enough to carry a cargo molecule while retaining the ability to bind a target protein with high affinity (e.g., TfR), but yet small enough to access cellular tissues, such as the center of cell agglomerates (e.g., solid tumors). In some cases, the peptides as described herein carry cargo molecules across the BBB into the CNS (e.g., the parenchyma) via vascular transcytosis. In some cases, the transcytosis is TfR-mediated.

Further described herein are methods and compositions for determining the nature of peptide-receptor interactions (e.g., using X-ray crystallography) as well as their pharmacodynamic and pharmacokinetic properties in vivo, including accumulation in the CNS (e.g., brain), or other affected organs and tissues. Some of the peptides described herein have the ability to target and accumulate in tumor cells. In some cases, the tumor cells overexpress TfR. In some aspects, the peptides of the present disclosure have high in vivo stabilities, e.g., high protease stability, high tolerability of reducing agents such as glutathione (GSH), and tolerate elevated temperatures (e.g., up to 95° C.).

The present disclosure provides, in some embodiments, a peptide or protein design approach based on the 3D protein or receptor structure for identifying peptides or proteins capable of binding such receptor. In some cases, the receptor is a transferrin receptor.

As used herein, the abbreviations for the natural L-enantiomeric amino acids are conventional and are as follows: alanine (A, Ala); arginine (R, Arg); asparagine (N, Asn); aspartic acid (D, Asp); cysteine (C, Cys); glutamic acid (E, Glu); glutamine (Q, Gln); glycine (G, Gly); histidine (H, His); isoleucine (I, Ile); leucine (L, Leu); lysine (K, Lys); methionine (M, Met); phenylalanine (F, Phe); proline (P, Pro); serine (S, Ser); threonine (T, Thr); tryptophan (W, Trp); tyrosine (Y, Tyr); valine (V, Val). Typically, Xaa can indicate any amino acid. In some embodiments, X can be asparagine (N), glutamine (Q), histidine (H), lysine (K), or arginine (R).

Some embodiments of the disclosure contemplate D-amino acid residues of any standard or non-standard amino acid or analogue thereof. When an amino acid sequence is represented as a series of three-letter or one-letter amino acid abbreviations, the left-hand direction is the amino terminal direction and the right-hand direction is the carboxy terminal direction, in accordance with standard usage and convention.

The terms “peptide”, “polypeptide”, “miniprotein”, “protein”, “hitchin”, “cystine-dense peptide”, “knotted peptides” or “CDP” can be used interchangeably herein to refer to a polymer of amino acid residues. In various embodiments, “peptides”, “polypeptides”, and “proteins” can be chains of amino acids whose alpha carbons are linked through peptide bonds. The terminal amino acid at one end of the chain (e.g., amino terminal, or N-terminal) therefore can have a free amino group, while the terminal amino acid at the other end of the chain (e.g., carboxy terminal, or C-terminal) can have a free carboxyl group. As used herein, the term “amino terminus” (e.g., abbreviated N-terminus) can refer to the free ca-amino group on an amino acid at the amino terminal of a peptide or to the ca-amino group (e.g., imino group when participating in a peptide bond) of an amino acid at any other location within the peptide. Similarly, the term “carboxy terminus” can refer to the free carboxyl group on the carboxy terminus of a peptide or the carboxyl group of an amino acid at any other location within the peptide. Peptides also include essentially any polyamino acid including, but not limited to, peptide mimetics such as amino acids joined by an ether or thioether as opposed to an amide bond.

As used herein, the term “peptide construct” can refer to a molecule comprising one or more peptides of the present disclosure that can be conjugated to, linked to, or fused to one or more peptides or cargo molecules. In some cases, cargo molecules are active agents. The term “active agent” can refer to any molecule, e.g., any molecule that is capable of eliciting a biological effect and/or a physical effect (e.g., emission of radiation) which can allow the localization, detection, or visualization of the respective peptide construct. In various embodiments, the term “active agent” refers to a therapeutic and/or diagnostic agent. A peptide construct of the present disclosure can comprise a TfR-binding peptide that is linked to one or more active agents via one or more linker moieties (e.g., cleavable or stable linker) as described herein.

As used herein, the term “peptide complex” can refer to one or more peptides of the present disclosure that are fused, linked, conjugated, or otherwise connected to form a complex. In some cases, the one or more peptides can comprise a TfR-binding peptide, a target-binding peptide, a half-life modifying peptide, a peptide that modifies pharmacodynamics and/or pharmacokinetic properties, or combinations thereof. For example, a peptide complex comprising a TfR-binding peptide and a target-binding peptide can be referred to herein as a selective depletion complex.

As used herein, the terms “comprising” and “having” can be used interchangeably. For example, the terms “a peptide comprising an amino acid sequence of SEQ ID NO: 32” and “a peptide having an amino acid sequence of SEQ ID NO: 32” can be used interchangeably.

As used herein, and unless otherwise stated, the term “TfR” or “transferrin receptor” is a class of protein used herein and can refer to a transferrin receptor from any species (e.g., human or murine TfR or any human or non-human animal TfR). In some cases, and as used herein, the term “TfR” or “transferrin receptor” refers to human TfR (hTfR) and can include TfR or any of the known TfR homologs or orthologs, including TfR1, TfR2, soluble TfR, or any combination or fragment (e.g., ectodomain) thereof.

As used herein, the terms “endosome,” “endosomal,” “endosomal compartment,” or “endocytic pathway” can be used interchangeably and may refer to any one or more components of the intracellular endosomal network or trans-Golgi network (TGN) that allows for the vesicular transcytosis or trafficking and transfer of peptides and cargoes between distinct membrane-bound compartments within a cell, including lysosomal degradation as well as recycling to the cell surface. It is understood that such pathway involves and includes the maturation and transition of vesicles commonly referred to as transport vesicles or early endosomes to late endosomes to lysosomes, and that endosomal compartment acidity increases upon acidification of the endosome throughout the maturation process. Lysosomes serving as the last vesicle in the matured endocytic pathway typically contain hydrolytic enzymes which digest the contents of the late endosomes. Other endosomes continue to a pathway of recycling endosomes, where the contents are recycled back to the cell surface.

As used herein “pH-independent,” when used in reference to a molecule or moiety, refer means that as the endosomal compartment is acidified, the binding affinity of the molecule or moiety to its target does not change sufficiently to enable dissociation in the endosome with the target. For example, the referenced molecule or moiety has the same or similar affinity to its target at extracellular pH and at an endosomal pH. It is also understood that pH-independent molecules or moieties do not include pH-dependent molecules or moieties, since the binding affinity of pH-dependent molecules or moieties to its target changes as it enters and proceeds through the endosomal pathway, for example, to enable dissociation in the endosome with the target to some degree, or the referenced molecule or moiety has a different affinity at extracellular pH and at an endosomal pH.

The term “engineered,” when applied to a polynucleotide, denotes that the polynucleotide has been removed from its natural genetic milieu and is thus free of other extraneous or unwanted coding sequences and is in a form suitable for use within genetically engineered protein production systems. Such engineered molecules are those that are separated from their natural environment and include cDNA and genomic clones (i.e., a prokaryotic or eukaryotic cell with a vector containing a fragment of DNA from a different organism). Engineered DNA molecules of the present invention are free of other genes with which they are ordinarily associated but can include naturally occurring or non-naturally occurring 5′ and 3′ untranslated regions such as enhancers, promoters and terminators.

An “engineered” polypeptide or protein is a polypeptide or protein that is found in a condition other than its native environment, such as apart from blood and animal tissue. In a preferred form, the engineered polypeptide is substantially free of other polypeptides, particularly other polypeptides of animal origin. It is preferred to provide the polypeptides in a highly purified form, e.g., greater than 90% pure, greater than 95% pure, more preferably greater than 98% pure or greater than 99% pure. When used in this context, the term “engineered” does not exclude the presence of the same polypeptide in alternative physical forms, such as dimers, heterodimers and multimers, heteromultimers, or alternatively glycosylated, carboxylated, modified, or derivatized forms.

An “engineered” peptide or protein is a polypeptide that is distinct from a naturally occurring polypeptide structure, sequence, or composition. Engineered peptides include non-naturally occurring, artificial, isolated, synthetic, designed, modified, or recombinantly expressed peptides. Provided herein are engineered TfR-binding peptides, variants, or fragments thereof. These engineered TfR-binding peptides can be further linked to a target-binding moiety or a half-life extending moiety, or can be further linked to an active agent or detectable agent, or any combination of the foregoing.

Polypeptides of the disclosure include polypeptides that have been modified in any way, for example, to: (1) reduce susceptibility to proteolysis, (2) reduce susceptibility to oxidation, (3) alter binding affinity for forming protein complexes, (4) alter binding affinities, (5) alter binding affinity at certain pH values, and (6) confer or modify other physicochemical or functional properties. For example, single or multiple amino acid substitutions (e.g., conservative amino acid substitutions) are made in the naturally occurring sequence (e.g., in the portion of the polypeptide outside the domain(s) forming intermolecular contacts). A “conservative amino acid substitution” can refer to the substitution in a polypeptide of an amino acid with a functionally similar amino acid. The following six groups each contain amino acids that can be conservative substitutions for one another: i) Alanine (A), Serine (S), and Threonine (T); ii) Aspartic acid (D) and Glutamic acid (E); iii) Asparagine (N) and Glutamine (Q); iv) Arginine (R) and Lysine (K); v) Isoleucine (I), Leucine (L), Methionine (M), and Valine (V); vi) Phenylalanine (F), Tyrosine (Y), and Tryptophan (W). In some embodiments, a conserved amino acid substitution can comprise a non-natural amino acid. For example, substitution of an amino acid for a non-natural derivative of the same amino acid can be a conserved substitution.

The terms “polypeptide fragment” and “truncated polypeptide” as used herein can refer to a polypeptide that has an amino-terminal and/or carboxy-terminal deletion as compared to a corresponding full-length peptide or protein. In various embodiments, fragments are at least 5, at least 10, at least 15, at least 20, at least 25, at least 30, at least 35, at least 40, at least 45, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can also be, e.g., at most 1000, at most 900, at most 800, at most 700, at most 600, at most 500, at most 450, at most 400, at most 350, at most 300, at most 250, at most 200, at most 150, at most 100, at most 50, at most 45, at most 40, at most 35, at most 30, at most 25, at most 20, at most 15, at most 10, or at most 5 amino acids in length. A fragment can further comprise, at either or both of its ends, one or more additional amino acids, for example, a sequence of amino acids from a different naturally-occurring protein (e.g., an Fc or leucine zipper domain) or an artificial amino acid sequence (e.g., an artificial linker sequence).

As used herein, the terms “peptide” or “polypeptide” in conjunction with “variant” “mutant” or “enriched mutant” or “permuted enriched mutant” can refer to a peptide or polypeptide that can comprise an amino acid sequence wherein one or more amino acid residues are inserted into, deleted from and/or substituted into the amino acid sequence relative to another polypeptide sequence. In various embodiments, the number of amino acid residues to be inserted, deleted, or substituted is at least 1, at least 2, at least 3, at least 4, at least 5, at least 10, at least 25, at least 50, at least 75, at least 100, at least 125, at least 150, at least 175, at least 200, at least 225, at least 250, at least 275, at least 300, at least 350, at least 400, at least 450 or at least 500 amino acids in length. Variants of the present disclosure include peptide conjugates or fusion molecules (e.g., peptide constructs or peptide complexes).

A “derivative” of a peptide or polypeptide can be a peptide or polypeptide that can have been chemically modified, e.g., conjugation to another chemical moiety such as, for example, polyethylene glycol, albumin (e.g., human serum albumin), phosphorylation, and glycosylation.

The term “% sequence identity” can be used interchangeably herein with the term “% identity” and can refer to the level of amino acid sequence identity between two or more peptide sequences or the level of nucleotide sequence identity between two or more nucleotide sequences, when aligned using a sequence alignment program. For example, as used herein, 80% identity means the same thing as 80% sequence identity determined by a defined algorithm, and means that a given sequence is at least 80% identical to another length of another sequence. In various embodiments, the % identity is selected from, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 99% or more up to 100% sequence identity to a given sequence. In various embodiments, the % identity is in the range of, e.g., about 60% to about 70%, about 70% to about 80%, about 80% to about 85%, about 85% to about 90%, about 90% to about 95%, or about 95% to about 99%.

The terms “% sequence homology” or “percent sequence homology” or “percent sequence identity” can be used interchangeably herein with the terms “% homology,” “% sequence identity,” or “% identity” and can refer to the level of amino acid sequence homology between two or more peptide sequences or the level of nucleotide sequence homology between two or more nucleotide sequences, when aligned using a sequence alignment program. For example, as used herein, 80% homology means the same thing as 80% sequence homology determined by a defined algorithm, and accordingly a homologue of a given sequence has greater than 80% sequence homology over a length of the given sequence. In various embodiments, the % homology is selected from, e.g., at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, or at least 99% or more up to 100% sequence homology to a given sequence. In various embodiments, the % homology is in the range of, e.g., about 60% to about 70%, about 70% to about 80%, about 80% to about 85%, about 85% to about 90%, about 90% to about 95%, or about 95% to about 99%.

A protein or polypeptide can be “substantially pure,” “substantially homogeneous”, or “substantially purified” when at least about 60% to 75% of a sample exhibits a single species of polypeptide. The polypeptide or protein can be monomeric or multimeric. A substantially pure polypeptide or protein can typically comprise about 50%, 60%, 70%, 80% or 90% W/W of a protein sample, more usually about 95%, and e.g., will be over 98% or 99% pure. Protein purity or homogeneity can be indicated by a number of means well known in the art, such as polyacrylamide gel electrophoresis of a protein sample, followed by visualizing a single polypeptide band upon staining the gel with a stain well known in the art. For certain purposes, higher resolution is provided by using high-pressure liquid chromatography (e.g., HPLC) or other high-resolution analytical techniques (e.g., LC-mass spectrometry).

As used herein, the term “pharmaceutical composition” can generally refer to a composition suitable for pharmaceutical use in a subject such as an animal (e.g., human or mouse). A pharmaceutical composition can comprise a pharmacologically effective amount of an active agent and a pharmaceutically acceptable carrier. The term “pharmacologically effective amount” can refer to that amount of an agent effective to produce the intended biological or pharmacological result.

As used herein, the term “pharmaceutically acceptable carrier” can refer to any of the standard pharmaceutical carriers, vehicles, buffers, and excipients, such as a phosphate buffered saline solution, or a buffered saline solution, 5% aqueous solution of dextrose, and emulsions, such as an oil/water or water/oil emulsion, and various types of wetting agents and/or adjuvants. Suitable pharmaceutical carriers and formulations are described in Remington's Pharmaceutical Sciences, 21^{st} Ed. 2005, Mack Publishing Co, Easton. A “pharmaceutically acceptable salt” can be a salt that can be formulated into a compound for pharmaceutical use including, e.g., metal salts (sodium, potassium, magnesium, calcium, etc.) and salts of ammonia or organic amines.

As used herein, the terms “treat”, “treating” and “treatment” can refer to a method of alleviating or abrogating a biological disorder and/or at least one of its attendant symptoms. As used herein, to “alleviate” a disease, disorder or condition, for example, means reducing the severity and/or occurrence frequency of the symptoms of the disease, disorder, or condition. Further, references herein to “treatment” can include references to curative, palliative, and prophylactic or diagnostic treatment.

Generally, a cell of the present disclosure can be a eukaryotic cell or a prokaryotic cell. A cell can be an epithelial cell. A cell can be a microorganism, bacterial, yeast, fungal or algae cell. A cell can be an animal cell or a plant cell. An animal cell can include a cell from a marine invertebrate, fish, insects, amphibian, reptile, or mammal. A mammalian cell can be obtained from a primate, ape, equine, bovine, porcine, canine, feline, or rodent. A mammal can be a primate, ape, dog, cat, rabbit, ferret, or the like. A rodent can be a mouse, rat, hamster, gerbil, hamster, chinchilla, or guinea pig. A bird cell can be from a canary, parakeet or parrots. A reptile cell can be from a turtles, lizard or snake. A fish cell can be from a tropical fish. For example, the fish cell can be from a zebrafish (e.g., Danino rerio). A worm cell can be from a nematode (e.g., C. elegans). An amphibian cell can be from a frog. An arthropod cell can be from a tarantula or hermit crab.

A mammalian cell can also include cells obtained from a primate (e.g., a human or a non-human primate). A mammalian cell can include a blood cell, a stem cell, an epithelial cell, connective tissue cell, hormone secreting cell, a nerve cell, a skeletal muscle cell, or an immune system cell.

As used herein, the term “vector,” generally refers to a DNA molecule capable of replication in a host cell and/or to which another DNA segment can be operatively linked so as to bring about replication of the attached segment. A plasmid is an exemplary vector.

As used herein, the term “subject,” generally refers to a human or to another animal. A subject can be of any age, for example, a subject can be an infant, a toddler, a child, a pre-adolescent, an adolescent, an adult, or an elderly individual.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are in relation to the other endpoint, and independently of the other endpoint. The term “about” as used herein refers to a range that is 15% plus or minus from a stated numerical value within the context of the particular usage. For example, about 10 can include a range from 8.5 to 11.5.

### Peptides

The selective depletion complexes of the present disclosure can comprise one or more peptides. For example, a selective depletion complex of the present disclosure can comprise a TfR-binding peptide and a target binding peptide. In some embodiments, two or more peptides can be connected via a linker. The peptides of the present disclosure (e.g., TfR-binding peptide, a target-binding peptide, or a peptide comprising a TfR-binding peptide linked to a target-binding peptide) can be used in a method of selectively depleting a target molecule. The peptides of the present disclosure (e.g., TfR-binding peptide, a target-binding peptide, or a peptide comprising a TfR-binding peptide linked to a target-binding peptide) can be recycled to the cell surface following endocytosis.

In some instances, a peptide as disclosed herein can contain only one lysine residue, or no lysine residues. In some instances, one or more or all of the lysine residues in the peptide are replaced with arginine residues. In some instances, one or more or all of the methionine residues in the peptide are replaced by leucine or isoleucine. One or more or all of the tryptophan residues in the peptide can be replaced by phenylalanine or tyrosine. In some instances, one or more or all of the asparagine residues in the peptide are replaced by glutamine. In some embodiments, one or more or all of the aspartic acid residues can be replaced by glutamic acid residues. In some instances, one or more or all of the lysine residues in the peptide are replaced by alanine or arginine. In some embodiments, the N-terminus of the peptide is blocked or protected, such as by an acetyl group or a tert-butyloxycarbonyl group. Alternatively or in combination, the C-terminus of the peptide can be blocked or protected, such as by an amide group or by the formation of an ester (e.g., a butyl or a benzyl ester). In some embodiments, the peptide is modified by methylation on free amines. For example, full methylation is accomplished through the use of reductive methylation with formaldehyde and sodium cyanoborohydride.

In some embodiments, the dipeptide GS can be added as the first two N-terminal amino acids, as shown in SEQ ID NO: 1-SEQ ID NO: 64, or such N-terminal dipeptide GS can be absent as shown in SEQ ID NO: 65-SEQ ID NO: 128, or can be substituted by any other one or two amino acids. In some embodiments, the dipeptide GS is used as a linker or used to couple to a linker to form a peptide conjugate or fusion molecules such as a peptide construct or peptide complex. In some embodiments, the linker comprises a GₓS_{y} (SEQ ID NO: 130) peptide, wherein x and y independently are any whole number, such as 1, 2, 3, 4, 5, 6, 7, 8, 9, and 10 and the G and S residues are arranged in any order. In some embodiments, the peptide linker comprises (GS)x (SEQ ID NO: 131), wherein x can be any whole number, such as 1, 2, 3, 4, 5, 6, 7, 8, 9, and 10. In some embodiments, the peptide linker comprises GGSSG (SEQ ID NO: 132), GGGGG (SEQ ID NO: 133), GSGSGSGS (SEQ ID NO: 134), GSGG (SEQ ID NO: 135), GGGGS (SEQ ID NO: 136), GGGS (SEQ ID NO: 129), GGS (SEQ ID NO: 137), GGGSGGGSGGGS (SEQ ID NO: 138), or a variant or fragment thereof or any number of repeats and combinations thereof. Additionally, KKYKPYVPVTTN (SEQ ID NO: 139) from DkTx, and EPKSSDKTHT (SEQ ID NO: 140) from human IgG3 can be used as a peptide linker or any number of repeats and combinations thereof. In some embodiments, the peptide linker comprises GGGSGGSGGGS (SEQ ID NO: 141) or a variant or fragment thereof or any number of repeats and combinations thereof. It is understood that any of the foregoing linkers or a variant or fragment thereof can be used with any number of repeats or any combinations thereof. It is also understood that other peptide linkers in the art or a variant or fragment thereof can be used with any number of repeats or any combinations thereof. The length of the linker can be tailored to maximize binding of the selective delivery complex to both TfR and the target at the same time including accounting for steric access. In some embodiments, the linker between the TfR-binding and target-binding peptides within the selective depletion complex is at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36 at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 residues incrementally up to 100 residues long, particularly for example if the target is not a soluble protein but rather a cell surface protein or cell receptor protein.

In some embodiments of the present disclosure, a peptide or peptide complex as described herein comprises an amino acid sequence set forth in any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. A peptide as disclosed herein can be a fragment comprising a contiguous fragment of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 that is at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36 at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58, at least 59, at least 60, at least 61, at least 62, at least 63, at least 64, at least 65 residues long, wherein the peptide fragment is selected from any portion of the peptide. In some embodiments, the peptide sequence is flanked by additional amino acids. One or more additional amino acids, for example, confer a particular in vivo charge, isoelectric point, chemical conjugation site, stability, or physiologic property to a peptide.

In some instances, the peptides as described herein that are capable of targeting and binding to a TfR comprise no more than 80 amino acids in length, or no more than 70, no more than 60, no more than 40, no more than 35, no more than 30, no more than 25, no more than 20, no more than 15, or no more than 10 amino acids in length. In some instances, the peptides as described herein that are capable of targeting and binding to a target molecule comprise no more than 80 amino acids in length, or no more than 70, no more than 60, no more than 50, no more than 40, no more than 35, no more than 30, no more than 25, no more than 24, no more than 23, no more than 22, no more than 21, no more than 20, no more than 19, no more than 18, no more than 17, no more than 16, no more than 15, no more than 14, no more than 13, no more than 12, no more than 11, or no more than 10 amino acids in length.

In other embodiments, peptides can be conjugated to, linked to, or fused to a carrier or a molecule with targeting or homing function for a cell of interest or a target cell. In other embodiments, peptides can be conjugated to, linked to, or fused to a molecule that extends half-life or modifies the pharmacodynamic and/or pharmacokinetic properties of the peptides, or any combination thereof.

In some instances, a peptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 positively charged residues, such as Arg or Lys, or any combination thereof. In some instances, one or more lysine residues in the peptide are replaced with arginine residues. In some embodiments, peptides comprise one or more Arg patches. In some embodiments, 1 or more, 2 or more, 3 or more, 4 or more, 5 or more, 6 or more, 7 or more, 8 or more, 9 or more, or 10 or more Arg or Lys residues are solvent exposed on a peptide. In some instances, a peptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 histidine residues.

The peptides of the present disclosure can further comprise neutral amino acid residues. In some embodiments, the peptide has 35 or fewer neutral amino acid residues. In other embodiments, the peptide has 81 or fewer neutral amino acid residues, 70 or fewer neutral amino acid residues, 60 or fewer neutral amino acid residues, 50 or fewer neutral amino acid residues, 40 or fewer neutral amino acid residues, 36 or fewer neutral amino acid residues, 33 or fewer neutral amino acid residues, 30 or fewer neutral amino acid residues, 25 or fewer neutral amino acid residues, or 10 or fewer neutral amino acid residues.

The peptides of the present disclosure can further comprise negative amino acid residues. In some embodiments the peptide has 6 or fewer negative amino acid residues, 5 or fewer negative amino acid residues, 4 or fewer negative amino acid residues, 3 or fewer negative amino acid residues, 2 or fewer negative amino acid residues, or 1 or fewer negative amino acid residues. While negative amino acid residues can be selected from any negatively charged amino acid residues, in some embodiments, the negative amino acid residues are either E, or D, or a combination of both E and D.

In some embodiments of the present disclosure, a three-dimensional or tertiary structure of a peptide is primarily comprised of beta-sheets and/or alpha-helix structures. In some embodiments, designed or engineered TfR-binding peptides or target-binding of the present disclosure are small, compact peptides or polypeptides stabilized by intra-chain disulfide bonds (e.g., mediated by cysteines) to form cystine and a hydrophobic core. In some embodiments, engineered TfR-binding peptides have structures comprising helical bundles with at least one disulfide bridge between each of the alpha helices, thereby stabilizing the peptides. In other embodiments, the engineered TfR-binding peptides or target-binding peptides comprise structures with three alpha helices and three intra-chain disulfide bonds, one between each of the three alpha helices in the bundle of alpha helices.

### Receptor-Binding Peptides

Disclosed herein are peptide sequences, such as those listed in TABLE 1 and TABLE 2, capable of binding to a receptor (e.g., a transferrin receptor or programmed death-ligand 1). The peptide capable of binding a receptor may be referred to as a receptor-binding peptide. In some embodiments, a receptor-binding peptide may bind to a recycled receptor that undergoes recycling via a recycling pathway. The recycled receptor may be endocytosed into an early endosome and packaged into a recycling endosome prior to maturation of the early endosome into a late endosome. The recycling endosome containing the recycled receptor may fuse with a cell membrane and return the recycled receptor to the cell surface. In some embodiments, a receptor-binding peptide of the present disclosure may remain bound to the receptor during the recycling process, thereby recycling the receptor-binding peptide as well. Examples of recycled receptors that may be targeted by a receptor-binding peptide include transferrin receptor and programmed death-ligand 1. In some embodiments, a receptor-binding peptide of the present disclosure may comprise a miniprotein, a nanobody, an antibody, an IgG, an antibody fragment, a Fab, a F(ab)2, an scFv, an (scFv)2, a DARPin, or an affibody. In some embodiments, the receptor-binding peptide may comprise a cystine-dense peptide, an affitin, an adnectin, an avimer, a Kunitz domain, a nanofittin, a fynomer, a bicyclic peptide, a beta-hairpin, or a stapled peptide.

In some embodiments, a receptor-binding peptide of the present disclosure can bind to the receptor (e.g., a recycled receptor) with an affinity that is pH-independent. For example, a receptor-binding peptide can bind the receptor at an extracellular pH (about pH 7.4) with an affinity that is substantially the same the binding affinity at an endocytic pH (such as about pH 5.5 or about pH 6.5). In some embodiments, a receptor-binding peptide can bind the receptor at an extracellular pH (about pH 7.4) with an affinity that is lower than the binding affinity at an endocytic pH (such as about pH 5.5 or about pH 6.5). In some embodiments, a receptor-binding peptide can bind the receptor at an extracellular pH (about pH 7.4) with an affinity that is higher than the binding affinity at an endocytic pH (such as about pH 5.5 or about pH 6.5). In some embodiments, the binding affinity of a receptor-binding peptide for the receptor at extracellular pH (about pH 7.4) and the binding affinity of a receptor-binding peptide for the receptor at endocytic pH (about pH 5.5) can differ by no more than about 1%, no more than about 2%, no more than about 3%, no more than about 4%, no more than about 5%, no more than about 6%, no more than about 7%, no more than about 8%, no more than about 9%, no more than about 10%, no more than about 12%, no more than about 15%, no more than about 17%, no more than about 20%, no more than about 25%, no more than about 30%, no more than about 35%, no more than about 40%, no more than about 45%, or no more than about 50%. In some embodiments, the affinity of the receptor-binding peptide for the receptor at pH 7.4 and at pH 5.5 can differ by no more than 2-fold, no more than 5-fold, no more than 10-fold, no more than 15-fold, no more than 20-fold, no more than 25-fold, no more than 30-fold, no more than 40-fold, or no more than 50-fold. In some embodiments, a receptor-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, SEQ ID NO: 1-SEQ ID NO: 64, SEQ ID NO: 187, SEQ ID NO: 233-SEQ ID NO: 239, SEQ ID NO: 400-SEQ ID NO: 456, or SEQ ID NO: 241) can be modified to remove one or more histidine amino acids in the TfR binding interface, thereby reducing the pH-dependence of the binding affinity of the receptor-binding peptide for the receptor. In some embodiments, a receptor-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, SEQ ID NO: 1-SEQ ID NO: 64, SEQ ID NO: 187, SEQ ID NO: 233-SEQ ID NO: 239, SEQ ID NO: 400-SEQ ID NO: 456, or SEQ ID NO: 241) can lack histidine amino acids in the receptor-binding interface.

In some embodiments, a receptor-binding peptide with pH-independent binding can bind to the receptor with a dissociation constant (K_{D}) of less than 50 μM, less than 5 μM, less than 500 nM, less than 100 nM, less than 40 nM, less than 30 nM, less than 20 nM, less than 10 nM, less than 5 nM, less than 2 nM, less than 1 nM, less than 0.5 nM, less than 0.4 nM, less than 0.3 nM, less than 0.2 nM, or less than 0.1 nM at extracellular pH (about pH 7.4). In some embodiments, a receptor-binding peptide with pH-independent binding can bind to the receptor with a dissociation constant (K_{D}) of less than 50 μM, less than 5 μM, less than 500 nM, less than 100 nM, less than 40 nM, less than 30 nM, less than 20 nM, less than 10 nM, less than 5 nM, less than 2 nM, less than 1 nM, less than 0.5 nM, less than 0.4 nM, less than 0.3 nM, less than 0.2 nM, or less than 0.1 nM at endosomal pH (about pH 5.5).

In some embodiments, the receptor-binding peptide can bind to the receptor with an affinity that is pH-dependent. For example, the receptor-binding molecule can bind to the receptor with higher affinity at extracellular pH (about pH 7.4) and with lower affinity at endosomal pH (about pH 5.5), thereby releasing the selective depletion complex from receptor upon internalization and acidification of the endosomal compartment.

In some embodiments, the recycling receptor may be TfR. A peptide capable of binding transferrin receptor (TfR) may bind TfR or any of the known TfR homologs, including TfR1, TfR2, soluble TfR, or any combination or fragment (e.g., ectodomain) thereof. A peptide capable of binding a transferrin receptor or a TfR homolog can be referred to herein as a transferrin receptor-binding peptide or a TfR-binding peptide. In some embodiments, peptides disclosed herein can penetrate, cross, or enter target cells in a TfR-mediated manner. These cell layers or cells can include TfR-expressing endothelial cells, epithelial cells, and TfR-expressing cells of various tissues or organs such as tumor cells, brain cells, cancerous or tumor cells, liver cells (e.g., hepatocytes (HCs), bepatic stellate cells (HSCs), Kupffer cells (KCs), or liver sinusoidal endothelial cells (LSECs)), pancreas cells, colon cells, ovarian cells, breast cells, spleen cells, bone marrow cells, and/or lung cells, or any combination thereof. In some embodiments, a TfR-binding peptide of the present disclosure may comprise a miniprotein, a nanobody, an antibody, an IgG, an antibody fragment, a Fab, a F(ab)2, an scFv, an (scFv)2, a DARPin, or an affibody. In some embodiments, the TfR-binding peptide may comprise a cystine-dense peptide, an affitin, an adnectin, an avimer, a Kunitz domain, a nanofittin, a fynomer, a bicyclic peptide, a beta-hairpin, or a stapled peptide.

In some embodiments, the peptides as discloses herein can cross cellular layers or barriers (e.g., BBB) or cell membranes via, for example, TfR-mediated vesicular transcytosis and TfR-mediated endocytosis, respectively. In addition to binding TfR and promote transcytosis and/or endocytosis, the peptides of the present disclosure can also bind to additional target proteins on cells such as cancer cells. In some cases, a peptide is a peptide or peptide complex comprising a TfR-binding peptide conjugated to, linked to, or fused to a targeting moiety or an active agent (e.g., a therapeutic or diagnostic agent) such as a small molecule or a peptide that has an affinity for an additional target protein (e.g., receptor or enzyme). In some cases, the TfR-binding peptide is linked to a target-binding peptide and enables or promotes TfR-mediated transcytosis of the target-binding peptide across the BBB or TfR-mediated endocytosis into a cell. In some instances, and subsequent to transcytosis, a peptide complex comprising the TfR-binding peptide and a target-binding peptide can target a specific cell or tissue in the CNS and exert a biological effect (e.g., binding a target protein) upon reaching said cell or tissue. In some cases, a peptide complex of the present disclosure exerts a biological effect that is mediated by the TfR-binding peptide, the target-binding peptide, an active agent, or a combination thereof. In some cases, a TfR-binding peptide complex of the present disclosure comprising one target-binding peptides can transport and/or deliver target molecules into cells that express TfR (e.g., deliver target molecules into endosomes). In some cases, the TfR-binding peptide accumulates in tissues in the CNS. In some cases, off-target effects are reduced due to CNS-specific accumulation. In some cases, the TfR-binding peptide accumulates in tissue outside of the CNS (e.g., liver, kidney, spleen, or skin). In some cases, the cells expressing TfR are tumor cells and the TfR-binding peptide complex delivers anti-tumor agents to these tumor cells. In some cases, the anti-tumor agents alone show no or only very limited therapeutic efficacy against the tumor cells; however, when the anti-tumor agents are combined with the TfR-binding peptides of the present disclosure as, for example, a peptide complex, the therapeutic efficacy of these anti-tumor agents is significantly improved.

In some embodiments, the TfR-binding peptides of the present disclosure (e.g., SEQ ID NO: 96, SEQ ID NO: 65-SEQ ID NO: 95, SEQ ID NO: 97-SEQ ID NO: 128, SEQ ID NO: 220-SEQ ID NO: 222, and SEQ ID NO: 1-SEQ ID NO: 64) can induce a biologically relevant response. For example, a TfR-binding peptide conjugated to a target-binding peptide can selectively deplete a soluble target molecule or a cell surface target molecule. In some embodiments, the biologically relevant response can be induced after intravenous, subcutaneous, peritoneal, intracranial, or intramuscular dose, and in some embodiments, after a single intravenous, subcutaneous, peritoneal, intracranial, or intramuscular dose. In some embodiments, the TfR-binding peptides can be used in combination with various other classes of therapeutic compounds used to treat and/or prevent pain, neuropathic pain or other neurological disorders such as neurodegenerative disorders, infectious diseases, immunological disorders (e.g., autoimmune diseases) or lysosomal storage diseases. Binding of the herein described peptides and peptide complexes (e.g., peptide conjugates, fusion peptides, or recombinantly produced peptide complexes) to TfR and subsequent transport across a cell layer or barrier such as the BBB (e.g., via TfR-mediated vesicular transcytosis) or a cell membrane (e.g., via TfR-mediated endocytosis) can have implications in a number of diseases, conditions, or disorders associated with over-expression or accumulation of a target molecule (e.g., cancer, neurodegeneration, or lysosomal storage diseases) or diseases associated with mutations (e.g., mutations causing constitutive activity, resistance to treatment, or dominant negative activity) in soluble or surface proteins in a subject (e.g., a human).

Binding of the herein described peptides and peptide complexes (e.g., peptide conjugates, fusion peptides, or recombinantly produced peptide complexes) to TfR and subsequent transport across a cell layer or barrier such as the BBB (e.g., via vesicular transcytosis) or a cell membrane (e.g., via endocytosis) can have implications in a number of diseases, conditions, or disorders associated with neurodegeneration. Neurodegenerative diseases that can treated, prevented, or diagnosed with the herein described selective depletion complexes comprising TfR-binding peptides can include Alzheimer's disease, Amyotrophic lateral sclerosis, Friedreich's ataxia, Huntington's disease, Lewy body disease, Parkinson's disease, Spinal muscular atrophy, Motor neuron disease, Lyme disease, Ataxia-telangiectasia, Autosomal dominant cerebellar ataxia, Batten disease, Corticobasal syndrome, Creutzfeldt-Jakob disease, Fragile X-associated tremor/ataxia syndrome, Kufor-Rakeb syndrome, Machado-Joseph disease, multiple sclerosis, chronic traumatic encephalopathy, or frontotemporal dementia.

In some embodiments, TfR-binding peptides of the present disclosure can bind to any of the known TfR homologs, including TfR1, TfR2, soluble TfR, or any combination or fragment (e.g., ectodomain) thereof. Thus, as used herein, “TfR” can refer to any known homolog, derivative, fragment, or member of the TfR family including TfR1, TfR2, and a soluble TfR. In other embodiments, peptides are capable of binding to one, one or more, or all TfR homologs. In some embodiments, peptides of the present disclosure can bind to a TfR and promote a particular biological effect such as vesicular transcytosis. In some embodiments, TfR-binding peptides of the present disclosure, including peptides and peptide complexes with amino acid sequences set forth in SEQ ID NO: 96, SEQ ID NO: 65-SEQ ID NO: 95, SEQ ID NO: 97-SEQ ID NO: 128, SEQ ID NO: 220-SEQ ID NO: 222, and SEQ ID NO: 1-SEQ ID NO: 64, and any derivatives or variant thereof, prevent or decrease the binding of endogenous TfR binders (e.g., transferrin or any derivatives such as apo-transferrin or holo-transferrin) to TfR. In some embodiments, peptides or peptide complexes of the present disclosure comprise derivatives and variants with at least 40% homology, at least 50% homology, at least 60% homology, at least 70% homology, at least 75% homology, at least 80% homology, at least 85% homology, at least 90% homology, at least 91% homology, at least 92% homology, at least 93% homology, at least 94% homology, at least 95% homology, at least 96% homology, at least 97% homology, at least 98% homology, or at least 99% homology or at least 100% homology to amino acid sequences set forth in SEQ ID NO: 96, SEQ ID NO: 65-SEQ ID NO: 95, SEQ ID NO: 97-SEQ ID NO: 128, SEQ ID NO: 220-SEQ ID NO: 222, and SEQ ID NO: 1-SEQ ID NO: 64.

In various embodiments, the interface residues of the TfR-binding peptides of the present disclosure (e.g., those amino acid residues that interact with TfR for receptor binding) can be divided between two largely helical domains of the peptide. In some cases, the interface residues can comprise residues corresponding to residues 5-25 (e.g., and comprising corresponding residues G5, A7, S8, M11, N14, L17, E18, and E21), with reference to SEQ ID NO: 32, or corresponding to residues 35-51 (e.g., and comprising corresponding residues L38, L41, L42, L45, D46, H47, H49, S50, and Q51), with reference to SEQ ID NO: 32, or both. For example, the interface residues can comprise residues corresponding to residues 5-25 (e.g., and comprising corresponding residues G5, A7, S8, M11, N14, L17, E18, and E21), with reference to SEQ ID NO: 32, or corresponding to residues 35-51 (e.g., and comprising corresponding residues L38, L41, L42, L45, D46, H47, H49, S50, and Q51), with reference to SEQ ID NO: 32. In some embodiments, a TfR-binding peptide can comprise a fragment of a peptide provided herein, wherein the fragment comprises the minimum interface residues for binding, for example residues corresponding to residues 5-25 (e.g., and comprising corresponding residues G5, A7, S8, M11, N14, L17, E18, and E21), with reference to SEQ ID NO: 32, or corresponding to residues 35-51 (e.g., and comprising corresponding residues L38, L41, L42, L45, D46, H47, H49, S50, and Q51), with reference to SEQ ID NO: 32. In some cases, the TfR-binding peptide is a peptide having the sequence set forth in SEQ ID NO: 32 comprising the TfR-binding residues corresponding to residues G5, A7, S8, M11, N14, L17, E18, and E21 of the domain and corresponding to residues L38, L41, L42, L45, D46, H47, H49, S50, and Q51 of the second domain, with reference to SEQ ID NO: 32.

In some embodiments, TfR-binding peptides bind to TfR with equal, similar, or greater affinity (e.g., lower dissociation constant K_{D}) as compared to endogenous molecules (e.g., transferrin, holotransferrin (iron-bound transferrin), apotransferrin (transferrin not bound to iron), or any other endogenous TfR ligands) or other exogenous molecules. In some embodiments, the peptide can have a K_{D} of less than 50 μM, less than 5 μM, less than 500 nM, less than 100 nM, less than 40 nM, less than 30 nM, less than 20 nM, less than 10 nM, less than 5 nM, less than 2 nM, less than 1 nM, less than 0.5 nM, less than 0.4 nM, less than 0.3 nM, less than 0.2 nM, or less than 0.1 nM. In some embodiments, peptide transport by TfR is improved by having a lower affinity (e.g., a higher dissociation constant K_{D}) as compared to endogenous molecules. In some embodiments, peptide transport by TfR is improved by having a faster off rate or higher k_{off} than endogenous molecules. In some embodiments, the off rate or k_{off} is similar to that of transferrin. In some embodiments, peptide transport is improved by having a faster on rate or a higher kₒₙ, optionally such as higher than that of transferrin. In other embodiments, one or more conserved residues at the transferrin (Tf)-TfR-binding interface are also present in the amino acid sequences of the peptides described herein. In some embodiments, a TfR-binding peptide has an off rate that is slower than the recycling rate of TfR, such that the TfR-binding peptide is likely to remain bound to TfR during the recycling process. In some embodiments, the TfR-binding peptide may have an off rate that is no faster than 1 minute, no faster than 2 minutes, no faster than 3 minutes, no faster than 4 minutes, no faster than 5 minutes, no faster than 7 minutes, no faster than 10 minutes, no faster than 15 minutes, or no faster than 20 minutes. In some embodiments, the TfR-binding peptide may have an off rate that is from about 1 minute to about 20 minutes, from about 2 minutes to about 15 minutes, from about 2 minutes to about 10 minutes, or from about 5 minutes to about 10 minutes.

In some embodiments, TfR-binding peptides that exhibit an improved TfR receptor binding show improved transcytosis function, improved endocytosis function, improved recycling, or combinations thereof. In some embodiments, TfR-binding peptides that exhibit an improved TfR receptor binding show no or small changes in transcytosis function, endocytosis function, recycling, or combinations thereof. In some embodiments, TfR-binding peptides that exhibit an improved TfR receptor binding show reduced transcytosis function, reduced endocytosis function, reduced recycling, or combinations thereof. In some embodiments, the TfR-binding peptide binds at a site of high homology between human and murine TfR, including one or more, or all, of the amino acid domains corresponding to residues 506-510, 523-531, and 611-662 of the human TfR (SEQ ID NO: 190, MMDQARSAFSNLFGGEPLSYTRFSLARQVDGDNSHVEMKLAVDEEENADNNTKANVT KPKRCSGSICYGTIAVIVFFLIGFMIGYLGYCKGVEPKTECERLAGTESPVREEPGEDFPA ARRLYWDDLKRKLSEKLDSTDFTGTIKLLNENSYVPREAGSQKDENLALYVENQFREF KLSKVWRDQHFVKIQVKDSAQNSVIIVDKNGRLVYLVENPGGYVAYSKAATVTGKLV HANFGTKKDFEDLYTPVNGSIVIVRAGKITFAEKVANAESLNAIGVLIYMDQTKFPIVNA ELSFFGHAHLGTGDPYTPGFPSFNHTQFPPSRSSGLPNIPVQTISRAAAEKLFGNMEGDCP SDWKTDSTCRMVTSESKNVKLTVSNVLKEIKILNIFGVIKGFVEPDHYVVVGAQRDAW GPGAAKSGVGTALLLKLAQMFSDMVLKDGFQPSRSIIFASWSAGDFGSVGATEWLEGY LSSLHLKAFTYINLDKAVLGTSNFKVSASPLLYTLIEKTMQNVKHPVTGQFLYQDSNWA SKVEKLTLDNAAFPFLAYSGIPAVSFCFCEDTDYPYLGTTMDTYKELIERIPELNKVARA AAEVAGQFVIKLTHDVELNLDYERYNSQLLSFVRDLNQYRADIKEMGLSLQWLYSARG DFFRATSRLTTDFGNAEKTDRFVMKKLNDRVMRVEYHFLSPYVSPKESPFRHVFWGSG SHTLPALLENLKLRKQNNGAFNETLFRNQLALATWTIQGAANALSGDVWDIDNEF). In some embodiments, the regions of TfR to which the peptides disclosed herein or variants thereof bind all or in part to such TfR domains. In some embodiments, the peptides disclosed herein bind to any one, any two, or all three of the TfR regions of high homology including the amino acid domains corresponding to residues 506-510, 523-531, and 611-662 of the human TfR (SEQ ID NO: 190). In some embodiments the peptides disclosed herein bind at least to the domain corresponding to residues 611-662 of the human TfR.

In some embodiments, the K_{A} and K_{D} values of a TfR-binding peptide can be modulated and optimized (e.g., via amino acid substitutions) to provide an optimal ratio of TfR-binding affinity and efficient transcytosis function.

In some embodiments, peptides disclosed herein or variants thereof bind to TfR at residues found in the binding interface (e.g., the binding domain or the binding pocket) of TfR with other exogenous or endogenous ligands (e.g., transferrin (Tf), Tf derivatives, or Tf-like peptides or proteins). In some embodiments, a peptide disclosed herein or a variant thereof, which binds to TfR, comprises at least 70% homology, at least 75% homology, at least 80% homology, at least 85% homology, at least 90% homology, at least 95% homology, at least 96% homology, at least 97% homology, at least 98% homology, or at least 99% homology or at least 100% homology to a sequence that binds residues of TfR, which makeup the binding pocket. In some embodiments, a peptide disclosed herein or a variant thereof, which binds to TfR, comprises at least 70% homology, at least 75% homology, at least 80% homology, at least 85% homology, at least 90% homology, at least 95% homology, at least 96% homology, at least 97% homology, at least 98% homology, or at least 99% homology or at least 100% homology to an endogenous or exogenous polypeptide known to bind TfR, for example, endogenous Transferrin or any one of the peptides listed in TABLE 1. In other embodiments, a peptide described herein binds to a protein of interest, which comprises at least 70% homology, at least 75% homology, at least 80% homology, at least 85% homology, at least 90% homology, at least 95% homology, at least 96% homology, at least 97% homology, at least 98% homology, or at least 99% homology or at least 100% homology to TfR, a fragment, homolog, or a variant thereof.

In some embodiments, peptides disclosed herein or variants thereof bind regions of TfR that comprise the amino acid residues corresponding to residues 506-510, 523-531, and 611-662 (the numbering of these amino acid residues is based on the following Uniprot reference protein sequence of endogenous human TFRC UniProtKB—P02786 (SEQ ID NO: 190, TFR1_HUMAN)). In some embodiments, the regions of TfR to which the peptides disclosed herein or variants thereof bind overlap with those of Tf, a fragment, homolog, or a variant thereof.

In other embodiments, a nucleic acid, vector, plasmid, or donor DNA comprises a sequence that encodes a peptide, peptide construct, a peptide complex, or variant or functional fragment thereof, as described in the present disclosure. In further embodiments, certain parts or fragments of TfR-binding motifs (e.g., conserved binding motifs) can be grafted onto a peptide or peptide complex with a sequence of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. In some embodiments, peptides can cause TfR to be degraded, prevent TfR from localizing to a cell's nucleus, or prevent TfR from interacting with transferrin or transferrin-like proteins.

In some embodiments, a peptide can be selected for further testing or use based upon its ability to bind to the certain amino acid residue or motif of amino acid residues. The certain amino acid residue or motif of amino acid residues in TfR can be identified an amino acid residue or sequence of amino acid residues that are involved in the binding of TfR to Tf. A certain amino acid residue or motif of amino acid residues can be identified from a crystal structure of the TfR:Tf complex. In some embodiments, peptides (e.g., CDPs) demonstrate the resistance to heat, protease (pepsin), and reduction.

The peptides, peptide complexes (e.g., peptide conjugates or fusion peptides), and selective delivery complexes comprising one or more of the amino acid sequences set forth in SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 can bind to a protein of interest. In some embodiments, the protein of interest is a TfR. In some embodiments, the peptides and peptide complexes (e.g., peptide conjugates or fusion peptides) that bind to a TfR comprise at least one of the amino acid sequences set forth in SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. In some embodiments, peptides, peptide complexes (e.g., peptide conjugates and fusion molecules) of the present disclosure that bind to a TfR comprise peptide derivatives or variants having at least 70% homology, at least 75% homology, at least 80% homology, at least 85% homology, at least 90% homology, at least 95% homology, at least 96% homology, at least 97% homology, at least 98% homology, or at least 99% homology or at least 100% homology to amino acid sequences set forth in SEQ ID NO: 96, SEQ ID NO: 65-SEQ ID NO: 95, SEQ ID NO: 97-SEQ ID NO: 128, SEQ ID NO: 220-SEQ ID NOD 222, or SEQ ID NO: 1-SEQ ID NO: 64. For example, peptides or peptide complexes (e.g., peptide conjugates and fusion molecules) of the present disclosure that bind to a TfR can comprise peptide derivatives or variants having at least 70% homology, at least 75% homology, at least 80% homology, at least 85% homology, at least 90% homology, at least 95% homology, at least 96% homology, at least 97% homology, at least 98% homology, or at least 99% homology or at least 100% homology to the amino acid sequence set forth in SEQ ID NO: 96.

TABLE 1 lists exemplary peptide sequences according to the methods and compositions of the present disclosure.

TABLE 1
Exemplary TfR-Binding Peptide Sequences

| SEQ ID NO | Amino Acid Sequence |
|---|---|
| SEQ ID NO: 96 | REGCASRCMKYNDELEKCEARMMSMSNTEEDCEQELEDLLYCLDHCHSQ |
| SEQ ID NO: 65 | REGCASRCTKYNAELEKCEARVSSMSNTEETCVQELFDLLHCVDHCVSQ |
| SEQ ID NO: 66 | REGCASRCTKYNAELEKCEARVMSMSNTEEDCEQELEDLLHCLDHCHSQ |
| SEQ ID NO: 67 | REGCASRCTKYNAELEKCEARVVSMSNTEEDCEQELEDLLHCLDHCHSQ |
| SEQ ID NO: 68 | REGCASRCTKYNAELEKCEARVMSMSNTEETCEQELEDLLHCLDHCHSQ |
| SEQ ID NO: 69 | REGCASRCTKYNAELEKCEARVMSMSNTEEDCVQELEDLLHCLDHCHSQ |
| SEQ ID NO: 70 | REGCASRCTKYNAELEKCEARVMSMSNTEEDCEQELFDLLHCLDHCHSQ |
| SEQ ID NO: 71 | REGCASRCTKYNAELEKCEARVMSMSNTEEDCEQELEDLLHCVDHCHSQ |
| SEQ ID NO: 72 | REGCASRCTKYNAELEKCEARVMSMSNTEEDCEQELEDLLHCLDHCVSQ |
| SEQ ID NO: 73 | REGCASRCTKYNAELEKCEARVVSMSNTEETCEQELEDLLHCLDHCHSQ |
| SEQ ID NO: 74 | REGCASRCTKYNAELEKCEARVVSMSNTEEDCVQELEDLLHCLDHCHSQ |
| SEQ ID NO: 75 | REGCASRCTKYNAELEKCEARVVSMSNTEEDCEQELFDLLHCLDHCHSQ |
| SEQ ID NO: 76 | REGCASRCTKYNAELEKCEARVVSMSNTEEDCEQELEDLLHCVDHCHSQ |
| SEQ ID NO: 77 | REGCASRCTKYNAELEKCEARVVSMSNTEEDCEQELEDLLHCLDHCVSQ |
| SEQ ID NO: 78 | REGCASRCTKYNAELEKCEARVMSMSNTEETCVQELEDLLHCLDHCHSQ |
| SEQ ID NO: 79 | REGCASRCTKYNAELEKCEARVMSMSNTEETCEQELFDLLHCLDHCHSQ |
| SEQ ID NO: 80 | REGCASRCTKYNAELEKCEARVMSMSNTEETCEQELEDLLHCVDHCHSQ |
| SEQ ID NO: 81 | REGCASRCTKYNAELEKCEARVMSMSNTEETCEQELEDLLHCLDHCVSQ |
| SEQ ID NO: 82 | REGCASRCTKYNAELEKCEARVMSMSNTEEDCVQELFDLLHCLDHCHSQ |
| SEQ ID NO: 83 | REGCASRCTKYNAELEKCEARVMSMSNTEEDCVQELEDLLHCVDHCHSQ |
| SEQ ID NO: 84 | REGCASRCTKYNAELEKCEARVMSMSNTEEDCVQELEDLLHCLDHCVSQ |
| SEQ ID NO: 85 | REGCASRCTKYNAELEKCEARVMSMSNTEEDCEQELFDLLHCVDHCHSQ |
| SEQ ID NO: 86 | REGCASRCTKYNAELEKCEARVMSMSNTEEDCEQELFDLLHCLDHCVSQ |
| SEQ ID NO: 87 | REGCASRCTKYNAELEKCEARVMSMSNTEEDCEQELEDLLHCVDHCVSQ |
| SEQ ID NO: 88 | REGCASRCMKYNAELEKCEARVMSMSNTEEDCEQELEDLLHCLDHCHSQ |
| SEQ ID NO: 89 | REGCASRCTKYNDELEKCEARVMSMSNTEEDCEQELEDLLHCLDHCHSQ |
| SEQ ID NO: 90 | REGCASRCTKYNAELEKCEARMMSMSNTEEDCEQELEDLLHCLDHCHSQ |
| SEQ ID NO: 91 | REGCASRCTKYNAELEKCEARVMSMSNTEEDCEQELEDLLYCLDHCHSQ |
| SEQ ID NO: 92 | REGCASRCMKYNDELEKCEARMMSMSNTEEDCEQELEDLLHCLDHCHSQ |
| SEQ ID NO: 93 | REGCASRCMKYNDELEKCEARVMSMSNTEEDCEQELEDLLYCLDHCHSQ |
| SEQ ID NO: 94 | REGCASRCMKYNAELEKCEARMMSMSNTEEDCEQELEDLLYCLDHCHSQ |
| SEQ ID NO: 95 | REGCASRCTKYNDELEKCEARMMSMSNTEEDCEQELEDLLYCLDHCHSQ |
| SEQ ID NO: 97 | REGCASRCTRYNAELERCEARVSSMSNTEETCVQELFDLLHCVDHCVSQ |
| SEQ ID NO: 98 | REGCASRCTRYNAELERCEARVMSMSNTEEDCEQELEDLLHCLDHCHSQ |
| SEQ ID NO: 99 | REGCASRCTRYNAELERCEARVVSMSNTEEDCEQELEDLLHCLDHCHSQ |
| SEQ ID NO: 100 | REGCASRCTRYNAELERCEARVMSMSNTEETCEQELEDLLHCLDHCHSQ |
| SEQ ID NO: 101 | REGCASRCTRYNAELERCEARVMSMSNTEEDCVQELEDLLHCLDHCHSQ |
| SEQ ID NO: 102 | REGCASRCTRYNAELERCEARVMSMSNTEEDCEQELFDLLHCLDHCHSQ |
| SEQ ID NO: 103 | REGCASRCTRYNAELERCEARVMSMSNTEEDCEQELEDLLHCVDHCHSQ |
| SEQ ID NO: 104 | REGCASRCTRYNAELERCEARVMSMSNTEEDCEQELEDLLHCLDHCVSQ |
| SEQ ID NO: 105 | REGCASRCTRYNAELERCEARVVSMSNTEETCEQELEDLLHCLDHCHSQ |
| SEQ ID NO: 106 | REGCASRCTRYNAELERCEARVVSMSNTEEDCVQELEDLLHCLDHCHSQ |
| SEQ ID NO: 107 | REGCASRCTRYNAELERCEARVVSMSNTEEDCEQELFDLLHCLDHCHSQ |
| SEQ ID NO: 108 | REGCASRCTRYNAELERCEARVVSMSNTEEDCEQELEDLLHCVDHCHSQ |
| SEQ ID NO: 109 | REGCASRCTRYNAELERCEARVVSMSNTEEDCEQELEDLLHCLDHCVSQ |
| SEQ ID NO: 110 | REGCASRCTRYNAELERCEARVMSMSNTEETCVQELEDLLHCLDHCHSQ |
| SEQ ID NO: 111 | REGCASRCTRYNAELERCEARVMSMSNTEETCEQELFDLLHCLDHCHSQ |
| SEQ ID NO: 112 | REGCASRCTRYNAELERCEARVMSMSNTEETCEQELEDLLHCVDHCHSQ |
| SEQ ID NO: 113 | REGCASRCTRYNAELERCEARVMSMSNTEETCEQELEDLLHCLDHCVSQ |
| SEQ ID NO: 114 | REGCASRCTRYNAELERCEARVMSMSNTEEDCVQELFDLLHCLDHCHSQ |
| SEQ ID NO: 115 | REGCASRCTRYNAELERCEARVMSMSNTEEDCVQELEDLLHCVDHCHSQ |
| SEQ ID NO: 116 | REGCASRCTRYNAELERCEARVMSMSNTEEDCVQELEDLLHCLDHCVSQ |
| SEQ ID NO: 117 | REGCASRCTRYNAELERCEARVMSMSNTEEDCEQELFDLLHCVDHCHSQ |
| SEQ ID NO: 118 | REGCASRCTRYNAELERCEARVMSMSNTEEDCEQELFDLLHCLDHCVSQ |
| SEQ ID NO: 119 | REGCASRCTRYNAELERCEARVMSMSNTEEDCEQELEDLLHCVDHCVSQ |
| SEQ ID NO: 120 | REGCASRCMRYNAELERCEARVMSMSNTEEDCEQELEDLLHCLDHCHSQ |
| SEQ ID NO: 121 | REGCASRCTRYNDELERCEARVMSMSNTEEDCEQELEDLLHCLDHCHSQ |
| SEQ ID NO: 122 | REGCASRCTRYNAELERCEARMMSMSNTEEDCEQELEDLLHCLDHCHSQ |
| SEQ ID NO: 123 | REGCASRCTRYNAELERCEARVMSMSNTEEDCEQELEDLLYCLDHCHSQ |
| SEQ ID NO: 124 | REGCASRCMRYNDELERCEARMMSMSNTEEDCEQELEDLLHCLDHCHSQ |
| SEQ ID NO: 125 | REGCASRCMRYNDELERCEARVMSMSNTEEDCEQELEDLLYCLDHCHSQ |
| SEQ ID NO: 126 | REGCASRCMRYNAELERCEARMMSMSNTEEDCEQELEDLLYCLDHCHSQ |
| SEQ ID NO: 127 | REGCASRCTRYNDELERCEARMMSMSNTEEDCEQELEDLLYCLDHCHSQ |
| SEQ ID NO: 128 | REGCASRCMRYNDELERCEARMMSMSNTEEDCEQELEDLLYCLDHCHSQ |
| SEQ ID NO: 32 | GSREGCASRCMKYNDELEKCEARMMSMSNTEEDCEQELEDLLYCLDHCHSQ |
| SEQ ID NO: 1 | GSREGCASRCTKYNAELEKCEARVSSMSNTEETCVQELFDLLHCVDHCVSQ |
| SEQ ID NO: 2 | GSREGCASRCTKYNAELEKCEARVMSMSNTEEDCEQELEDLLHCLDHCHSQ |
| SEQ ID NO: 3 | GSREGCASRCTKYNAELEKCEARVVSMSNTEEDCEQELEDLLHCLDHCHSQ |
| SEQ ID NO: 4 | GSREGCASRCTKYNAELEKCEARVMSMSNTEETCEQELEDLLHCLDHCHSQ |
| SEQ ID NO: 5 | GSREGCASRCTKYNAELEKCEARVMSMSNTEEDCVQELEDLLHCLDHCHSQ |
| SEQ ID NO: 6 | GSREGCASRCTKYNAELEKCEARVMSMSNTEEDCEQELFDLLHCLDHCHSQ |
| SEQ ID NO: 7 | GSREGCASRCTKYNAELEKCEARVMSMSNTEEDCEQELEDLLHCVDHCHSQ |
| SEQ ID NO: 8 | GSREGCASRCTKYNAELEKCEARVMSMSNTEEDCEQELEDLLHCLDHCVSQ |
| SEQ ID NO: 9 | GSREGCASRCTKYNAELEKCEARVVSMSNTEETCEQELEDLLHCLDHCHSQ |
| SEQ ID NO: 10 | GSREGCASRCTKYNAELEKCEARVVSMSNTEEDCVQELEDLLHCLDHCHSQ |
| SEQ ID NO: 11 | GSREGCASRCTKYNAELEKCEARVVSMSNTEEDCEQELFDLLHCLDHCHSQ |
| SEQ ID NO: 12 | GSREGCASRCTKYNAELEKCEARVVSMSNTEEDCEQELEDLLHCVDHCHSQ |
| SEQ ID NO: 13 | GSREGCASRCTKYNAELEKCEARVVSMSNTEEDCEQELEDLLHCLDHCVSQ |
| SEQ ID NO: 14 | GSREGCASRCTKYNAELEKCEARVMSMSNTEETCVQELEDLLHCLDHCHSQ |
| SEQ ID NO: 15 | GSREGCASRCTKYNAELEKCEARVMSMSNTEETCEQELFDLLHCLDHCHSQ |
| SEQ ID NO: 16 | GSREGCASRCTKYNAELEKCEARVMSMSNTEETCEQELEDLLHCVDHCHSQ |
| SEQ ID NO: 17 | GSREGCASRCTKYNAELEKCEARVMSMSNTEETCEQELEDLLHCLDHCVSQ |
| SEQ ID NO: 18 | GSREGCASRCTKYNAELEKCEARVMSMSNTEEDCVQELFDLLHCLDHCHSQ |
| SEQ ID NO: 19 | GSREGCASRCTKYNAELEKCEARVMSMSNTEEDCVQELEDLLHCVDHCHSQ |
| SEQ ID NO: 20 | GSREGCASRCTKYNAELEKCEARVMSMSNTEEDCVQELEDLLHCLDHCVSQ |
| SEQ ID NO: 21 | GSREGCASRCTKYNAELEKCEARVMSMSNTEEDCEQELFDLLHCVDHCHSQ |
| SEQ ID NO: 22 | GSREGCASRCTKYNAELEKCEARVMSMSNTEEDCEQELFDLLHCLDHCVSQ |
| SEQ ID NO: 23 | GSREGCASRCTKYNAELEKCEARVMSMSNTEEDCEQELEDLLHCVDHCVSQ |
| SEQ ID NO: 24 | GSREGCASRCMKYNAELEKCEARVMSMSNTEEDCEQELEDLLHCLDHCHSQ |
| SEQ ID NO: 25 | GSREGCASRCTKYNDELEKCEARVMSMSNTEEDCEQELEDLLHCLDHCHSQ |
| SEQ ID NO: 26 | GSREGCASRCTKYNAELEKCEARMMSMSNTEEDCEQELEDLLHCLDHCHSQ |
| SEQ ID NO: 27 | GSREGCASRCTKYNAELEKCEARVMSMSNTEEDCEQELEDLLYCLDHCHSQ |
| SEQ ID NO: 28 | GSREGCASRCMKYNDELEKCEARMMSMSNTEEDCEQELEDLLHCLDHCHSQ |
| SEQ ID NO: 29 | GSREGCASRCMKYNDELEKCEARVMSMSNTEEDCEQELEDLLYCLDHCHSQ |
| SEQ ID NO: 30 | GSREGCASRCMKYNAELEKCEARMMSMSNTEEDCEQELEDLLYCLDHCHSQ |
| SEQ ID NO: 31 | GSREGCASRCTKYNDELEKCEARMMSMSNTEEDCEQELEDLLYCLDHCHSQ |
| SEQ ID NO: 33 | GSREGCASRCTRYNAELERCEARVSSMSNTEETCVQELFDLLHCVDHCVSQ |
| SEQ ID NO: 34 | GSREGCASRCTRYNAELERCEARVMSMSNTEEDCEQELEDLLHCLDHCHSQ |
| SEQ ID NO: 35 | GSREGCASRCTRYNAELERCEARVVSMSNTEEDCEQELEDLLHCLDHCHSQ |
| SEQ ID NO: 36 | GSREGCASRCTRYNAELERCEARVMSMSNTEETCEQELEDLLHCLDHCHSQ |
| SEQ ID NO: 37 | GSREGCASRCTRYNAELERCEARVMSMSNTEEDCVQELEDLLHCLDHCHSQ |
| SEQ ID NO: 38 | GSREGCASRCTRYNAELERCEARVMSMSNTEEDCEQELFDLLHCLDHCHSQ |
| SEQ ID NO: 39 | GSREGCASRCTRYNAELERCEARVMSMSNTEEDCEQELEDLLHCVDHCHSQ |
| SEQ ID NO: 40 | GSREGCASRCTRYNAELERCEARVMSMSNTEEDCEQELEDLLHCLDHCVSQ |
| SEQ ID NO: 41 | GSREGCASRCTRYNAELERCEARVVSMSNTEETCEQELEDLLHCLDHCHSQ |
| SEQ ID NO: 42 | GSREGCASRCTRYNAELERCEARVVSMSNTEEDCVQELEDLLHCLDHCHSQ |
| SEQ ID NO: 43 | GSREGCASRCTRYNAELERCEARVVSMSNTEEDCEQELFDLLHCLDHCHSQ |
| SEQ ID NO: 44 | GSREGCASRCTRYNAELERCEARVVSMSNTEEDCEQELEDLLHCVDHCHSQ |
| SEQ ID NO: 45 | GSREGCASRCTRYNAELERCEARVVSMSNTEEDCEQELEDLLHCLDHCVSQ |
| SEQ ID NO: 46 | GSREGCASRCTRYNAELERCEARVMSMSNTEETCVQELEDLLHCLDHCHSQ |
| SEQ ID NO: 47 | GSREGCASRCTRYNAELERCEARVMSMSNTEETCEQELFDLLHCLDHCHSQ |
| SEQ ID NO: 48 | GSREGCASRCTRYNAELERCEARVMSMSNTEETCEQELEDLLHCVDHCHSQ |
| SEQ ID NO: 49 | GSREGCASRCTRYNAELERCEARVMSMSNTEETCEQELEDLLHCLDHCVSQ |
| SEQ ID NO: 50 | GSREGCASRCTRYNAELERCEARVMSMSNTEEDCVQELFDLLHCLDHCHSQ |
| SEQ ID NO: 51 | GSREGCASRCTRYNAELERCEARVMSMSNTEEDCVQELEDLLHCVDHCHSQ |
| SEQ ID NO: 52 | GSREGCASRCTRYNAELERCEARVMSMSNTEEDCVQELEDLLHCLDHCVSQ |
| SEQ ID NO: 53 | GSREGCASRCTRYNAELERCEARVMSMSNTEEDCEQELFDLLHCVDHCHSQ |
| SEQ ID NO: 54 | GSREGCASRCTRYNAELERCEARVMSMSNTEEDCEQELFDLLHCLDHCVSQ |
| SEQ ID NO: 55 | GSREGCASRCTRYNAELERCEARVMSMSNTEEDCEQELEDLLHCVDHCVSQ |
| SEQ ID NO: 56 | GSREGCASRCMRYNAELERCEARVMSMSNTEEDCEQELEDLLHCLDHCHSQ |
| SEQ ID NO: 57 | GSREGCASRCTRYNDELERCEARVMSMSNTEEDCEQELEDLLHCLDHCHSQ |
| SEQ ID NO: 58 | GSREGCASRCTRYNAELERCEARMMSMSNTEEDCEQELEDLLHCLDHCHSQ |
| SEQ ID NO: 59 | GSREGCASRCTRYNAELERCEARVMSMSNTEEDCEQELEDLLYCLDHCHSQ |
| SEQ ID NO: 60 | GSREGCASRCMRYNDELERCEARMMSMSNTEEDCEQELEDLLHCLDHCHSQ |
| SEQ ID NO: 61 | GSREGCASRCMRYNDELERCEARVMSMSNTEEDCEQELEDLLYCLDHCHSQ |
| SEQ ID NO: 62 | GSREGCASRCMRYNAELERCEARMMSMSNTEEDCEQELEDLLYCLDHCHSQ |
| SEQ ID NO: 63 | GSREGCASRCTRYNDELERCEARMMSMSNTEEDCEQELEDLLYCLDHCHSQ |
| SEQ ID NO: 64 | GSREGCASRCMRYNDELERCEARMMSMSNTEEDCEQELEDLLYCLDHCHSQ |

In some embodiments, a TfR-binding peptide disclosed herein comprises GSREGCAX₁RCX₂KYX₄DEX₂X₃KCX₃ARIMIMSMSNTEEDCEQEX₂EDX₂X₂YCX₂X₃X₅CX₅X₁X₄ (SEQ ID NO: 148) or REGCAX₁RCX₂KYX₄DEX₂X₃KCX₃ARIMMSMSNTEEDCEQEX₂EDX₂X₂YCX₂X₃X₅CX₅X₁X₄ (SEQ ID NO: 167), wherein X₁ can be independently selected from S, T, D, or N, X₂ can be independently selected from A, M, I, L, or V, X₃ can be independently selected from D, E, N, Q, S, or T, X₄ can be independently selected from D, E, H, K, R, N, Q, S, or T, and X₅ can be independently selected from H, K, R, N, Q, S, or T.

In some embodiments, a TfR-binding peptide disclosed herein comprises GSREX₁CX₂X₃RCX₄KYX₅DEX₆X₇KCX₈ARMMSMSNTEEDCEQELEDLLYCLDHCHSQ (SEQ ID NO: 149) or REX₁CX₂X₃RCX₄KYX₅DEX₆X₇KCXSARMMSMSNTEEDCEQELEDLLYCLDHCHSQ (SEQ ID NO: 168), wherein X₁, X₂, X₃, X₄, X₅, X₆, X₇ and X₈ are TfR binding interface residues and can independently be any amino acid. In some embodiments, a TfR-binding peptide disclosed herein comprises GSREGCASRCMKYNDELEKCEARMMSMSNTEEDCEQEXEDX₂X₃YCX₄X₅X₆CX₇X₈X₉ (SEQ ID NO: 150) or REGCASRCMKYNDELEKCEARMMSMSNTEEDCEQEXEDX₂X₃YCX₄X₅X₆CX₇X₈X₉ (SEQ ID NO: 169), wherein X₁, X₂, X₃, X₄, X₅, X₆, X₇, X₈, and X₉ are TfR binding interface residues and can independently be any amino acid. In some embodiments, a TfR-binding peptide disclosed herein comprises GSREX₁CX₂X₃RCX₄KYX₅DEX₆X₇KCX₈ARMMSMSNTEEDCEQEX₉EDX₁₀X₁₁YCX₁₂X₁₃X₁₃CX₁₅X₁₆X₁₇ (SEQ ID NO: 151) or REX₁CX₂X₃RCX₄KYX₅DEX₆X₇KCX₈ARMMSMSNTEEDCEQEX₉EDX₁₀X₁₁YCX₁₂X₁₃X₁₃C X₁₅X₁₆X₁₇ (SEQ ID NO: 170), wherein X₁, X₂, X₃, X₄, X₅, X₆, X₇, X₈, X₉, X₁₀, X₁₁, X₁₂, X₁₃, X₁₄, X₁₅, X₁₆ and X₁₇ are TfR binding interface residues and can independently be any amino acid. In some embodiments, a TfR-binding peptide disclosed herein comprises GSREGCASRCMKYNDELEKCEARMMSMSNTEEDCEQELEDLLYCLDHCHSQ (SEQ ID NO: 32).

In some embodiments, a TfR-binding peptide disclosed herein comprises X₁X₂X₃X₄GX₅ASX₆X₇MX₈X₉NX₁₀X₁₁LEX₁₂X₁₃EX₁₄X₁₅X₁₆X₁₇X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇X₃₈X₃₉X₄₀X₄₁X₄₂X₄₃ (SEQ ID NO: 152), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, X₄₂, and X₄₃ can independently be any amino acid.

In some embodiments, a TfR-binding peptide disclosed herein comprises X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂X₂₉X₃₀X₃₁X₃₂X₃₃X₃₄X₃₅X₃₆X₃₇LX₃₈X₃₉LLX₄₀X₄₁LDHX₄₂HSQ (SEQ ID NO: 153), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, and X₄₂ can independently be any amino acid.

In some embodiments, a TfR-binding peptide disclosed herein comprises X₁X₂X₃X₄GX₅ASX₆X₇MX₈X₉NX₁₀X₁₁LEX₁₂X₁₃EX₁₄X₁₅X₁₆X₁₇X₁₈X₁₉X₂₀X₂₁X₂₂X₂₃X₂₄X₂₅X₂₆X₂₇X₂₈X₂₉LX₃₀X₃₁LLX₃₂X₃₃LDHX₃₄HSQ (SEQ ID NO: 154), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, and X₃₄ can independently be any amino acid.

In some embodiments, a TfR-binding peptide or peptide complex disclosed herein comprises at least 60%, at least 65%, at least 70%, at least 75%, at least 80%, at least 85%, at least 90%, at least 91%, at least 92%, at least 93%, at least 94%, at least 95%, at least 96%, at least 97%, at least 98%, at least 99%, or at least 100% sequence homology to any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64, or any variant, homolog, or functional fragment thereof. In some embodiments, a TfR-binding peptide or peptide complex disclosed herein comprises any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64, or any variant, homolog, or functional fragment thereof. In some embodiments, a peptide that binds to a TfR comprises the amino acid sequence set forth in SEQ ID NO: 32.

In some embodiments, a TfR-binding peptide comprises canonical amino acid residues as surface interface residues at any one of the corresponding positions 5, 7, 8, 14, 17, 18, 21, 38, 42, 45, 46, 47, 50, 51, with reference to SEQ ID NO: 32 or a combination thereof. In some embodiments, a TfR-binding peptide comprises canonical amino acid residues as surface interface residues at any one of the corresponding positions G5, A7, S8, N14, L17, E18, E21, L38, L42, L45, D46, H47, S50, Q51, with reference to SEQ ID NO: 32 or a combination thereof. In some embodiments, the peptide or peptide complex of the present disclosure comprises at least one or more of these corresponding residues in SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. Such peptides can accordingly be engineered with enhanced binding to TfR. In some embodiments, a TfR-binding peptide disclosed herein comprises X₁X₂X₃X₄GX₅ASX₆X₇X₈X₉X₁₀NX₁₁X₁₂LEX₁₃X₁₄EX₁₅X₁₆X₁₇X₁₈X₁₉X₂₀X₂₁X₂₂X₂₃X₂₄X₂₅X₂₆X₂₇X₂₈X₂₉X₃₀LX₃₁X₃₂X₃₃LX₃₄X₃₅LDHX₃₆X₃₇SQ (SEQ ID NO: 155), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, and X₃₇ can independently be any amino acid.

In some embodiments, surface-distal hydrophilic amino acid residues (e.g., D, E, H, K, R, N, Q, S, or T) present in the amino acid sequence of a peptide contribute to peptide solubility. In some embodiments, a peptide as disclosed herein comprises a hydrophilic amino acid residue at any one of the corresponding positions 3, 4, 9, 11, 15, 16, 19, 23, 26, 28, 29, 30, 31, 32, 33, 35, 36, 37, 39, 40, with reference to SEQ ID NO: 32, or any combination thereof. In some instances, a peptide of the present disclosure comprises hydrophilic amino acid residues at the following corresponding positions: R3, E4, R9, K12, D15, E16, K19, R23, S26, S28, N29, T30, E31, E32, D33, E35, Q36, E37, E39, D40, with reference to SEQ ID NO: 32, or any combination thereof. In some embodiments, any one of or any combination of corresponding positions R3, E4, R9, K12, D15, E16, K19, R23, S26, S28, N29, T30, E31, E32, D33, E35, Q36, E37, E39, D40 with reference to SEQ ID NO: 32, can be mutated to another hydrophilic residue without significantly impacting solubility or TfR-binding. In some embodiments, a TfR-binding peptide disclosed herein comprises X₁X₂REX₃X₄X₅X₆RX₇X₈KX₉X₁₀DEX₁₁X₁₂KX₁₃X₁₄X₁₅RX₁₆X₁₇SX₁₈SNTEEDX₁₉EQEX₂₀EDX₂₁X₂₂X₂₃X₂₄X₂₅X₂₆X₂₇X₂₈X₂₉X₃₀X₃₁ (SEQ ID NO: 156), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, and X₃₁ can independently be any amino acid. In some embodiments, a TfR-binding peptide disclosed herein comprises GSX₁X₂GCASX₃CMX₄YNX₅X₆LEX₇CEAX₈MMX₉MX₁₀X₁₁X₁₂X₁₃X₁₄X₁₅CX₁₆X₁₇X₁₈LX₁₉X₂₀LLYCLDHCHSQ (SEQ ID NO: 157) or X₁X₂GCASX₃CMX₄YNX₅X₆LEX₇CEAX₈MMX₉MX₁₀X₁₁X₁₂X₁₃X₁₄X₁₅CX₁₆X₁₇X₁₈LX₁₉X₂₀L LYCLDHCHSQ (SEQ ID NO: 171), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, and X₂₀ can be independently selected from D, E, H, K, R, N, Q, S, or T.

In some embodiments, a peptide of the present disclosure comprises cysteine amino acid residues at corresponding positions 4, 8, 18, 32, 42, and 46 with reference to SEQ ID NO: 96. In some embodiments, a peptide of the present disclosure comprises cysteine amino acid residues at corresponding positions 6, 10, 20, 34, 44, and 48 with reference to SEQ ID NO: 32. In some embodiments, a peptide of the present disclosure comprises hydrophilic residues (e.g., D, E, H, K, R, N, Q, S, or T) at corresponding positions 15, 35, 39, 49, with reference to SEQ ID NO: 32, or any combination thereof. In some instances, a peptide of the present disclosure comprises hydrophilic amino acid residues at the following corresponding positions: D15, E35, E39, H49, with reference to SEQ ID NO: 32, or any combination thereof. In some embodiments, any one of or any combination of corresponding positions D15, E35, E39, H49 with reference to SEQ ID NO: 32, can be mutated to another hydrophilic residue without significantly impacting solubility or TfR-binding. In some embodiments, a TfR-binding peptide disclosed herein comprises. In some embodiments, a TfR-binding peptide disclosed herein comprises X₁X₂X₃X₄X₅X₆X₇X₈X₉X₁₀X₁₁X₁₂X₁₃X₁₄DX₁₅X₁₆X₁₇X₁₈X₁₉X₂₀X₂₁X₂₂X₂₃X₂₄X₂₅X₂₆X₂₇X₂₈X₂₉X₃₀X₃₁X₃₂X₃₃EX₃₄X₃₅X₃₆EX₃₇X₃₈X₃₉X₄₀X₄₁X₄₂X₄₃X₄₄X₄₅HX₄₆X₄₇ (SEQ ID NO: 158), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, X₄₂, X₄₃, X₄₄, X₄₅, X₄₆, and X₄₇ can independently be any amino acid. In some embodiments, a TfR-binding peptide disclosed herein comprises GSREGCASRCMKYNX₁ELEKCEARMMSMSNTEEDCX₂QELX₃DLLYCLDHCX₄SQ (SEQ ID NO: 159) or REGCASRCMKYNX₁ELEKCEARMMSMSNTEEDCX₂QELX₃DLLYCLDHCX₄SQ (SEQ ID NO: 172), wherein X₁, X₂, X₃, and X₄ can be independently selected from D, E, H, K, R, N, Q, S, or T.

In some embodiments, a peptide of the present disclosure comprises hydrophobic residues (e.g., A, M, I, L, V, F, W, or Y) at corresponding positions 15, 35, 39, 49, with reference to SEQ ID NO: 32, or any combination thereof. In some embodiments, a TfR-binding peptide disclosed herein comprises GSREGCASRCMKYNX₁ELEKCEARMMSMSNTEEDCX₂QELX₃DLLYCLDHCX₄SQ (SEQ ID NO: 160) or REGCASRCMKYNX₁ELEKCEARMMSMSNTEEDCX₂QELX₃DLLYCLDHCX₄SQ (SEQ ID NO: 173), wherein X₁, X₂, X₃, and X₄ can be independently selected from A, M, I, L, V, F, W, or Y. In some embodiments, hydrophilic amino acid residues at any one of the corresponding positions 15, 35, 39, and 49, with reference to SEQ ID NO: 32, are associated with higher binding affinity for TfR (e.g., target engagement) and higher solubility. In some embodiments, mutation of an amino acid residue at any one of the corresponding positions 15, 35, 39, and 49, with reference to SEQ ID NO: 32, from a hydrophobic to a hydrophilic residue can lead to higher binding affinity for TfR (e.g., target engagement) and higher solubility.

In some embodiments, a peptide of the present disclosure comprises hydrophobic residues (e.g., A, M, I, L, V, F, W, or Y) at corresponding positions 11, 25, 27, with reference to SEQ ID NO: 32, or any combination thereof. In some embodiments, a peptide of the present disclosure comprises hydrophilic residues (e.g., D, E, H, K, R, N, Q, S, or T) at corresponding positions 11, 25, 27, with reference to SEQ ID NO: 32, or any combination thereof. In some embodiments, hydrophobic amino acid residues at any one of the corresponding positions 11, 25, and 27, with reference to SEQ ID NO: 32, are associated with higher binding affinity for TfR (e.g., target engagement) and higher solubility. In some embodiments, mutation of an amino acid residue at any one of the corresponding positions 11, 25, and 27, with reference to SEQ ID NO: 32, from a hydrophilic residue to a hydrophobic residue can lead to higher binding affinity for TfR (e.g., target engagement) and higher solubility. In some embodiments, a peptide of the present disclosure comprises hydrophobic amino acid residues at the corresponding positions M11, M25, M27, with reference to SEQ ID NO: 32, or any combination thereof. In some instances, a peptide comprises the hydrophobic amino acid residues at the corresponding positions M11, M25, and M27, with reference to SEQ ID NO: 32. In some embodiments, any combination of the corresponding positions M11, M25, and M27, with reference to SEQ ID NO: 32, can be mutated to another hydrophobic residue without significantly impacting solubility or TfR-binding. In some embodiments, a TfR-binding peptide disclosed herein comprises X₁X₂X₃X₄X₅X₆X₇X₈X₉X₁₀MX₁₁X₁₂X₁₃X₁₄X₁₅X₁₆X₁₇X₁₈X₁₉X₂₀X₂₁X₂₂X₂₃MX₂₄MX₂₅X₂₆X₂₇X₂₈X₂₉X₃₀X₃₁X₃₂X₃₃X₃₄X₃₅X₃₆X₃₇X₃₈X₃₉X₄₀X₄₁X₄₂X₄₃X₄₄X₄₅X₄₆X₄₇X₄₈ (SEQ ID NO: 161), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, X₄₂, X₄₃, X₄₄, X₄₅, X₄₆, X₄₇, and X₄₈ can independently be any amino acid. In some embodiments, a TfR-binding peptide disclosed herein comprises GSREGCASRCX₁KYNDELEKCEARMX₂SX₃SNTEEDCEQELEDLLYCLDHCHSQ (SEQ ID NO: 162) or REGCASRCX₁KYNDELEKCEARMX₂SX₃SNTEEDCEQELEDLLYCLDHCHSQ (SEQ ID NO: 174), wherein X₁, X₂, and X₃ can be independently selected from A, M, I, L, V, F, W, or Y.

In some embodiments, a TfR-binding peptide disclosed herein comprises GSREGCASRCX₁KYNDELEKCEARMX₂SX₃SNTEEDCEQELEDLLYCLDHCHSQ (SEQ ID NO: 163) or REGCASRCX₁KYNDELEKCEARMX₂SX₃SNTEEDCEQELEDLLYCLDHCHSQ (SEQ ID NO: 175), wherein X₁, X₂, and X₃ can be independently selected from D, E, H, K, R, N, Q, S, or T.

In some embodiments, a peptide of the present disclosure comprises an aliphatic amino acid residue (e.g., A, M, I, L, or V) at corresponding position 45, with reference to SEQ ID NO: 32. In some embodiments, a peptide of the present disclosure comprises an aromatic amino acid residue (e.g., F, W, or Y) at corresponding position 45. In some embodiments, an aliphatic amino acid residue at corresponding position 45 is associated with higher binding affinity to TfR. In some instances, a peptide comprises the aliphatic amino acid residue corresponding to L45, with reference to SEQ ID NO: 32. In some embodiments, mutation of an amino acid residue at corresponding position 45 from an aromatic residue to an aliphatic reside can lead to higher binding affinity for TfR (e.g., target engagement) and higher solubility. In some embodiments, mutating corresponding position L45 to another aliphatic residue may not significantly impact solubility or TfR-binding. In some embodiments, a TfR-binding peptide disclosed herein comprises X₁X₂X₃X₄X₅X₆X₇X₈X₉X₁₀X₁₁X₁₂X₁₃X₁₄X₁₅X₁₆X₁₇X₁₈X₁₉X₂₀X₂₁X₂₂X₂₃X₂₄X₂₅X₂₆X₂₇X₂₈X₂₉X₃₀X₃₁X₃₂X₃₃X₃₄X₃₅X₃₆X₃₇X₃₈X₃₉X₄₀X₄₁X₄₂X₄₃X₄₄LX₄₅X₄₆X₄₇X₄₈X₄₉X₅₀ (SEQ ID NO: 164), wherein X₁, X₂, X₃, X₄, X₅, X₆, X₇, X₈, X₉, X₁₀, X₁₁, X₁₂, X₁₃, X₁₄, X₁₅, X₁₆, X₁₇, X₁₈, X₁₉, X₂₀, X₂₁, X₂₂, X₂₃, X₂₄, X₂₅, X₂₆, X₂₇, X₂₈, X₂₉, X₃₀, X₃₁, X₃₂, X₃₃, X₃₄, X₃₅, X₃₆, X₃₇, X₃₈, X₃₉, X₄₀, X₄₁, X₄₂, X₄₃, X₄₄, X₄₅, X₄₆, X₄₇, X₄₈, X₄₉, and X₅₀ can independently be any amino acid. In some embodiments, a TfR-binding peptide disclosed herein comprises GSREGCASRCMKYNDELEKCEARMMSMSNTEEDCEQELEDLLYCXIDHCHSQ (SEQ ID NO: 165) or REGCASRCMKYNDELEKCEARMMSMSNTEEDCEQELEDLLYCXIDHCHSQ (SEQ ID NO: 176), wherein X₁ can be independently selected from A, M, I, L, or V.

In some embodiments, a peptide of the present disclosure comprises GSREGCASRCMX₁YNDELEX₂CEARMMSMSNTEEDCEQELEDLLYCLDHCHSQ (SEQ ID NO: 166) or REGCASRCMX₁YNDELEX₂CEARMMSMSNTEEDCEQELEDLLYCLDHCHSQ (SEQ ID NO: 177), wherein X₁ and X₂ can be independently selected from K or R. In some embodiments, these residues at corresponding position 12 and 19, with reference to SEQ ID NO: 32, can be used for chemical conjugation to another molecule (e.g., an active or a detectable agent). In some embodiments, X₁ and X₂ are both R and chemical conjugation occurs at the N-terminus of the peptide.

In some embodiments, a receptor-binding peptide may be derived from an antibody or antibody fragment. For example, a receptor-binding peptide may be derived from a single chain antibody fragment (scFv). Examples of TfR-binding peptides that may be incorporated into a selective depletion complex of the present disclosure include SEQ ID NO: 220 (QVQLQESGGGVVQPGRSLRLSCAASRFTFSSYAMHWVRQAPGKGLEWVAVISYDGSN KYYADSVKGRFTISRDNSKNTLYLQMNSLRAEDTAVYYCARDLSGYGDYPDYWGQGT LVTVSSGGGGSGGGGSGGGGSSELTQDPAVSVALGQTVRITCQGDSLRSYYASWYQQK PGQAPVLVMYGRNERPSGVPDRFSGSKSGTSASLAISGLQPEDEANYYCAGWDDSLTG PVFGGGTKLTVLG), SEQ ID NO: 221 (QVQLQESGGGVVQPGRSLRLSCAASRFTFNNYAMHWVRQAPGKGLEWVAVISYDGS NKYYADSVKGRFTISRDNSKNTLYLQMNSLRAEDTAVYYCARDL SGYGDYPDYWGQ GTLVTVSSGGGGSGGGGSGGGGSSELTQDPAVSVALGQTVRITCQGDSLRSYYASWYQ QKPGQAPVLVMYGRNERPSGVPDRFSGSKSGTSASLAISGLQPEDEANYYCAGWDDSL TGPVFGGGTKLTVLG), and SEQ ID NO: 222 (QVQLQESGGGVVQPGRSLRLSCAASRYPFHHHIDHHWVRQAPGKGLEWVAVISYDGS NKYYADSVKGRFTISRDNSKNTLYLQMNSLRAEDTAVYYCARDLSGYGDYPDYWGQ GTLVTVSSGGGGSGGGGSGGGGSSELTQDPAVSVALGQTVRITCQGDSLRSYYASWYQ QKPGQAPVLVMYGRNERPSGVPDRFSGSKSGTSASLAISGLQPEDEANYYCAGWDDSL TGPVFGGGTKLTVLG). In some embodiments, a TfR-binding peptide may have a sequence of any one of SEQ ID NO: 220-SEQ ID NO: 222, or a fragment thereof. In some embodiments, a TfR-binding peptide may have a sequence that has at least 80%, at least 90%, at least 92%, at least 93%, at least 94%, at least 95%, at least 96%, at least 97%, at least 98%, or at least 99% sequence identity with any one of SEQ ID NO: 220-SEQ ID NO: 222, or a fragment thereof. In some embodiments, a peptide of SEQ ID NO: 220 or SEQ ID NO: 221 may function as a pH-independent TfR-binding peptide. In some embodiments, a peptide of SEQ ID NO: 222 may function as a pH-dependent TfR-binding peptide.

In some embodiments, mutations in any one or more of the amino acid residues of a peptide of the present disclosure can improve binding affinity of the peptide to TfR. In some embodiments, mutations in 5-80% of amino acid residues of a peptide of the present disclosure improve the binding affinity of the peptide to TfR. In some embodiments, mutations in 1-100%, 5-100%, or 5-50% of amino acid residues of a peptide of the present disclosure improve binding affinity of the peptide to TfR. In some embodiments, mutations in 15-50% of amino acid residues of a peptide of the present disclosure improve binding affinity of the peptide to TfR. In some embodiments, mutations in 15-30% of amino acid residues of a peptide of the present disclosure improve binding affinity of the peptide to TfR. In some embodiments, mutations in 25-30% of amino acid residues of a peptide of the present disclosure improve binding affinity of the peptide to TfR. For example, mutations in 14 of the 51 amino acid residues (27.5%) of a peptide having a sequence of SEQ ID NO: 32 can improve binding affinity of the peptide to TfR.

In some embodiments, mutations in any one or more of the amino acid residues of a peptide of the present disclosure can lie at the binding interface of TfR. In some embodiments, a mutation to a peptide can improve binding affinity, which can be beneficial to binding and transcytosis of a peptide or peptide complex disclosed herein. In some embodiments, the peptides provided herein can have many mutations or few mutations to obtain optimal activity, wherein optimal activity is sufficient binding for engagement of the TfR, but not necessarily binding that is so strong as to preclude release of the peptide and/or peptide complex after transcytosis. Thus, peptides of the present disclosure can comprise a number of mutations (also referred to as % mutated amino acid residues) that tune binding affinity and off rate to obtain optimal binding, function (e.g., transcytosis, BBB-penetration, cell membrane penetration, transport across a biological barrier, endocytosis, recycling, or combinations thereof), and release of the peptide or peptide complex. Thus, mutations that result in the highest possible affinity may not necessarily correlate to a superior peptide having optimal binding and transcytosis.

In some embodiments, 1-100% or 5-100% of amino acid residues of a peptide of the present disclosure lie at the binding interface of TfR. In some embodiments, 10-90% of amino acid residues of a peptide of the present disclosure lie at the binding interface of TfR. In some embodiments, 20-80% of amino acid residues of a peptide of the present disclosure lie at the binding interface of TfR. In some embodiments, 30-70% of amino acid residues of a peptide of the present disclosure lie at the binding interface of TfR. In some embodiments, 40-60% of amino acid residues of a peptide of the present disclosure lie at the binding interface of TfR. In some embodiments, 30-35% of amino acid residues of a peptide of the present disclosure lie at the binding interface of TfR. For example, 17 of the 51 amino acid residues (33%) of a peptide having a sequence of SEQ ID NO: 32 can lie at the binding interface of TfR.

In some embodiments, mutations in any one or more of the amino acid residues of a peptide of the present disclosure that lie at the binding interface of TfR can improve binding affinity of the peptide to TfR. In some embodiments, mutations in 1-100% or 5-100% of amino acid residues of a peptide of the present disclosure that lie at the binding interface of TfR improve binding affinity of the peptide to TfR. In some embodiments, mutations in 5-80% of amino acid residues of a peptide of the present disclosure that lie at the binding interface of TfR improve binding affinity of the peptide to TfR. In some embodiments, mutations in 10-70% of amino acid residues of a peptide of the present disclosure that lie at the binding interface of TfR improve binding affinity of the peptide to TfR. In some embodiments, mutations in 15-60% of amino acid residues of a peptide of the present disclosure that lie at the binding interface of TfR improve binding affinity of the peptide to TfR. In some embodiments, mutations in 20-50% of amino acid residues of a peptide of the present disclosure that lie at the binding interface of TfR improve binding affinity of the peptide to TfR. In some embodiments, mutations in 25-30% of amino acid residues of a peptide of the present disclosure that lie at the binding interface of TfR improve binding affinity of the peptide to TfR. For example, mutations in 5 of the 17 amino acid residues (29%) of a peptide having a sequence of SEQ ID NO: 32 that lie at the binding interface of TfR and can improve binding affinity of the peptide to TfR.

In some embodiments, mutations in any one or more of the amino acid residues of a peptide of the present disclosure are distal to the binding interface of TfR. In some embodiments, 1-100% or 5-100% of amino acid residues of a peptide of the present disclosure are distal to the binding interface of TfR. In some embodiments, 10-90% of amino acid residues of a peptide of the present disclosure are distal to the binding interface of TfR. In some embodiments, 20-80% of amino acid residues of a peptide of the present disclosure are distal to the binding interface of TfR. In some embodiments, 30-70% of amino acid residues of a peptide of the present disclosure are distal to the binding interface of TfR. In some embodiments, 40-60% of amino acid residues of a peptide of the present disclosure are distal to the binding interface of TfR. In some embodiments, 65-70% of amino acid residues of a peptide of the present disclosure are distal to the binding interface of TfR. For example, 34 of the 51 amino acid residues (66%) of a peptide having a sequence of SEQ ID NO: 32 can lie at the binding interface of TfR.

In some embodiments, mutations in any one or more of the amino acid residues of a peptide of the present disclosure are distal to the binding interface of TfR improve binding affinity of the peptide to TfR. In some embodiments, mutations in 1-100% or 5-100% of amino acid residues of a peptide of the present disclosure that are distal to the binding interface of TfR improve binding affinity of the peptide to TfR. In some embodiments, mutations in 5-80% of amino acid residues of a peptide of the present disclosure that are distal to the binding interface of TfR improve binding affinity of the peptide to TfR. In some embodiments, mutations in 10-70% of amino acid residues of a peptide of the present disclosure that are distal to the binding interface of TfR improve binding affinity of the peptide to TfR. In some embodiments, mutations in 15-60% of amino acid residues of a peptide of the present disclosure that are distal to the binding interface of TfR improve binding affinity of the peptide to TfR. In some embodiments, mutations in 20-50% of amino acid residues of a peptide of the present disclosure that are distal to the binding interface of TfR improve binding affinity of the peptide to TfR. In some embodiments, mutations in 25-30% of amino acid residues of a peptide of the present disclosure that are distal to the binding interface of TfR improve binding affinity of the peptide to TfR. For example, mutations in 5 of the 17 amino acid residues that are distal to the binding interface of TfR can improve binding affinity of the peptide to TfR. For example, mutations in 9 of the 34 amino acid residues (26.5%) of a peptide having a sequence of SEQ ID NO: 32 that are distal to the binding interface of TfR can improve binding affinity of the peptide to TfR. In some embodiments, and without being bound to any theory, one or more mutations in the amino acid residues of the peptide that are distal to the binding interface of TfR can improve protein folding, enhance protein solubility, and/or alter the backbone geometry that can improve binding through an optimized interface shape complementarity.

In some embodiments, a receptor-binding peptide of the present disclosure may be a PD-L1-binding peptide. The PD-L1-binding peptide may be incorporated into a selective depletion complex of the present disclosure to facilitate selective depletion of a target molecule via PD-L1-mediated endocytosis. In some embodiments, the PD-L1-binding peptide that is a receptor-binding peptide may bind PD-L1 with an affinity that is pH-independent (for example, a similar affinity at extracellular pH and at an endosomal pH) or may bind PD-L1 with an affinity that is pH-dependent (for example, a higher affinity at extracellular pH and a lower affinity at an endosomal pH). Examples of PD-L1-binding peptides are provided in TABLE 2.

TABLE 2
Exemplary PD-L1-Binding Peptides

| SEQ ID NO | Target-Binding Peptide Sequence |
|---|---|
| SEQ ID NO: 187 | EEDCKVHCVKEWMAGKACAERQKSYTIGRAHCSGQKFDVFKCLDHCAAP |
| SEQ ID NO: 233 | EHDCKVHCVKEWMAGHACAERQKSYTIGRAHCSGQKFDVFKCLDHCAAP |
| SEQ ID NO: 234 | EHDCKVHCVKEWMAGKACAERQKSYTIGRAHCSGQKFDVFKCLDHCAAP |
| SEQ ID NO: 235 | EEDCKVHCVKEWHAGKACAERQKSYTIGRAHCSGQKFDVFKCLDHCAAP |
| SEQ ID NO: 236 | EEDCKVHCVKEWMAGHACAERQKSYTIGRAHCSGQKFDVFKCLDHCAAP |
| SEQ ID NO: 237 | EHDCKVHCVKEWHAGKACAERQKSYTIGRAHCSGQKFDVFKCLDHCAAP |
| SEQ ID NO: 238 | EEDCKVHCVKEWHAGHACAERQKSYTIGRAHCSGQKFDVFKCLDHCAAP |
| SEQ ID NO: 239 | EHDCKVHCVKEWHAGHACAERQKSYTIGRAHCSGQKFDVFKCLDHCAAP |
| SEQ ID NO: 400 | EEDCKVHCVKEWMAGKACAERDKSYTIGRAHCSGQKFDVFKCLDHCAAP |
| SEQ ID NO: 401 | EEDCKVHCVKEWMAGKACAERNKSYTIGRAHCSGQKFDVFKCLDHCAAP |
| SEQ ID NO: 402 | EEDCKVHCVKEWAAYKACAERIKSYTIGRAHCSGQYFDVWKCLDHCAAP |
| SEQ ID NO: 403 | ARTCESQSHRFKGPCVSMTNCASVCRTERFSGGHCRGFRRRCLCTKHC |
| SEQ ID NO: 404 | ARTCESQSHRFKGPCVSDTNCASVCYTERFSGGHCRGFRRRCLCTKHC |
| SEQ ID NO: 405 | ARTCESQSHRFKGPCVSDTNCASVCRTERFSGGHCMGFRRRCLCTKHC |
| SEQ ID NO: 406 | EEDCKVHCVKWWMAGKACAERNKSYTIGRAHCSGQKFDVFKCLDHCAAP |
| SEQ ID NO: 407 | EEDCKVHCVKWWAAGKACAERNKSYTIGRAHCSGQKFDVFKCLDHCAAP |
| SEQ ID NO: 408 | EEDCKVHCVKWWMAYKACAERNKSYTIGRAHCSGQKFDVFKCLDHCAAP |
| SEQ ID NO: 409 | EEDCKVHCVKWWMAGKACAERIKSYTIGRAHCSGQKFDVFKCLDHCAAP |
| SEQ ID NO: 410 | EEDCKVHCVKWWMAGKACAERNKSYTIGRAHCSGQYFDVFKCLDHCAAP |
| SEQ ID NO: 411 | EEDCKVHCVKWWMAGKACAERNKSYTIGRAHCSGQKFDVWKCLDHCAAP |
| SEQ ID NO: 412 | EEDCKVHCVKWWAAYKACAERIKSYTIGRAHCSGQYFDVWKCLDHCAAP |
| SEQ ID NO: 413 | EEDCKVHCVKEWMAYKACAERIKSYTIGRAHCSGQYFDVWKCLDHCAAP |
| SEQ ID NO: 414 | EEDCKVHCVKEWAAGKACAERIKSYTIGRAHCSGQYFDVWKCLDHCAAP |
| SEQ ID NO: 415 | EEDCKVHCVKEWAAYKACAERNKSYTIGRAHCSGQYFDVWKCLDHCAAP |
| SEQ ID NO: 416 | EEDCKVHCVKEWAAYKACAERIKSYTIGRAHCSGQKFDVWKCLDHCAAP |
| SEQ ID NO: 417 | EEDCKVHCVKEWAAYKACAERIKSYTIGRAHCSGQYFDVFKCLDHCAAP |
| SEQ ID NO: 418 | EEDCKVHCVKEWMAGKACAERIKSYTIGRAHCSGQYFDVWKCLDHCAAP |
| SEQ ID NO: 419 | EEDCKVHCVKEWMAYKACAERNKSYTIGRAHCSGQYFDVWKCLDHCAAP |
| SEQ ID NO: 420 | EEDCKVHCVKEWMAYKACAERIKSYTIGRAHCSGQKFDVWKCLDHCAAP |
| SEQ ID NO: 421 | EEDCKVHCVKEWMAYKACAERIKSYTIGRAHCSGQYFDVFKCLDHCAAP |
| SEQ ID NO: 422 | EEDCKVHCVKEWAAGKACAERNKSYTIGRAHCSGQYFDVWKCLDHCAAP |
| SEQ ID NO: 423 | EEDCKVHCVKEWAAGKACAERIKSYTIGRAHCSGQKFDVWKCLDHCAAP |
| SEQ ID NO: 424 | EEDCKVHCVKEWAAGKACAERIKSYTIGRAHCSGQYFDVFKCLDHCAAP |
| SEQ ID NO: 425 | EEDCKVHCVKEWAAYKACAERNKSYTIGRAHCSGQKFDVWKCLDHCAAP |
| SEQ ID NO: 426 | EEDCKVHCVKEWAAYKACAERNKSYTIGRAHCSGQYFDVFKCLDHCAAP |
| SEQ ID NO: 427 | EEDCKVHCVKEWAAYKACAERIKSYTIGRAHCSGQKFDVFKCLDHCAAP |
| SEQ ID NO: 428 | EEDCKVHCVKEWMAGKACAERQKSDTTGQAHCSGQKFDVFKCLDHCAAP |
| SEQ ID NO: 429 | EEDCKVHCVKEWMAGKACAERNKSDTTGQAHCSGQKFDVFKCLDHCAAP |
| SEQ ID NO: 430 | EEDCKVHCVKEWAAYKACAERIKSDTTGQAHCSGQYFDVWKCLDHCAAP |
| SEQ ID NO: 431 | EEDCKVHCVKWWMAGKACAERNKSDTTGQAHCSGQKFDVFKCLDHCAAP |
| SEQ ID NO: 432 | EEDCKVHCVKWWAAGKACAERNKSDTTGQAHCSGQKFDVFKCLDHCAAP |
| SEQ ID NO: 433 | EEDCKVHCVKWWMAYKACAERNKSDTTGQAHCSGQKFDVFKCLDHCAAP |
| SEQ ID NO: 434 | EEDCKVHCVKWWMAGKACAERIKSDTTGQAHCSGQKFDVFKCLDHCAAP |
| SEQ ID NO: 435 | EEDCKVHCVKWWMAGKACAERNKSDTTGQAHCSGQYFDVFKCLDHCAAP |
| SEQ ID NO: 436 | EEDCKVHCVKWWMAGKACAERNKSDTTGQAHCSGQKFDVWKCLDHCAAP |
| SEQ ID NO: 437 | EEDCKVHCVKWWAAYKACAERIKSDTTGQAHCSGQYFDVWKCLDHCAAP |
| SEQ ID NO: 438 | EEDCKVHCVKEWMAYKACAERIKSDTTGQAHCSGQYFDVWKCLDHCAAP |
| SEQ ID NO: 439 | EEDCKVHCVKEWAAGKACAERIKSDTTGQAHCSGQYFDVWKCLDHCAAP |
| SEQ ID NO: 440 | EEDCKVHCVKEWAAYKACAERNKSDTTGQAHCSGQYFDVWKCLDHCAAP |
| SEQ ID NO: 441 | EEDCKVHCVKEWAAYKACAERIKSDTTGQAHCSGQKFDVWKCLDHCAAP |
| SEQ ID NO: 442 | EEDCKVHCVKEWAAYKACAERIKSDTTGQAHCSGQYFDVFKCLDHCAAP |
| SEQ ID NO: 443 | EEDCKVHCVKEWMAGKACAERIKSDTTGQAHCSGQYFDVWKCLDHCAAP |
| SEQ ID NO: 444 | EEDCKVHCVKEWMAYKACAERNKSDTTGQAHCSGQYFDVWKCLDHCAAP |
| SEQ ID NO: 445 | EEDCKVHCVKEWMAYKACAERIKSDTTGQAHCSGQKFDVWKCLDHCAAP |
| SEQ ID NO: 446 | EEDCKVHCVKEWMAYKACAERIKSDTTGQAHCSGQYFDVFKCLDHCAAP |
| SEQ ID NO: 447 | EEDCKVHCVKEWAAGKACAERNKSDTTGQAHCSGQYFDVWKCLDHCAAP |
| SEQ ID NO: 448 | EEDCKVHCVKEWAAGKACAERIKSDTTGQAHCSGQKFDVWKCLDHCAAP |
| SEQ ID NO: 449 | EEDCKVHCVKEWAAGKACAERIKSDTTGQAHCSGQYFDVFKCLDHCAAP |
| SEQ ID NO: 450 | EEDCKVHCVKEWAAYKACAERNKSDTTGQAHCSGQKFDVWKCLDHCAAP |
| SEQ ID NO: 451 | EEDCKVHCVKEWAAYKACAERNKSDTTGQAHCSGQYFDVFKCLDHCAAP |
| SEQ ID NO: 452 | EEDCKVHCVKEWAAYKACAERIKSDTTGQAHCSGQKFDVFKCLDHCAAP |
| SEQ ID NO: 453 | EESCKPQCVKAWLEYQACAERVEKDESGEAHCTGQYFDLWGCVDKCVAP |
| SEQ ID NO: 454 | ARTCESQSHRFKGPCVSDTMCASVCRTERFSGGHCRGFRRRCLCSKHC |
| SEQ ID NO: 455 | EERCMPQCVKSLYEYEKCLKRVENDDTGHKHCTGHYFDYWSCIDKCVAS |
| SEQ ID NO: 456 | EEDCRVHCVREWMAGRACAERDRSYTIGRAHCSGQRFDVFRCLDHCAAP |

In some embodiments, a PD-L1-binding peptide disclosed herein comprises a sequence of X¹X²X³CS⁴X⁵X⁶CX⁷X⁸X⁹X¹⁰X¹¹X¹²X¹³X¹⁴X¹⁵CX¹⁶X¹⁷X¹⁸X¹⁹X²⁰X²¹X²²X²³X²⁴XR²⁵X²⁶X²⁷X²⁸C X²⁹X³⁰X³¹X³²X³³X³⁴X³⁵X³⁶X³⁷CX³⁸X³⁹X⁴⁰CX⁴¹X⁴²X⁴³ (SEQ ID NO: 392), wherein X¹ can independently be selected from E, M, V, or W; X² can independently be selected from G, E, L, or F; X³ can independently be selected from D, E, or 5; X⁴ can independently be selected from K, R, or V; X⁵ can independently be selected from E, Q, 5, M, L, or V; X⁶ can independently be selected from D, E, H, K, R, N, Q, 5, or Y; X⁷ can independently be selected from D, M, or V; X⁸ can independently be selected from A, K, R, Q, 5, or T; X⁹ can independently be selected from A, D, E, H, Q, 5, T, M, I, L, V, or W; X¹⁰ can independently be selected from A, E, R, Q, 5, T, W, or P; X¹¹ can independently be selected from A, E, K, R, N, Q, T, M, I, L, V, or W; X¹² can independently be selected from G, A, E, K, N, T, or Y; X¹³ can independently be selected from G, A, D, E, H, K, R, N, Q, 5, T, M, I, L, V, W, Y, or P; X¹⁴ can independently be selected from D, K, R, N, L, or V; X¹⁵ can independently be selected from G, A, D, T, L, W, or P; X¹⁶ can independently be selected from G, A, E, H, K, N, 5, F, or P; X¹⁷ can independently be selected from G, A, D, E, N, or P; X¹⁸ can independently be selected from G, D, H, K, R, N, Q, S, T, V, or Y; X¹⁹ can independently be selected from G, D, E, H, K, N, Q, S, T, M, I, F, W, Y, or P; X²¹ can independently be selected from G, A, D, E, H, K, R, N, Q, S, Y, or P; X²¹ can independently be selected from G, A, D, H, N, Q, S, V, F, or P; X²² can independently be selected from A, D, H, N, Q, S, T, M, I, V, Y, or P; X²³ can independently be selected from G, A, D, K, R, T, W, or Y; X²⁴ can independently be selected from G, A, E, N, Q, T, I, V, or P; X²⁵ can independently be selected from G, D, N, Q, T, L, V, F, or P; X²⁶ can independently be selected from G, A, E, K, R, N, Q, S, T, I, Y, or P; X²⁷ can independently be selected from A, D, N, or I; X²⁸ can independently be selected from G, D, E, H, N, F, or W; X²⁹ can independently be selected from G, A, E, N, S, Y, or P; X³¹ can independently be selected from G, M, or L; X³¹ can independently be selected from G, A, D, K, N, Q, or W; X³² can independently be selected from D, E, H, K, N, Q, S, T, L, V, F, Y, or P; X³³ can independently be selected from G, E, Q, or F; X³⁴ can independently be selected from D or K; X³⁵ can independently be selected from G, V, or P; X³⁶ can independently be selected from G, H, R, V, F, W, or P; X³⁷ can independently be selected from A, D, or K; X³⁸ can independently be selected from E, H, Q, L, or F; X³⁹ can independently be selected from D, E, R, S, T, M, L, or F; X⁴⁰ can independently be selected from G, A, D, E, H, K, R, M, L, or P; X⁴¹ can independently be selected from G, A, K, S, I, or L; X⁴² can independently be selected from G, A, D, E, R, Q, T, or F; and X⁴³ can independently be selected from A, H, N, Q, S, F, or P.

In some embodiments, a binding peptide disclosed herein comprises a sequence of EEDCKVX¹CVX¹X¹X¹X¹X²X³KX¹CX¹EX¹X⁴X¹X¹X¹X¹X¹X¹X¹AX¹CX¹GX¹X⁵FX⁶VFX⁶CLX¹X¹CX¹X¹X¹ (SEQ ID NO: 393), wherein X¹ can independently be selected from any non-cysteine amino acid; X² can independently be selected from M, I, L, or V; X³ can independently be selected from Y, A, H, K, R, N, Q, S, or T; X⁴ can independently be selected from D, E, N, Q, or P; X⁵ can independently be selected from K or P; and X⁶ can independently be selected from D or K.

A PD-L1-binding peptide may comprise a PD-L1-binding motif that forms part or all of a binding interface with PD-L1. One or more residues of a PD-L1-binding motif may interact with one or more residues of PD-L1 at the binding interface between the PD-L1-binding peptide and PD-L1. In some embodiments, multiple PD-L1-binding motifs may be present in a PD-L1-binding peptide. A PD-L1-binding motif may comprise a sequence of CX¹X²X³CX⁴X⁵X⁶X⁷X⁸X⁹X¹⁰X¹¹X¹²C (SEQ ID NO: 394), wherein X¹ can independently be selected from K, R, or V; X² can independently be selected from E, Q, S, M, L, or V; X³ can independently be selected from D, E, H, K, R, N, Q, S, or Y; X⁴ can independently be selected from D, M, or V; X⁵ can independently be selected from A, K, R, Q, S, or T; X⁶ can independently be selected from A, D, E, H, Q, S, T, M, I, L, V, or W; X⁷ can independently be selected from A, E, R, Q, S, T, W, or P; X⁸ can independently be selected from A, E, K, R, N, Q, T, M, I, L, V, or W; X⁹ can independently be selected from G, A, E, K, N, T, or Y; X¹⁰ can independently be selected from G, A, D, E, H, K, R, N, Q, S, T, M, I, L, V, W, Y, or P; X¹¹ can independently be selected from D, K, R, N, L, or V; and X¹² can independently be selected from G, A, D, T, L, W, or P. In some embodiments, a PD-L1-binding motif may comprise a sequence of CKVX¹CVX¹X¹X¹X¹X²X³KX¹C (SEQ ID NO: 396), wherein X¹ can independently be selected from any non-cysteine amino acid; X² can independently be selected from M, I, L, or V; and X³ can independently be selected from Y, A, H, K, R, N, Q, S, or T. In some embodiments, a PD-L1-binding motif may comprise a sequence of CKVHCVKEWMAGKAC (SEQ ID NO: 398). In some embodiments, a PD-L1-binding motif may comprise at least about 70%, at least about 75%, at least about 80%, at least about 85%, at least about 90%, at least about 95%, at least about 97%, or at least about 99% identity to SEQ ID NO: 398.

A PD-L1-binding motif may comprise a sequence of X¹X²X³X⁴X⁵X⁶CX⁷X⁸X⁹C (SEQ ID NO: 395), wherein X¹ can independently be selected from D, E, H, K, N, Q, S, T, L, V, F, Y, or P; X² can independently be selected from G, E, Q, or F; X³ can independently be selected from D or K; X⁴ can independently be selected from G, V, or P; X⁵ can independently be selected from G, H, R, V, F, W, or P; X⁶ can independently be selected from A, D, or K; X⁷ can independently be selected from E, H, Q, L, or F; X⁸ can independently be selected from D, E, R, S, T, M, L, or F; and X⁹ can independently be selected from G, A, D, E, H, K, R, M, L, or P. In some embodiments, a PD-L1-binding motif may comprise a sequence of XFX²VFX²CLX³X³C (SEQ ID NO: 397), wherein X¹ can independently be selected from K or P; X² can independently be selected from D or K; and X³ can independently be selected from any non-cysteine amino acid. In some embodiments, a PD-L1-binding motif may comprise a sequence of KFDVFKCLDHC (SEQ ID NO: 399). In some embodiments, a PD-L1-binding motif may comprise at least about 70%, at least about 75%, at least about 80%, at least about 85%, at least about 90%, at least about 95%, at least about 97%, or at least about 99% identity to SEQ ID NO: 399.

A PD-L1-binding peptide (e.g., any one of SEQ ID NO: 187, SEQ ID NO: 233-SEQ ID NO: 239, SEQ ID NO: 400-SEQ ID NO: 456, or SEQ ID NO: 240, or a pH-independent variant thereof) with high affinity PD-L1-binding at endosomal pH may be complexed with a target-binding peptide as described herein to form a selective depletion complex for selective depletion of the target molecule. The selective depletion complex can be used to selectively deliver a target molecule across a cellular layer or membrane. For example, the selective depletion complex can be used to selectively deliver the target molecule to an endocytic compartment via PD-L1-mediated endocytosis. The target molecule can be selectively depleted upon binding to the target-binding peptide of the selective depletion complex and endocytosis via PD-L1-mediated endocytosis as described.

Selective depletion of a target molecule using PD-L1-mediated endocytosis may be used to selectively deplete the target molecule specifically in tissues that express PD-L1. In some embodiments, a selective depletion complex comprising a receptor-binding peptide that binds PD-L1 may be used to selectively deplete a target molecule in a PD-L1 positive cancer, a lung tissue, a pancreatic islet tissue, a lymphoid tissue, a gastrointestinal tissue, a bone marrow tissue, a reproductive tissue, a muscle tissue, an adipose tissue, or any other PD-L1 positive tissue. For example, a selective depletion complex comprising a PD-L1-binding peptide and an ACE2-binding peptide may be used to selectively deplete ACE2 in lung tissue to prevent a viral infection (e.g., a SARS-CoV-2 infection). In another example, a selective depletion complex comprising a PD-L1-binding peptide and an HLA-binding peptide may be used to selectively deplete HLA in pancreatic islet cells to prevent T-cell attack of insulin-expressing cells in type I diabetes.

A PD-L1-binding peptide (e.g., any one of SEQ ID NO: 187, SEQ ID NO: 233-SEQ ID NO: 239, SEQ ID NO: 400-SEQ ID NO: 456, or SEQ ID NO: 240) may function as a target-binding peptide or a receptor-binding peptide in a selective depletion complex. In some embodiments, a selective depletion complex to selectively deplete PD-L1 may comprise a receptor-binding peptide that does not bind PD-L1 (e.g., a TfR-binding peptide) and a PD-L1-binding peptide (e.g., a pH dependent PD-L1-binding peptide). In some embodiments, a selective depletion complex to selective deplete a target that is not PD-L1 may comprise a target-binding peptide that binds the target (e.g., an EGFR-binding peptide) and a PD-L1-binding peptide (e.g., a pH-independent PD-L1-binding peptide).

### Target-Binding Peptides

Peptides, peptide complexes, or selective depletion complexes of the present disclosure can comprise a target-binding peptide. The target-binding peptide can be capable of binding a target molecule (e.g., a target protein). In some embodiments, the target-binding peptide can bind to the target molecule with an affinity that is pH-dependent. For example, the target-binding peptide can bind the target molecule with a higher affinity at an extracellular pH (such as about pH 7.4) than at an endosomal pH (such as about pH 5.5). A target-binding peptide can be conjugated to a receptor-binding peptide of the present disclosure (e.g., a TfR-binding peptide any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 or a PD-L1-binding peptide of any one of SEQ ID NO: 187, SEQ ID NO: 233-SEQ ID NO: 239, SEQ ID NO: 400-SEQ ID NO: 456, or SEQ ID NO: 241) to form a selective depletion complex. The selective depletion complex can be used to selectively deliver a target molecule across a cellular layer or membrane (e.g., BBB or cell membrane). For example, the selective depletion complex can be used to selectively deliver the target molecule to an endocytic compartment via receptor-mediated endocytosis (e.g., PD-L1-mediated endocytosis or TfR-mediated endocytosis). The target molecule can be selectively depleted upon binding to the target-binding peptide of the selective depletion complex and endocytosis via receptor-mediated endocytosis.

The target molecule can be a soluble molecule. For example, the target molecule can be a secreted peptide or protein, a cell signaling molecule, an extracellular matrix macromolecule (e.g., collagen, elastin, microfibrillar protein, or proteoglycan), a neurotransmitter, a cytokine, a growth factor, a tumor associated antigen, a tumor specific antigen, or a hormone. The target molecule can be a cell surface molecule. For example, the target molecule can be a transmembrane protein, a receptor, including a growth factor receptor, a checkpoint inhibitor, an immune checkpoint inhibitor, an inhibitory immune receptor, a ligand of an inhibitory immune receptor, a macrophage surface protein (e.g., CD14 or CD16), a lipopolysaccharide, or an antibody. An inhibitory immune receptor may be CD200R, CD300a, CD300f, CEACAM1, FcgRiib, ILT-2, ILT-3, ILT-4, ILT-5, LAIR-1, PECAM-1, PILR-alpha, SIRL-1, and SIRP-alpha, CLEC4A, Ly49Q, MICL. Selective depletion of a cell surface molecule using a selective depletion complex comprising a target-binding peptide that binds to the cell surface molecule can result in a reduction of the cell surface molecule (e.g., a surface exposed protein). The surface exposed protein can be associated with a disease or a condition. In some embodiments, a selective depletion complex of the present disclosure can comprise two or more target-binding peptides to promote dimerization of a target molecule. Promoting dimerization can increase internalization of the target molecule, resulting in selective depletion of the target molecule. For example, a selective depletion complex comprising two copies of a target-binding peptide can promote homodimerization of the target molecule. In some embodiments, a target-binding peptide of the present disclosure may comprise a miniprotein, a nanobody, an antibody, an IgG, an antibody fragment, a Fab, a F(ab)2, an scFv, an (scFv)2, a DARPin, or an affibody. In some embodiments, the target-binding peptide may comprise a cystine-dense peptide, an affitin, an adnectin, an avimer, a Kunitz domain, a nanofittin, a fynomer, a bicyclic peptide, a beta-hairpin, or a stapled peptide.

In some embodiments, a target-binding peptide of the present disclosure can bind to the target molecule with an affinity that is pH-dependent. For example, the target-binding peptide can bind the target molecule at an extracellular pH (such as about pH 7.4) with an affinity that is higher than the binding affinity at an endocytic pH (such as about pH 7.0, pH 6.5, pH 6.0, or pH 5.5). In some embodiments, the binding affinity of the target-binding peptide for the target molecule at an extracellular pH (about pH 7.4) can be at least about 1.1-fold, at least about 1.2-fold, at least about 1.3-fold, at least about 1.4-fold, at least about 1.5-fold, at least about 1.6-fold, at least about 1.7-fold, at least about 1.8-fold, at least about 1.9-fold, at least about 2-fold, at least about 2.5-fold, at least about 3-fold, at least about 3.5-fold, at least about 4-fold, at least about 4.5-fold, at least about 5-fold, at least about 6-fold, at least about 7-fold, at least about 8-fold, at least about 9-fold, at least about 10-fold, at least about 15-fold, at least about 20-fold, at least about 25-fold, at least about 30-fold, at least about 35-fold, at least about 40-fold, at least about 45-fold, at least about 50-fold, at least about 60-fold, at least about 70-fold, at least about 80-fold, at least about 90-fold, at least about 100-fold, at least about 200-fold, at least about 500-fold, at least about 1000-fold, at least about 10,000-fold the binding affinity of the target-binding peptide for the target molecule at an endosomal pH (such as about pH 7.0, pH 6.5, pH 6.0, pH 5.5, or pH 5.0). In some embodiments, the affinity of the target-binding peptide for the target at pH 6.5 or pH 5.5 is no greater than about 0.1%, about 0.5%, about 1%, about 5%, about 10%, about 20%, about 30%, about 40%, or about 50% the affinity of the target binding peptide for the target at pH 7.4. In some embodiments, the affinity of the target-binding peptide for the target at pH 7.4 is at least 2-fold, at least 3-fold, at least 4-fold, at least 5-fold, at least 6-fold, at least 7-fold, at least 8-fold, at least 9-fold, at least 10-fold, at least 15-fold, or at least 20-fold greater than the affinity of the target-binding peptide for the target at pH 6.5 or pH 5.5

In some embodiments, a target-binding peptide with pH-dependent binding can bind a target molecule with a dissociation constant (K_{D}) of less than 50 μM, less than 5 μM, less than 500 nM, less than 100 nM, less than 40 nM, less than 30 nM, less than 20 nM, less than 10 nM, less than 5 nM, less than 2 nM, less than 1 nM, less than 0.5 nM, less than 0.4 nM, less than 0.3 nM, less than 0.2 nM, or less than 0.1 nM at extracellular pH (such as about pH 7.4). In some embodiments, a target-binding peptide with pH-dependent binding can bind a target molecule with a dissociation constant (K_{D}) of at least 1 nM, at least 2 nM, at least 5 nM, at least 10 nM, at least 20 nM, at least 50 nM, at least 100 nM, at least 200 nM, at least 500 nM, at least 1 μM, at least 2 μM, at least 5 μM, at least 10 μM, at least 20 μM, at least 50 μM, at least 100 μM, at least 500 μM, at least 1 mM, at least 2 mM, at least 5 mM, at least 10 mM, at least 20 mM, at least 50 mM, at least 100 mM, at least 200 mM, at least 500 mM, or at least 1 M at endosomal pH (about pH 5.5 or about pH 6.5).

In some embodiments, the target-binding molecule can release the target molecule upon internalization into an endosomal compartment and acidification of the endosome. Such release the target molecule upon acidification of the endosome can occur at about pH 7.3, pH 7.2, pH 7.1, pH 7.0, pH 6.9, pH 6.8, pH 6.7, pH 6.6, pH 6.5, pH 6.4, pH 6.3, pH 6.2, pH 6.1, pH 6.0, pH 5.9, pH 5.8, pH 5.7, pH 5.6, pH 5.5, pH 5.4, pH 5.3, pH 5.2, pH 5.1, pH 5.0, pH 4.9, pH 4.8, pH 4.7, pH 4.6, pH 4.5, or lower. In some embodiments, release of the target molecule can occur at a pH of from about pH 7.0 to about pH 4.5, from about pH 6.5 to about pH 5.0, or from about pH 6.0 to about pH 5.5 or lower.

Target-binding peptides with pH-dependent binding affinity can be engineered by selective integration of histidine (His) amino acid residues in the target binding interface. In some instances, a target-binding peptide with pH-dependent binding affinity comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 histidine residues in the target binding interface. Since the side chain of histidine is predominantly uncharged at pH between about 6.0 and about 9.2 and predominantly positively charged at pH below about 6.0, selectively inserting or removing His residues in a target-binding peptide can impart pH-dependent binding properties. A target-binding peptide (e.g., a target-binding peptide with pH-dependent binding affinity) can comprise a cystine-dense peptide (CDP), an affibody, a DARPin, a centyrin, a nanofittin, or an adnectin. A target-binding CDP, a target-binding affibody, a target-binding adnectin can be stable at low pH (e.g., at endosomal pH). In some embodiments, a target-binding peptide can comprise an antibody (e.g., IgG or other antibody), an antibody fragment, (e.g., scFv, scFv2, Fab, F(ab)2, or other antibody fragment), or a nanobody (e.g., a VHH-domain nanobody or VNAR-domain nanobody from camelids or sharks), which can be stable at a low pH.

In some embodiments, release of the target molecule by the target-binding peptide upon internalization into an endosomal compartment can be affected by differences in the ionic strength between the extracellular physiologic environment and endosomal cellular compartments. In some embodiments, the ionic strength of the endosomal compartment is higher than the ionic strength of the extracellular physiologic environment. Ionic strength, which varies with salt concentration, may depend on the concentrations of various electrolytes in solution, for example hydrogen (H⁺), hydroxide (OH®), hydronium (H₃O⁺), sodium (Na⁺), potassium (K⁺), calcium (Ca²⁺), magnesium (Mg²⁺), manganese (Mn²⁺), chloride (Cl⁻), carbonate (CO₃²⁻), cobalt (Co²⁺), phosphate (PO₄³⁻), or nitrate (NO₃⁻). In some embodiments, target-binding peptides with salt-dependent or ionic strength-dependent binding affinity can be engineered by selective integration of salt labile moieties (e.g., polar or charged amino acid side chains) in the target binding interface that would enable dissociation of the target-binding molecule in the endosome. For example, the target binding interface of the target-binding peptide may form one or more polar or charge-charge interactions with the target-binding peptide that can be disrupted as the ionic strength of the environment increases.

In some instances, a target-binding peptide with a binding affinity dependent on ionic strength (e.g., dependent on hydrogen, hydroxide, hydronium, sodium, potassium, calcium, magnesium, manganese, chloride, carbonate, cobalt, phosphate, and/or nitrate concentration) could dissociate over a range of ionic strengths, for example ionic strengths from about 30 mM to about 1 M. In some embodiments, an ionic strength-dependent target-binding peptide with a binding affinity dependent on ionic strength could dissociate at an ionic strength of from about 50 mM to about from about 50 mM to about 1 M, from about 60 mM to about 950 mM, from about 70 mM to about 900 mM, from about 80 mM to about 850 mM, from about 90 mM to about 800 mM, from about 100 mM to about 750 mM, from about 110 mM to about 700 mM, from about 120 mM to about 650 mM, from about 130 mM to about 600 mM, from about 140 mM to about 550 mM, from about 150 mM to about 500 mM, from about 160 mM to about 450 mM, from about 170 mM to about 400 mM, from about 180 mM to about 350 mM, from about 190 mM to about 300 mM, or from about 200 mM to about 250 mM. In some embodiments, the ionic strength-dependent target-binding peptide comprises at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, or at least 20 polar or charge-charge interactions in the target binding interface.

A target-binding peptide of the present disclosure may bind to a target molecule, such as a target molecule with clinical relevance. In some embodiments, a target molecule may be a soluble molecule, extracellular molecule, or cell-surface molecule. In some embodiments, the target molecule is a protein, peptide, lipid, carbohydrate, a nucleic acid, or glycan. In some embodiments, a target molecule may be a protein that is over-expressed or over-activated in a disease or condition. For example, a target molecule may be a transmembrane protein involved in oncogenic signaling, immune suppression, or pro-inflammatory signaling. Examples of target molecules that may be targeted by a target-binding peptide of the present disclosure include but are not limited to CD3, CD47, CD28, CD137, CD89, CD16, CD29, CD44, CD71, CD73, CD90, CD105, CD166, CD27, CD39, CD24, CD25, CD74, CD40L, MUC1, MUC16, MUC2, MUC5AC, MUC4, OX40, 4-1BB, HLA-G, LAG3, Tim3, TIGIT, GITR, TCR, TNF-α, EGFR, EGFRvIII, TKI-resistant EGFR, HER2, ERBB3, PDGFR, FGF, VEGF, VEGFR, IGFR1, CTLA4, STRO1, complement factor C4, complement factor C1q, complement factor C1s, complement factor C1r, complement factor C3, complement factor C3a, complement factor C3b, complement factor C5, complement factor C5a, TGFβ, PCSK9, P2Y6, HER3, RANK, tau, amyloid ß, huntingtin, α-synuclein, glucocerebrosidase, α-glucosidase, IL-1, IL-1R, IL-1β, IL-2, IL-2R, IL-4, IL-5, IL-6, IL-6R, IL-10, IL-10R, IL-17, IL-23, IL-12, p40, a member of the B7 family, c-Met, SIGLEC, MCP-1, an MHC, an MHC I, an MHC II, PD-1, and PD-L1. Additional examples of target molecules include mannose-6-phosphate glycans, glucose-6-phosphate, and sugar-specific receptors (e.g., lectins). Additional examples of target molecules include autoantibodies, such as rheumatoid factor, antinuclear antibody, antineutrophil cytoplasmic antiboides, anti-dsDNA, anticentromere antibodies, anithistone antibodies, cyclic citriullinated peptide antibodies, extractable nuclear antigen antibodies, cardiolipin antibodies, beta-2 glycoprotein 1 antibodies, antiphospholipid antibodies, lupus anticoagulants, diabetes-related autoantibodies, anti-tissue translugtaminase, anti-gliadin antibodies, intrinsic factor antibodies, parietal cell antibodies, thyroid autoantibodies, smooth muscle antibodies, antimitochronrial antibodies, liver kidney microsome type 1 antibodies, anti-glomerular basement membrane, acetylcholine receptor antibodies. The target molecule (e.g., CD3, CD47, CD28, CD137, CD89, CD16, CD29, CD44, CD71, CD73, CD90, CD105, CD166, CD27, CD39, CD24, CD25, CD74, CD40L, MUC1, MUC16, MUC2, MUC5AC, MUC4, OX40, 4-1BB, HLA-G, LAG3, Tim3, TIGIT, GITR, TCR, TNF-α, EGFR, EGFRvIII, TKI-resistant EGFR, HER2, ERBB3, PDGFR, FGF, VEGF, VEGFR, IGFR1, CTLA4, STRO1, complement factor C4, complement factor C1q, complement factor C1s, complement factor C1r, complement factor C3, complement factor C3a, complement factor C3b, complement factor C5, complement factor C5a, TGFβ, PCSK9, P2Y6, HER3, RANK, tau, amyloid ß, huntingtin, α-synuclein, glucocerebrosidase, α-glucosidase, IL-1, IL-1R, IL-1α, IL-1β, IL-2, IL-2R, IL-4, IL-5, IL-6, IL-6R, IL-10, IL-10R, IL-17, IL-23, IL-12, p40, a member of the B7 family, c-Met, SIGLEC, MCP-1, an MHC, an MHC I, an MHC II, PD-1, or PD-L1) may be endocytosed and degraded upon binding to the target-binding peptide of a selective depletion complex.

In some embodiments, a target molecule may be a transmembrane protein, such as a receptor tyrosine kinase. Examples of receptor tyrosine kinases that may be targeted using a selective depletion complex include EGF receptor, ErbB, Insulin receptor, PDGF receptor, VEGF receptor, FGF receptor, CCK receptor, NGF receptor, HGF receptor, Eph receptor, AXL receptor, TIE receptor, RYK receptor, DDR receptor, RET receptor, ROS receptor, LTK receptor, ROR receptor, MuSK receptor, and LMR receptor. Targeting the transmembrane protein using a selective depletion complex may lead to internalization and degradation of the transmembrane protein. In some embodiments, a target molecule may be a pathogen (e.g., a virus or a bacteria) or a pathogen surface molecule (e.g., a protein or a glycoprotein). For example, the target molecule may be a coronavirus spike protein, an influenza virus hemagglutinin, or a herpes simplex virus glycoprotein M. Targeting the pathogen or the pathogen surface protein using a selective depletion complex may lead to internalization and degradation of the pathogen, thereby treating or preventing an infection caused by the pathogen.

Endocytosis and subsequent degradation of the target molecule may treat (e.g., eliminate, reduce, slow progression of, or treat symptoms of) a disease or condition associated with the target molecule. In some embodiments, targeting and degradation of a receptor tyrosine kinase with a selective depletion complex may be beneficial in treating a variant of cancers. For example, targeting and degrading EGFR with a selective depletion complex comprising an EGFR-binding peptide may be beneficial in treating cancers, such as non-small-cell lung cancer, primary non-small-cell lung cancer, metastatic non-small-cell lung cancer, head and neck cancer, head and neck squamous cell carcinoma, glioblastoma, brain cancer, metastatic brain cancer, colorectal cancer, colon cancer, tyrosine kinase inhibitor (TKI)-resistant cancer, cetuximab-resistant cancer, necitumumab-resistant cancer, panitumumab-resistant cancer, local cancer, regionally advanced cancer, recurrent cancer, metastatic cancer, refractory cancer, KRAS wildtype cancer, KRAS mutant cancers, or exon20 mutant non-small-cell lung cancer. In another example, targeting and degrading TNF-α with a selective depletion complex comprising a TNF-α-binding peptide may be beneficial in treating inflammatory or neurological conditions, including those in the CNS, such as neuroinflammation, neuroinflammatory disease, stroke, traumatic brain injury, Alzheimer's disease, or other tauopathies including neurofibrillary tangle dementia, chronic traumatic encephalopathy (CTE), aging-related tau astrogliopathy, frontotemporal dementia, parkinsonism, progressive supranuclear palsy, corticobasal degeneration, lytico-bodig disease, ganglioglioma, meningioangiomatosis, or subacute sclerosing panencephalitis. For example, targeting and degrading TNF-α with a selective depletion complex comprising a TNF-α-binding peptide may also be beneficial in treating inflammatory conditions that may not be localized to the CNS (e.g., ankylosing spondylitis, antiphospholipid antibody syndrome, gout, inflammatory arthritis center, myositis, rheumatoid arthritis, scleroderma, Sjogren's disease, systemic lupus erythematosus (lupus), vasculitis, psoriasis, inflammatory bowel disease, Crohn's disease, or ulcerative colitis). A selective depletion complex of the present disclosure can be used to target pathogenic immune complexes, such as those in circulation. Circulating antigen-antibody complexes can be involved in autoimmune and inflammatory diseases as well as in malignancy. This can include glomerulonephritis, systemic lupus erythematosus (lupus), rheumatoid arthritis, and cutaneous vasculitis.

A selective depletion complex of the present disclosure can be used to target a complement pathway in a complement-mediated disease, such as facioscapulohumeral muscular dystrophy (FSHD) or schizophrenia. Such selective depletion complexes may be well-suited for treatment of FSDH since TfR is highly expressed on muscle cells, so efficient degradation of complement pathway component(s) would be expected. In some embodiments, targeting and degrading complement factor C4, or factors upstream (e.g., complement factor C1q, complement factor C1s, or complement factor C1r) or downstream (e.g., complement factor C3, complement factor C3a, complement factor C3b, complement factor C5, or complement factor C5a) of C4 in the complement pathway, in the CNS may treat schizophrenia. C4 is subsequently used as an exemplar of this pathway with the understanding that other complement components regulating the activation of C4 or executing the continuation of this pathway have equal standing for regulating the biological consequences of the increased activity of this pathway. As schizophrenia affects nearly 1% of humans with onset most often during adolescence, a composition comprising a selective depletion complex to treat schizophrenia would be beneficial. The complement pathway may serve as a common pathway in schizophrenia, and therapies comprising the selective depletion complexes of the present disclosure promoting degradation of C4 or a downstream complement pathway would be beneficial to patients. In some embodiments, a selective depletion complex of the present disclosure may be used to target complement-mediated diseases in the central nervous system. For example, a selective depletion complex comprising a peptide that binds one or more C4A forms could be used to target C4A long (e.g., including HERV incorporation) or short forms for degradation as described herein. Additional target molecules that may be targeted and depleted using a selective depletion complex for treatment of schizophrenia include molecules encoded by the extended MHC complex on chromosome 6, molecules encoded by the complement C4 locus (e.g., encoded by the C4Along locus or the c4Ashort locus), molecules encoded by sequences containing a single nucleotide polymorphisms in CUB and Sushi multiple domains 1 (CSMD1) gene on chromosome 8, complement factor C4, complement factor C3, or C3 receptor. Targeted degradation of complement factor C4, complement factor C3, or molecules that prevent degradation of complement factor C4 or complement factor C3 may be beneficial in treating schizophrenia. For example, a selective depletion complex of the present disclosure may treat schizophrenia by reducing excessive synaptic pruning, preventing reduction in gray matter, and preventing psychotic symptoms in patients that are predisposed to schizophrenia by polymorphisms in C4, CSMD1 or other genes. A selective depletion complex for treatment of schizophrenia (e.g., comprising a complement factor C4-binding peptide) may provide a narrow and precise form of immunosuppression which may prevent toxicities that occur when broad immunosuppression is used for long periods of time, as is common for chronic illnesses such as schizophrenia. In some embodiments, a selective depletion complex for treatment of schizophrenia may be administered in combination with an additional drug (e.g., minocycline, doxycycline, steroids, an inhibitor of C4 degradation, or an anti-psychotic agent). Additionally, the selective depletion complexes of the present disclosure may be well-suited for treatment of CNS-associated disorders such a schizophrenia due to the ability of the selective depletion complexes to penetrate the blood-brain barrier (BBB) and access the CNS via TfR-binding. A selective depletion complex (e.g., comprising a TfR-binding peptide) may facilitate higher BBB

In some embodiments, binding and subsequently depleting a target molecule using a selective depletion complex of the present disclosure comprising a target-binding peptide may be used to treat a disease or condition wherein the target molecule is a cell-based or soluble moiety associated with a disease or condition and is expressed or present in diseased tissues or cells. In some embodiments, depletion of the target molecule may be cell type or tissue dependent. For example, depletion of a target molecule may be specific to cells or tissues expressing both the target molecule targeted by the target-binding peptide of the selective depletion complex and the cell surface receptor targeted by the receptor-binding peptide of the selective depletion complex. The degradation and depletion and of the target molecule using a selective depletion complex may prevent, treat, or ameliorate the disease or condition.

In some embodiments, a target-binding peptide may comprise a sequence of any one of SEQ ID NO: 187, SEQ ID NO: 219, SEQ ID NO: 233-SEQ ID NO: 244, or SEQ ID NO: 400-SEQ ID NO: 456. In some embodiments, a target-binding peptide may comprise a sequence having at least 80%, at least 90%, at least 92%, at least 93%, at least 94%, at least 95%, at least 96, at least 97% at least 98, or at least 99% sequence identity with any one of SEQ ID NO: 233, SEQ ID NO: 187, or SEQ ID NO: 234-SEQ ID NO: 244, or a fragment thereof. For example, a target binding peptide may comprise a sequence having at least 800%, at least 900%, at least 920%, at least 9300 at least 9400 at least 9500 at least 960%, at least 9700 at least 980%, or at least 991 sequence identity with SEQ ID NO: 233, or the target binding peptide may comprise a sequence of SEQ ID NO: 233. Examples of target-binding peptides and their corresponding target molecules are provided in TABLE 3.

TABLE 3
Exemplary Target-Binding Peptides

| Target | SEQ ID NO | Target-Binding Peptide Sequence |
|---|---|---|
| PD-L1 | SEQ ID NO: 187 | EEDCKVHCVKEWMAGKACAERQKSYTIGRAHCSGQKFDVFK |
| | | CLDHCAAP |
| PD-L1 | SEQ ID NO: 233 | EHDCKVHCVKEWMAGHACAERQKSYTIGRAHCSGQKFDVFK |
| | | CLDHCAAP |
| PD-L1 | SEQ ID NO: 234 | EHDCKVHCVKEWMAGKACAERQKSYTIGRAHCSGQKFDVFK |
| | | CLDHCAAP |
| PD-L1 | SEQ ID NO: 235 | EEDCKVHCVKEWHAGKACAERQKSYTIGRAHCSGQKFDVFKC |
| | | LDHCAAP |
| PD-L1 | SEQ ID NO: 236 | EEDCKVHCVKEWMAGHACAERQKSYTIGRAHCSGQKFDVFK |
| | | CLDHCAAP |
| PD-L1 | SEQ ID NO: 237 | EHDCKVHCVKEWHAGKACAERQKSYTIGRAHCSGQKFDVFKC |
| | | LDHCAAP |
| PD-L1 | SEQ ID NO: 238 | EEDCKVHCVKEWHAGHACAERQKSYTIGRAHCSGQKFDVFKC |
| | | LDHCAAP |
| PD-L1 | SEQ ID NO: 239 | EHDCKVHCVKEWHAGHACAERQKSYTIGRAHCSGQKFDVFKC |
| | | LDHCAAP |
| PD-L1 | SEQ ID NO: 241 | DIVLTQSPATLSLSPGERATLSCRATESVEYYGTSLVQWYQQKP |
| | | GQPPKLLIYAASSVDSGVPSRFSGSGSGTDFTLTINSLEAEDAAT |
| | | YFCQQSRRVPYTFGQGTKLEIKGGGGSGGGGSGGGGSEVQLVQ |
| | | SGAEVKKPGASVKMSCKASGYTFTSYVMHWVKQAPGQRLEW |
| | | IGYVNPFNDGTKYNEMFKGRATLTSDKSTSTAYMELSSLRSED |
| | | TAVYYCARQAWGYPWGQGTLVTVSS |
| PD-L1 | SEQ ID NO: 400 | EEDCKVHCVKEWMAGKACAERDKSYTIGRAHCSGQKFDVFK |
| | | CLDHCAAP |
| PD-L1 | SEQ ID NO: 401 | EEDCKVHCVKEWMAGKACAERNKSYTIGRAHCSGQKFDVFK |
| | | CLDHCAAP |
| PD-L1 | SEQ ID NO: 402 | EEDCKVHCVKEWAAYKACAERIKSYTIGRAHCSGQYFDVWKC |
| | | LDHCAAP |
| PD-L1 | SEQ ID NO: 403 | ARTCESQSHRFKGPCVSMTNCASVCRTERFSGGHCRGFRRRCL |
| | | CTKHC |
| PD-L1 | SEQ ID NO: 404 | ARTCESQSHRFKGPCVSDTNCASVCYTERFSGGHCRGFRRRCL |
| | | CTKHC |
| PD-L1 | SEQ ID NO: 405 | ARTCESQSHRFKGPCVSDTNCASVCRTERFSGGHCMGFRRRCL |
| | | CTKHC |
| PD-L1 | SEQ ID NO: 406 | EEDCKVHCVKWWMAGKACAERNKSYTIGRAHCSGQKFDVFK |
| | | CLDHCAAP |
| PD-L1 | SEQ ID NO: 407 | EEDCKVHCVKWWAAGKACAERNKSYTIGRAHCSGQKFDVFK |
| | | CLDHCAAP |
| PD-L1 | SEQ ID NO: 408 | EEDCKVHCVKWWMAYKACAERNKSYTIGRAHCSGQKFDVFK |
| | | CLDHCAAP |
| PD-L1 | SEQ ID NO: 409 | EEDCKVHCVKWWMAGKACAERIKSYTIGRAHCSGQKFDVFKC |
| | | LDHCAAP |
| PD-L1 | SEQ ID NO: 410 | EEDCKVHCVKWWMAGKACAERNKSYTIGRAHCSGQYFDVFK |
| | | CLDHCAAP |
| PD-L1 | SEQ ID NO: 411 | EEDCKVHCVKWWMAGKACAERNKSYTIGRAHCSGQKFDVW |
| | | KCLDHCAAP |
| PD-L1 | SEQ ID NO: 412 | EEDCKVHCVKWWAAYKACAERIKSYTIGRAHCSGQYFDVWK |
| | | CLDHCAAP |
| PD-L1 | SEQ ID NO: 413 | EEDCKVHCVKEWMAYKACAERIKSYTIGRAHCSGQYFDVWK |
| | | CLDHCAAP |
| PD-L1 | SEQ ID NO: 414 | EEDCKVHCVKEWAAGKACAERIKSYTIGRAHCSGQYFDVWKC |
| | | LDHCAAP |
| PD-L1 | SEQ ID NO: 415 | EEDCKVHCVKEWAAYKACAERNKSYTIGRAHCSGQYFDVWK |
| | | CLDHCAAP |
| PD-L1 | SEQ ID NO: 416 | EEDCKVHCVKEWAAYKACAERIKSYTIGRAHCSGQKFDVWKC |
| | | LDHCAAP |
| PD-L1 | SEQ ID NO: 417 | EEDCKVHCVKEWAAYKACAERIKSYTIGRAHCSGQYFDVFKC |
| | | LDHCAAP |
| PD-L1 | SEQ ID NO: 418 | EEDCKVHCVKEWMAGKACAERIKSYTIGRAHCSGQYFDVWK |
| | | CLDHCAAP |
| PD-L1 | SEQ ID NO: 419 | EEDCKVHCVKEWMAYKACAERNKSYTIGRAHCSGQYFDVWK |
| | | CLDHCAAP |
| PD-L1 | SEQ ID NO: 420 | EEDCKVHCVKEWMAYKACAERIKSYTIGRAHCSGQKFDVWK |
| | | CLDHCAAP |
| PD-L1 | SEQ ID NO: 421 | EEDCKVHCVKEWMAYKACAERIKSYTIGRAHCSGQYFDVFKC |
| | | LDHCAAP |
| PD-L1 | SEQ ID NO: 422 | EEDCKVHCVKEWAAGKACAERNKSYTIGRAHCSGQYFDVWK |
| | | CLDHCAAP |
| PD-L1 | SEQ ID NO: 423 | EEDCKVHCVKEWAAGKACAERIKSYTIGRAHCSGQKFDVWKC |
| | | LDHCAAP |
| PD-L1 | SEQ ID NO: 424 | EEDCKVHCVKEWAAGKACAERIKSYTIGRAHCSGQYFDVFKC |
| | | LDHCAAP |
| PD-L1 | SEQ ID NO: 425 | EEDCKVHCVKEWAAYKACAERNKSYTIGRAHCSGQKEDVWK |
| | | CLDHCAAP |
| PD-L1 | SEQ ID NO: 426 | EEDCKVHCVKEWAAYKACAERNKSYTIGRAHCSGQYFDVFKC |
| | | LDHCAAP |
| PD-L1 | SEQ ID NO: 427 | EEDCKVHCVKEWAAYKACAERIKSYTIGRAHCSGQKFDVFKC |
| | | LDHCAAP |
| PD-L1 | SEQ ID NO: 428 | EEDCKVHCVKEWMAGKACAERQKSDTTGQAHCSGQKFDVFK |
| | | CLDHCAAP |
| PD-L1 | SEQ ID NO: 429 | EEDCKVHCVKEWMAGKACAERNKSDTTGQAHCSGQKFDVFK |
| | | CLDHCAAP |
| PD-L1 | SEQ ID NO: 430 | EEDCKVHCVKEWAAYKACAERIKSDTTGQAHCSGQYFDVWK |
| | | CLDHCAAP |
| PD-L1 | SEQ ID NO: 431 | EEDCKVHCVKWWMAGKACAERNKSDTTGQAHCSGQKFDVF |
| | | KCLDHCAAP |
| PD-L1 | SEQ ID NO: 432 | EEDCKVHCVKWWAAGKACAERNKSDTTGQAHCSGQKFDVFK |
| | | CLDHCAAP |
| PD-L1 | SEQ ID NO: 433 | EEDCKVHCVKWWMAYKACAERNKSDTTGQAHCSGQKFDVF |
| | | KCLDHCAAP |
| PD-L1 | SEQ ID NO: 434 | EEDCKVHCVKWWMAGKACAERIKSDTTGQAHCSGQKFDVFK |
| | | CLDHCAAP |
| PD-L1 | SEQ ID NO: 435 | EEDCKVHCVKWWMAGKACAERNKSDTTGQAHCSGQYFDVF |
| | | KCLDHCAAP |
| PD-L1 | SEQ ID NO: 436 | EEDCKVHCVKWWMAGKACAERNKSDTTGQAHCSGQKFDVW |
| | | KCLDHCAAP |
| PD-L1 | SEQ ID NO: 437 | EEDCKVHCVKWWAAYKACAERIKSDTTGQAHCSGQYFDVWK |
| | | CLDHCAAP |
| PD-L1 | SEQ ID NO: 438 | EEDCKVHCVKEWMAYKACAERIKSDTTGQAHCSGQYFDVWK |
| | | CLDHCAAP |
| PD-L1 | SEQ ID NO: 439 | EEDCKVHCVKEWAAGKACAERIKSDTTGQAHCSGQYFDVWK |
| | | CLDHCAAP |
| PD-L1 | SEQ ID NO: 440 | EEDCKVHCVKEWAAYKACAERNKSDTTGQAHCSGQYFDVWK |
| | | CLDHCAAP |
| PD-L1 | SEQ ID NO: 441 | EEDCKVHCVKEWAAYKACAERIKSDTTGQAHCSGQKFDVWK |
| | | CLDHCAAP |
| PD-L1 | SEQ ID NO: 442 | EEDCKVHCVKEWAAYKACAERIKSDTTGQAHCSGQYFDVFKC |
| | | LDHCAAP |
| PD-L1 | SEQ ID NO: 443 | EEDCKVHCVKEWMAGKACAERIKSDTTGQAHCSGQYFDVWK |
| | | CLDHCAAP |
| PD-L1 | SEQ ID NO: 444 | EEDCKVHCVKEWMAYKACAERNKSDTTGQAHCSGQYFDVW |
| | | KCLDHCAAP |
| PD-L1 | SEQ ID NO: 445 | EEDCKVHCVKEWMAYKACAERIKSDTTGQAHCSGQKFDVWK |
| | | CLDHCAAP |
| PD-L1 | SEQ ID NO: 446 | EEDCKVHCVKEWMAYKACAERIKSDTTGQAHCSGQYFDVFKC |
| | | LDHCAAP |
| PD-L1 | SEQ ID NO: 447 | EEDCKVHCVKEWAAGKACAERNKSDTTGQAHCSGQYFDVWK |
| | | CLDHCAAP |
| PD-L1 | SEQ ID NO: 448 | EEDCKVHCVKEWAAGKACAERIKSDTTGQAHCSGQKFDVWK |
| | | CLDHCAAP |
| PD-L1 | SEQ ID NO: 449 | EEDCKVHCVKEWAAGKACAERIKSDTTGQAHCSGQYFDVFKC |
| | | LDHCAAP |
| PD-L1 | SEQ ID NO: 450 | EEDCKVHCVKEWAAYKACAERNKSDTTGQAHCSGQKFDVWK |
| | | CLDHCAAP |
| PD-L1 | SEQ ID NO: 451 | EEDCKVHCVKEWAAYKACAERNKSDTTGQAHCSGQYFDVFK |
| | | CLDHCAAP |
| PD-L1 | SEQ ID NO: 452 | EEDCKVHCVKEWAAYKACAERIKSDTTGQAHCSGQKFDVFKC |
| | | LDHCAAP |
| PD-L1 | SEQ ID NO: 453 | EESCKPQCVKAWLEYQACAERVEKDESGEAHCTGQYFDLWGC |
| | | VDKCVAP |
| PD-L1 | SEQ ID NO: 454 | ARTCESQSHRFKGPCVSDTMCASVCRTERFSGGHCRGFRRRCL |
| | | CSKHC |
| PD-L1 | SEQ ID NO: 455 | EERCMPQCVKSLYEYEKCLKRVENDDTGHKHCTGHYFDYWSC |
| | | IDKCVAS |
| PD-L1 | SEQ ID NO: 456 | EEDCRVHCVREWMAGRACAERDRSYTIGRAHCSGQRFDVFRC |
| | | LDHCAAP |
| EGFR | SEQ ID NO: 219 | QVKLEESGGGSVQTGGSLRLTCAASGRTSRSYGMGWFRQAPG |
| | | KEREFVSGISWRGDSTGYADSVKGRFTISRDNAKNTVDLQMNS |
| | | LKPEDTAIYYCAAAAGSAWYGTLYEYDYWGQGTQVTVSS |
| EGFR | SEQ ID NO: 242 | QVKLEESGGGSVQTGGSLRLTCAASGHTSRSYGMGWFRQAPG |
| | | KEREFVSGISWRGDSTGYADSVKGRFTISRDNAKNTVDLQMNS |
| | | LKPEDTAIYYCAAAAGSAWYHTLYEYDYWGQGTQVTVSS |
| EGFR | SEQ ID NO: 243 | QVKLEESGGGSVQTGGSLRLTCAASGHTSRSYGMHWFRQAPG |
| | | KEREFVSGISWRGDSTGYADSVKGRFTISRDNAKNTVDLQMNS |
| | | LKPEDTAIYYCAAAAGSHWYGTHYEYDYWGQGTQVTVSS |
| EGFR | SEQ ID NO: 244 | NSDSECPLSHDGYCLHGGVCMYIKAVDRYACNCVVGYIGERC |
| | | QYRDLTWWGPR |

### Cystine-Dense Peptides

In some embodiments, TfR-binding peptides or target-binding peptides of the present disclosure comprise one or more Cys, or one or more disulfide bonds. In some embodiments, the TfR-binding peptides or the target-binding peptides are derived from cystine-dense peptides (CDPs), knotted peptides, or hitchins. As used herein, the term “peptide” is considered to be interchangeable with the terms “knotted peptide”, “cystine-dense peptide”, “CDP”, and “hitchin”. (See e.g., Correnti et al. Screening, large-scale production, and structure-based classification for cystine-dense peptides. Nat Struct Mol Biol. 2018 March; 25(3): 270-278).

The TfR-binding peptides of the present disclosure, or derivatives, fragments, or variants thereof, can be have an affinity and selectively for TfR, or a derivative or analog thereof. The target-binding peptides of the present disclosure, or derivatives, fragments, or variants thereof, can be have an affinity and selectively for a target molecule. In some cases, the TfR-binding peptides of the present disclosure can be engineered using site-saturation mutagenesis (SSM) to exhibit improved TfR-binding properties or promote transcytosis or endocytosis more effectively. In some cases, the target-binding peptides of the present disclosure can be engineered using site-saturation mutagenesis (SSM) to exhibit improved target-binding properties. In some cases, the peptides of the present disclosure are cystine-dense peptides (CDPs), related to knotted peptides or hitchin-derived peptides or knottin-derived peptides. The TfR-binding peptides can be cystine-dense peptides (CDPs). Hitchins can be a subclass of CDPs wherein six cysteine residues form disulfide bonds according to the connectivity [1-4], 2-5, 3-6 indicating that the first cysteine residue forms a disulfide bond with the fourth residue, the second with the fifth, and the third cysteine residue with the sixth. The brackets in this nomenclature indicate cysteine residues form the knotting disulfide bond. (See e.g., Correnti et al. Screening, large-scale production, and structure-based classification for cystine-dense peptides. Nat Struct Mol Biol. 2018 March; 25(3): 270-278). Knottins can be a subclass of CDPs wherein six cysteine residues form disulfide bonds according to the connectivity 1-4, 2-5, [3-6]. Knottins are a class of peptides, usually ranging from about 20 to about 80 amino acids in length that are often folded into a compact structure. Knottins are typically assembled into a complex tertiary structure that is characterized by a number of intramolecular disulfide crosslinks and can contain beta strands and other secondary structures. The presence of the disulfide bonds gives knottins and hitchins remarkable environmental stability, allowing them to withstand extremes of temperature and pH and to resist the proteolytic enzymes of the blood stream. In some cases, the peptides described herein can be derived from knotted peptides. The amino acid sequences of peptides as disclosed herein can comprise a plurality of cysteine residues. In some cases, at least cysteine residues of the plurality of cysteine residues present within the amino acid sequence of a peptide participate in the formation of disulfide bonds. In some cases, all cysteine residues of the plurality of cysteine residues present within the amino acid sequence of a peptide participate in the formation of disulfide bonds. As described herein, the term “knotted peptide” can be used interchangeably with the terms “cystine-dense peptide”, “CDP”, or “peptide”.

Provide herein are methods of identification, maturation, characterization, and utilization of CDPs that bind the transferrin receptor and allow selection, optimization and characterization of CDP-TfR binding peptides that can be used in selective depletion complexes, including for use as bioactive molecules at therapeutically relevant concentrations in a subject (e.g., a human or non-human animal). This disclosure demonstrates the utility of CDPs as a diverse scaffold family that can be screened for applicability to modern drug discovery strategies. CDPs comprise alternatives to existing biologics, primarily antibodies, which can bypass some of the liabilities of the immunoglobulin scaffold, including poor tissue permeability, immunogenicity, and long serum half-life that can become problematic if toxicities arise. Peptides of the present disclosure in the 20-80 amino acid range represent medically relevant therapeutics that are mid-sized, with many of the favorable binding specificity and affinity characteristics of antibodies but with improved stability, reduced immunogenicity, and simpler manufacturing methods. The intramolecular disulfide architecture of CDPs provides particularly high stability metrics, reducing fragmentation and immunogenicity, while their smaller size could improve tissue penetration or cell penetration and facilitate tunable serum half-life. Disclosed herein are peptides representing candidate peptides that can serve as vehicles for delivering target molecules to endocytic compartments.

In some embodiments, TfR-binding peptides can be engineered peptides. An engineered peptide can be a peptide that is non-naturally occurring, artificial, isolated, synthetic, designed, or recombinantly expressed. In some embodiments, the TfR-binding peptides of the present disclosure comprise one or more properties of CDPs, knotted peptides, or hitchins, such as stability, resistance to proteolysis, resistance to reducing conditions, and/or ability to cross the blood brain barrier. In some embodiments, the target-binding peptides of the present disclosure comprise one or more properties of CDPs, knotted peptides, or hitchins, such as stability, resistance to proteolysis, or resistance to reducing conditions.

CDPs can be advantageous for delivery to the CNS, as compared to other molecules such as antibodies due to smaller size, greater tissue or cell penetration, lack of Fc function, and quicker clearance from serum, and as compared to smaller peptides due to resistance to proteases (both for stability and for immunogenicity reduction). In some embodiments, the TfR-binding peptides or target-binding peptides of the present disclosure (e.g., CDPs, knotted peptides, or hitchins), selective depletion complexes (e.g., comprising one or more TfR-binding peptides and one or more target-binding peptides), or engineered TfR-binding fusion peptides (e.g., comprising one or more TfR-binding peptides and one or more peptides) can have properties that are superior to TfR-binding antibodies or target-binding antibodies. For example, the peptides and complexes described herein can provide superior, deeper, and/or faster tissue or cell penetration to cells and targeted tissues (e.g., brain parenchyma penetration, solid tumor penetration) and faster clearance from non-targeted tissues and serum. The TfR-binding peptides, target-binding peptides, selective depletion complexes, or TfR-binding fusion peptides of this disclosure can have lower molecular weights than TfR-binding antibodies or target-binding antibodies. The lower molecular weight can confer advantageous properties on the TfR-binding peptides, target-binding peptides, selective depletion complexes, or TfR-binding fusion peptides of this disclosure as compared to TfR-binding antibodies or target-binding antibodies. For example, the TfR-binding peptides, selective depletion complexes, or TfR-binding fusion peptides of this disclosure can penetrate a cell or tissue more readily than an anti-TfR antibody or can have lower molar dose toxicity than an anti-TfR antibody. The TfR-binding peptides, target-binding peptides, selective depletion complexes, or TfR-binding fusion peptides of this disclosure can be advantageous for lacking the Fc function of an antibody. The TfR-binding peptides, target-binding peptides, selective depletion complexes, or TfR-binding fusion peptides of this disclosure can be advantageous for allowing higher concentrations, on a molar basis, of formulations.

In some embodiments, CDPs or knotted peptides, including engineered, non-naturally occurring CDPs and those found in nature (e.g., a target-binding peptide), can be conjugated to, linked to, or fused to the TfR-binding peptides of the present disclosure, such as those described in TABLE 1, to selectively deliver a target molecule to an endocytic compartment of cell. The cell can be a cancer cell, pancreatic cell, liver cell, colon cell, ovarian cell, breast cell, lung cell, spleen cell, bone marrow cell, or any combination thereof. The cell can be any cell that expresses TfR. An engineered peptide can be a peptide that is non-naturally occurring, artificial, synthetic, designed, or recombinantly expressed. In some embodiments, a TfR-binding peptide of the present disclosure, or a complex comprising a TfR-binding peptide (e.g., a selective depletion complex), enables TfR-mediated transcytosis and/or cellular endocytosis, and the additional CDP or knotted peptide that is conjugated to, linked to, or fused to TfR-binding peptide can selectively target a molecule (e.g., an enzyme or other protein of interest) in a cell associated with a disease or condition. In some cases, the cell is a cancer cell. Cancers can include breast cancer, liver cancer, colon cancer, brain cancer, leukemia, lymphoma, non-Hodgkin lymphoma, myeloma, blood-cell-derived cancer, spleen cancer, cancers of the salivary gland, kidney cancer, muscle cancers, ovarian cancer, prostate cancer, pancreatic cancer, gastric cancer, sarcoma, glioblastoma, astrocytoma, glioma, medulloblastoma, ependymoma, choroid plexus carcinoma, midline glioma, diffuse intrinsic pontine glioma, lung cancer, bone marrow cell cancers, or skin cancer, genitourinary cancer, osteosarcoma, muscle-derived sarcoma, melanoma, head and neck cancer, a neuroblastoma, glioblastoma, astrocytoma, glioma, medulloblastoma, ependymoma, choroid plexus carcinoma, midline glioma, and diffuse intrinsic pontine glioma (DIPG), or a CMYC-overexpressing cancer. In some cases, other CDP or knotted peptides (e.g., those found in nature) are conjugated to, linked to, or fused to TfR-binding peptides and are capable of localizing TfR-binding peptides across the blood brain barrier to deliver TfR-binding peptides to target cells in the central nervous system.

CDPs (e.g., knotted peptides or hitchins) are a class of peptides, usually ranging from about 11 to about 81 amino acids in length that are often folded into a compact structure. Knotted peptides are typically assembled into a complex tertiary structure that is characterized by a number of intramolecular disulfide crosslinks and can contain beta strands, alpha helices, and other secondary structures. The presence of the disulfide bonds gives knotted peptides remarkable environmental stability, allowing them to withstand extremes of temperature and pH and to resist the proteolytic enzymes of the blood stream. The presence of a disulfide knot can provide resistance to reduction by reducing agents. The rigidity of knotted peptides also allows them to bind to targets without paying the “entropic penalty” that a floppy peptide accrues upon binding a target. For example, binding is adversely affected by the loss of entropy that occurs when a peptide binds a target to form a complex. Therefore, “entropic penalty” is the adverse effect on binding, and the greater the entropic loss that occurs upon this binding, the greater the “entropic penalty.” Furthermore, unbound molecules that are flexible lose more entropy when forming a complex than molecules that are rigidly structured, because of the loss of flexibility when bound up in a complex. However, rigidity in the unbound molecule also generally increases specificity by limiting the number of complexes that molecule can form. The peptides can bind targets with antibody-like affinity, or with nanomolar or picomolar affinity. A wider examination of the sequence structure and sequence identity or homology of knotted peptides reveals that they have arisen by convergent evolution in all kinds of animals and plants. In animals, they are often found in venoms, for example, the venoms of spiders and scorpions and have been implicated in the modulation of ion channels. The knotted proteins of plants can inhibit the proteolytic enzymes of animals or have antimicrobial activity, suggesting that knotted peptides can function in molecular defense systems found in plants.

A peptide of the present disclosure (e.g., a target-binding peptide, a TfR-binding peptide, or a selective depletion complex) can comprise a cysteine amino acid residue. In some embodiments, the peptide has at least 1, at least 2, at least 3, at least 4, at least 5, at least 6, at least 7, at least 8, at least 9, or at least 10 cysteine amino acid residues. In some embodiments, the peptide has at least 6 cysteine amino acid residues. In some embodiments, the peptide has at least 8 cysteine amino acid residues. In other embodiments, the peptide has at least 10 cysteine amino acid residues, at least 12 cysteine amino acid residues, at least 14 cysteine amino acid residues or at least 16 cysteine amino acid residues.

A knotted peptide can comprise disulfide bridges. A knotted peptide can be a peptide wherein 5% or more of the residues are cysteines forming intramolecular disulfide bonds. A disulfide-linked peptide can be a drug scaffold. In some embodiments, the disulfide bridges form a knot. A disulfide bridge can be formed between cysteine residues, for example, between cysteines 1 and 4, 2 and 5, or, 3 and 6. In some embodiments, one disulfide bridge passes through a loop formed by the other two disulfide bridges, for example, to form the knot. In other embodiments, the disulfide bridges can be formed between any two cysteine residues.

The present disclosure further includes peptide scaffolds that, e.g., can be used as a starting point for generating additional peptides. In some embodiments, these scaffolds can be derived from a variety of knotted peptides (such as CDPs or knotted peptides or hitchins). In certain embodiments, CDPs (e.g., knotted peptides or hitchins) are assembled into a complex tertiary structure that is characterized by a number of intramolecular disulfide crosslinks, and optionally contain beta strands and other secondary structures such as an alpha helix. For example, CDPs (e.g., knotted peptides) include, in some embodiments, small disulfide-rich proteins characterized by a disulfide through disulfide knot. This knot can be, e.g., obtained when one disulfide bridge crosses the macrocycle formed by two other disulfides and the interconnecting backbone. In some embodiments, the knotted peptides can include growth factor cysteine knots or inhibitor cysteine knots. Other possible peptide structures include peptide having two parallel helices linked by two disulfide bridges without β-sheets (e.g., hefutoxin).

Some peptides of the present disclosure can comprise at least one amino acid residue in an L configuration. A peptide can comprise at least one amino acid residue in D configuration. In some embodiments, a peptide is 15-75 amino acid residues long. In other embodiments, a peptide is 11-55 amino acid residues long. In still other embodiments, a peptide is 11-65 amino acid residues long. In further embodiments, a peptide is at least 20 amino acid residues long.

Some CDPs (e.g., knotted peptides) can be derived or isolated from a class of proteins known to be present or associated with toxins or venoms. In some cases, the peptide can be derived from toxins or venoms associated with scorpions or spiders. The peptide can be derived from venoms and toxins of spiders and scorpions of various genus and species. For example, the peptide can be derived from a venom or toxin of the Leiurus quinquestriatus hebraeus, Buthus occitanus tunetanus, Hottentotta judaicus, Mesobuthus eupeus, Buthus occitanus israelis, Hadrurus gertschi, Androctonus australis, Centruroides noxius, Heterometrus laoticus, Opistophthalmus carinatus, Haplopelma schmidti, Isometrus maculatus, Haplopelma huwenum, Haplopelma hainanum, Haplopelma schmidti, Agelenopsis aperta, Haydronyche versuta, Selenocosmia huwena, Heteropoda venatoria, Grammostola rosea, Ornithoctonus huwena, Hadronyche versuta, Atrax robustus, Angelenopsis aperta, Psalmopoeus cambridgei, Hadronyche infensa, Paracoelotes luctosus, and Chilobrachys jingzhaoor another suitable genus or species of scorpion or spider. In some cases, a peptide can be derived from a Buthus martensii Karsh (scorpion) toxin.

In some embodiments, a peptide of the present disclosure (e.g., a TfR-binding peptide, a target-binding peptide, or a selective depletion complex) can comprise a sequence having cysteine residues at one or more of corresponding positions 11, 12, 13, 14, 19, 20, 21, 22, 36, 38, 39, 41, for example with reference to SEQ ID NO: 96. In some embodiments, a peptide comprises Cys at corresponding positions 11, 12, 19, 20, 36, 39, or any combination thereof. For example, in certain embodiments, a peptide can comprise a sequence having a cysteine residue at corresponding position 11. In certain embodiments, a peptide can comprise a sequence having a cysteine residue at corresponding position 12. In certain embodiments, a peptide can comprise a sequence having a cysteine residue at corresponding position 13. In certain embodiments, a peptide can comprise a sequence having a cysteine residue at corresponding position 14. In certain embodiments, a peptide can comprise a sequence having a cysteine residue at corresponding position 19. In certain embodiments, a peptide can comprise a sequence having a cysteine residue at corresponding position 20. In certain embodiments, a peptide can comprise a sequence having a cysteine residue at corresponding position 21. In certain embodiments, a peptide can comprise a sequence having a cysteine residue at corresponding position 22. In certain embodiments, a peptide can comprise a sequence having a cysteine residue at corresponding position 36. In certain embodiments, a peptide can comprise a sequence having a cysteine residue at corresponding position 38. In certain embodiments, a peptide can comprise a sequence having a cysteine residue at corresponding position 39. In certain embodiments, a peptide can comprise a sequence having a cysteine residue at corresponding position 41. In some embodiments, the first cysteine residue in the sequence can be disulfide bonded with the 4th cysteine residue in the sequence, the 2nd cysteine residue in the sequence can be disulfide bonded to the 5th cysteine residue in the sequence, and the 3rd cysteine residue in the sequence can be disulfide bonded to the 6th cysteine residue in the sequence. Optionally, a peptide can comprise one disulfide bridge that passes through a ring formed by two other disulfide bridges, also known as a “two-and-through” structure system. In some embodiments, the peptides disclosed herein can have one or more cysteines mutated to serine.

In some embodiments, peptides of the present disclosure (e.g., TfR-binding peptides, target-binding peptides, or selective depletion complexes) comprise at least one cysteine residue. In some embodiments, peptides of the present disclosure comprise at least two cysteine residues. In some embodiments, peptides of the present disclosure comprise at least three cysteine residues. In some embodiments, peptides of the present disclosure comprise at least four cysteine residues. In some embodiments, peptides of the present disclosure comprise at least five cysteine residues. In some embodiments, peptides of the present disclosure comprise at least six cysteine residues. In some embodiments, peptides of the present disclosure comprise at least ten cysteine residues. In some embodiments, a peptide of the present disclosure comprises six cysteine residues. In some embodiments, a peptide of the present disclosure comprises seven cysteine residues. In some embodiments, a peptide of the present disclosure comprises eight cysteine residues.

In some embodiments, a peptide of the present disclosure (e.g., a TfR-binding peptide, a target-binding peptide, or a selective depletion complex) comprises an amino acid sequence having cysteine residues at one or more positions, for example with reference to SEQ ID NO: 96. In some embodiments, the one or more cysteine residues are located at any one of the corresponding amino acid positions 6, 10, 20, 34, 44, 48, or any combination thereof. In some aspects of the present disclosure, the one or more cysteine (C) residues participate in disulfide bonds with various pairing patterns (e.g., C₁₀-C₂₀). In some embodiments, the corresponding pairing patterns are C₆-C₄₈, C₁₀-C₄₄, and C₂₀-C₃₄. In some embodiments, the peptides as described herein comprise at least one, at least two, or at least three disulfide bonds. In some embodiments, at least one, at least two, or at least three disulfide bonds are arranged according to the corresponding C₆-C₄₈, C₁₀-C₄₄, and C₂₀-C₃₄ pairing patterns, or a combination thereof. In some embodiments, peptides as described herein comprise three disulfide bonds with the corresponding pairing patterns C₆-C₄₈, C₁₀-C₄₄, and C₂₀-C₃₄.

In certain embodiments, a peptide (e.g., a TfR-binding peptide, a target-binding peptide, or a selective depletion complex) comprises a sequence having a cysteine residue at corresponding position 6. In certain embodiments, a peptide comprises a sequence having a cysteine residue at corresponding position 10. In certain embodiments, a peptide comprises a sequence having a cysteine residue at corresponding position 20. In certain embodiments, a peptide comprises a sequence having a cysteine residue at corresponding position 34. In certain embodiments, a peptide comprises a sequence having a cysteine residue at corresponding position 44. In certain embodiments, a peptide comprises a sequence having a cysteine residue at corresponding position 50. In some embodiments, the first cysteine residue in the sequence is disulfide bonded with the last cysteine residue in the sequence. In some embodiments, the second cysteine residue in the sequence is disulfide bonded with the second to the last cysteine residue in the sequence. In some embodiments, the third cysteine residue in the sequence is disulfide bonded with the third to the last cysteine residue in the sequence and so forth.

In some embodiments, the first cysteine residue in the sequence is disulfide bonded with the 6th cysteine residue in the sequence, the 2nd cysteine residue in the sequence is disulfide bonded to the 5th cysteine residue in the sequence, and the 3rd cysteine residue in the sequence is disulfide bonded to the 4th cysteine residue in the sequence. Optionally, a peptide can comprise one disulfide bridge that passes through a ring formed by two other disulfide bridges, also known as a “two-and-through” structure system. In some embodiments, the peptides disclosed herein have one or more cysteines mutated to serine.

In some embodiments, a peptide (e.g., a TfR-binding peptide, a target-binding peptide, or a selective depletion complex) comprises no cysteine or disulfides. In some embodiments, a peptide comprises 1 or more, 2 or more, 3 or more, 4 or more, 5 or more, 6 or more, 7 or more, 8 or more, 9 or more, 10 or more, 11 or more, 12 or more, 13 or more, 14 or more, or 15 or more cysteine or disulfides. In other embodiments, 1 or more, 2 or more, 3 or more, 4 or more, 5 or more, 6 or more, 7 or more, 8 or more, 9 or more, 10 or more cysteine residues have been replaced with serine residues. In some embodiments, 1 or more, 2 or more, 3 or more, 4 or more, 5 or more, 6 or more, 7 or more, 8 or more, 9 or more, 10 or more cysteine residues have been replaced with threonine residues.

In some embodiments, a peptide (e.g., a TfR-binding peptide, a target-binding peptide, or a selective depletion complex) comprises no Cys or disulfides. In some embodiments, a peptide comprises 1 or more, 2 or more, 3 or more, 4 or more, 5 or more, 6 or more, 7 or more, 8 or more, 9 or more, 10 or more, 11 or more, 12 or more, 13 or more, 14 or more, or 15 or more Cys or disulfides. In other embodiments, 1 or more, 2 or more, 3 or more, 4 or more, 5 or more, 6 or more, 7 or more, 8 or more, 9 or more, 10 or more Cys residues have been replaced with Ser residues. In some embodiments, 1 or more, 2 or more, 3 or more, 4 or more, 5 or more, 6 or more, 7 or more, 8 or more, 9 or more, 10 or more Cys residues have been replaced with Thr residues.

In some instances, one or more or all of the methionine residues in the peptide are replaced by leucine or isoleucine. In some instances, one or more or all of the tryptophan residues in the peptide are replaced by phenylalanine or tyrosine. In some instances, one or more or all of the asparagine residues in the peptide are replaced by glutamine. In some embodiments, the N-terminus of the peptide is blocked, such as by an acetyl group. Alternatively or in combination, in some instances, the C-terminus of the peptide is blocked, such as by an amide group. In some embodiments, the peptide is modified by methylation on free amines.

For example, full methylation can be accomplished through the use of reductive methylation with formaldehyde and sodium cyanoborohydride.

In some embodiments, the peptides or peptide complexes as described herein target and/or penetrate a TfR-expressing cellular layer or barrier and/or the membrane of a TfR-expressing cell. In some embodiments, a peptide targets and/or penetrates a cell membrane of a cell, wherein said cell is located in the CNS such as the brain. For example, a peptide complex comprising a TfR-binding peptide and one or more active agents (e.g., a therapeutic or diagnostic compound) crosses a cellular barrier (e.g., BBB) via vesicular transcytosis, and subsequently targets and/or penetrates the cell membrane of a cell located within the CNS to deliver said one or more active agents to that cell.

In various embodiments, a selective depletion complex comprising a TfR-binding peptide and a target-binding peptide binds a TfR-expressing cell located in the gastrointestinal tract, spleen, liver, kidney, muscle, bone marrow, brain, or skin. In some cases, the TfR-expressing cell is a tumor cell, an immune cell, an erythrocyte, an erythrocyte precursor cell, a stem cell, a bone marrow cell, or stem cell. In some cases, the TfR-binding peptide is responsible for targeting the cell, e.g., in cases where the cell is overexpressing a TfR. In various embodiments, a peptide complex as described herein comprising a TfR-binding peptide conjugated to, linked to, or fused to a target-binding peptide binds a cell located within various organs such as the spleen, brain, liver, kidney, muscle, bone marrow, gastrointestinal tract, or skin.

In some cases, the target-biding peptides promotes endocytosis of a target molecule. In some aspects, a peptide or peptide complex (e.g., peptide conjugate or fusion peptide) of the present disclosure is used to target a target molecule in order to exert a certain biological (e.g., therapeutic) effect. In some aspects, a selective depletion complex (e.g., a complex comprising a TfR-binding peptide and a target-binding peptide) of the present disclosure is used to promote endocytosis of a target molecule into said cell to exert a certain biological effect (e.g., selective depletion of the target molecule).

### Peptide Linkers

The peptides of the presented disclosure (e.g., TfR-binding peptides, target-binding peptides, selective depletion complexes, or combinations thereof) can be dimerized in numerous ways. For example, a TfR-binding peptide can be dimerized with a target-binding peptide via a peptide linker to form a selective depletion complex. In some embodiments, a peptide linker does not disturb the independent folding of peptide domains (e.g., a TfR-binding peptide or a target-binding peptide). In some embodiments, a peptide linker can comprise sufficient length to the peptide complex so as to facilitate contact between a target molecule and a TfR via the peptide complex (e.g., a selective depletion complex). In some embodiments, a peptide linker does not negatively impact manufacturability (synthetic or recombinant) of the peptide complex (e.g., the selective depletion complex). In some embodiments, a peptide linker does not impair post-synthesis chemical alteration (e.g. conjugation of a fluorophore or albumin-binding chemical group) of the peptide complex (e.g., the selective depletion complex).

In some embodiments, a peptide linker can connect the C-terminus of a first peptide (e.g., a target-binding peptide, a TfR-binding peptide, or a half-life modifying peptide) to the N-terminus of a second peptide (e.g., a target-binding peptide, a TfR-binding peptide, or a half-life modifying peptide). In some embodiments, a peptide linker can connect the C-terminus of the second peptide (e.g., a target-binding peptide, a TfR-binding peptide, or a half-life modifying peptide) to the N-terminus of a third peptide (e.g., a target-binding peptide, a TfR-binding peptide, or a half-life modifying peptide). For example, a linker (e.g., any one of SEQ ID NO: 129-SEQ ID NO: 141 or SEQ ID NO: 195-SEQ ID NO: 218) can connect the C-terminus of a target-binding peptide to the N-terminus of a TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) to form a selective depletion complex. In another example, a linker (e.g., any one of SEQ ID NO: 129-SEQ ID NO: 141 or SEQ ID NO: 195-SEQ ID NO: 218) can connect the C-terminus of a TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) to the N-terminus of a target-binding peptide to form a selective depletion complex. In another example, a linker (e.g., any one of SEQ ID NO: 129-SEQ ID NO: 141 or SEQ ID NO: 195-SEQ ID NO: 218) can connect the C-terminus of a TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) to the N-terminus of a half-life extending peptide (e.g., SEQ ID NO: 178, SEQ ID NO: 179, or SEQ ID NO: 192) and the C-terminus of the half-life extending peptide to the N-terminus of a target binding peptide to form a selective depletion complex. In another example, a linker (e.g., any one of SEQ ID NO: 129-SEQ ID NO: 141 or SEQ ID NO: 195-SEQ ID NO: 218) can connect the C-terminus of a target-binding peptide to the N-terminus of a half-life extending peptide (e.g., SEQ ID NO: 178, SEQ ID NO: 179, or SEQ ID NO: 192) and a second linker (e.g., any one of SEQ ID NO: 129-SEQ ID NO: 141 or SEQ ID NO: 195-SEQ ID NO: 218) can connect the C-terminus of the half-life extending peptide to the N-terminus of a TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) to form a selective depletion complex. In another example, a first linker (e.g., any one of SEQ ID NO: 129-SEQ ID NO: 141 or SEQ ID NO: 195-SEQ ID NO: 218) can connect the C-terminus of a target-binding peptide to the N-terminus of a half-life extending peptide (e.g., SEQ ID NO: 178, SEQ ID NO: 179, or SEQ ID NO: 192) and a second linker (e.g., any one of SEQ ID NO: 129-SEQ ID NO: 141 or SEQ ID NO: 195-SEQ ID NO: 218) can connect the C-terminus of the half-life extending peptide to the N-terminus of a TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) to form a selective depletion complex. In another example, a linker (e.g., any one of SEQ ID NO: 129-SEQ ID NO: 141 or SEQ ID NO: 195-SEQ ID NO: 218) can connect the C-terminus of a half-life extending peptide (e.g., SEQ ID NO: 178, SEQ ID NO: 179, or SEQ ID NO: 192) to the N-terminus of a target-binding peptide and a second linker (e.g., any one of SEQ ID NO: 129-SEQ ID NO: 141 or SEQ ID NO: 195-SEQ ID NO: 218) can connect the C-terminus of the target-binding peptide to the N-terminus of a TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) to form a selective depletion complex.

In some embodiments, a linker can comprise a Tau-theraphotoxin-Hs1a, also known as DkTx (double-knot toxin), extracted from a native knottin-knottin dimer from Haplopelma schmidti (e.g., SEQ ID NO: 139). The linker can lack structural features that would interfere with dimerizing independently functional CDPs (e.g., a TfR-binding CDP and a target-binding CDP). In some embodiments, a linker can comprise a glycine-serine (Gly-Ser or GS) linker (e.g., SEQ ID NO: 129-SEQ ID NO: 138 or SEQ ID NO: 141 or SEQ ID NO: 195-SEQ ID NO: 218). Gly-Ser linkers can have minimal chemical reactivity and can impart flexibility to the linker. Serines can increase the solubility of the linker or the peptide complex, as the hydroxyl on the side chain is hydrophilic. In some embodiments, a linker can be derived from a peptide that separates the Fc from the Fv domains in a heavy chain of human immunoglobulin G (e.g., SEQ ID NO: 140). In some embodiments, a linker derived from a peptide from the heavy chain of human IgG can comprise a cysteine to serine mutation relative to the native IgG peptide.

In some embodiments, peptides of the present disclosure can be dimerized using an immunoglobulin heavy chain Fc domain. These Fc domains can be used to dimerize functional domains (e.g., a TfR-binding peptide and a target-binding peptide), either based on antibodies or other otherwise soluble functional domains. In some embodiments, dimerization can be homodimeric if the Fc sequences are native. In some embodiments, dimerization can be heterodimeric by mutating the Fc domain to generate a “knob-in-hole” format where one Fc CH3 domain contains novel residues (knob) designed to fit into a cavity (hole) on the other Fc CH3 domain. A first peptide domain (e.g., a TfR-binding peptide or a target-binding peptide) can be coupled to the knob, and a second peptide domain (e.g., a TfR-binding peptide or target-binding peptide) can be coupled to the hole. Knob+knob dimers can be highly energetically unfavorable. A purification tag can be added to the “knob” side to remove hole+hole dimers and select for knob+hole dimers.

The peptide peptides of the present disclosure (e.g., the target-binding peptides, TfR-binding peptides, or selective depletion complexes) can be linked to another peptide (e.g., a target-binding peptide, a TfR-binding peptide, a selective depletion complex, or a half-life modifying peptide) at the N-terminus or C-terminus. In some embodiments, one or more peptides can be linked or fused via a peptide linker (e.g., a peptide linker comprising a sequence of any one of SEQ ID NO: 129-SEQ ID NO: 141 or SEQ ID NO: 195-SEQ ID NO: 218). For example, a TfR-binding peptide can be fused to a target-binding peptide via a peptide linker of any one of SEQ ID NO: 129-SEQ ID NO: 141 or SEQ ID NO: 195-SEQ ID NO: 218. A peptide linker (e.g., a linker connecting a TfR-binding peptide, a target-binding peptide, a half-life modifying peptide, or combinations thereof) can have a length of about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25, about 30, about 35, about 40, about 45, or about 50 amino acid residues. A peptide linker (e.g., a linker connecting a TfR-binding peptide, a target-binding peptide, a half-life modifying peptide, or combinations thereof) can have a length of from about 2 to about 5, from about 2 to about 10, from about 2 to about 20, from about 3 to about 5, from about 3 to about 10, from about 3 to about 15, from about 3 to about 20, from about 3 to about 25, from about 5 to about 10, from about 5 to about 15, from about 5 to about 20, from about 5 to about 25, from about 10 to about 15, from about 10 to about 20, from about 10 to about 25, from about 15 to about 20, from about 15 to about 25, from about 20 to about 25, from about 20 to about 30, from about 20 to about 35, from about 20 to about 40, from about 20 to about 45, from about 20 to about 50, from about 3 to about 50, from about 3 to about 40, from about 3 to about 30, from about 10 to about 40, from about 10 to about 30, from about 50 to about 100, from about 100 to about 200, from about 200 to about 300, from about 300 to about 400, from about 400 to about 500, or from about 500 to about 600 amino acid residues.

In some embodiments, a first peptide (e.g., a TfR-binding peptide) and a second peptide (e.g., a target-binding peptide) can be connected by a flexible peptide linker. A flexible linker can provide rotational freedom between the first peptide and the second peptide and can allow the first peptide and the second peptide to bind their respective targets (e.g., a transferrin receptor and a target molecule) with minimal strain. In some embodiments, a peptide linker can have a persistence length of no more than 6 Å, no more than 7 Å, no more than 8 Å, no more than 9 Å, no more than 10 Å, no more than 12 Å, no more than 15 Å, no more than 20 Å, no more than 25 Å, no more than 30 Å, no more than 40 Å, or no more than 50 Å. In some embodiments, a peptide linker can have a persistence length of from about 4 Å to about 100 Å, from about 4 Å to about 50 Å, from about 4 Å to about 20 Å, from about 4 Å to about 10 Å, from about 10 Å to about 20 Å, from about 20 Å to about 30 Å, from about 30 Å to about 50 Å, or from about 50 Å to about 100 Å. The persistence length of the linker can be a measure of the flexibility of the peptide linker and can be quantified as the peptide length over which correlations in the direction of the tangent are lost.

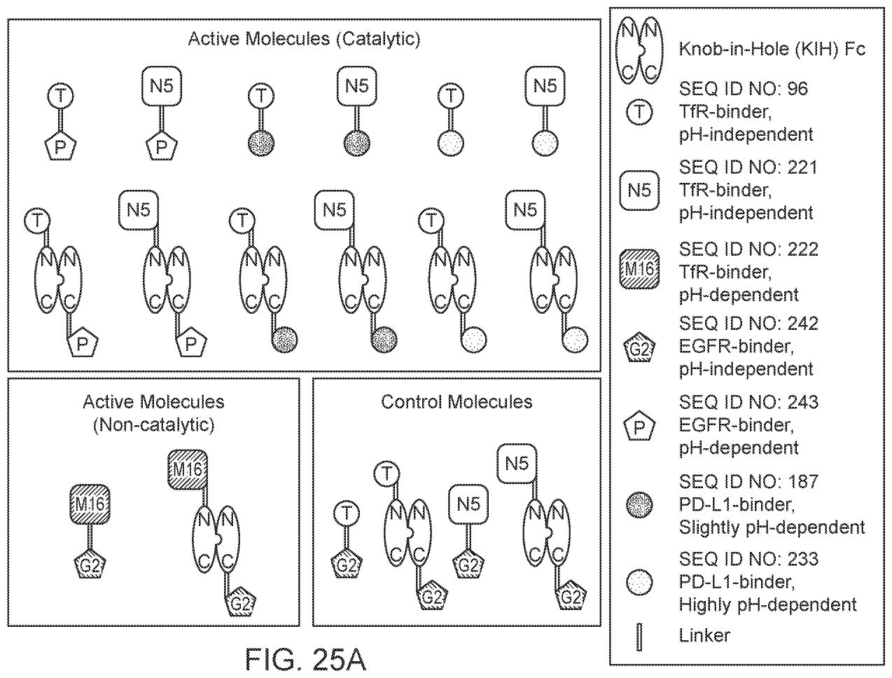

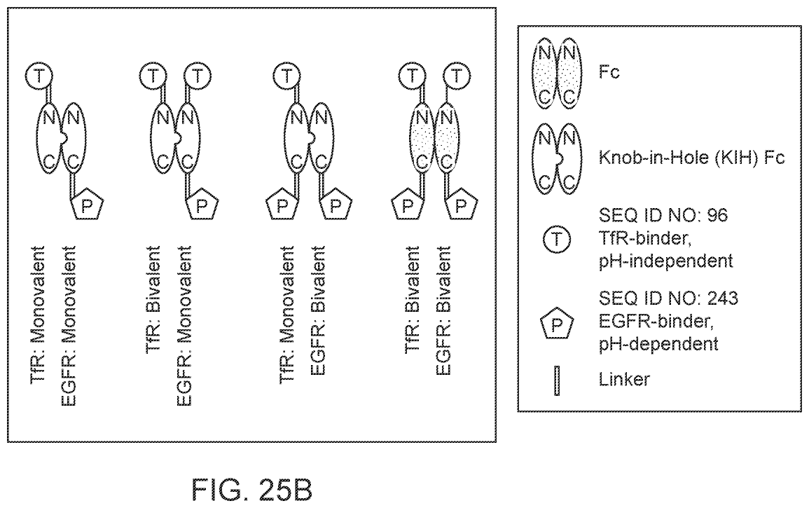

In some embodiments, a peptide linker can be selected based on a desired linker length, hydrodynamic radius, chromatographic mobility, posttranslational modification propensity, or combinations thereof. In some embodiments, a linker separating two or more functional domains of a peptide complex (e.g., separating a TfR-binding peptide and a target-binding peptide) can comprise a large, stable, globular domain, for example to reduce a propensity for glomerular filtration. In some embodiments, a linker separating two or more functional domains of a peptide complex (e.g., separating a TfR-binding peptide and a target-binding peptide) can comprise a small, flexible linker, for example to reduce the hydrodynamic radius of the complex for use in tight spaces like dense-core tumor stroma. Examples of selective depletion complexes formed from a single polypeptide chain comprising a target-binding peptide and a receptor-binding peptide connected via a peptide linker are illustrated in FIG. 25A and FIG. 25B. In some embodiments, a peptide linker can support independent folding of the two or more functional domains and may not inhibit interactions between the two or more functional domains and their binding targets (e.g., between a TfR-binding peptide and TfR or between a target-binding peptide and a target molecule).

In some embodiments, a peptide can be appended to the N-terminus of any peptide of the present disclosure following an N-terminal GS dipeptide and preceding, for example, a GGGS (SEQ ID NO: 129) spacer. In some embodiments, a peptide (e.g., a target-binding peptide) can be appended to either the N-terminus or C-terminus of any peptide disclosed herein (e.g., a TfR-binding peptide) using a peptide linker such as GₓS_{y} (SEQ ID NO: 130) peptide linker, wherein x and y can be any whole number, such as 1, 2, 3, 4, 5, 6, 7, 8, 9, and 10. In some embodiments, the peptide linker comprises (GS)x (SEQ ID NO: 131), wherein x can be any whole number, such as 1, 2, 3, 4, 5, 6, 7, 8, 9, and 10. In some embodiments, the peptide linker comprises GGSSG (SEQ ID NO: 132), GGGGG (SEQ ID NO: 133), GSGSGSGS (SEQ ID NO: 134), GSGG (SEQ ID NO: 135), GGGGS (SEQ ID NO: 136), GGGS (SEQ ID NO: 129), GGS (SEQ ID NO: 137), GGGSGGGSGGGS (SEQ ID NO: 138), or a variant or fragment thereof. Additionally, KKYKPYVPVTTN (SEQ ID NO: 139) from DkTx, and EPKSSDKTHT (SEQ ID NO: 140) from human IgG3 can be used as a peptide linker. In some embodiments, the peptide linker comprises GGGSGGSGGGS (SEQ ID NO: 141). In some embodiments, the peptide linker comprises a linker of any one of SEQ ID NO: 195-SEQ ID NO: 218. Examples of peptide linkers compatible with the target depletion complexes of the present disclosure are provided in TABLE 4. It is understood that any of the foregoing linkers or a variant or fragment thereof can be used with any number of repeats or any combinations thereof. It is also understood that other peptide linkers in the art or a variant or fragment thereof can be used with any number of repeats or any combinations thereof.

In some embodiments, a tag peptide (e.g., a peptide of any one of SEQ ID NO: 142-SEQ ID NO: 147) can be appended to the peptide (e.g., a target-binding peptide, a TfR-binding peptide, or a selective depletion complex) at any amino acid residue. In further embodiments, the tag peptide (e.g., a peptide of any one of SEQ ID NO: 142-SEQ ID NO: 147) can be appended to the peptide at any amino acid residue without interfering with TfR-binding activity, target-binding activity, selective depletion activity, or a combination thereof. In some embodiments, the tag peptide is appended via conjugation, linking, or fusion techniques. In other embodiments, a peptide (e.g., a target-binding peptide) can be appended to a second peptide (e.g., a TfR-binding peptide) at any amino acid residue. In further embodiments, the peptide (e.g., a target-binding peptide) can be appended to the second peptide (e.g., a TfR-binding peptide) at any amino acid residue without interfering with TfR-binding activity, target-binding activity, selective depletion activity, or a combination thereof. In some embodiments, the peptide is appended via conjugation, linking, or fusion techniques. In other embodiments, the peptide (e.g., a target binding peptide) can be appended to the second peptide (e.g., a TfR-binding peptide) at any amino acid residue.

TABLE 4
Peptide Linkers

| SEQ ID NO | Sequence |
|---|---|
| SEQ ID NO: 129 | GGGS |
| SEQ ID NO: 130 | GₓS_{y} |
| SEQ ID NO: 131 | (GS)x |
| SEQ ID NO: 132 | GGSSG |
| SEQ ID NO: 133 | GGGGG |
| SEQ ID NO: 134 | GSGSGSGS |
| SEQ ID NO: 135 | GSGG |
| SEQ ID NO: 136 | GGGGS |
| SEQ ID NO: 137 | GGS |
| SEQ ID NO: 138 | GGGSGGGSGGGS |
| SEQ ID NO: 139 | KKYKPYVPVTTN |
| SEQ ID NO: 140 | EPKSSDKTHT |
| SEQ ID NO: 141 | GGGSGGSGGGS |
| SEQ ID NO: 195 | AGSGGSGGSGGSPVPSTPPTNSSSTPPTPSPSPVPSTPPTNSSSTPPTP |
| | SPSPVPSTPPTNSSSTPPTPSPSAS |
| SEQ ID NO: 196 | AGSGGSGGSGGSPVPSTPPTPSPSTPPTPSPSPVPSTPPTNSSSTPPTP |
| | SPSPVPSTPPTPSPSTPPTPSPSAS |
| SEQ ID NO: 197 | AGSGGSGGSGGSPVPSTPPTPSPSTPPTPSPSGGSGNSSGSGGSPVPS |
| | TPPTPSPSTPPTPSPSAS |
| SEQ ID NO: 198 | AGSGGSGGSGGSPVPSTPPTPSPSTPPTPSPSPVPSTPPTPSPSTPPTPS |
| | PSPVPSTPPTPSPSTPPTPSPSAS |
| SEQ ID NO: 199 | AGSGGSGGSGGSPVPSTPPTPSPSTPPTPSPSIQRTPKIQVYSRHPAE |
| | NGKSNFLNCYVSGFHPSDIEVDLLKNGERIEKVEHSDLSFSKDWSF |
| | YLLYYTEFTPTEKDEYACRVNHVTLSQPKIVKWDRDPVPSTPPTPS |
| | PSTPPTPSPSAS |
| SEQ ID NO: 200 | AGSGGSGGSGGSPVPSTPPTPSPSTPPTPSPSIQRTPKIQVYSRHPAE |
| | NGKSNFLNCYVSGFHPSDIEVDLLKNGERIEKVEHSDLSFSKDWSF |
| | YLLYYTEFTPTEKDEYACRVNHVTLSQPKIVKWDRDGGSGGSGGS |
| | GGSAS |
| SEQ ID NO: 201 | AGPVPSTPPTPSPSTPPTPSPSIQRTPKIQVYSRHPAENGKSNFLNCY |
| | VSGFHPSDIEVDLLKNGERIEKVEHSDLSFSKDWSFYLLYYTEFTPT |
| | EKDEYACRVNHVTLSQPKIVKWDRDGGSGGSGGSGGSIQRTPKIQ |
| | VYSRHPAENGKSNFLNCYVSGFHPSDIEVDLLKNGERIEKVEHSDL |
| | SFSKDWSFYLLYYTEFTPTEKDEYACRVNHVTLSQPKIVKWDRDG |
| | GSGGSGGSGAS |
| SEQ ID NO: 202 | AGSGGSGGSGGSPVPSTPPTPSPSTPPTPSPSIQRTPKIQVYSRHPAE |
| | NGKSNFLNCYVSGFHPSDIEVDLLKNGERIEKVEHSDLSFSKDWSF |
| | YLLYYTEFTPTEKDEYACRVNHVTLSQPKIVKWDRDGGSGGSGGS |
| | GGSIQRTPKIQVYSRHPAENGKSNFLNCYVSGFHPSDIEVDLLKNG |
| | ERIEKVEHSDLSFSKDWSFYLLYYTEFTPTEKDEYACRVNHVTLSQ |
| | PKIVKWDRDPVPSTPPTPSPSTPPTPSPSAS |
| SEQ ID NO: 203 | AGSGGSGGSGGSPVPSTPPTPSPSTPPTPSPSQIFVKTLTGKTITLEV |
| | EPSDTIENVKAKIQDKEGIPPDQQRLIFAGKQLEDGRTLSDYNIQKE |
| | STLHLVLRLRGGGGSGGSGGSGGSAS |
| SEQ ID NO: 204 | AGSGGSGGSGGSPVPSTPPTPSPSTPPTPSPSDGRYSLTYIYTGLSKH |
| | VEDVPAFQALGSLNDLQFFRYNSKDRKSQPMGLWRQVEGMEDW |
| | KQDSQLQKAREDIFMETLKDIVEYYNDSNGSHVLQGRFGCEIENN |
| | RSSGAFWKYYYDGKDYIEFNKEIPAWVPFDPAAQITKQKWEAEPV |
| | YVQRAKAYLEEECPATLRKYLKYSKNILDRQDPPSVVVTSHQAPG |
| | EKKKLKCLAYDFYPGKIDVHWTRAGEVQEPELRGDVLHNGNGTY |
| | QSWVVVAVPPQDTAPYSCHVQHSSLAQPLVVPWEASPVPSTPPTP |
| | SPSTPPTPSAS |
| SEQ ID NO: 205 | AGSGGSGGSGGSGGSGGSGGSGGSDGRYSLTYIYTGLSKHVEDVP |
| | AFQALGSLNDLQFFRYNSKDRKSQPMGLWRQVEGMEDWKQDSQ |
| | LQKAREDIFMETLKDIVEYYNDSNGSHVLQGRFGCEIENNRSSGAF |
| | WKYYYDGKDYIEFNKEIPAWVPFDPAAQITKQKWEAEPVYVQRA |
| | KAYLEEECPATLRKYLKYSKNILDRQDPPSVVVTSHQAPGEKKKL |
| | KCLAYDFYPGKIDVHWTRAGEVQEPELRGDVLHNGNGTYQSWV |
| | VVAVPPQDTAPYSCHVQHSSLAQPLVVPWEASGGSGGSGGSGGS |
| | DGRYSLTYIYTGLSKHVEDVPAFQALGSLNDLQFFRYNSKDRKSQ |
| | PMGLWRQVEGMEDWKQDSQLQKAREDIFMETLKDIVEYYNDSN |
| | GSHVLQGRFGCEIENNRSSGAFWKYYYDGKDYIEFNKEIPAWVPF |
| | DPAAQITKQKWEAEPVYVQRAKAYLEEECPATLRKYLKYSKNILD |
| | RQDPPSVVVTSHQAPGEKKKLKCLAYDFYPGKIDVHWTRAGEVQ |
| | EPELRGDVLHNGNGTYQSWVVVAVPPQDTAPYSCHVQHSSLAQP |
| | LVVPWEASPVPSTPPTPSPSTPPTPSPSAS |
| SEQ ID NO: 206 | AGSGNSSGSGGSGGSGNSSGSGGSPVPSTPPTPSPSTPPTPSPSAS |
| SEQ ID NO: 207 | KLSGGGGSGGGGSGGGGSAEAWYNLGNAYYKQGDYQKAIEYYQ |
| | KALELDPNNAEAWYNLGNAYYKQGDYQKAIEYYQKALELDPNN |
| | AEAWYNLGNAYYKQGDYQKAIEDYQKALELDPNNLQRSAGGGG |
| | SGGGGSGGGGAS |
| SEQ ID NO: 208 | KLSGGGGSGGGGSGGGGSAEAWYNLGNAYYKQGDYQKAIEYYQ |
| | KALELDPNNAEAWYNLGNAYYKQGDYQKAIEYYQKALELDPNN |
| | AEAWYNLGNAYYKQGDYQKAIEDYQKALELDPNNLQAEAWKN |
| | LGNAYYKQGDYQKAIEYYQKALELDPNNASAWYNLGNAYYKQG |
| | DYQKAIEYYQKALELDPNNAKAWYRRGNAYYKQGDYQKAIEDY |
| | QKALELDPNNRSRSAGGGGSGGGGSGGGGAS |
| SEQ ID NO: 209 | KLSGGGGSGGGGSGGGGSAEAWYNLGNAYYKQGDYQKAIEYYQ |
| | KALELDPNNAEAWYNLGNAYYKQGDYQKAIEYYQKALELDPNN |
| | AEAWYNLGNAYYKQGDYQKAIEDYQKALELDPNNLQAEAWKN |
| | LGNAYYKQGDYQKAIEYYQKALELDPNNASAWYNLGNAYYKQG |
| | DYQKAIEYYQKALELDPNNAKAWYRRGNAYYKQGDYQKAIEDY |
| | QKALELDPNNRSAEAWYNLGNAYYKQGDYQKAIEYYQKALELD |
| | PNNAEAWYNLGNAYYKQGDYQKAIEYYQKALELDPNNAEAWY |
| | NLGNAYYKQGDYQKAIEDYQKALELDPNNLQRSAGGGGSGGGG |
| | SGGGGAS |
| SEQ ID NO: 210 | KLSGGGGSGGGGGGGGSAEAWYNLGNAYYKQGDYQKAIEYYQ |
| | KALELDPNNAEAWYNLGNAYYKQGDYQKAIEYYQKALELDPNN |
| | AEAWYNLGNAYYKQGDYQKAIEDYQKALELDPNNLQAEAWKN |
| | LGNAYYKQGDYQKAIEYYQKALELDPNNASAWYNLGNAYYKQG |
| | DYQKAIEYYQKALELDPNNAKAWYRRGNAYYKQGDYQKAIEDY |
| | QKALELDPNNRSAEAWYNLGNAYYKQGDYQKAIEYYQKALELD |
| | PNNAEAWYNLGNAYYKQGDYQKAIEYYQKALELDPNNAEAWY |
| | NLGNAYYKQGDYQKAIEDYQKALELDPNNLQAEAWKNLGNAYY |
| | KQGDYQKAIEYYQKALELDPNNASAWYNLGNAYYKQGDYQKAI |
| | EYYQKALELDPNNAKAWYRRGNAYYKQGDYQKAIEDYQKALEL |
| | DPNNRSAGGGGSGGGGSGGGGAS |
| SEQ ID NO: 211 | GGGGSAS |
| SEQ ID NO: 212 | GGGGSGGGGSAS |
| SEQ ID NO: 213 | GGGGSGGGGSGGGGSAS |
| SEQ ID NO: 214 | GGGGSGGGGSGGGGSGGGGSGGGGSAS |
| SEQ ID NO: 215 | GGGGSGGGGSGGGGSGGGGSGGGGSGGGGSAS |
| SEQ ID NO: 216 | AGGGSGGGGSGGGGSGGGGSGGGGSGGGGSGGGGSAS |
| SEQ ID NO: 217 | AGGGSGGGGSGGGGSGGGGSGGGGSGGGGSGGGGSGGGGSAS |
| SEQ ID NO: 218 | AGGGSGGGGSGGGGSGGGGSGGGGSGGGGSGGGGSGGGGSGGGGSAS |
| SEQ ID NO: 223 | GGGGSGGGGSGGGGS |
| SEQ ID NO: 224 | GGGGSGGGGSGGGGSGGGGS |
| SEQ ID NO: 225 | GGGGS |
| SEQ ID NO: 226 | GSPVPSTPPTPSPSTPPTPSPSAS |
| SEQ ID NO: 227 | LKEAKEKAIEELKKAGITSDYYFDLINKAKAVEGVNALKDEILKA |
| SEQ ID NO: 194 | LKEAKEKAIEELKKAGITSDYYFDLINKAKTVEGVNALKDEILKA |
| SEQ ID NO: 391 | PVPSTPPTPSPSTPPTPSPS |

In some embodiments, a selective depletion complex may comprise two or more polypeptide chains. For example, a target-binding peptide and a receptor-binding peptide may be complexed via a dimerization domain to form a selective depletion complex. The dimerization domain may be a heterodimerization domain or a homodimerization domain. Examples of selective depletion complexes comprising a target-binding peptide and a receptor-binding peptide connected via a dimerization domain (e.g., an Fc homodimerization domain or a knob-in-hole heterodimerization domain) are illustrated in FIG. 25A, FIG. 25B, and FIG. 25C.

A target-binding peptide and a receptor-binding peptide may be complexed by forming a heterodimer via a heterodimerization domain. The target-binding peptide may be linked or fused to a first heterodimerization domain and the receptor-binding peptide may be linked or fused to a second heterodimerization domain. The first heterodimerization domain may bind to the second heterodimerization domain to form a heterodimeric complex comprising the target-binding peptide and the receptor-binding peptide. For example, the receptor-binding peptide may be linked or fused to an Fc “knob” peptide (e.g., SEQ ID NO: 260) and the immune cell targeting agent may be linked or fused to an Fc “hole” peptide (e.g., SEQ ID NO: 261). In another example, the receptor-binding peptide may be linked or fused to an Fc “hole” peptide (e.g., SEQ ID NO: 261) and the target-binding may be linked or fused to an Fc “knob” peptide (e.g., SEQ ID NO: 260). In some embodiments, a receptor-binding peptide (e.g., any one of SEQ ID NO: 1-SEQ ID NO: 222) may form a heterodimer with target-binding peptide via a heterodimerization domain provided in TABLE 5. For example, the receptor-binding peptide may be fused to chain 1 of an Fc pair (e.g., SEQ ID NO: 260) and the target-binding peptide may be fused to chain 2 of the Fc pair (e.g., SEQ ID NO: 261). In another example, the receptor-binding peptide may be fused to chain 2 of an Fc pair (e.g., SEQ ID NO: 263) and the target-binding peptide may be fused to chain 1 of the Fc pair (e.g., SEQ ID NO: 262). A selective depletion complex comprising a heterodimerization domain may form a monovalent selective depletion complex, as shown in FIG. 25B, or a selective depletion complex comprising a heterodimerization domain may form a multivalent selective depletion complex, as shown in FIG. 25C.

TABLE 5
Exemplary Heterodimerization Domains

| Name | SEQ ID NO | Sequence |
|---|---|---|
| Chain 1 | SEQ ID NO: 260 | EPKSCDKTHTCPPCPAPELLGGPSVFLFPPKPKDTLMISRTPE |
| Pair 1 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| Chain 2 | SEQ ID NO: 261 | EPKSCDKTHTCPPCPAPELLGGPSVFLFPPKPKDTLMISRTPE |
| Pair 1 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTKNQVSLSCAVKGFYPSDIAVEW |
| | | ESNGQPENNYKTTPPVLDSDGSFFLVSKLTVDKSRWQQGNV |
| | | FSCSVMHEALHNHYTQKSLSLSPGK |
| Chain 1 | SEQ ID NO: 262 | EPKSCDKTHTCPPCPAPELLGGPSVFLFPPKPKDTLMISRTPE |
| Pair 2 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPCRDELTKNQVSLWCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| Chain 2 | SEQ ID NO: 263 | EPKSCDKTHTCPPCPAPELLGGPSVFLFPPKPKDTLMISRTPE |
| Pair 2 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVCTLPPSRDELTKNQVSLSCAVKGFYPSDIAVEW |
| | | ESNGQPENNYKTTPPVLDSDGSFFLVSKLTVDKSRWQQGNV |
| | | FSCSVMHEALHNHYTQKSLSLSPGK |
| Chain 1 | SEQ ID NO: 264 | EPKSCDKTHTCPPCPAPELLGGPSVFLFPPKPKDTLMISRTPE |
| Pair 3 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTKNQVSLTCLVKGFYPSDIAVEW |
| | | ESNGQPENNYDTTPPVLDSDGSFFLYSDLTVDKSRWQQGNV |
| | | FSCSVMHEALHNHYTQKSLSLSPGK |
| Chain 2 | SEQ ID NO: 265 | EPKSCDKTHTCPPCPAPELLGGPSVFLFPPKPKDTLMISRTPE |
| Pair 3 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRKELTKNQVSLTCLVKGFYPSDIAVEW |
| | | ESNGQPENNYKTTPPVLKSDGSFFLYSKLTVDKSRWQQGNV |
| | | FSCSVMHEALHNHYTQKSLSLSPGK |
| Chain 1 | SEQ ID NO: 266 | EPKSCDKTHTCPPCPAPELLGGPSVFLFPPKPKDTLMISRTPE |
| Pair 4 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTKNQVHLTCLVKGFYPSDIAVEW |
| | | ESNGQPENNYKTTPPVLDSDGSFALYSKLTVDKSRWQQGNV |
| | | FSCSVMHEALHNHYTQKSLSLSPGK |
| Chain 2 | SEQ ID NO: 267 | EPKSCDKTHTCPPCPAPELLGGPSVFLFPPKPKDTLMISRTPE |
| Pair 4 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVTTLPPSRDELTKNQVSLTCLVKGFYPSDIAVEW |
| | | ESNGQPENNYKTFPPVLDSDGSFFLYSKLTVDKSRWQQGNV |
| | | FSCSVMHEALHNHYTQKSLSLSPGK |
| Chain 1 | SEQ ID NO: 268 | EPKSCEKTHTCPECPAPELLGGPSVFLFPPKPKDTLMISRTPEV |
| Pair 5 | | TCVVVDVSHEDPEVKFNWYVDGVEVHNAKTKPREEQYNST |
| | | YRVVSVLTVLHQDWLNGKEYKCKVSNKALPAPIEKTISKAK |
| | | GQPREPQVYTLPPSRDELTKNQVSLTCEVKGFYPSDIAVEWE |
| | | SNGQPENNYKTTPPVLDSDGSFFLYSKLTVDKSRWQQGNVF |
| | | SCSVMHEALHNHYTQKSLSLSPGK |
| Chain 2 | SEQ ID NO: 269 | EPKSCRKTHTCPRCPAPELLGGPSVFLFPPKPKDTLMISRTPE |
| Pair 5 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTKNQVSLTCLVKGFYPSDIAVEW |
| | | ESNGQPENNYKTTPPVLDSDGSFFLYSRLTVDKSRWQQGNV |
| | | FSCSVMHEALHNHYTQKSLSLSPGK |
| Chain 1 | SEQ ID NO: 270 | EPKSCDKTHTCPPCPAPELLGGPSVFLFPPKPKDTLMISRTPE |
| Pair 6 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTKNQVSLTCLVKGFYPSDIAVEW |
| | | ESNGQPENNYKTTPPVLDSDGSFLLYSKLTVDKSRWQQGNV |
| | | FSCSVMHEALHNHYTQKSLSLSPGK |
| Chain 2 | SEQ ID NO: 271 | EPKSCDKTHTCPPCPAPELLGGPSVFLFPPKPKDTLMISRTPE |
| Pair 6 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTKNQVSLTCLVKGFYPSDIAVEW |
| | | ESNGQPENNYKTTPPVLDSDGSFFLYSRLTVDKSRWQQGNV |
| | | FSCSVMHEALHNHYTQKSLSLSPGK |
| Chain 1 | SEQ ID NO: 272 | EPKSCDKTHTCPPCPAPELLGGPSVFLFPPKPKDTLMISRTPE |
| Pair 7 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYVLPPSRDELTKNQVSLLCLVKGFYPSDIAVEW |
| | | ESNGQPENNYLTWPPVLDSDGSFFLYSKLTVDKSRWQQGNV |
| | | FSCSVMHEALHNHYTQKSLSLSPGK |
| Chain 2 | SEQ ID NO: 273 | EPKSCDKTHTCPPCPAPELLGGPSVFLFPPKPKDTLMISRTPE |
| Pair 7 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYVYPPSRDELTKNQVSLTCLVKGFYPSDIAVE |
| | | WESNGQPENNYKTTPPVLDSDGSFALVSKLTVDKSRWQQG |
| | | NVFSCSVMHEALHNHYTQKSLSLSPGK |
| Chain 1 | SEQ ID NO: 274 | EPKSCDKTHTCPPCPAPELLGGPSVFLFPPKPKDTLMISRTPE |
| Pair 8 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTENQVSLTCLVKGFYPSDIAVEW |
| | | ESNGQPENNYKTTPPVLDSDGSFFLYSWLTVDKSRWQQGNV |
| | | FSCSVMHEALHNHYTQKSLSLSPGK |
| Chain 2 | SEQ ID NO: 275 | EPKSCDKTHTCPPCPAPELLGGPSVFLFPPKPKDTLMISRTPE |
| Pair 8 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPRVYTLPPSRDELTKNQVSLTCLVKGFYPSDIAVEW |
| | | ESNGQPENNYKTTPPVLVSDGSFTLYSKLTVDKSRWQQGNV |
| | | FSCSVMHEALHNHYTQKSLSLSPGK |
| Chain 1 | SEQ ID NO: 276 | EPKSCDKTHTCPPCPAPELLGGPSVFLFPPKPKDTLMISRTPE |
| Pair 9 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVCTLPPSRDELTENQVSLTCLVKGFYPSDIAVEW |
| | | ESNGQPENNYKTTPPVLDSDGSFFLYSWLTVDKSRWQQGNV |
| | | FSCSVMHEALHNHYTQKSLSLSPGK |
| Chain 2 | SEQ ID NO: 277 | EPKSCDKTHTCPPCPAPELLGGPSVFLFPPKPKDTLMISRTPE |
| Pair 9 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPRVYTLPPCRDELTKNQVSLTCLVKGFYPSDIAVEW |
| | | ESNGQPENNYKTTPPVLVSDGSFTLYSKLTVDKSRWQQGNV |
| | | FSCSVMHEALHNHYTQKSLSLSPGK |
| Chain 1 | SEQ ID NO: 278 | EPKSCDKTHTCPPCPAPELLGGPSVFLFPPKPKDTLMISRTPE |
| Pair 10 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTKNQVSLTCLVEGFYPSDIAVEW |
| | | ESNGQPENNYKTTPPVLDSDGSFFLYSWLTVDKSRWQQGNV |
| | | FSCSVMHEALHNHYTQKSLSLSPGK |
| Chain 2 | SEQ ID NO: 279 | EPKSCDKTHTCPPCPAPELLGGPSVFLFPPKPKDTLMISRTPE |
| Pair 10 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDNLTKNQVSLTCLVKGFYPSDIAVEW |
| | | ESNGQPENNYKTTPPVLVSDGSFTLYSKLTVDKSRWQQGNV |
| | | FSCSVMHEALHNHYTQKSLSLSPGK |
| Chain 1 | SEQ ID NO: 280 | EPKSCDKTHTCPPCPAPELLGGPSVFLFPPKPKDTLMISRTPE |
| Pair 11 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTDNQVSLTCLVKGFYPSDIAVEW |
| | | ESNGQPENNYKTTPPVLMSDGSFFLASKLTVDKSRWQQGNV |
| | | FSCSVMHEALHNHYTQKSLSLSPGK |
| Chain 2 | SEQ ID NO: 281 | EPKSCDKTHTCPPCPAPELLGGPSVFLFPPKPKDTLMISRTPE |
| Pair 11 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPRRPRVYTLPPSRDELTKNQVSLVCLVKGFYPSDIAVEW |
| | | ESNGQPENNYKTTPPVLDSDGSFFLYSVLTVDKSRWQQGNV |
| | | FSCSVMHEALHNHYTQKSLSLSPGK |
| Chain 1 | SEQ ID NO: 282 | EPKSCDKTHTCPPCPAPELLGGPSVFLFPPKPKDTLMISRTPE |
| Pair 12 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVSTLPPSRDELTKNQVSLMCLVYGFYPSDIAVEW |
| | | ESNGQPENNYKTTPPVLDSDGSFFLYSVLTVDKSRWQQGNV |
| | | FSCSVMHEALHNHYTQKSLSLSPGK |
| Chain 2 | SEQ ID NO: 283 | EPKSCDKTHTCPPCPAPELLGGPSVFLFPPKPKDTLMISRTPE |
| Pair 12 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRGDLTKNQVQLTCLVKGFYPSDIAVE |
| | | WESNGQPENNYKTTPPVLDSDGSFFLASKLTVDKSRWQQGN |
| | | VFSCSVMHEALHNHYTQKSLSLSPGK |
| Chain 1 | SEQ ID NO: 284 | EPKSCDKTHTCPPCPAPELLGGPSVFLFPPKPKDTLMISRTPE |
| Pair 13 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTDPPSRDELTKNQVSLTCEVKGFYPSDIAVEW |
| | | ESNGQPENNYKTTPPVLDSDGSFFLYSKLTVDKSRWQQGNV |
| | | FSCSVMHEALHNHYTQKSLSLSPGK |
| Chain 2 | SEQ ID NO: 285 | EPKSCDKTHTCPPCPAPELLGGPSVFLFPPKPKDTLMISRTPE |
| Pair 13 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTKPPSRDELTKNQVSLKCLVKGFYPSDIAVE |
| | | WESNGQPENNYKTTPPVLDSDGSFFLYSKLTVDKSRWQQGN |
| | | VFSCSVMHEALHNHYTQKSLSLSPGK |
| Chain 1 | SEQ ID NO: 286 | EPKSCDKTHTCPPCPAPELLGGPSVFLFPPKPKDTLMISRTPE |
| Pair 14 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDELTKNQVSLTCDVSGFYPSDIAVEW |
| | | ESNGQPENNYKTTPPVLDSDGSFFLYSKLTVDKSRWQQGNV |
| | | FSCSVMHEALHNHYTQKSLSLSPGK |
| Chain 2 | SEQ ID NO: 287 | EPKSCDKTHTCPPCPAPELLGGPSVFLFPPKPKDTLMISRTPE |
| Pair 14 | | VTCVVVDVSHEDPEVKFNWYVDGVEVHNAKTKPREEQYNS |
| | | TYRVVSVLTVLHQDWLNGKEYKCKVSNKALPAPIEKTISKA |
| | | KGQPREPQVYTLPPSRDQLTKNQVKLTCLVKGFYPSDIAVE |
| | | WESNGQPENNYKTTPPVLDSDGSFFLYSKLTVDKSRWQQGN |
| | | VFSCSVMHEALHNHYTQKSLSLSPGK |

In some embodiments, a target-binding peptide and a receptor-binding peptide may form a selective depletion complex comprising a homodimer complexed via a homodimerization domain. The target-binding peptide may be linked or fused to the N-terminus of the homodimerization domain and the receptor-binding peptide may be linked or fused to the C-terminus of the homodimerization domain. In some embodiments, the target-binding peptide may be linked or fused to the C-terminus of the homodimerization domain and the receptor-binding peptide may be linked or fused to the N-terminus of the homodimerization domain. In some embodiments, the target-binding peptide and the receptor-binding peptide may both be fused on the N-terminal, or both be fused on the C-terminal end of the homodimerization domain. A selective depletion complex comprising a homodimerization domain may form a multivalent selective depletion complex, as shown in FIG. 25C. Examples of homodimerization domains that may be used to link or fuse a target-binding peptide to a receptor-binding peptide are provided in TABLE 6.

TABLE 6
Exemplary Homodimerization Domains

| SEQ ID NO | Sequence |
|---|---|
| SEQ ID NO: 245 | EPKSCDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLH |
| | QDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 246 | EPKSSDKTHTCPPCPAPELLGGPSVFLFPPKPKDTLMISRTPEVTCVVVD |
| | VSHEDPEVKFNWYVDGVEVHNAKTKPREEQYNSTYRVVSVLTVLHQ |
| | DWLNGKEYKCKVSNKALPAPIEKTISKAKGQPREPQVYTLPPSRDELT |
| | KNQVSLTCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 247 | EPKSCDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLH |
| | QDWLNGKEYKCKVSNKALGAPIEKTISKAKGQPREPQVYTLPPSRDEL |
| | TKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 248 | EPKSCDKTHTCPPCPAPELLGGPSVFLFPPKPKDTLMISRTPEVTCVVV |
| | DVSHEDPEVKFNWYVDGVEVHNAKTKPREEQYASTYRVVSVLTVLH |
| | QDWLNGKEYKCKVSNKALPAPIEKTISKAKGQPREPQVYTLPPSRDEL |
| | TKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 249 | EPKSCDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 250 | EPKSSDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLH |
| | QDWLNGKEYKCKVSNKALGAPIEKTISKAKGQPREPQVYTLPPSREEM |
| | TKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 251 | EPKSSDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLH |
| | QDWLNGKEYKCKVSNKALGAPIEKTISKAKGQPREPQVYTLPPSREEM |
| | TKNQVSLWCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 252 | EPKSSDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLH |
| | QDWLNGKEYKCKVSNKALGAPIEKTISKAKGQPREPQVYTLPPSREEM |
| | TKNQVSLSCAVKGFYPSDIAVEWESNGQPENNYKTTPPVLDSDGSFFL |
| | VSKLTVDKSRWQQGNVFSCSVMHEALHNHYTQKSLSLSPGK |
| SEQ ID NO: 253 | EPKSSDKTHTCPPCPAPEAAGGPSVFLFPPKPKDTLMISRTPEVTCVVV |
| | DVSHEDPEVKFNWYVDGVEVHNAKTKPREEQYASTYRVVSVLTVLH |
| | QDWLNGKEYKCKVSNKALGAPIEKTISKAKGQPREPQVYTLPPSREEM |
| | TKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 254 | EPKSSDKTHTCPPCPAPEAAGGPSVFLFPPKPKDTLMISRTPEVTCVVV |
| | DVSHEDPEVKFNWYVDGVEVHNAKTKPREEQYASTYRVVSVLTVLH |
| | QDWLNGKEYKCKVSNKALGAPIEKTISKAKGQPREPQVYTLPPSREEM |
| | TKNQVSLWCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 255 | EPKSSDKTHTCPPCPAPEAAGGPSVFLFPPKPKDTLMISRTPEVTCVVV |
| | DVSHEDPEVKFNWYVDGVEVHNAKTKPREEQYASTYRVVSVLTVLH |
| | QDWLNGKEYKCKVSNKALGAPIEKTISKAKGQPREPQVYTLPPSREEM |
| | TKNQVSLSCAVKGFYPSDIAVEWESNGQPENNYKTTPPVLDSDGSFFL |
| | VSKLTVDKSRWQQGNVFSCSVMHEALHNHYTQKSLSLSPGK |
| SEQ ID NO: 256 | EPKSCDKTHTCPPCPAPELLGGPSVFLFPPKPKDTLMISRTPEVTCVVV |
| | DVSHEDPEVKFNWYVDGVEVHNAKTKPREEQYASTYRVVSVLTVLH |
| | QDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 257 | EPKSCDKTHTCPPCPAPELLGGPSVFLFPPKPKDTLMISRTPEVTCVVV |
| | DVSHEDPEVKFNWYVDGVEVHNAKTKPREEQYNSTYRVVSVLTVLH |
| | QDWLNGKEYKCKVSNKALPAPIEKTISKAKGQPREPQVYTLPPSREEM |
| | TKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| SEQ ID NO: 258 | EPKSSDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQ |
| | DWLNGKEYKCKVSNKALGAPIEKTISKAKGQPREPQVYTLPPSRDELT |
| | KNQVSLWCLVKGFYPSDIAVEWESNGQPENNYKTTPPVLDSDGSFFLY |
| | SKLTVDKSRWQQGNVFSCSVMHEALHNHYTQKSLSLSPGK |
| SEQ ID NO: 259 | EPKSSDKTHTCPPCPAPELLGGPSVFLFPPKPKDTLMISRTPEVTCVVVD |
| | VSHEDPEVKFNWYVDGVEVHNAKTKPREEQYASTYRVVSVLTVLHQ |
| | DWLNGKEYKCKVSNKALGAPIEKTISKAKGQPREPQVYTLPPSRDELT |
| | KNQVSLSCAVKGFYPSDIAVEWESNGQPENNYKTTPPVLDSDGSFFLV |
| | SKLTVDKSRWQQGNVFSCSVMHEALHNHYTQKSLSLSPGK |

### Modification of Peptides

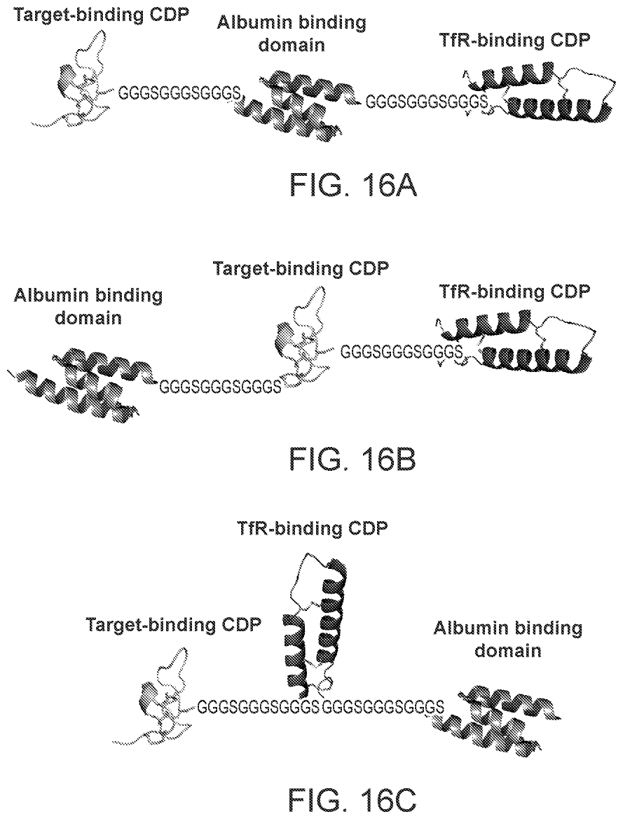

A peptide can be modified (e.g., chemically modified) one or more of a variety of ways. In some embodiments, the peptide can be mutated to add function, delete function, or modify the in vivo behavior. One or more loops between the disulfide linkages of a peptide (e.g., a TfR-binding peptide, a target-binding peptide, or a selective depletion complex) can be modified or replaced to include active elements from other peptides (such as described in Moore and Cochran, Methods in Enzymology, 503, p. 223-251, 2012). In some embodiments, the peptides of the present disclosure (e.g., TfR-binding peptides, target-binding peptides, or selective depletion complexes) can be further functionalized and multimerized by adding an additional functional domain. For example, an albumin-binding domain (ABD) from a Finegoldia magna peptostreptococcal albumin-binding protein (SEQ ID NO: 192, MKLNKKLLMAALAGAIVVGGGVNTFAADEPGAIKVDKAPEAPSQELKLTKEEAEKAL KKEKPIAKERLRRLGITSEFILNQIDKATSREGLESLVQTIKQSYLKDHPIKEEKTEETPKY NNLFDKHELGGLGKDKGPGRFDENGWENNEHGYETRENAEKAAVKALGDKEINK SYT ISQGVDGRYYYVL SREEAETPKKPEEKKPEDKRPKMTIDQWLLKNAKEDAIAELKKAGI TSDFYFNAINKAKTVEEVNALKNEILKAHAGKEVNPSTPEVTPSVPQNHYHENDYANIG AGEGTKEDGKKENSKEGIKRKTAREEKPGKEEKPAKEDKKENKKKENTDSPNKKKKE KAALPEAGRRKAEILTLAAASLSSVAGAFISLKKRK). For example, an albumin-binding domain of SEQ ID NO: 193 (LKNAKEDAIAELKKAGITSDFYFNAINKAKTVEEVNALKNEILKA) can be added to a peptide of the present disclosure. In some embodiments, a peptide of the present disclosure can be functionalized with an albumin-binding domain that has been modified for improved albumin affinity, improved stability, reduced immunogenicity, improved manufacturability, or a combination thereof. For example, a peptide can be functionalized with a modified albumin-binding domain of SEQ ID NO: 194 (LKEAKEKAIEELKKAGITSDYYFDLINKAKTVEGVNALKDEILKA) or SEQ ID NO: 227 (LKEAKEKAIEELKKAGITSDYYFDLINKAKAVEGVNALKDEILKA) having high thermostability and improved serum half-life compared to the albumin binding domain of SEQ ID NO: 193. In some embodiments, an albumin-binding peptide may be selected based on a desired off rate for albumin. For example, an albumin-binding peptide of SEQ ID NO: 227 may be selected for its faster off rate relative to SEQ ID NO: 194. The albumin-binding domain comprises a simple three-helical structure that would be unlikely to disturb the independent folding of the other peptide domains (e.g., CDP domains). In some embodiments, a functional domain (e.g., an albumin-binding domain) can increase the serum half-life of a peptide or peptide complex of the present disclosure. A functional domain (e.g., an albumin-binding domain) can be included in any orientation relative to the TfR-binding peptide or the target-binding peptide. For example, a functional domain can be linked to the TfR-binding peptide, the target-binding peptide, or in between the TfR-binding peptide and the target-binding peptide, as illustrated in FIG. 16A-FIG. 16C. In some embodiments, an albumin binding peptide (e.g., SEQ ID NO: 194 or SEQ ID NO: 227) may be used to link a target-binding peptide to a receptor-binding peptide. An additional functional domain can be linked to one or more peptides (e.g., a TfR-binding peptide or a target-binding peptide) via a linker (e.g., any one of SEQ ID NO: 129-SEQ ID NO: 141 or SEQ ID NO: 195-SEQ ID NO: 218).

A peptide of the present disclosure (e.g., a TfR-binding peptide, a receptor-binding peptide, a target-binding peptide, or a selective depletion complex) may be modified with a signal peptide to mark the peptide for secretion. For example, a peptide may be modified with a signal peptide corresponding to SEQ ID NO: 230 (METDTLLLWVLLLWVPGSTG). In some embodiments, the signal peptide may be appended to an N-terminus or a C-terminus of the peptide. A peptide may be modified for additional stability during translation or secretion. For example, a peptide may be modified with a sidrocalin with a furin cleavage site corresponding to SEQ ID NO: 229 (GSQDSTSDLIPAPPL SKVPLQQNFQDNQFQGKWYVVGLAGNAILREDKDPQKMYATIY ELKEDKSYNVTSVLFRKKKCDYWIRTFVPGSQPGEFTLGNIKSYPGLTSYLVRVVSTNY NQHAMVFFKKVSQNREYFKITLYGRTKELTSELKENFIRFSKSLGLPENHIVFPVPIDQCI DGGGSRRRRKRSGS). In some embodiments, the sidrocalin with the furin cleavage site may be appended to an N-terminus or a C-terminus of the peptide. A peptide may be modified with a signal peptide to mark the peptide for secretion and for additional stability during translation or secretion. For example, a peptide may be modified with a signal peptide and a sidrocalin with a furin cleavage site corresponding to SEQ ID NO: 231 (METDTLLLWVLLLWVPGSTGGSQDSTSDLIPAPPLSKVPLQQNFQDNQFQGKWYVVG LAGNAILREDKDPQKMYATIYELKEDKSYNVTSVLFRKKKCDYWIRTFVPGSQPGEFTL GNIKSYPGLTSYLVRVVSTNYNQHAMVFFKKVSQNREYFKITLYGRTKELTSELKENFI RFSKSLGLPENHIVFPVPIDQCIDGGGSRRRRKRSGS). In some embodiments, the signal peptide and the sidrocalin with the furin cleavage site may be appended to an N-terminus or a C-terminus of the peptide.

Amino acids of a peptide or a peptide complex (e.g., a TfR-binding peptide, a receptor-binding peptide, a target-binding peptide, or a selective depletion complex) can also be mutated, such as to increase half-life, modify, add or delete binding behavior in vivo, add new targeting function, modify surface charge and hydrophobicity, or allow conjugation sites. N-methylation is one example of methylation that can occur in a peptide of the disclosure. In some embodiments, the peptide is modified by methylation on free amines. For example, full methylation can be accomplished through the use of reductive methylation with formaldehyde and sodium cyanoborohydride.

The peptides can be modified to add function, such as to graft loops or sequences from other proteins or peptides onto peptides of this disclosure. Likewise, domains, loops, or sequences from this disclosure can be grafted onto other peptides or proteins such as antibodies that have additional function.

In some embodiments, a selective depletion complex can comprise a tissue targeting domain and can accumulate in the target tissue upon administration to a subject. For example, selective depletion complexes can be conjugated to, linked to, or fused to a molecule (e.g., small molecule, peptide, or protein) with targeting or homing function for a cell of interest or a target protein located on the surface or inside said cell. In some embodiments, selective depletion complexes can be conjugated to, linked to, or fused to a molecule that extends the plasma and/or biological half-life, or modifies the pharmacodynamic (e.g., enhanced binding to a target protein) and/or pharmacokinetic properties (e.g., rate and mode of clearance) of the peptides, or any combination thereof.

A chemical modification can, for instance, extend the half-life of a peptide or change the biodistribution or pharmacokinetic profile. A chemical modification can comprise a polymer, a polyether, polyethylene glycol, a biopolymer, a polyamino acid, a fatty acid, a dendrimer, an Fc region, a simple saturated carbon chain such as palmitate or myristolate, or albumin. A polyamino acid can include, for example, a poly amino acid sequence with repeated single amino acids (e.g., poly glycine), and a poly amino acid sequence with mixed poly amino acid sequences (e.g., gly-ala-gly-ala; SEQ ID NO: 457) that can or may not follow a pattern, or any combination of the foregoing.

The peptides of the present disclosure can be modified such that the modification increases the stability and/or the half-life of the peptides. The attachment of a hydrophobic moiety, such as to the N-terminus, the C-terminus, or an internal amino acid, can be used to extend half-life of a peptide of the present disclosure. The peptides can also be modified to increase or decrease the gut permeability or cellular permeability of the peptide. In some cases, the peptides of the present disclosure show high accumulation in glandular cells of the intestine, demonstrating applicability in the treatment and-or prevention of diseases or conditions of the intestines, such as Crohn's disease or more generally inflammatory bowel diseases. The peptide of the present disclosure can include post-translational modifications (e.g., methylation and/or amidation and/or glycosylation), which can affect, e.g., serum half-life. In some embodiments, simple carbon chains (e.g., by myristoylation and/or palmitylation) can be conjugated to, linked to, the fusion proteins or peptides. The simple carbon chains can render the fusion proteins or peptides easily separable from the unconjugated material. For example, methods that can be used to separate the fusion proteins or peptides from the unconjugated material include, but are not limited to, solvent extraction and reverse phase chromatography. Lipophilic moieties can extend half-life through reversible binding to serum albumin. Conjugated moieties can, e.g., be lipophilic moieties that extend half-life of the peptides through reversible binding to serum albumin. In some embodiments, the lipophilic moiety can be cholesterol or a cholesterol derivative including cholestenes, cholestanes, cholestadienes and oxysterols. In some embodiments, the peptides can be conjugated to, linked to, myristic acid (tetradecanoic acid) or a derivative thereof. In other embodiments, the peptides of the present disclosure can be coupled (e.g., conjugated, linked, or fused) to a half-life modifying agent. Examples of half-life modifying agents can include, but is not limited to: a polymer, a polyethylene glycol (PEG), a hydroxyethyl starch, polyvinyl alcohol, a water soluble polymer, a zwitterionic water soluble polymer, a water soluble poly(amino acid), a water soluble polymer of proline, alanine and serine, a water soluble polymer containing glycine, glutamic acid, and serine, an Fc region, a fatty acid, palmitic acid, or a molecule that binds to albumin. In some embodiments, the half-life modifying agent can be a serum albumin binding peptide, for example SA21 (SEQ ID NO: 178, RLIEDICLPRWGCLWEDD). In some embodiments, a SA21 peptide can be conjugated or fused to the CDPs of the present disclosure (e.g., any of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64). A SA21 fusion peptide can include the SA21 TfR-binding peptide complexes disclosed herein (e.g., SEQ ID NO: 181 or SEQ ID NO: 184). The SA21 peptide can comprise a linker sequence for conjugation to, or fusion between, one or more peptides (e.g., SEQ ID NO: 179, GGGGSGGGGSRLIEDICLPRWGCLWEDDGGGGSGGGGS). Exemplary SA21 peptides, fusion peptides, and linkers are provided in TABLE 5. A control SA21 fusion peptide can comprise a control peptide fused to SA21 (e.g., SEQ ID NO: 180 (GSRLIEDICLPRWGCLWEDDGGGGSGGGGSKCLPPGKPCYGATQKIPCCGVCSHNNCT), SEQ ID NO: 183 (RLIEDICLPRWGCLWEDDGGGGSGGGGSKCLPPGKPCYGATQKIPCCGVCSHNNCT), SEQ ID NO: 182 (GSRLIEDICLPRWGCLWEDDGGGGSGGGGSVRIPVSCKHSGQCLKPCKDAGMRFGKC MNGKCDCTPK), or SEQ ID NO: 185 (RLIEDICLPRWGCLWEDDGGGGSGGGGSVRIPVSCKHSGQCLKPCKDAGMRFGKCMN GKCDCTPK)). Additionally, conjugation of the peptide to a near infrared dye, such as Cy5.5, or to an albumin binder such as Albu-tag can extend serum half-life of any peptide as described herein. In some embodiments, immunogenicity is reduced by using minimal non-human protein sequences to extend serum half-life of the peptide.

TABLE 7
Exemplary TfR-Binding Peptide Complexes Comprising Serum Albumin
Binding Peptides

| SEQ ID NO | Amino Acid Sequence |
|---|---|
| SEQ ID NO: 181 | GSRLIEDICLPRWGCLWEDDGGGGSGGGGSREGCASRCMKYNDELE |
| | KCEARMMSMSNTEEDCEQELEDLLYCLDHCHSQ |
| SEQ ID NO: 184 | RLIEDICLPRWGCLWEDDGGGGSGGGGSREGCASRCMKYNDELEKC |
| | EARMMSMSNTEEDCEQELEDLLYCLDHCHSQ |

In some embodiments, the first two N-terminal amino acids (GS) of SEQ ID NO: 1-SEQ ID NO: 64 serve as a spacer or linker in order to facilitate conjugation or fusion to another molecule, as well as to facilitate cleavage of the peptide from such conjugated to, linked to, or fused molecules. In some embodiments, the fusion proteins or peptides of the present disclosure can be conjugated to, linked to, or fused to other moieties that, e.g., can modify or effect changes to the properties of the peptides.

In some embodiments, peptides or peptide complexes of the present disclosure can also be conjugated to, linked to, or fused to other affinity handles. Other affinity handles can include genetic fusion affinity handles. Genetic fusion affinity handles can include 6×His (HHHHHH (SEQ ID NO: 142) or GGGGSHHHHHH (SEQ ID NO: 228); immobilized metal affinity column purification possible), FLAG (DYKDDDDK (SEQ ID NO: 143); anti-FLAG immunoprecipitation), “shorty” FLAG (DYKDE (SEQ ID NO: 144); anti-FLAG immunoprecipitation), hemagglutinin (YPYDVPDYA (SEQ ID NO: 145); anti-HA immunoprecipitation), and streptavidin binding peptide (DVEAWLGAR (SEQ ID NO: 146); streptavidin-mediated precipitation). In some embodiments, peptides or peptide complexes of the present disclosure can also be conjugated to, linked to, or fused to an expression tag or sequence to improve protein expression and/or purification. Such expression tags can include genetic fusion expression tags. Genetic fusion expression tags can include siderocalin (SEQ ID NO: 147,

METDTLLLWVLLLWVPGSTGDYKDEHHHHHHGGSQDSTSDLIPAPPLSK
VPLQQNFQDNQFQGKWYVVGLAGNAILREDKDPQKMYATIYELKEDKSY
NVTSVLFRKKKCDYWIRTFVPGSQPGEFTLGNIKSYPGLTSYLVRVVST
NYNQHAMVFFKKVSQNREYFKITLYGRTKELTSELKENFIRFSKSLGLP
ENHIVFPVPIDQCIDGGGSENLYFQ).

Additionally, more than one peptide sequence (e.g., a peptide derived from a toxin or knotted venom protein) can be present on, conjugated to, linked to, or fused with a particular peptide. A peptide can be incorporated into a biomolecule by various techniques. A peptide can be incorporated by a chemical transformation, such as the formation of a covalent bond, such as an amide bond. A peptide can be incorporated, for example, by solid phase or solution phase peptide synthesis. A peptide can be incorporated by preparing a nucleic acid sequence encoding the biomolecule, wherein the nucleic acid sequence includes a subsequence that encodes the peptide. The subsequence can be in addition to the sequence that encodes the biomolecule or can substitute for a subsequence of the sequence that encodes the biomolecule.

### Selective Depletion Complexes

In some embodiments, one or more peptides of the present disclosure can form a selective depletion complex (SDC). A selective depletion complex may comprise a target-binding peptide that binds a target molecule and a receptor-binding peptide that binds a cellular receptor (e.g., a cell surface receptor). In some embodiments, the cell surface receptor is a receptor that is endocytosed (e.g., a transferrin receptor or a programmed death-ligand 1). In some embodiments, the cell surface receptor is a receptor that is recycled back to the cell surface following endocytosis. A receptor-binding peptide of the present disclosure may be a transferrin receptor (TfR)-binding peptide or a programmed death ligand 1 (PD-L1)-binding peptide. For example, a selective depletion complex can comprise a TfR-binding peptide and a target-binding peptide. In some embodiments, the receptor-binding peptide (e.g., the TfR-binding peptide or the PD-L1-binding peptide) and the target-binding peptide can be connected via a linker (e.g., a peptide linker). In some embodiments, the receptor-binding peptide and the target-binding peptide can be directly connected without a linker. In some embodiments, the receptor-binding peptide and the target-binding peptide can be connected via a heterodimerization domain. The receptor-binding peptide can bind the receptor (e.g., TfR or PD-L1) with high affinity at both extracellular pH (such as about pH 7.4) and at endosomal pH (such as about pH 5.5). In some embodiments, the receptor-binding peptide of a selective depletion complex may be a TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64). In some embodiments, the receptor-binding peptide of a selective depletion complex may be a PD-L1-binding peptide (e.g., any one of SEQ ID NO: 187, SEQ ID NO: 233-SEQ ID NO: 239, SEQ ID NO: 400-SEQ ID NO: 456, or SEQ ID NO: 141).

The target-binding peptide can bind a target molecule with an affinity that is pH-dependent. For example, the target-binding molecule can bind to the target molecule with higher affinity at extracellular pH (about pH 7.4) and with lower affinity at a lower endosomal pH (such as about pH 5.5 or about pH 6.5). In some embodiments, the target-binding molecule can release the target molecule upon internalization into an endosomal compartment and acidification of the endosome. Such release of the target molecule upon acidification of the endosome can occur at about pH 7.3, pH 7.2, pH 7.1, pH 7.0, pH 6.9, pH 6.8, pH 6.7, pH 6.6, pH 6.5, pH 6.4, pH 6.3, pH 6.2, pH 6.1, pH 6.0, pH 5.9, pH 5.8, pH 5.7, pH 5.6, pH 5.5, pH 5.4, pH 5.3, pH 5.2, pH 5.1, pH 5.0, pH 4.9, pH 4.8, pH 4.7, pH 4.6, pH 4.5, or lower. In some embodiments, release of the target molecule can occur at a pH of from about pH 7.0 to about pH 4.5, from about pH 6.5 to about pH 5.0, or from about pH 6.0 to about pH 5.5. In some embodiments, the receptor-binding peptide binds a receptor (e.g., a receptor that undergoes recycling) with pH-independent binding (e.g., high affinity at extracellular pH and high affinity at endosomal pH) and the target-binding peptide binds the target with pH-dependent binding (e.g., high affinity at extracellular pH and low affinity at endosomal pH). A selective depletion complex (SDC) comprising a pH-independent receptor-binding peptide and a pH-dependent target-binding peptide may be catalytic (e.g., reused). The SDC may stay bound to the receptor through multiple rounds of endocytosis and has the potential to carry another target molecule in each round and leave the target molecule in the endosome/lysosome for degradation. Thus, one catalytic SDC molecule may cause the degradation of multiple target molecules.

In some embodiments, the receptor-binding peptide can bind to the receptor with an affinity that is pH dependent. For example, the receptor-binding molecule can bind to the receptor with higher affinity at extracellular pH (such as about pH 7.4) and with lower affinity at a lower endosomal pH (such as about pH 5.5 or about pH 6.5), thereby releasing the selective depletion complex from the receptor upon internalization and acidification of the endosomal compartment. In some embodiment, the receptor-binding peptide can bind the receptor with an affinity that is pH dependent and the target-binding peptide can bind the target with an affinity that is pH dependent or that is pH-independent. A selective depletion molecule can be used to selectively deplete a target molecule (e.g., a soluble protein or a cell surface protein). For example, a selective depletion complex comprising a receptor-binding peptide and a target-binding peptide can bind to the receptor via the receptor-binding peptide and to a target molecule (e.g., a soluble protein or a cell surface protein). The target molecule can be delivered to an endocytic compartment via receptor-mediated endocytosis of the receptor and the selective depletion molecule. In the endocytic compartment, the selective depletion complex can remain bound to the receptor, and the target molecule can be released from the selective depletion complex as the endocytic compartment acidifies. The selective depletion molecule can be recycled to the cell surface along with the receptor, and the target molecule can continue to the lysosome where it is degraded. In some embodiments, the target molecule can remain in the lysosome without being degraded, resulting in enrichment of the target molecule in the lysosome. The selective depletion complexes of the present disclosure can have a low molecular weight compared to target-binding antibodies and can be used to bind and deplete a target without requiring a supply and distribution cold chain.

In some embodiments, a receptor-binding peptide (a TfR-binding peptide or a PD-L1-binding peptide) may bind to a cellular receptor (e.g., TfR or PD-L1) with an equilibrium dissociation constant (K_{D}) of less than 50 μM, less than 5 μM, less than 500 nM, less than 100 nM, less than 40 nM, less than 30 nM, less than 20 nM, less than 10 nM, less than 5 nM, less than 2 nM, less than 1 nM, less than 0.5 nM, less than 0.4 nM, less than 0.3 nM, less than 0.2 nM, or less than 0.1 nM. In some embodiments, a receptor-binding peptide has an off rate that is slower than the recycling rate of the cellular receptor, such that the receptor-binding peptide is likely to remain bound to receptor during the recycling process. In some embodiments, the receptor-binding peptide may have an off rate that is no faster than 1 minute, no faster than 2 minutes, no faster than 3 minutes, no faster than 4 minutes, no faster than 5 minutes, no faster than 7 minutes, no faster than 10 minutes, no faster than 15 minutes, or no faster than 20 minutes. In some embodiments, the receptor-binding peptide may have an off rate that is from about 1 minute to about 20 minutes, from about 2 minutes to about 15 minutes, from about 2 minutes to about 10 minutes, or from about 5 minutes to about 10 minutes.

The selective depletion complexes of the present disclosure can be used to treat a disease or a condition by selectively depleting a target molecule that is associated with the disease or the condition. For example, a selective depletion complex can be used to selectively deplete a soluble or cell surface protein that accumulates, contains a disease-associated mutation (e.g., a mutation causing constitutive activity, resistance to treatment, or dominant negative activity), or is over-expressed in a disease state. In some embodiments, the selective depletion complexes of the present disclosure can be used for the treatment and prevention of various neurological diseases including but not limited to epilepsy, schizophrenia, depression, anxiety, bipolar disorder, developmental brain disorders (e.g., autism spectrum), or mood disorder.

Binding of the herein described selective depletion complexes (e.g., peptide conjugates, fusion peptides, or recombinantly produced peptide complexes) to TfR and subsequent transport across a cell layer or barrier such as the BBB (e.g., via vesicular transcytosis) or a cell membrane (e.g., via endocytosis) can have implications in a number of diseases, conditions, or disorders associated with neurodegeneration. Neurodegenerative diseases that can treated or prevented with the herein described selective depletion complexes can include Alzheimer's disease, Amyotrophic lateral sclerosis, Friedreich's ataxia, Huntington's disease, Lewy body disease, Parkinson's disease, multiple system atrophy (MSA), Spinal muscular atrophy, Motor neuron disease, Lyme disease, Ataxia-telangiectasia, Autosomal dominant cerebellar ataxia, Batten disease, Corticobasal syndrome, Creutzfeldt-Jakob disease, Fragile X-associated tremor/ataxia syndrome, Kufor-Rakeb syndrome, Machado-Joseph disease, multiple sclerosis, chronic traumatic encephalopathy, or frontotemporal dementia. In some embodiments, the TfR-binding peptide can be used in combination with BACE inhibitors, galantamine, amantadine, benztropine, biperiden, bromocriptin, carbidopa, donepezil, entacapone, levodopa, pergolie, pramipexole, procyclidine, rivastigmine, ropinirole, selegiline, tacrine, tolcapone, or trihexyphenidyl to treat and/or prevent a neurodegenerative disease. For example, a selective depletion complex comprising a target-binding peptide that binds a protein associated with neurodegeneration (e.g., tau, amyloid ß (Aß), huntingtin, or α-synuclein) can be used to treat a neurodegenerative disease.

Binding of the herein described selective depletion complexes (e.g., peptide conjugates, fusion peptides, or recombinantly produced peptide complexes) to TfR and subsequent transport across a cell layer or barrier such as the BBB (e.g., via vesicular transcytosis) or a cell membrane (e.g., via endocytosis) can have implications in various cancers. Cancers that can treated or prevented with the herein described selective depletion complexes can include breast cancer, liver cancer, colon cancer, brain cancer, leukemia, lymphoma, non-Hodgkin lymphoma, myeloma, blood-cell-derived cancer, spleen cancer, lung cancer, pancreatic cancer, prostate cancer, sarcoma, stomach cancer, esophageal cancer, gastrointestinal (GI) cancers, thyroid cancer, endometrial cancer, bladder cancer, cancers of the salivary gland, kidney cancer, muscle cancers, ovarian cancer, glioblastoma, astrocytoma, glioma, medulloblastoma, ependymoma, choroid plexus carcinoma, midline glioma, diffuse intrinsic pontine glioma, lung cancer, bone marrow cell cancers, skin cancer, melanoma, genitourinary cancer, osteosarcoma, muscle-derived sarcoma, melanoma, head and neck cancer, a neuroblastoma, glioblastoma, astrocytoma, glioma, medulloblastoma, ependymoma, choroid plexus carcinoma, midline glioma, and diffuse intrinsic pontine glioma (DIPG), or a CMYC-overexpressing cancer. For example, a selective depletion complex comprising a target-binding peptide that binds a protein associated with cancer (e.g., HER2, EGFR, FGFR-1, PD-L1, VEGF, PD-1, CD38, GD2, SLAMF7, CTLA-4, CCR4, CD20, PDGFRα, VEGFR2, CD33, CD30, CD22, CD79B, Nectin-4, or TROP2) can be used to treat a cancer. In some embodiments, a selective depletion complex for treatment of a cancer can comprise a target-binding peptide that binds an extracellular, soluble, or cell surface protein associated with cell growth, cell division, avoidance of cell death, immune evasion, suppression of inflammatory responses, promotion of vascular growth, or protection from hypoxia. In some embodiments, a selective depletion complex of the present disclosure can be used to deplete anti-inflammatory stimuli (e.g., molecules associated with N2-polarized macrophages or molecules associated with microglia or regulatory T-cells) and promote tumor targeting abilities of abilities of the innate and adaptive immune systems. Selective depletion complexes comprising a target-binding peptide that binds molecules associated with the anti-inflammatory stimuli can augment therapies that otherwise are prone to immune exhaustion (e.g. ionizing radiation or CAR-T cell therapies).

In some embodiments, the selective depletion complex may be used to reduce immune suppression or suppress pro-inflammatory signaling, such as in immune-mediated diseases. For example, a selective depletion complex may comprise a target-binding peptide that binds a protein associated with immune suppression or pro-inflammatory signaling (e.g., CD47, CD39, CD24, CD25, CD74, TNF-α, IL-1, IL-1R, IL-2, IL-2R, IL-6, IL-6R, IL-10, IL-10R, IL-23, IL-12, PD-1, PD-L1). In some embodiments, a selective depletion complex may be used to treat an inflammatory or neurological condition (e.g., neuroinflammation, neuroinflammatory disease, stroke, traumatic brain injury, Alzheimer's disease, or other tauopathies including neurofibrillary tangle dementia, chronic traumatic encephalopathy (CTE), aging-related tau astrogliopathy, frontotemporal dementia, parkinsonism, progressive supranuclear palsy, corticobasal degeneration, lytico-bodig disease, ganglioglioma, meningioangiomatosis, or subacute sclerosing panencephalitis). For example, a selective depletion complex comprising a TNF-α-binding peptide may be used to treat neuroinflammation, neuroinflammatory disease, stroke, traumatic brain injury, or Alzheimer's disease.

Binding of the herein described selective depletion complexes (e.g., peptide conjugates, fusion peptides, or recombinantly produced peptide complexes) to TfR and subsequent transport across a cell layer or barrier such as the BBB (e.g., via vesicular transcytosis) or a cell membrane (e.g., via endocytosis) can have implications in a number of diseases, conditions, or disorders associated with harmful inflammation. Harmful inflammation that can be treated or prevented with the herein described selective depletion complexes can include rheumatoid arthritis, psoriasis, multiple sclerosis, lupus, ankylosing spondylitis, antiphospholipid antibody syndrome, gout, inflammatory arthritis center, myositis, scleroderma, Sjogren's disease, vasculitis, inflammatory bowel disease, ulcerative colitis, Crohn's disease, graft-vs-host disease, cytokine storms, cystic fibrosis, inflammation-associated neurodegeneration (e.g., age-associated tauopathy or Alzheimer's Disease), or autoimmune disorders. For example, a selective depletion complex comprising a target-binding peptide that binds a target associated with acute or chronic inflammation (e.g., apolipoprotein E4, TNF-α, IL-1, IL-6, IL-7, IL-12, and IL-23) to selectively deplete inflammatory cytokines or chemokines. In some embodiments, a selective depletion complex may target autoantibodies, for example autoantibodies associated with disease, such as diabetes, thyroid disease, inflammatory disease, systemic lupus erythematosus (SLE or lupus), muscular function, skin disease, organ disease, kidney disease, or rheumatoid arthritis. In some embodiments, a selective depletion complex comprising a target-binding peptide that binds IL-6 can be used to treat inflammation associated with a coronavirus infection (e.g., SARS-CoV-2). Selective depletion complexes that selectively deplete IL-6-elimiating can decrease IL-6 signaling. Apolipoprotein E4 can be associated with Alzheimer's disease.

Binding of the herein described selective depletion complexes (e.g., peptide conjugates, fusion peptides, or recombinantly produced peptide complexes) to TfR and subsequent transport across a cell layer or barrier such as the BBB (e.g., via vesicular transcytosis) or a cell membrane (e.g., via endocytosis) can have implications in various lysosomal storage diseases. Lysosomal storage diseases that can treated or prevented with the herein described selective depletion complexes can include Gaucher's Disease (deficiency of glucocerebrosidase) or Pompe Disease (deficiency of α-glucosidase). A lysosomal storage enzyme can be administered to the patient such that it is available in the serum or other extracellular fluids. In some embodiments, a selective depletion complex of the present disclosure can be used to selectively recruit lysosomal enzymes to the lysosome, thereby treating a lysosomal storage disease associated with decreased expression of a lysosomal enzyme. The selective depletion complex comprising a target-binding peptide that binds a lysosomal enzyme (e.g., glucocerebrosidase or α-glucosidase) can selectively recruit the lysosomal enzyme into an endocytic compartment via TfR-mediated endocytosis. The selective depletion complex can be recycled to the cell surface, and the lysosomal enzyme target can be delivered to the lysosome, thereby enriching the lysosomal enzyme in the lysosome and treating the lysosomal storage disease.

In some embodiments, a selective depletion complex (e.g., comprising a target-binding peptide and a cellular receptor-binding peptide) or a selective depletion complex component (e.g., comprising a target-binding peptide or a cellular receptor-binding peptide and a dimerization domain) may comprise a sequence of any one of SEQ ID NO: 288-SEQ ID NO: 313, SEQ ID NO: 315-SEQ ID NO: 348, SEQ ID NO: 351, SEQ ID NO: 352, SEQ ID NO: 355, SEQ ID NO: 356, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 371, SEQ ID NO: 373, SEQ ID NO: 376, SEQ ID NO: 378, SEQ ID NO: 382, SEQ ID NO: 384, SEQ ID NO: 387, or SEQ ID NO: 389, or a fragment thereof. In some embodiments, a selective depletion complex (e.g., comprising a target-binding peptide and a cellular receptor-binding peptide) may comprise a sequence that has at least 80%, at least 90%, at least 92%, at least 93%, at least 94%, at least 95%, at least 96%, at least 97%, at least 98%, or at least 99% sequence identity with SEQ ID NO: 96, or at least 80%, at least 90%, at least 92%, at least 93%, at least 94%, at least 95%, at least 96%, at least 97%, at least 98%, or at least 99% sequence identity to any one of SEQ ID NO: 288-SEQ ID NO: 313, SEQ ID NO: 315-SEQ ID NO: 348, SEQ ID NO: 351, SEQ ID NO: 352, SEQ ID NO: 355, SEQ ID NO: 356, SEQ ID NO: 358, SEQ ID NO: 359, SEQ ID NO: 360, SEQ ID NO: 361, SEQ ID NO: 362, SEQ ID NO: 363, SEQ ID NO: 364, SEQ ID NO: 365, SEQ ID NO: 371, SEQ ID NO: 373, SEQ ID NO: 376, SEQ ID NO: 378, SEQ ID NO: 382, SEQ ID NO: 384, SEQ ID NO: 387, or SEQ ID NO: 389, or a fragment thereof. Examples of selective depletion complexes and selection depletion complex components, and their corresponding targets or cellular receptors, are provided in TABLE 8.

TABLE 8
Exemplary Selective Depletion Complexes and Complex Components

| | | SEQ ID | |
|---|---|---|---|
| Target | Receptor | NO | Sequence |
| EGFR | TfR | SEQ ID | REGCASRCMKYNDELEKCEARMMSMSNTEEDCEQE |
| | | NO: 288 | LEDLLYCLDHCHSQGGGGSGGGGSGGGGSQVKLEES |
| | | | GGGSVQTGGSLRLTCAASGHTSRSYGMHWFRQAPG |
| | | | KEREFVSGISWRGDSTGYADSVKGRFTISRDNAKNTV |
| | | | DLQMNSLKPEDTAIYYCAAAAGSHWYGTHYEYDYW |
| | | | GQGTQVTVSS |
| EGFR | TfR | SEQ ID | QVQLQESGGGVVQPGRSLRLSCAASRFTFNNYAMH |
| | | NO: 289 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSGG |
| | | | GGSGGGGSQVKLEESGGGSVQTGGSLRLTCAASGHT |
| | | | SRSYGMHWFRQAPGKEREFVSGISWRGDSTGYADSV |
| | | | KGRFTISRDNAKNTVDLQMNSLKPEDTAIYYCAAAA |
| | | | GSHWYGTHYEYDYWGQGTQVTVSS |
| PD-L1 | TfR | SEQ ID | REGCASRCMKYNDELEKCEARMMSMSNTEEDCEQE |
| | | NO: 290 | LEDLLYCLDHCHSQGGGGSGGGGSGGGGSEHDCKV |
| | | | HCVKEWMAGHACAERQKSYTIGRAHCSGQKFDVFK |
| | | | CLDHCAAP |
| PD-L1 | TfR | SEQ ID | QVQLQESGGGVVQPGRSLRLSCAASRFTFNNYAMH |
| | | NO: 291 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSGG |
| | | | GGSGGGGSEHDCKVHCVKEWMAGHACAERQKSYTI |
| | | | GRAHCSGQKFDVFKCLDHCAAP |
| | TfR | SEQ ID | REGCASRCMKYNDELEKCEARMMSMSNTEEDCEQE |
| | | NO: 292 | LEDLLYCLDHCHSQGGGGSGGGGSGGGGSEPKSSDK |
| | | | THTCPPCPAPEAAGGPSVFLFPPKPKDTLMISRTPEVT |
| | | | CVVVDVSHEDPEVKFNWYVDGVEVHNAKTKPREEQ |
| | | | YNSTYRVVSVLTVLHQDWLNGKEYKCKVSNKALGA |
| | | | PIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQK |
| | | | SLSLSPGK |
| EGFR | TfR | SEQ ID | REGCASRCMKYNDELEKCEARMMSMSNTEEDCEQE |
| | | NO: 293 | LEDLLYCLDHCHSQGGGGSGGGGSGGGGSEPKSSDK |
| | | | THTCPPCPAPEAAGGPSVFLFPPKPKDTLMISRTPEVT |
| | | | CVVVDVSHEDPEVKFNWYVDGVEVHNAKTKPREEQ |
| | | | YNSTYRVVSVLTVLHQDWLNGKEYKCKVSNKALGA |
| | | | PIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQK |
| | | | SLSLSPGKGGGGSGGGGSGGGGSQVKLEESGGGSVQ |
| | | | TGGSLRLTCAASGHTSRSYGMHWFRQAPGKEREFVS |
| | | | GISWRGDSTGYADSVKGRFTISRDNAKNTVDLQMNS |
| | | | LKPEDTAIYYCAAAAGSHWYGTHYEYDYWGQGTQV |
| | | | TVSS |
| PD-L1 | TfR | SEQ ID | REGCASRCMKYNDELEKCEARMMSMSNTEEDCEQE |
| | | NO: 294 | LEDLLYCLDHCHSQGGGGSGGGGSGGGGSEPKSSDK |
| | | | THTCPPCPAPEAAGGPSVFLFPPKPKDTLMISRTPEVT |
| | | | CVVVDVSHEDPEVKFNWYVDGVEVHNAKTKPREEQ |
| | | | YNSTYRVVSVLTVLHQDWLNGKEYKCKVSNKALGA |
| | | | PIEKTISKAKGQPREPQVYTLPPSREEMTKNQVSLWC |
| | | | LVKGFYPSDIAVEWESNGQPENNYKTTPPVLDSDGSF |
| | | | FLYSKLTVDKSRWQQGNVFSCSVMHEALHNHYTQK |
| | | | SLSLSPGKGGGGSGGGGSGGGGSEHDCKVHCVKEW |
| | | | MAGHACAERQKSYTIGRAHCSGQKFDVFKCLDHCA |
| | | | AP |
| | TfR | SEQ ID | QVQLQESGGGVVQPGRSLRLSCAASRFTENNYAMH |
| | | NO: 295 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSEP |
| | | | KSSDKTHTCPPCPAPEAAGGPSVFLFPPKPKDTLMISR |
| | | | TPEVTCVVVDVSHEDPEVKFNWYVDGVEVHNAKTK |
| | | | PREEQYNSTYRVVSVLTVLHQDWLNGKEYKCKVSN |
| | | | KALGAPIEKTISKAKGQPREPQVYTLPPSREEMTKNQ |
| | | | VSLWCLVKGFYPSDIAVEWESNGQPENNYKTTPPVL |
| | | | DSDGSFFLYSKLTVDKSRWQQGNVFSCSVMHEALHN |
| | | | HYTQKSLSLSPGK |
| EGFR | TfR | SEQ ID | QVQLQESGGGVVQPGRSLRLSCAASRFTFNNYAMH |
| | | NO: 296 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSEP |
| | | | KSSDKTHTCPPCPAPEAAGGPSVFLFPPKPKDTLMISR |
| | | | TPEVTCVVVDVSHEDPEVKFNWYVDGVEVHNAKTK |
| | | | PREEQYNSTYRVVSVLTVLHQDWLNGKEYKCKVSN |
| | | | KALGAPIEKTISKAKGQPREPQVYTLPPSREEMTKNQ |
| | | | VSLWCLVKGFYPSDIAVEWESNGQPENNYKTTPPVL |
| | | | DSDGSFFLYSKLTVDKSRWQQGNVFSCSVMHEALHN |
| | | | HYTQKSLSLSPGKGGGGSGGGGSGGGGSQVKLEESG |
| | | | GGSVQTGGSLRLTCAASGHTSRSYGMHWFRQAPGK |
| | | | EREFVSGISWRGDSTGYADSVKGRFTISRDNAKNTVD |
| | | | LQMNSLKPEDTAIYYCAAAAGSHWYGTHYEYDYWG |
| | | | QGTQVTVSS |
| PD-L1 | TfR | SEQ ID | QVQLQESGGGVVQPGRSLRLSCAASRFTFNNYAMH |
| | | NO: 297 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSEP |
| | | | KSSDKTHTCPPCPAPEAAGGPSVFLFPPKPKDTLMISR |
| | | | TPEVTCVVVDVSHEDPEVKFNWYVDGVEVHNAKTK |
| | | | PREEQYNSTYRVVSVLTVLHQDWLNGKEYKCKVSN |
| | | | KALGAPIEKTISKAKGQPREPQVYTLPPSREEMTKNQ |
| | | | VSLWCLVKGFYPSDIAVEWESNGQPENNYKTTPPVL |
| | | | DSDGSFFLYSKLTVDKSRWQQGNVFSCSVMHEALHN |
| | | | HYTQKSLSLSPGKGGGGSGGGGSGGGGSEHDCKVHC |
| | | | VKEWMAGHACAERQKSYTIGRAHCSGQKFDVFKCL |
| | | | DHCAAP |
| | TfR | SEQ ID | QVQLQESGGGVVQPGRSLRLSCAASRYPFHHHDHH |
| | | NO: 298 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSEP |
| | | | KSSDKTHTCPPCPAPEAAGGPSVFLFPPKPKDTLMISR |
| | | | TPEVTCVVVDVSHEDPEVKFNWYVDGVEVHNAKTK |
| | | | PREEQYNSTYRVVSVLTVLHQDWLNGKEYKCKVSN |
| | | | KALGAPIEKTISKAKGQPREPQVYTLPPSREEMTKNQ |
| | | | VSLWCLVKGFYPSDIAVEWESNGQPENNYKTTPPVL |
| | | | DSDGSFFLYSKLTVDKSRWQQGNVFSCSVMHEALHN |
| | | | HYTQKSLSLSPGK |
| EGFR | TfR | SEQ ID | QVQLQESGGGVVQPGRSLRLSCAASRYPFHHHDHH |
| | | NO: 299 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSEP |
| | | | KSSDKTHTCPPCPAPEAAGGPSVFLFPPKPKDTLMISR |
| | | | TPEVTCVVVDVSHEDPEVKFNWYVDGVEVHNAKTK |
| | | | PREEQYNSTYRVVSVLTVLHQDWLNGKEYKCKVSN |
| | | | KALGAPIEKTISKAKGQPREPQVYTLPPSREEMTKNQ |
| | | | VSLWCLVKGFYPSDIAVEWESNGQPENNYKTTPPVL |
| | | | DSDGSFFLYSKLTVDKSRWQQGNVFSCSVMHEALHN |
| | | | HYTQKSLSLSPGKGGGGSGGGGSGGGGSQVKLEESG |
| | | | GGSVQTGGSLRLTCAASGHTSRSYGMGWFRQAPGK |
| | | | EREFVSGISWRGDSTGYADSVKGRFTISRDNAKNTVD |
| | | | LQMNSLKPEDTAIYYCAAAAGSAWYHTLYEYDYWG |
| | | | QGTQVTVSS |
| PD-L1 | TfR | SEQ ID | QVQLQESGGGVVQPGRSLRLSCAASRYPFHHHDHH |
| | | NO: 300 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSEP |
| | | | KSSDKTHTCPPCPAPEAAGGPSVFLFPPKPKDTLMISR |
| | | | TPEVTCVVVDVSHEDPEVKFNWYVDGVEVHNAKTK |
| | | | PREEQYNSTYRVVSVLTVLHQDWLNGKEYKCKVSN |
| | | | KALGAPIEKTISKAKGQPREPQVYTLPPSREEMTKNQ |
| | | | VSLWCLVKGFYPSDIAVEWESNGQPENNYKTTPPVL |
| | | | DSDGSFFLYSKLTVDKSRWQQGNVFSCSVMHEALHN |
| | | | HYTQKSLSLSPGKGGGGSGGGGSGGGGSEEDCKVHC |
| | | | VKEWMAGKACAERQKSYTIGRAHCSGQKFDVFKCL |
| | | | DHCAAP |
| EGFR | | SEQ ID | EPKSSDKTHTCPPCPAPEAAGGPSVFLFPPKPKDTLMI |
| | | NO: 301 | SRTPEVTCVVVDVSHEDPEVKFNWYVDGVEVHNAK |
| | | | TKPREEQYNSTYRVVSVLTVLHQDWLNGKEYKCKV |
| | | | SNKALGAPIEKTISKAKGQPREPQVYTLPPSREEMTK |
| | | | NQVSLSCAVKGFYPSDIAVEWESNGQPENNYKTTPP |
| | | | VLDSDGSFFLVSKLTVDKSRWQQGNVFSCSVMHEAL |
| | | | HNHYTQKSLSLSPGKGGGGSGGGGSGGGGSQVKLEE |
| | | | SGGGSVQTGGSLRLTCAASGHTSRSYGMGWFRQAPG |
| | | | KEREFVSGISWRGDSTGYADSVKGRFTISRDNAKNTV |
| | | | DLQMNSLKPEDTAIYYCAAAAGSAWYHTLYEYDYW |
| | | | GQGTQVTVSS |
| EGFR | | SEQ ID | EPKSSDKTHTCPPCPAPEAAGGPSVFLFPPKPKDTLMI |
| | | NO: 302 | SRTPEVTCVVVDVSHEDPEVKFNWYVDGVEVHNAK |
| | | | TKPREEQYNSTYRVVSVLTVLHQDWLNGKEYKCKV |
| | | | SNKALGAPIEKTISKAKGQPREPQVYTLPPSREEMTK |
| | | | NQVSLSCAVKGFYPSDIAVEWESNGQPENNYKTTPP |
| | | | VLDSDGSFFLVSKLTVDKSRWQQGNVFSCSVMHEAL |
| | | | HNHYTQKSLSLSPGKGGGGSGGGGSGGGGSQVKLEE |
| | | | SGGGSVQTGGSLRLTCAASGHTSRSYGMHWFRQAPG |
| | | | KEREFVSGISWRGDSTGYADSVKGRFTISRDNAKNTV |
| | | | DLQMNSLKPEDTAIYYCAAAAGSHWYGTHYEYDYW |
| | | | GQGTQVTVSS |
| PD-L1 | | SEQ ID | EPKSSDKTHTCPPCPAPEAAGGPSVFLFPPKPKDTLMI |
| | | NO: 303 | SRTPEVTCVVVDVSHEDPEVKFNWYVDGVEVHNAK |
| | | | TKPREEQYNSTYRVVSVLTVLHQDWLNGKEYKCKV |
| | | | SNKALGAPIEKTISKAKGQPREPQVYTLPPSREEMTK |
| | | | NQVSLSCAVKGFYPSDIAVEWESNGQPENNYKTTPP |
| | | | VLDSDGSFFLVSKLTVDKSRWQQGNVFSCSVMHEAL |
| | | | HNHYTQKSLSLSPGKGGGGSGGGGSGGGGSEEDCKV |
| | | | HCVKEWMAGKACAERQKSYTIGRAHCSGQKFDVFK |
| | | | CLDHCAAP |
| PD-L1 | | SEQ ID | EPKSSDKTHTCPPCPAPEAAGGPSVFLFPPKPKDTLMI |
| | | NO: 304 | SRTPEVTCVVVDVSHEDPEVKFNWYVDGVEVHNAK |
| | | | TKPREEQYNSTYRVVSVLTVLHQDWLNGKEYKCKV |
| | | | SNKALGAPIEKTISKAKGQPREPQVYTLPPSREEMTK |
| | | | NQVSLSCAVKGFYPSDIAVEWESNGQPENNYKTTPP |
| | | | VLDSDGSFFLVSKLTVDKSRWQQGNVFSCSVMHEAL |
| | | | HNHYTQKSLSLSPGKGGGGSGGGGSGGGGSEHDCK |
| | | | VHCVKEWMAGHACAERQKSYTIGRAHCSGQKFDVF |
| | | | KCLDHCAAP |
| EGFR | TfR | SEQ ID | REGCASRCMKYNDELEKCEARMMSMSNTEEDCEQE |
| | | NO: 305 | LEDLLYCLDHCHSQGGGGSGGGGSGGGGSEPKSSDK |
| | | | THTCPPCPAPEAAGGPSVFLFPPKPKDTLMISRTPEVT |
| | | | CVVVDVSHEDPEVKFNWYVDGVEVHNAKTKPREEQ |
| | | | YNSTYRVVSVLTVLHQDWLNGKEYKCKVSNKALGA |
| | | | PIEKTISKAKGQPREPQVYTLPPSREEMTKNQVSLSCA |
| | | | VKGFYPSDIAVEWESNGQPENNYKTTPPVLDSDGSFF |
| | | | LVSKLTVDKSRWQQGNVFSCSVMHEALHNHYTQKS |
| | | | LSLSPGKGGGGSGGGGSGGGGSQVKLEESGGGSVQT |
| | | | GGSLRLTCAASGHTSRSYGMHWFRQAPGKEREFVSG |
| | | | ISWRGDSTGYADSVKGRFTISRDNAKNTVDLQMNSL |
| | | | KPEDTAIYYCAAAAGSHWYGTHYEYDYWGQGTQVT |
| | | | VSS |
| PD-L1 | TfR | SEQ ID | REGCASRCMKYNDELEKCEARMMSMSNTEEDCEQE |
| | | NO: 306 | LEDLLYCLDHCHSQGGGGSGGGGSGGGGSEPKSSDK |
| | | | THTCPPCPAPEAAGGPSVFLFPPKPKDTLMISRTPEVT |
| | | | CVVVDVSHEDPEVKFNWYVDGVEVHNAKTKPREEQ |
| | | | YNSTYRVVSVLTVLHQDWLNGKEYKCKVSNKALGA |
| | | | PIEKTISKAKGQPREPQVYTLPPSREEMTKNQVSLSCA |
| | | | VKGFYPSDIAVEWESNGQPENNYKTTPPVLDSDGSFF |
| | | | LVSKLTVDKSRWQQGNVFSCSVMHEALHNHYTQKS |
| | | | LSLSPGKGGGGSGGGGSGGGGSEHDCKVHCVKEWM |
| | | | AGHACAERQKSYTIGRAHCSGQKFDVFKCLDHCAAP |
| EGFR | TfR | SEQ ID | REGCASRCMKYNDELEKCEARMMSMSNTEEDCEQE |
| | | NO: 307 | LEDLLYCLDHCHSQGGGGSGGGGSGGGGSEPKSSDK |
| | | | THTCPPCPAPEAAGGPSVFLFPPKPKDTLMISRTPEVT |
| | | | CVVVDVSHEDPEVKFNWYVDGVEVHNAKTKPREEQ |
| | | | YNSTYRVVSVLTVLHQDWLNGKEYKCKVSNKALGA |
| | | | PIEKTISKAKGQPREPQVYTLPPSREEMTKNQVSLTCL |
| | | | VKGFYPSDIAVEWESNGQPENNYKTTPPVLDSDGSFF |
| | | | LYSKLTVDKSRWQQGNVFSCSVMHEALHNHYTQKS |
| | | | LSLSPGKGGGGSGGGGSGGGGSQVKLEESGGGSVQT |
| | | | GGSLRLTCAASGHTSRSYGMHWFRQAPGKEREFVSG |
| | | | ISWRGDSTGYADSVKGRFTISRDNAKNTVDLQMNSL |
| | | | KPEDTAIYYCAAAAGSHWYGTHYEYDYWGQGTQVT |
| | | | VSS |
| PD-L1 | TfR | SEQ ID | REGCASRCMKYNDELEKCEARMMSMSNTEEDCEQE |
| | | NO: 308 | LEDLLYCLDHCHSQGGGGSGGGGSGGGGSEPKSSDK |
| | | | THTCPPCPAPEAAGGPSVFLFPPKPKDTLMISRTPEVT |
| | | | CVVVDVSHEDPEVKFNWYVDGVEVHNAKTKPREEQ |
| | | | YNSTYRVVSVLTVLHQDWLNGKEYKCKVSNKALGA |
| | | | PIEKTISKAKGQPREPQVYTLPPSREEMTKNQVSLTCL |
| | | | VKGFYPSDIAVEWESNGQPENNYKTTPPVLDSDGSFF |
| | | | LYSKLTVDKSRWQQGNVFSCSVMHEALHNHYTQKS |
| | | | LSLSPGKGGGGSGGGGSGGGGSEHDCKVHCVKEWM |
| | | | AGHACAERQKSYTIGRAHCSGQKFDVFKCLDHCAAP |
| EGFR | | SEQ ID | QVKLEESGGGSVQTGGSLRLTCAASGHTSRSYGMH |
| | | NO: 309 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGGGGSGGGGSGGGGSEPK |
| | | | SSDKTHTCPPCPAPEAAGGPSVFLFPPKPKDTLMISRT |
| | | | PEVTCVVVDVSHEDPEVKFNWYVDGVEVHNAKTKP |
| | | | REEQYNSTYRVVSVLTVLHQDWLNGKEYKCKVSNK |
| | | | ALGAPIEKTISKAKGQPREPQVYTLPPSREEMTKNQV |
| | | | SLWCLVKGFYPSDIAVEWESNGQPENNYKTTPPVLD |
| | | | SDGSFFLYSKLTVDKSRWQQGNVFSCSVMHEALHNH |
| | | | YTQKSLSLSPGK |
| EGFR | TfR | SEQ ID | QVKLEESGGGSVQTGGSLRLTCAASGHTSRSYGMH |
| | | NO: 310 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGGGGSGGGGSGGGGSEPK |
| | | | SSDKTHTCPPCPAPEAAGGPSVFLFPPKPKDTLMISRT |
| | | | PEVTCVVVDVSHEDPEVKFNWYVDGVEVHNAKTKP |
| | | | REEQYNSTYRVVSVLTVLHQDWLNGKEYKCKVSNK |
| | | | ALGAPIEKTISKAKGQPREPQVYTLPPSREEMTKNQV |
| | | | SLWCLVKGFYPSDIAVEWESNGQPENNYKTTPPVLD |
| | | | SDGSFFLYSKLTVDKSRWQQGNVFSCSVMHEALHNH |
| | | | YTQKSLSLSPGKGGGGSGGGGSGGGGSREGCASRCM |
| | | | KYNDELEKCEARMMSMSNTEEDCEQELEDLLYCLD |
| | | | HCHSQ |
| | TfR | SEQ ID | EPKSSDKTHTCPPCPAPEAAGGPSVFLFPPKPKDTLMI |
| | | NO: 311 | SRTPEVTCVVVDVSHEDPEVKFNWYVDGVEVHNAK |
| | | | TKPREEQYNSTYRVVSVLTVLHQDWLNGKEYKCKV |
| | | | SNKALGAPIEKTISKAKGQPREPQVYTLPPSREEMTK |
| | | | NQVSLSCAVKGFYPSDIAVEWESNGQPENNYKTTPP |
| | | | VLDSDGSFFLVSKLTVDKSRWQQGNVFSCSVMHEAL |
| | | | HNHYTQKSLSLSPGKGGGGSGGGGSGGGGSREGCAS |
| | | | RCMKYNDELEKCEARMMSMSNTEEDCEQELEDLLY |
| | | | CLDHCHSQ |
| EGFR | TfR | SEQ ID | QVKLEESGGGSVQTGGSLRLTCAASGHTSRSYGMH |
| | | NO: 312 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGGGGSGGGGSGGGGSEPK |
| | | | SSDKTHTCPPCPAPEAAGGPSVFLFPPKPKDTLMISRT |
| | | | PEVTCVVVDVSHEDPEVKFNWYVDGVEVHNAKTKP |
| | | | REEQYNSTYRVVSVLTVLHQDWLNGKEYKCKVSNK |
| | | | ALGAPIEKTISKAKGQPREPQVYTLPPSREEMTKNQV |
| | | | SLSCAVKGFYPSDIAVEWESNGQPENNYKTTPPVLDS |
| | | | DGSFFLVSKLTVDKSRWQQGNVFSCSVMHEALHNH |
| | | | YTQKSLSLSPGKGGGGSGGGGSGGGGSREGCASRCM |
| | | | KYNDELEKCEARMMSMSNTEEDCEQELEDLLYCLD |
| | | | HCHSQ |
| EGFR | TfR | SEQ ID | QVKLEESGGGSVQTGGSLRLTCAASGHTSRSYGMH |
| | | NO: 313 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGGGGSGGGGSGGGGSEPK |
| | | | SSDKTHTCPPCPAPEAAGGPSVFLFPPKPKDTLMISRT |
| | | | PEVTCVVVDVSHEDPEVKFNWYVDGVEVHNAKTKP |
| | | | REEQYNSTYRVVSVLTVLHQDWLNGKEYKCKVSNK |
| | | | ALGAPIEKTISKAKGQPREPQVYTLPPSREEMTKNQV |
| | | | SLTCLVKGFYPSDIAVEWESNGQPENNYKTTPPVLDS |
| | | | DGSFFLYSKLTVDKSRWQQGNVFSCSVMHEALHNH |
| | | | YTQKSLSLSPGKGGGGSGGGGSGGGGSREGCASRCM |
| | | | KYNDELEKCEARMMSMSNTEEDCEQELEDLLYCLD |
| | | | HCHSQ |
| | TfR | SEQ ID | REGCASRCMKYNDELEKCEARMMSMSNTEEDCEQE |
| | | NO: 315 | LEDLLYCLDHCHSQGTGGGGSGGGGSGGGGSGGGG |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLWCLVKGFYPSDIAVEWESNGQPENNYKTTP |
| | | | PVLDSDGSFFLYSKLTVDKSRWQQGNVFSCSVMHEA |
| | | | LHNHYTQKSLSLSPGK |
| | TfR | SEQ ID | REGCASRCMKYNDELEKCEARMMSMSNTEEDCEQE |
| | | NO: 316 | LEDLLYCLDHCHSQGTGSPVPSTPPTPSPSTPPTPSPSA |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLWCLVKGFYPSDIAVEWESNGQPENNYKTTP |
| | | | PVLDSDGSFFLYSKLTVDKSRWQQGNVFSCSVMHEA |
| | | | LHNHYTQKSLSLSPGK |
| PD-L1 | TfR | SEQ ID | EHDCKVHCVKEWMAGHACAERQKSYTIGRAHCSGQ |
| | | NO: 317 | KFDVFKCLDHCAAPGTGGGGSGGGGSGGGGSGGGG |
| | | | SLKEAKEKAIEELKKAGITSDYYFDLINKAKTVEGVN |
| | | | ALKDEILKAGGGGSGGGGSGGGGSGGGGSREGCASR |
| | | | CMKYNDELEKCEARMMSMSNTEEDCEQELEDLLYC |
| | | | LDHCHSQ |
| PD-L1 | TfR | SEQ ID | EHDCKVHCVKEWMAGHACAERQKSYTIGRAHCSGQ |
| | | NO: 318 | KFDVFKCLDHCAAPGTGGGGSGGGGSGGGGSGGGG |
| | | | SLKEAKEKAIEELKKAGITSDYYFDLINKAKAVEGVN |
| | | | ALKDEILKAGGGGSGGGGSGGGGSGGGGSREGCASR |
| | | | CMKYNDELEKCEARMMSMSNTEEDCEQELEDLLYC |
| | | | LDHCHSQ |
| PD-L1 | | SEQ ID | EHDCKVHCVKEWMAGHACAERQKSYTIGRAHCSGQ |
| | | NO: 319 | KFDVFKCLDHCAAPGTGGGGSGGGGSGGGGSGGGG |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLSCAVKGFYPSDIAVEWESNGQPENNYKTTP |
| | | | PVLDSDGSFFLVSKLTVDKSRWQQGNVFSCSVMHEA |
| | | | LHNHYTQKSLSLSPGK |
| PD-L1 | TfR | SEQ ID | EHDCKVHCVKEWMAGHACAERQKSYTIGRAHCSGQ |
| | | NO: 320 | KFDVFKCLDHCAAPGTGGGGSGGGGSGGGGSGGGG |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLSCAVKGFYPSDIAVEWESNGQPENNYKTTP |
| | | | PVLDSDGSFFLVSKLTVDKSRWQQGNVFSCSVMHEA |
| | | | LHNHYTQKSLSLSPGKGGGGSGGGGSGGGGSREGCA |
| | | | SRCMKYNDELEKCEARMMSMSNTEEDCEQELEDLL |
| | | | YCLDHCHSQ |
| PD-L1 | | SEQ ID | EHDCKVHCVKEWMAGHACAERQKSYTIGRAHCSGQ |
| | | NO: 321 | KFDVFKCLDHCAAPGTGGGGSGGGGSGGGGSGGGG |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLWCLVKGFYPSDIAVEWESNGQPENNYKTTP |
| | | | PVLDSDGSFFLYSKLTVDKSRWQQGNVFSCSVMHEA |
| | | | LHNHYTQKSLSLSPGK |
| PD-L1 | TfR | SEQ ID | EHDCKVHCVKEWMAGHACAERQKSYTIGRAHCSGQ |
| | | NO: 322 | KFDVFKCLDHCAAPGTGSPVPSTPPTPSPSTPPTPSPSA |
| | | | SLKEAKEKAIEELKKAGITSDYYFDLINKAKTVEGVN |
| | | | ALKDEILKAGSPVPSTPPTPSPSTPPTPSPSASREGCAS |
| | | | RCMKYNDELEKCEARMMSMSNTEEDCEQELEDLLY |
| | | | CLDHCHSQ |
| PD-L1 | TfR | SEQ ID | EHDCKVHCVKEWMAGHACAERQKSYTIGRAHCSGQ |
| | | NO: 323 | KFDVFKCLDHCAAPGTGSPVPSTPPTPSPSTPPTPSPSA |
| | | | SLKEAKEKAIEELKKAGITSDYYFDLINKAKAVEGVN |
| | | | ALKDEILKAGSPVPSTPPTPSPSTPPTPSPSASREGCAS |
| | | | RCMKYNDELEKCEARMMSMSNTEEDCEQELEDLLY |
| | | | CLDHCHSQ |
| PD-L1 | | SEQ ID | EHDCKVHCVKEWMAGHACAERQKSYTIGRAHCSGQ |
| | | NO: 324 | KFDVFKCLDHCAAPGTGSPVPSTPPTPSPSTPPTPSPSA |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLSCAVKGFYPSDIAVEWESNGQPENNYKTTP |
| | | | PVLDSDGSFFLVSKLTVDKSRWQQGNVFSCSVMHEA |
| | | | LHNHYTQKSLSLSPGK |
| PD-L1 | TfR | SEQ ID | EHDCKVHCVKEWMAGHACAERQKSYTIGRAHCSGQ |
| | | NO: 325 | KFDVFKCLDHCAAPGTGSPVPSTPPTPSPSTPPTPSPSA |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLSCAVKGFYPSDIAVEWESNGQPENNYKTTP |
| | | | PVLDSDGSFFLVSKLTVDKSRWQQGNVFSCSVMHEA |
| | | | LHNHYTQKSLSLSPGKGGGGSGGGGSGGGGSREGCA |
| | | | SRCMKYNDELEKCEARMMSMSNTEEDCEQELEDLL |
| | | | YCLDHCHSQ |
| PD-L1 | | SEQ ID | EHDCKVHCVKEWMAGHACAERQKSYTIGRAHCSGQ |
| | | NO: 326 | KFDVFKCLDHCAAPGTGSPVPSTPPTPSPSTPPTPSPSA |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLWCLVKGFYPSDIAVEWESNGQPENNYKTTP |
| | | | PVLDSDGSFFLYSKLTVDKSRWQQGNVFSCSVMHEA |
| | | | LHNHYTQKSLSLSPGK |
| EGFR | TfR | SEQ ID | NSDSECPLSHDGYCLHGGVCMYIKAVDRYACNCVV |
| | | NO: 327 | GYIGERCQYRDLTWWGPRGTGGGGSGGGGSGGGGS |
| | | | GGGGSLKEAKEKAIEELKKAGITSDYYFDLINKAKTV |
| | | | EGVNALKDEILKAGGGGSGGGGSGGGGSGGGGSRE |
| | | | GCASRCMKYNDELEKCEARMMSMSNTEEDCEQELE |
| | | | DLLYCLDHCHSQ |
| EGFR | TfR | SEQ ID | NSDSECPLSHDGYCLHGGVCMYIKAVDRYACNCVV |
| | | NO: 328 | GYIGERCQYRDLTWWGPRGTGGGGSGGGGSGGGGS |
| | | | GGGGSLKEAKEKAIEELKKAGITSDYYFDLINKAKAV |
| | | | EGVNALKDEILKAGGGGSGGGGSGGGGSGGGGSRE |
| | | | GCASRCMKYNDELEKCEARMMSMSNTEEDCEQELE |
| | | | DLLYCLDHCHSQ |
| EGFR | | SEQ ID | NSDSECPLSHDGYCLHGGVCMYIKAVDRYACNCVV |
| | | NO: 329 | GYIGERCQYRDLTWWGPRGTGGGGSGGGGSGGGGS |
| | | | GGGGSEPKSCDKTHTCPPCPAPEAAGGPSVFLFPPKP |
| | | | KDTLMISRTPEVTCVVVDVSHEDPEVKFNWYVDGVE |
| | | | VHNAKTKPREEQYNSTYRVVSVLTVLHQDWLNGKE |
| | | | YKCKVSNKALGAPIEKTISKAKGQPREPQVYTLPPSR |
| | | | DELTKNQVSLSCAVKGFYPSDIAVEWESNGQPENNY |
| | | | KTTPPVLDSDGSFFLVSKLTVDKSRWQQGNVFSCSV |
| | | | MHEALHNHYTQKSLSLSPGK |
| EGFR | TfR | SEQ ID | NSDSECPLSHDGYCLHGGVCMYIKAVDRYACNCVV |
| | | NO: 330 | GYIGERCQYRDLTWWGPRGTGGGGSGGGGSGGGGS |
| | | | GGGGSEPKSCDKTHTCPPCPAPEAAGGPSVFLFPPKP |
| | | | KDTLMISRTPEVTCVVVDVSHEDPEVKFNWYVDGVE |
| | | | VHNAKTKPREEQYNSTYRVVSVLTVLHQDWLNGKE |
| | | | YKCKVSNKALGAPIEKTISKAKGQPREPQVYTLPPSR |
| | | | DELTKNQVSLSCAVKGFYPSDIAVEWESNGQPENNY |
| | | | KTTPPVLDSDGSFFLVSKLTVDKSRWQQGNVFSCSV |
| | | | MHEALHNHYTQKSLSLSPGKGGGGSGGGGSGGGGS |
| | | | REGCASRCMKYNDELEKCEARMMSMSNTEEDCEQE |
| | | | LEDLLYCLDHCHSQ |
| EGFR | | SEQ ID | NSDSECPLSHDGYCLHGGVCMYIKAVDRYACNCVV |
| | | NO: 331 | GYIGERCQYRDLTWWGPRGTGGGGSGGGGSGGGGS |
| | | | GGGGSEPKSCDKTHTCPPCPAPEAAGGPSVFLFPPKP |
| | | | KDTLMISRTPEVTCVVVDVSHEDPEVKFNWYVDGVE |
| | | | VHNAKTKPREEQYNSTYRVVSVLTVLHQDWLNGKE |
| | | | YKCKVSNKALGAPIEKTISKAKGQPREPQVYTLPPSR |
| | | | DELTKNQVSLWCLVKGFYPSDIAVEWESNGQPENNY |
| | | | KTTPPVLDSDGSFFLYSKLTVDKSRWQQGNVFSCSV |
| | | | MHEALHNHYTQKSLSLSPGK |
| EGFR | TfR | SEQ ID | NSDSECPLSHDGYCLHGGVCMYIKAVDRYACNCVV |
| | | NO: 332 | GYIGERCQYRDLTWWGPRGTGSPVPSTPPTPSPSTPPT |
| | | | PSPSASLKEAKEKAIEELKKAGITSDYYFDLINKAKTV |
| | | | EGVNALKDEILKAGSPVPSTPPTPSPSTPPTPSPSASRE |
| | | | GCASRCMKYNDELEKCEARMMSMSNTEEDCEQELE |
| | | | DLLYCLDHCHSQ |
| EGFR | TfR | SEQ ID | NSDSECPLSHDGYCLHGGVCMYIKAVDRYACNCVV |
| | | NO: 333 | GYIGERCQYRDLTWWGPRGTGSPVPSTPPTPSPSTPPT |
| | | | PSPSASLKEAKEKAIEELKKAGITSDYYFDLINKAKAV |
| | | | EGVNALKDEILKAGSPVPSTPPTPSPSTPPTPSPSASRE |
| | | | GCASRCMKYNDELEKCEARMMSMSNTEEDCEQELE |
| | | | DLLYCLDHCHSQ |
| EGFR | | SEQ ID | NSDSECPLSHDGYCLHGGVCMYIKAVDRYACNCVV |
| | | NO: 334 | GYIGERCQYRDLTWWGPRGTGSPVPSTPPTPSPSTPPT |
| | | | PSPSASEPKSCDKTHTCPPCPAPEAAGGPSVFLFPPKP |
| | | | KDTLMISRTPEVTCVVVDVSHEDPEVKFNWYVDGVE |
| | | | VHNAKTKPREEQYNSTYRVVSVLTVLHQDWLNGKE |
| | | | YKCKVSNKALGAPIEKTISKAKGQPREPQVYTLPPSR |
| | | | DELTKNQVSLSCAVKGFYPSDIAVEWESNGQPENNY |
| | | | KTTPPVLDSDGSFFLVSKLTVDKSRWQQGNVFSCSV |
| | | | MHEALHNHYTQKSLSLSPGK |
| EGFR | TfR | SEQ ID | NSDSECPLSHDGYCLHGGVCMYIKAVDRYACNCVV |
| | | NO: 335 | GYIGERCQYRDLTWWGPRGTGSPVPSTPPTPSPSTPPT |
| | | | PSPSASEPKSCDKTHTCPPCPAPEAAGGPSVFLFPPKP |
| | | | KDTLMISRTPEVTCVVVDVSHEDPEVKFNWYVDGVE |
| | | | VHNAKTKPREEQYNSTYRVVSVLTVLHQDWLNGKE |
| | | | YKCKVSNKALGAPIEKTISKAKGQPREPQVYTLPPSR |
| | | | DELTKNQVSLSCAVKGFYPSDIAVEWESNGQPENNY |
| | | | KTTPPVLDSDGSFFLVSKLTVDKSRWQQGNVFSCSV |
| | | | MHEALHNHYTQKSLSLSPGKGGGGSGGGGSGGGGS |
| | | | REGCASRCMKYNDELEKCEARMMSMSNTEEDCEQE |
| | | | LEDLLYCLDHCHSQ |
| EGFR | | SEQ ID | NSDSECPLSHDGYCLHGGVCMYIKAVDRYACNCVV |
| | | NO: 336 | GYIGERCQYRDLTWWGPRGTGSPVPSTPPTPSPSTPPT |
| | | | PSPSASEPKSCDKTHTCPPCPAPEAAGGPSVFLFPPKP |
| | | | KDTLMISRTPEVTCVVVDVSHEDPEVKFNWYVDGVE |
| | | | VHNAKTKPREEQYNSTYRVVSVLTVLHQDWLNGKE |
| | | | YKCKVSNKALGAPIEKTISKAKGQPREPQVYTLPPSR |
| | | | DELTKNQVSLWCLVKGFYPSDIAVEWESNGQPENNY |
| | | | KTTPPVLDSDGSFFLYSKLTVDKSRWQQGNVFSCSV |
| | | | MHEALHNHYTQKSLSLSPGK |
| EGFR | TfR | SEQ ID | QVKLEESGGGSVQTGGSLRLTCAASGHTSRSYGMH |
| | | NO: 337 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGGGGSGGGGSGGGGSG |
| | | | GGGSLKEAKEKAIEELKKAGITSDYYFDLINKAKTVE |
| | | | GVNALKDEILKAGGGGSGGGGSGGGGSGGGGSREG |
| | | | CASRCMKYNDELEKCEARMMSMSNTEEDCEQELED |
| | | | LLYCLDHCHSQ |
| EGFR | TfR | SEQ ID | QVKLEESGGGSVQTGGSLRLTCAASGHTSRSYGMH |
| | | NO: 338 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGGGGSGGGGSGGGGSG |
| | | | GGGSLKEAKEKAIEELKKAGITSDYYFDLINKAKAVE |
| | | | GVNALKDEILKAGGGGSGGGGSGGGGSGGGGSREG |
| | | | CASRCMKYNDELEKCEARMMSMSNTEEDCEQELED |
| | | | LLYCLDHCHSQ |
| EGFR | | SEQ ID | QVKLEESGGGSVQTGGSLRLTCAASGHTSRSYGMH |
| | | NO: 339 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGGGGSGGGGSGGGGSG |
| | | | GGGSEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLSCAVKGFYPSDIAVEWESNGQPENNYK |
| | | | TTPPVLDSDGSFFLVSKLTVDKSRWQQGNVFSCSVM |
| | | | HEALHNHYTQKSLSLSPGK |
| EGFR | TfR | SEQ ID | QVKLEESGGGSVQTGGSLRLTCAASGHTSRSYGMH |
| | | NO: 340 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGGGGSGGGGSGGGGSG |
| | | | GGGSEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLSCAVKGFYPSDIAVEWESNGQPENNYK |
| | | | TTPPVLDSDGSFFLVSKLTVDKSRWQQGNVFSCSVM |
| | | | HEALHNHYTQKSLSLSPGKGGGGSGGGGSGGGGSRE |
| | | | GCASRCMKYNDELEKCEARMMSMSNTEEDCEQELE |
| | | | DLLYCLDHCHSQ |
| EGFR | | SEQ ID | QVKLEESGGGSVQTGGSLRLTCAASGHTSRSYGMH |
| | | NO: 341 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGGGGSGGGGSGGGGSG |
| | | | GGGSEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLWCLVKGFYPSDIAVEWESNGQPENNYK |
| | | | TTPPVLDSDGSFFLYSKLTVDKSRWQQGNVFSCSVM |
| | | | HEALHNHYTQKSLSLSPGK |
| EGFR | TfR | SEQ ID | QVKLEESGGGSVQTGGSLRLTCAASGHTSRSYGMH |
| | | NO: 342 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGSPVPSTPPTPSPSTPPTP |
| | | | SPSASLKEAKEKAIEELKKAGITSDYYFDLINKAKTVE |
| | | | GVNALKDEILKAGSPVPSTPPTPSPSTPPTPSPSASREG |
| | | | CASRCMKYNDELEKCEARMMSMSNTEEDCEQELED |
| | | | LLYCLDHCHSQ |
| EGFR | TfR | SEQ ID | QVKLEESGGGSVQTGGSLRLTCAASGHTSRSYGMH |
| | | NO: 343 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGSPVPSTPPTPSPSTPPTP |
| | | | SPSASLKEAKEKAIEELKKAGITSDYYFDLINKAKAVE |
| | | | GVNALKDEILKAGSPVPSTPPTPSPSTPPTPSPSASREG |
| | | | CASRCMKYNDELEKCEARMMSMSNTEEDCEQELED |
| | | | LLYCLDHCHSQ |
| EGFR | | SEQ ID | QVKLEESGGGSVQTGGSLRLTCAASGHTSRSYGMH |
| | | NO: 344 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGSPVPSTPPTPSPSTPPTP |
| | | | SPSASEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLSCAVKGFYPSDIAVEWESNGQPENNYK |
| | | | TTPPVLDSDGSFFLVSKLTVDKSRWQQGNVFSCSVM |
| | | | HEALHNHYTQKSLSLSPGK |
| EGFR | TfR | SEQ ID | QVKLEESGGGSVQTGGSLRLTCAASGHTSRSYGMH |
| | | NO: 345 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGSPVPSTPPTPSPSTPPTP |
| | | | SPSASEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLSCAVKGFYPSDIAVEWESNGQPENNYK |
| | | | TTPPVLDSDGSFFLVSKLTVDKSRWQQGNVFSCSVM |
| | | | HEALHNHYTQKSLSLSPGKGGGGSGGGGSGGGGSRE |
| | | | GCASRCMKYNDELEKCEARMMSMSNTEEDCEQELE |
| | | | DLLYCLDHCHSQ |
| EGFR | | SEQ ID | QVKLEESGGGSVQTGGSLRLTCAASGHTSRSYGMH |
| | | NO: 346 | WFRQAPGKEREFVSGISWRGDSTGYADSVKGRFTISR |
| | | | DNAKNTVDLQMNSLKPEDTAIYYCAAAAGSHWYGT |
| | | | HYEYDYWGQGTQVTVSSGTGSPVPSTPPTPSPSTPPTP |
| | | | SPSASEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLWCLVKGFYPSDIAVEWESNGQPENNYK |
| | | | TTPPVLDSDGSFFLYSKLTVDKSRWQQGNVFSCSVM |
| | | | HEALHNHYTQKSLSLSPGK |
| EGFR | TfR | SEQ ID | QVQLQESGGGVVQPGRSLRLSCAASRYPFHHHDHH |
| | | NO: 347 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSGG |
| | | | GGSGGGGSQVKLEESGGGSVQTGGSLRLTCAASGHT |
| | | | SRSYGMGWFRQAPGKEREFVSGISWRGDSTGYADSV |
| | | | KGRFTISRDNAKNTVDLQMNSLKPEDTAIYYCAAAA |
| | | | GSAWYHTLYEYDYWGQGTQVTVSS |
| PD-L1 | TfR | SEQ ID | QVQLQESGGGVVQPGRSLRLSCAASRYPFHHHDHH |
| | | NO: 348 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSGG |
| | | | GGSGGGGSEEDCKVHCVKEWMAGKACAERQKSYTI |
| | | | GRAHCSGQKFDVFKCLDHCAAP |
| PD-L1 | TfR | SEQ ID | REGCASRCMKYNDELEKCEARMMSMSNTEEDCEQE |
| | | NO: 351 | LEDLLYCLDHCHSQGGGGSGGGGSGGGGSEEDCKV |
| | | | HCVKEWMAGKACAERQKSYTIGRAHCSGQKFDVFK |
| | | | CLDHCAAP |
| EGFR | PD-L1 | SEQ ID | QVQLQESGGGVVQPGRSLRLSCAASRFTFNNYAMH |
| | | NO: 352 | WVRQAPGKGLEWVAVISYDGSNKYYADSVKGRFTIS |
| | | | RDNSKNTLYLQMNSLRAEDTAVYYCARDLSGYGDY |
| | | | PDYWGQGTLVTVSSGGGGSGGGGSGGGGSSELTQDP |
| | | | AVSVALGQTVRITCQGDSLRSYYASWYQQKPGQAPV |
| | | | LVMYGRNERPSGVPDRFSGSKSGTSASLAISGLQPED |
| | | | EANYYCAGWDDSLTGPVFGGGTKLTVLGGGGGSGG |
| | | | GGSGGGGSEEDCKVHCVKEWMAGKACAERQKSYTI |
| | | | GRAHCSGQKFDVFKCLDHCAAP |
| PD-L1 | TfR | SEQ ID | EEDCKVHCVKEWMAGKACAERQKSYTIGRAHCSGQ |
| | | NO: 355 | KFDVFKCLDHCAAPGTGGGGSGGGGSGGGGSGGGG |
| | | | SLKEAKEKAIEELKKAGITSDYYFDLINKAKTVEGVN |
| | | | ALKDEILKAGGGGSGGGGSGGGGSGGGGSREGCASR |
| | | | CMKYNDELEKCEARMMSMSNTEEDCEQELEDLLYC |
| | | | LDHCHSQ |
| PD-L1 | TfR | SEQ ID | EEDCKVHCVKEWMAGKACAERQKSYTIGRAHCSGQ |
| | | NO: 356 | KFDVFKCLDHCAAPGTGGGGSGGGGSGGGGSGGGG |
| | | | SLKEAKEKAIEELKKAGITSDYYFDLINKAKAVEGVN |
| | | | ALKDEILKAGGGGSGGGGSGGGGSGGGGSREGCASR |
| | | | CMKYNDELEKCEARMMSMSNTEEDCEQELEDLLYC |
| | | | LDHCHSQ |
| PD-L1 | | SEQ ID | EEDCKVHCVKEWMAGKACAERQKSYTIGRAHCSGQ |
| | | NO: 358 | KFDVFKCLDHCAAPGTGGGGSGGGGSGGGGSGGGG |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLSCAVKGFYPSDIAVEWESNGQPENNYKTTP |
| | | | PVLDSDGSFFLVSKLTVDKSRWQQGNVFSCSVMHEA |
| | | | LHNHYTQKSLSLSPGK |
| PD-L1 | TfR | SEQ ID | EEDCKVHCVKEWMAGKACAERQKSYTIGRAHCSGQ |
| | | NO: 359 | KFDVFKCLDHCAAPGTGGGGSGGGGSGGGGSGGGG |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLSCAVKGFYPSDIAVEWESNGQPENNYKTTP |
| | | | PVLDSDGSFFLVSKLTVDKSRWQQGNVFSCSVMHEA |
| | | | LHNHYTQKSLSLSPGKGGGGGGGGSGGGGSREGCA |
| | | | SRCMKYNDELEKCEARMMSMSNTEEDCEQELEDLL |
| | | | YCLDHCHSQ |
| PD-L1 | | SEQ ID | EEDCKVHCVKEWMAGKACAERQKSYTIGRAHCSGQ |
| | | NO: 360 | KFDVFKCLDHCAAPGTGGGGSGGGGSGGGGSGGGG |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLWCLVKGFYPSDIAVEWESNGQPENNYKTTP |
| | | | PVLDSDGSFFLYSKLTVDKSRWQQGNVFSCSVMHEA |
| | | | LHNHYTQKSLSLSPGK |
| PD-L1 | TfR | SEQ ID | EEDCKVHCVKEWMAGKACAERQKSYTIGRAHCSGQ |
| | | NO: 361 | KFDVFKCLDHCAAPGTGSPVPSTPPTPSPSTPPTPSPSA |
| | | | SLKEAKEKAIEELKKAGITSDYYFDLINKAKTVEGVN |
| | | | ALKDEILKAGSPVPSTPPTPSPSTPPTPSPSASREGCAS |
| | | | RCMKYNDELEKCEARMMSMSNTEEDCEQELEDLLY |
| | | | CLDHCHSQ |
| PD-L1 | TfR | SEQ ID | EEDCKVHCVKEWMAGKACAERQKSYTIGRAHCSGQ |
| | | NO: 362 | KFDVFKCLDHCAAPGTGSPVPSTPPTPSPSTPPTPSPSA |
| | | | SLKEAKEKAIEELKKAGITSDYYFDLINKAKAVEGVN |
| | | | ALKDEILKAGSPVPSTPPTPSPSTPPTPSPSASREGCAS |
| | | | RCMKYNDELEKCEARMMSMSNTEEDCEQELEDLLY |
| | | | CLDHCHSQ |
| PD-L1 | | SEQ ID | EEDCKVHCVKEWMAGKACAERQKSYTIGRAHCSGQ |
| | | NO: 363 | KFDVFKCLDHCAAPGTGSPVPSTPPTPSPSTPPTPSPSA |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLSCAVKGFYPSDIAVEWESNGQPENNYKTTP |
| | | | PVLDSDGSFFLVSKLTVDKSRWQQGNVFSCSVMHEA |
| | | | LHNHYTQKSLSLSPGK |
| PD-L1 | TfR | SEQ ID | EEDCKVHCVKEWMAGKACAERQKSYTIGRAHCSGQ |
| | | NO: 364 | KFDVFKCLDHCAAPGTGSPVPSTPPTPSPSTPPTPSPSA |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLSCAVKGFYPSDIAVEWESNGQPENNYKTTP |
| | | | PVLDSDGSFFLVSKLTVDKSRWQQGNVFSCSVMHEA |
| | | | LHNHYTQKSLSLSPGKGGGGSGGGGSGGGGSREGCA |
| | | | SRCMKYNDELEKCEARMMSMSNTEEDCEQELEDLL |
| | | | YCLDHCHSQ |
| PD-L1 | | SEQ ID | EEDCKVHCVKEWMAGKACAERQKSYTIGRAHCSGQ |
| | | NO: 365 | KFDVFKCLDHCAAPGTGSPVPSTPPTPSPSTPPTPSPSA |
| | | | SEPKSCDKTHTCPPCPAPEAAGGPSVFLFPPKPKDTL |
| | | | MISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLWCLVKGFYPSDIAVEWESNGQPENNYKTTP |
| | | | PVLDSDGSFFLYSKLTVDKSRWQQGNVFSCSVMHEA |
| | | | LHNHYTQKSLSLSPGK |
| EGFR | | SEQ ID | QVKLEESGGGSVQTGGSLRLTCAASGRTSRSYGMGW |
| | | NO: 371 | FRQAPGKEREFVSGISWRGDSTGYADSVKGRFTISRD |
| | | | NAKNTVDLQMNSLKPEDTAIYYCAAAAGSAWYGTL |
| | | | YEYDYWGQGTQVTVSSGTGGGGSGGGGSGGGGSGG |
| | | | GGSEPKSCDKTHTCPPCPAPEAAGGPSVFLFPPKPKDT |
| | | | LMISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLSCAVKGFYPSDIAVEWESNGQPENNYKTTP |
| | | | PVLDSDGSFFLVSKLTVDKSRWQQGNVFSCSVMHEA |
| | | | LHNHYTQKSLSLSPGK |
| EGFR | | SEQ ID | QVKLEESGGGSVQTGGSLRLTCAASGRTSRSYGMGW |
| | | NO: 373 | FRQAPGKEREFVSGISWRGDSTGYADSVKGRFTISRD |
| | | | NAKNTVDLQMNSLKPEDTAIYYCAAAAGSAWYGTL |
| | | | YEYDYWGQGTQVTVSSGTGGGGSGGGGSGGGGSGG |
| | | | GGSEPKSCDKTHTCPPCPAPEAAGGPSVFLFPPKPKDT |
| | | | LMISRTPEVTCVVVDVSHEDPEVKFNWYVDGVEVHN |
| | | | AKTKPREEQYNSTYRVVSVLTVLHQDWLNGKEYKC |
| | | | KVSNKALGAPIEKTISKAKGQPREPQVYTLPPSRDELT |
| | | | KNQVSLWCLVKGFYPSDIAVEWESNGQPENNYKTTP |
| | | | PVLDSDGSFFLYSKLTVDKSRWQQGNVFSCSVMHEA |
| | | | LHNHYTQKSLSLSPGK |
| EGFR | | SEQ ID | QVKLEESGGGSVQTGGSLRLTCAASGRTSRSYGMGW |
| | | NO: 376 | FRQAPGKEREFVSGISWRGDSTGYADSVKGRFTISRD |
| | | | NAKNTVDLQMNSLKPEDTAIYYCAAAAGSAWYGTL |
| | | | YEYDYWGQGTQVTVSSGTGSPVPSTPPTPSPSTPPTPS |
| | | | PSASEPKSCDKTHTCPPCPAPEAAGGPSVFLFPPKPKD |
| | | | TLMISRTPEVTCVVVDVSHEDPEVKFNWYVDGVEVH |
| | | | NAKTKPREEQYNSTYRVVSVLTVLHQDWLNGKEYK |
| | | | CKVSNKALGAPIEKTISKAKGQPREPQVYTLPPSRDEL |
| | | | TKNQVSLSCAVKGFYPSDIAVEWESNGQPENNYKTT |
| | | | PPVLDSDGSFFLVSKLTVDKSRWQQGNVFSCSVMHE |
| | | | ALHNHYTQKSLSLSPGK |
| EGFR | | SEQ ID | QVKLEESGGGSVQTGGSLRLTCAASGRTSRSYGMGW |
| | | NO: 378 | FRQAPGKEREFVSGISWRGDSTGYADSVKGRFTISRD |
| | | | NAKNTVDLQMNSLKPEDTAIYYCAAAAGSAWYGTL |
| | | | YEYDYWGQGTQVTVSSGTGSPVPSTPPTPSPSTPPTPS |
| | | | PSASEPKSCDKTHTCPPCPAPEAAGGPSVFLFPPKPKD |
| | | | TLMISRTPEVTCVVVDVSHEDPEVKFNWYVDGVEVH |
| | | | NAKTKPREEQYNSTYRVVSVLTVLHQDWLNGKEYK |
| | | | CKVSNKALGAPIEKTISKAKGQPREPQVYTLPPSRDEL |
| | | | TKNQVSLWCLVKGFYPSDIAVEWESNGQPENNYKTT |
| | | | PPVLDSDGSFFLYSKLTVDKSRWQQGNVFSCSVMHE |
| | | | ALHNHYTQKSLSLSPGK |
| EGFR | | SEQ ID | QVKLEESGGGSVQTGGSLRLTCAASGHTSRSYGMG |
| | | NO: 382 | WFRQAPGKEREFVSGISWRGDSTGYADSVKGRFTISR |
| | | | DNAKNTVDLQMNSLKPEDTAIYYCAAAAGSAWYHT |
| | | | LYEYDYWGQGTQVTVSSGTGGGGSGGGGSGGGGSG |
| | | | GGGSEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLSCAVKGFYPSDIAVEWESNGQPENNYK |
| | | | TTPPVLDSDGSFFLVSKLTVDKSRWQQGNVFSCSVM |
| | | | HEALHNHYTQKSLSLSPGK |
| EGFR | | SEQ ID | QVKLEESGGGSVQTGGSLRLTCAASGHTSRSYGMG |
| | | NO: 384 | WFRQAPGKEREFVSGISWRGDSTGYADSVKGRFTISR |
| | | | DNAKNTVDLQMNSLKPEDTAIYYCAAAAGSAWYHT |
| | | | LYEYDYWGQGTQVTVSSGTGGGGSGGGGSGGGGSG |
| | | | GGGSEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLWCLVKGFYPSDIAVEWESNGQPENNYK |
| | | | TTPPVLDSDGSFFLYSKLTVDKSRWQQGNVFSCSVM |
| | | | HEALHNHYTQKSLSLSPGK |
| EGFR | | SEQ ID | QVKLEESGGGSVQTGGSLRLTCAASGHTSRSYGMG |
| | | NO: 387 | WFRQAPGKEREFVSGISWRGDSTGYADSVKGRFTISR |
| | | | DNAKNTVDLQMNSLKPEDTAIYYCAAAAGSAWYHT |
| | | | LYEYDYWGQGTQVTVSSGTGSPVPSTPPTPSPSTPPTP |
| | | | SPSASEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLSCAVKGFYPSDIAVEWESNGQPENNYK |
| | | | TTPPVLDSDGSFFLVSKLTVDKSRWQQGNVFSCSVM |
| | | | HEALHNHYTQKSLSLSPGK |
| EGFR | | SEQ ID | QVKLEESGGGSVQTGGSLRLTCAASGHTSRSYGMG |
| | | NO: 389 | WFRQAPGKEREFVSGISWRGDSTGYADSVKGRFTISR |
| | | | DNAKNTVDLQMNSLKPEDTAIYYCAAAAGSAWYHT |
| | | | LYEYDYWGQGTQVTVSSGTGSPVPSTPPTPSPSTPPTP |
| | | | SPSASEPKSCDKTHTCPPCPAPEAAGGPSVFLFPPKPK |
| | | | DTLMISRTPEVTCVVVDVSHEDPEVKFNWYVDGVEV |
| | | | HNAKTKPREEQYNSTYRVVSVLTVLHQDWLNGKEY |
| | | | KCKVSNKALGAPIEKTISKAKGQPREPQVYTLPPSRD |
| | | | ELTKNQVSLWCLVKGFYPSDIAVEWESNGQPENNYK |
| | | | TTPPVLDSDGSFFLYSKLTVDKSRWQQGNVFSCSVM |
| | | | HEALHNHYTQKSLSLSPGK |

### Sequence Identity and Homology

Percent (%) sequence identity or homology is determined by conventional methods. (See e.g., Altschul et al. (1986), Bull. Math. Bio. 48:603 (1986), and Henikoff and Henikoff (1992), Proc. Natl. Acad. Sci. USA 89:10915). Briefly, two amino acid sequences can be aligned to optimize the alignment scores using a gap opening penalty of 10, a gap extension penalty of 1, and the “BLOSUM62” scoring matrix of Henikoff and Henikoff (Id.). The sequence identity or homology is then calculated as: ([Total number of identical matches]/[length of the longer sequence plus the number of gaps introduced into the longer sequence in order to align the two sequences])(100).

Various methods and software programs can be used to determine the homology between two or more peptides, such as NCBI BLAST, Clustal W, MAFFT, Clustal Omega, AlignMe, Praline, or another suitable method or algorithm. Pairwise sequence alignment can be used to identify regions of similarity that can indicate functional, structural and/or evolutionary relationships between two biological sequences (e.g., amino acid or nucleic acid sequences). In addition, multiple sequence alignment (MSA) is the alignment of three or more biological sequences. From the output of MSA applications, homology can be inferred and the evolutionary relationship between the sequences assessed. As used herein, “sequence homology” and “sequence identity” and “percent (%) sequence identity” and “percent (%) sequence homology” are used interchangeably to mean the sequence relatedness or variation, as appropriate, to a reference polynucleotide or amino acid sequence.

Additionally, there are several established algorithms available to align two amino acid sequences. For example, the “FASTA” similarity search algorithm of Pearson and Lipman can be a suitable protein alignment method for examining the level of sequence identity or homology shared by an amino acid sequence of a peptide disclosed herein and the amino acid sequence of a peptide variant. The FASTA algorithm is described, for example, by Pearson and Lipman, Proc. Nat'l Acad. Sci. USA 85:2444 (1988), and by Pearson, Meth. Enzymol. 183:63 (1990). Briefly, FASTA first characterizes sequence similarity by identifying regions shared by the query sequence (e.g., SEQ ID NO: 1) and a test sequence that has either the highest density of identities (if the ktup variable is 1) or pairs of identities (if ktup=2), without considering conservative amino acid substitutions, insertions, or deletions. The ten regions with the highest density of identities are then rescored by comparing the similarity of all paired amino acids using an amino acid substitution matrix, and the ends of the regions are “trimmed” to include only those residues that contribute to the highest score. If there are several regions with scores greater than the “cutoff” value (calculated by a predetermined formula based upon the length of the sequence and the ktup value), then the trimmed initial regions are examined to determine whether the regions can be joined to form an approximate alignment with gaps. Finally, the highest scoring regions of the two amino acid sequences are aligned using a modification of the Needleman-Wunsch-Sellers algorithm (Needleman and Wunsch, J Mol. Biol. 48:444 (1970); Sellers, Siam J. Appl. Math. 26:787 (1974)), which allows for amino acid insertions and deletions. For example, illustrative parameters for FASTA analysis are: ktup=1, gap opening penalty=10, gap extension penalty=1, and substitution matrix=BLOSUM62. These parameters can be introduced into a FASTA program by modifying the scoring matrix file (“SMATRIX”), as explained in Appendix 2 of Pearson, Meth. Enzymol. 183:63 (1990).

FASTA can also be used to determine the sequence identity or homology of nucleic acid sequences or molecules using a ratio as disclosed above. For nucleic acid sequence comparisons, the ktup value can range between one to six, preferably from three to six, most preferably three, with other parameters set as described herein.

Some examples of common amino acids that are a “conservative amino acid substitution” are illustrated by a substitution among amino acids within each of the following groups: (1) glycine, alanine, valine, leucine, and isoleucine, (2) phenylalanine, tyrosine, and tryptophan, (3) serine and threonine, (4) aspartate and glutamate, (5) glutamine and asparagine, and (6) lysine, arginine and histidine. The BLOSUM62 table is an amino acid substitution matrix derived from about 2,000 local multiple alignments of protein sequence segments, representing highly conserved regions of more than 500 groups of related proteins (Henikoff and Henikoff, Proc. Nat'l Acad. Sci. USA 89:10915 (1992)). Accordingly, the BLOSUM62 substitution frequencies can be used to define conservative amino acid substitutions that can be introduced into the amino acid sequences of the present invention. Although it is possible to design amino acid substitutions based solely upon chemical properties (as discussed above), the language “conservative amino acid substitution” preferably refers to a substitution represented by a BLOSUM62 value of greater than −1. For example, an amino acid substitution is conservative if the substitution is characterized by a BLOSUM62 value of 0, 1, 2, or 3. According to this system, preferred conservative amino acid substitutions are characterized by a BLOSUM62 value of at least 1 (e.g., 1, 2 or 3), while more preferred conservative amino acid substitutions are characterized by a BLOSUM62 value of at least 2 (e.g., 2 or 3).

Determination of amino acid residues that are within regions or domains that are critical to maintaining structural integrity can be determined. Within these regions one can determine specific residues that can be more or less tolerant of change and maintain the overall tertiary structure of the molecule. Methods for analyzing sequence structure include, but are not limited to, alignment of multiple sequences with high amino acid or nucleotide identity or homology and computer analysis using available software (e.g., the Insight II® viewer and homology modeling tools; MSI, San Diego, Calif.), secondary structure propensities, binary patterns, complementary packing and buried polar interactions (Barton, G. J., Current Opin. Struct. Biol. 5:372-6 (1995) and Cordes, M. H. et al., Current Opin. Struct. Biol. 6:3-10 (1996)). In general, when designing modifications to molecules or identifying specific fragments, determination of structure can typically be accompanied by evaluating activity of modified molecules.

### Engineered Binding Peptides

A peptide of the present disclosure (e.g., a TfR-binding peptide, a target-binding peptide, or a selective depletion complex) can be engineered to improve or alter a property of the peptide. For example, a peptide can be modified to alter the affinity of the peptide for a binding partner (e.g., a target molecule or a TfR). In some embodiments, a peptide can be modified to alter binding affinity in a pH-dependent manner. A peptide can be modified my introducing one or more amino acid variations into the peptide sequence and testing the effect of the variation on peptide properties (e.g., binding affinity).

In some embodiments, a peptide or a library of peptides is designed in silico without derivation from a naturally occurring scaffold of a knotted peptide. In other embodiments, a peptide or a library of peptides is designed in silico by derivation, grafting relevant protein-binding residues, or conserved residues in the protein-binding interface a naturally occurring peptide or protein known to bind to a protein or receptor of interest. In some embodiments, the peptide (e.g., a TfR-binding peptide of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) is a simple helix-turn-helix. In some embodiments, the helix-turn-helix can be used for pharmacophore transfer onto other scaffolds, for example engraftment of the required TfR-engaging surface onto the helix-turn-helix scaffold using fusion tagging.

In some embodiments, a peptide comprising SEQ ID NO: 1 is used as a scaffold or base sequence for further modifications, including addition, deletion, or amino acid substitution. In some embodiments, short sequences of amino acid residues such as GS are added at the N-terminus of a peptide. In some embodiments, peptides lack GS at the N-terminus. In some instances, peptides undergo one or more post-translational modifications.

In some embodiments, a peptide capable of binding TfR and transcytosis across a cell membrane comprises a sequence with at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with any one of the exemplary peptide sequences listed in TABLE 1 (SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64), or a functional fragment thereof. Two or more peptides can share a degree of sequence identity or homology and share similar properties in vivo. For instance, a peptide can share a degree of sequence identity or homology with any one of the peptides of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. In some embodiments, one or more peptides of the present disclosure have up to about 20% pairwise sequence identity or homology, up to about 25% pairwise sequence identity or homology, up to about 30% pairwise sequence identity or homology, up to about 35% pairwise sequence identity or homology, up to about 40% pairwise sequence identity or homology, up to about 45% pairwise sequence identity or homology, up to about 50% pairwise sequence identity or homology, up to about 55% pairwise sequence identity or homology, up to about 60% pairwise sequence identity or homology, up to about 65% pairwise sequence identity or homology, up to about 70% pairwise sequence identity or homology, up to about 75% pairwise sequence identity or homology, up to about 80% pairwise sequence identity or homology, up to about 85% pairwise sequence identity or homology, up to about 90% pairwise sequence identity or homology, up to about 95% pairwise sequence identity or homology, up to about 96% pairwise sequence identity or homology, up to about 97% pairwise sequence identity or homology, up to about 98% pairwise sequence identity or homology, up to about 99% pairwise sequence identity or homology, up to about 99.5% pairwise sequence identity or homology, or up to about 99.9% pairwise sequence identity or homology. In some embodiments, one or more peptides of the disclosure have at least about 20% pairwise sequence identity or homology, at least about 25% pairwise sequence identity or homology, at least about 30% pairwise sequence identity or homology, at least about 35% pairwise sequence identity or homology, at least about 40% pairwise sequence identity or homology, at least about 45% pairwise sequence identity or homology, at least about 50% pairwise sequence identity or homology, at least about 55% pairwise sequence identity or homology, at least about 60% pairwise sequence identity or homology, at least about 65% pairwise sequence identity or homology, at least about 70% pairwise sequence identity or homology, at least about 75% pairwise sequence identity or homology, at least about 80% pairwise sequence identity or homology, at least about 85% pairwise sequence identity or homology, at least about 90% pairwise sequence identity or homology, at least about 95% pairwise sequence identity or homology, at least about 96% pairwise sequence identity or homology, at least about 97% pairwise sequence identity or homology, at least about 98% pairwise sequence identity or homology, at least about 99% pairwise sequence identity or homology, at least about 99.5% pairwise sequence identity or homology, at least about 99.9% pairwise sequence identity or homology with a second peptide.

In some embodiments, peptides that exhibit an improved TfR receptor binding show improved transcytosis function. In some cases, peptides that exhibit an improved TfR receptor binding show no or small changes in transcytosis function. In some cases, peptides that exhibit an improved TfR receptor binding show reduced transcytosis function. In some embodiments, the K_{A} and K_{D} values of a TfR-binding peptide can be modulated and optimized (e.g., via amino acid substitutions) to provide an optimal ratio of TfR-binding affinity and efficient transcytosis function.

In some instances, the peptide or peptide complex is any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64, or a functional fragment thereof. In other embodiments, the peptide or peptide complex of the disclosure further comprises a peptide with 99%, 95%, 90%, 85%, or 80% sequence identity or homology to any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 or functional fragment thereof.

In other instances, the peptide or peptide complex can be a peptide that is homologous to any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64, or a functional fragment thereof. As further described herein, the term “homologous” can be used herein to denote peptides or peptide complexes having at least 70%, at least 80%, at least 90%, at least 95%, or greater than 95% sequence identity or homology to a sequence of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 or a functional fragment thereof. In various embodiments, a fragment can be least 5, at least 10, at least 15, at least 20, at least 25, at least 30, at least 35, at least 40, at least 45, at least 50, at least 100, at least 150, at least 200, at least 250, at least 300, at least 350, at least 400, at least 450, at least 500, at least 600, at least 700, at least 800, at least 900 or at least 1000 amino acids in length. In various embodiments, fragments can be at most 1000, at most 900, at most 800, at most 700, at most 600, at most 500, at most 450, at most 400, at most 350, at most 300, at most 250, at most 200, at most 150, at most 100, at most 50, at most 45, at most 40, at most 35, at most 30, at most 25, at most 20, at most 15, at most 10, or at most 5 amino acids in length. In some embodiments, a fragment can be from about 5 to about 50, from about 10 to about 50, from about 10 to about 40, from about 10 to about 30, or from about 10 to about 20 amino acids in length.

In still other instances, the nucleic acid molecules that encode a peptide or peptide complex of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 can be identified by either a determination of the sequence identity or homology of the encoded peptide amino acid sequence with the amino acid sequence of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64, or by a nucleic acid hybridization assay. Such peptide variants or peptide complex variants of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 can be characterized as nucleic acid molecules (1) that remain hybridized with a nucleic acid molecule having the nucleotide sequence of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 (or its complement) under highly stringent washing conditions, in which the wash stringency is equivalent to 0.1×−0.2×SSC with 0.1% SDS at 50-65° C., and (2) that encode a peptide having at least 70%, at least 80%, at least 90%, at least 95% or greater than 95% sequence identity or homology to the amino acid sequence of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64.

### Affinity Maturation

A peptide of the present disclosure (e.g., a target-binding peptide, TfR-binding peptide, or a selective depletion complex) can be identified or modified through affinity maturation. For example, a target-binding peptide that binds a target of interest can be identified by affinity maturation of a binding peptide (e.g., a CDP, a nanobody, an affibody, a DARPin, a centyrin, a nanofittin, an adnectin, or an antibody fragment). A binding peptide can undergo affinity maturation by generating a library of every possible point mutation, or in the case of a CDP, every possible non-cysteine point mutation. The variant library can be expressed via surface display (e.g., in yeast or mammalian cells) and screened for binding to a binding partner (e.g., a target molecule or TfR). Library members with increased binding affinity relative to the initial peptide or relative to other members of the variant library can undergo subsequent rounds of maturation. During each round, a variant library of every possible non-cysteine point mutation is generated and screened. In some embodiments, a peptide can undergo 1, 2, 3, 4, 5, 6, 7, 8, 9, or 10 rounds of affinity maturation to identify a peptide with improved binding affinity to the binding partner of interest (e.g., a target molecule or TfR). Variants can be identified by Sanger sequencing, next generation sequencing, or high throughput sequencing (e.g., Illumina sequencing).

In some embodiments, a peptide (e.g., a TfR-binding peptide or a target-binding peptide) can be selected for pH-independent binding. For example, a peptide can be selected for high affinity binding to a binding partner (e.g., a target molecule or a TfR) at both extracellular pH (about pH 7.4) and at endosomal pH (such as about pH 5.5). A peptide with pH-independent binding can bind to a binding partner with a dissociation constant (K_{D}) of less than 50 μM, less than 5 μM, less than 500 nM, less than 100 nM, less than 40 nM, less than 30 nM, less than 20 nM, less than 10 nM, less than 5 nM, less than 2 nM, less than 1 nM, less than 0.5 nM, less than 0.4 nM, less than 0.3 nM, less than 0.2 nM, or less than 0.1 nM at extracellular pH (about pH 7.4). In some embodiments, a target-binding peptide with pH-dependent binding can bind a target molecule with a dissociation constant (K_{D}) of less than 50 μM, less than 5 μM, less than 500 nM, less than 100 nM, less than 40 nM, less than 30 nM, less than 20 nM, less than 10 nM, less than 5 nM, less than 2 nM, less than 1 nM, less than 0.5 nM, less than 0.4 nM, less than 0.3 nM, less than 0.2 nM, or less than 0.1 nM at endosomal pH (such as about pH 5.5). In some embodiments the TfR-binding peptides are stable at endosomal pH, and do not release in the endosome for example under acidic conditions, such as pH 6.9, pH 6.8, pH 6.7, pH 6.6, pH 6.5, pH 6.4, pH 6.3, pH 6.2, pH 6.1, pH 6.0, pH 5.9, pH 5.8, pH 5.7, pH 5.6, pH 5.5, pH 5.4, pH 5.3, pH 5.2, pH 5.1, pH 5.0, pH 4.9, pH 4.8, pH 4.7, pH 4.6, pH 4.5, or lower. Conversely, a peptide that has high affinity for binding to a selected target and used in selective depletion complexes as the peptide or peptide complex that binds such selected target and is released in the endosome for degradation within the cell can be a pH-dependent target-binding CDP such that it is released in the endosome. In some embodiments the target-binding peptides are less stable at endosomal pH, and release wholly or in part in the endosome for example under acidic conditions, such as pH7.3, pH7.2, pH7.1, pH7.0, pH6.9, pH6.8, pH6.7, pH6.6, pH6.5, pH6.4, pH6.3, pH6.2, pH 6.1, pH6.0, pH5.9, pH5.8, pH5.7, pH5.6, pH5.5, pH5.4, pH5.3, pH5.2, pH5.1, pH5.0, pH4.9, pH4.8, pH4.7, pH4.6, pH4.5, or lower.

pH-Dependent Binding

The peptides of the present disclosure (e.g., a target binding peptide or a TfR-binding peptide) can be modified for pH-dependent binding properties. Imparting pH-dependent binding to a target-binding peptide (e.g., a target-binding CDP) can be performed in three stages. First, a library of peptide variant containing histidine (His) point mutations can be designed. Histidine amino acids are introduced into the target-binding peptide because His is the only natural amino acid whose side chain has a pKₐ value between neutral (pH 7.4) and acidic (pH<6) endosomal conditions, and this change of charge as pH changes can alter binding, either directly (e.g., changing charge-charge interaction upon formation of a positive charge at low pH) or indirectly (e.g., the change in charge imparts a subtle change in the structure of the target-binding peptide, disrupting an interface between the target molecule and the target-binding peptide). In some embodiments, a variant screen of the target-binding peptide can be implemented by generating double-His doped libraries. For example, a double-His doped library of a target-binding CDP can comprise a library where every non-Cys, non-His residue is substituted with a His amino acid one- or two-at-a-time. A variant library can be expressed in cells (e.g., yeast cells or mammalian cells) via surface display, with each target-binding peptide variant containing one or two His substitutions. Target-binding peptide variants can be tested for maintenance of binding under neutral pH (about pH 7.4), and for reduced binding under low pH (about pH 6.0 or about pH 5.5). Variants that demonstrated reduced binding affinity under low pH as compared to neutral pH can be identified as target-binding peptides with pH-dependent binding.

In some embodiments, the target-binding peptides of the present disclosure (e.g., histidine-containing or histidine-enriched target-binding peptides) can have a high target binding affinity at physiologic extracellular pH but a significantly reduced binding affinity at lower pH levels such as endosomal pH of 5.5. In some cases, the target-binding peptides of the present disclosure can be optimized for improved intra-vesicular (e.g., intra-endosomal) and/or intracellular delivery function while retaining high target binding capabilities. In some cases, histidine scans and comparative binding experiments can be performed to develop and screen for such peptides. In some embodiments, an amino acid residue in a peptide of the present disclosure is substituted with a different amino acid residue to alter a pH-dependent binding affinity to a target molecule. The amino acid substitution can increase a binding affinity at low pH, increase a binding affinity at high pH, decrease a binding affinity at low pH, decrease a binding affinity at high pH, or a combination thereof.

In some embodiments, a target-binding peptide with pH-dependent binding can bind a target molecule with a dissociation constant (K_{D}) of less than 50 μM, less than 5 μM, less than 500 nM, less than 100 nM, less than 40 nM, less than 30 nM, less than 20 nM, less than 10 nM, less than 5 nM, less than 2 nM, less than 1 nM, less than 0.5 nM, less than 0.4 nM, less than 0.3 nM, less than 0.2 nM, or less than 0.1 nM at extracellular pH (such as about pH 7.4). In some embodiments, a target-binding peptide with pH-dependent binding can bind a target molecule with a dissociation constant (K_{D}) at least 1 nM, at least 2 nM, at least 5 nM, at least 10 nM, at least 20 nM, at least 50 nM, of at least 100 nM, at least 200 nM, at least 500 nM, at least 1 μM, at least 2 μM, at least 5 μM, at least 10 μM, at least 20 μM, at least 50 μM, at least 100 μM, at least 500 μM, at least 1 mM, at least 2 mM, at least 5 mM, at least 10 mM, at least 20 mM, at least 50 mM, at least 100 mM, at least 200 mM, at least 500 mM, or at least 1 M at endosomal pH (such as about pH 5.5). In some embodiments the TfR-binding peptides are stable at endosomal pH, and do not release in the endosome for example under acidic conditions, such as pH 6.9, pH 6.8, pH 6.7, pH 6.6, pH 6.5, pH 6.4, pH 6.3, pH 6.2, pH 6.1, pH 6.0, pH 5.9, pH 5.8, pH 5.7, pH 5.6, pH 5.5, pH 5.4, pH 5.3, pH 5.2, pH 5.1, pH 5.0, pH 4.9, pH 4.8, pH 4.7, pH 4.6, pH 4.5, or lower. Conversely, a peptide that has high affinity for binding to a selected target and used in selective depletion complexes as the peptide or peptide complex that binds such selected target and is released in the endosome for degradation within the cell can be a pH-dependent target-binding CDP such that it is released in the endosome. In some embodiments the target-binding peptides are less stable at endosomal pH, and release wholly or in part in the endosome for example under acidic conditions, such as pH 7.3, pH 7.2, pH 7.1, pH 7.0, pH 6.9, pH 6.8, pH 6.7, pH 6.6, pH 6.5, pH 6.4, pH 6.3, pH 6.2, pH 6.1, pH 6.0, pH 5.9, pH 5.8, pH 5.7, pH 5.6, pH 5.5, pH 5.4, pH 5.3, pH 5.2, pH 5.1, pH 5.0, pH 4.9, pH 4.8, pH 4.7, pH 4.6, pH 4.5, or lower.

### Methods of Using Selective Depletion Complexes

The selective depletion complexes of the present disclosure may be used to exert an effect on a cell, tissue, or subject. The effect may be a therapeutic, pharmacological, biological, or biochemical effect. In some embodiments, the effect may result from selective depletion of a target molecule to which the selective depletion complex binds. In some embodiments, the effect may result from ternary complex formation between a target, a receptor, and a selective depletion complex that binds the target and the receptor.

### Selective Depletion of Target Molecules

Described herein are methods of selectively depleting a target molecule using a composition of the present disclosure (e.g., a selective depletion complex). In some embodiments, a method of the present disclosure can comprise selectively recruiting a molecule to an endocytic compartment via transferrin receptor-mediated endocytosis and enriching the target molecule in the lysosome. A selective depletion complex (e.g., a complex comprising a receptor-binding peptide conjugated to a target-binding peptide) can bind to the receptor via the receptor-binding peptide and to a target molecule (e.g., a soluble protein, an extracellular protein, or a cell surface protein). The target molecule can be delivered to an endocytic compartment via receptor-mediated endocytosis of the receptor and the selective depletion molecule. In the endocytic compartment, the selective depletion complex can remain bound to the receptor, and the target molecule can be released from the selective depletion complex as the endocytic compartment acidifies. The selective depletion molecule can be recycled to the cell surface along with the receptor, and the target molecule can continue to the lysosome where it is degraded. In some embodiments, the target molecule can remain in the lysosome without being degraded, resulting in enrichment of the target molecule in the lysosome, such as lysosomal enzymes in lysosomal storage diseases.

The methods of the present disclosure for selectively depleting a target molecule or for selectively enriching a target molecule in the lysosome can be used to treat a disease or condition associated with the target molecule. For example, selective depletion of a target molecule associated with neurodegeneration can be used to treat a neurodegenerative disease. In another example, selective depletion of a target molecule associated with cancer can be used to treat the cancer. Depletion of a cell surface molecule can allow the cancer cell to be targeted by the immune system, to lose checkpoint inhibition, can disable survival signaling, or remove drug resistance pumps. In another example, selective depletion of an inflammatory molecule can be used to treat harmful inflammatory signaling. In another example, selective enrichment in the lysosome of a lysosomal enzyme associated with a lysosomal storage disease can be used to treat the lysosomal storage disease. In this example, a lysosomal enzyme can be administered in co-therapy with the target-depleting complex, such that the target depleting complex drives the lysosomal enzyme into the lysosomal compartment. A method of treating a disease or condition can comprise contacting a cell (e.g., a cell expressing the receptor) with a selective depletion complex of the present disclosure. In some embodiments, the selective depletion complex can be administered to a subject (e.g., a human subject) having a disease or condition (e.g., a neurodegenerative disease, a cancer, harmful inflammation, or a lysosomal storage disease).

TfR is a ubiquitous protein, as all mammalian cells require iron and therefore take up transferrin through this constitutive pathway. By this mechanism, virtually any target tissue would be amenable to the selective depletion methods or selective enrichment methods of the present disclosure comprising a TfR-binding peptide. Tumor tissue can be particularly well-suited for the methods of the present disclosure as most tumors are enriched for TfR, which can impart natural tumor selectivity in the selective depletion molecules.

Liver tissue can also be highly enriched for TfR and thus be a favorable tissue for selective depletion methods. In some embodiments, the selective depletion complexes of the present disclosure (e.g., selective depletion complexes comprising a CDP) can be stable in the liver for extended periods of time. For example, a selective depletion complex of the present disclosure can have a half-life in the liver of at least about 1, at least about 2, at least about 3, at least about 4, at least about 5, at least about 6, at least about 7, at least about 8, at least about 9, or at least about 10 hours. Serum proteins, which can already largely be subject to hepatic metabolism as a class, could be targeted for selective depletion with relatively low doses of selective depletion complexes. Serum half-life of the selective depletion complexes of the present disclosure could be improved to create a molecule that requires infrequent dosing, for example by addition of a serum half-life extension peptide. Selective depletion complexes with a shorter half-life can serve as an acute target elimination drug, for example to treat harmful inflammatory signaling.

A selective depletion complex can be administered to a subject systemically or peripherally and can accumulate in tissue with high levels of TfR expression (e.g., tumor tissue, kidney tissue, spleen, bone marrow, or liver tissue). In some embodiments, a selective depletion complex can be administered to a subject systemically or peripherally and can accumulate in kidney tissue or liver tissue. In some embodiments, a selective depletion complex can comprise a tissue targeting domain and can accumulate in the target tissue upon administration to a subject. For example, selective depletion complexes can be conjugated to, linked to, or fused to a molecule (e.g., small molecule, peptide, or protein) with targeting or homing function for a cell of interest or a target protein located on the surface or inside said cell. In some embodiments, a selective depletion complex can be administered to a subject orally and can reach the gastrointestinal tract. Orally administered selective depletion complexes can be used for clearance of disease-associated proteins in the gastrointestinal tract.

In some embodiments, a selective depletion complex of the present disclosure can be genetically encoded into a benign cell with a secretory phenotype. The selective depletion complex can be expressed by the secretory cell and administered as a secreted molecule in a localized cellular therapy. In some embodiments, a gene encoding a selective depletion complex can be delivered as a gene therapy to a tissue of interest (e.g., liver, hematopoietic, kidney, skin, tumor, central nervous system (CNS), or neurons).

In some embodiments, a target-binding peptide of a selective depletion construct may comprise a miniprotein, a nanobody, an antibody, an IgG, an antibody fragment, a Fab, a F(ab)2, an scFv, an (scFv)2, a DARPin, or an affibody. In some embodiments, the target-binding peptide may comprise a cystine-dense peptide, an affitin, an adnectin, an avimer, a Kunitz domain, a nanofittin, a fynomer, a bicyclic peptide, a beta-hairpin, or a stapled peptide. For example, the target-binding peptide may comprise an antibody single chain variable fragment (scFv) that binds PD-L1, FGFR-1, VEGF, PD-1, EGFR, CD38, GD2, SLAMF7, CTLA-4, CCR4, CD20, PDGFRα, VEGFR2, HER2, CD33, CD30, CD22, CD79B, Nectin-4, or TROP2 and has been modified for pH-dependent binding. A target-binding peptide of a selective depletion complex may bind to a target molecule, such as a target molecule with clinical relevance. In some embodiments, a target molecule may be a protein that is over-expressed or over-activated in a disease or condition. For example, a target molecule may be a transmembrane protein involved in oncogenic signaling, immune suppression, or pro-inflammatory signaling. Examples of target molecules that may be targeted by a target-binding peptide of the present disclosure include but are not limited to CD3, CD47, CD28, CD137, CD89, CD16, CD29, CD44, CD71, CD73, CD90, CD105, CD166, CD27, CD39, CD24, CD25, CD74, CD40L, MUC1, MUC16, MUC2, MUC5AC, MUC4, OX40, 4-1BB, HLA-G, LAG3, Tim3, TIGIT, GITR, TCR, TNF-α, EGFR, EGFRvIII, TKI-resistant EGFR, HER2, ERBB3, PDGFR, FGF, VEGF, VEGFR, IGFR1, CTLA4, STRO1, complement factor C4, complement factor C1q, complement factor C1s, complement factor C1r, complement factor C3, complement factor C3a, complement factor C3b, complement factor C5, complement factor C5a, TGFβ, PCSK9, P2Y6, HER3, RANK, tau, amyloid ß, huntingtin, α-synuclein, glucocerebrosidase, α-glucosidase, IL-1, IL-1R, IL-1α, IL-1β, IL-2, IL-2R, IL-4, IL-5, IL-6, IL-6R, IL-10, IL-10R, IL-17, IL-23, IL-12, p40, a member of the B7 family, c-Met, SIGLEC, MCP-1, an MHC, an MHC I, an MHC II, PD-1, and PD-L1.

Endocytosis and subsequent degradation of the target molecule by a selective depletion complex may treat (e.g., eliminate, reduce, slow progression of, or treat symptoms of) a disease or condition associated with the target molecule (e.g., CD3, CD47, CD28, CD137, CD89, CD16, CD29, CD44, CD71, CD73, CD90, CD105, CD166, CD27, CD39, CD24, CD25, CD74, CD40L, MUC1, MUC16, MUC2, MUC5AC, MUC4, OX40, 4-1BB, HLA-G, LAG3, Tim3, TIGIT, GITR, TCR, TNF-α, EGFR, EGFRvIII, TKI-resistant EGFR, HER2, ERBB3, PDGFR, FGF, VEGF, VEGFR, IGFR1, CTLA4, STRO1, complement factor C4, complement factor C1q, complement factor C1s, complement factor C1r, complement factor C3, complement factor C3a, complement factor C3b, complement factor C5, complement factor C5a, TGFβ, PCSK9, P2Y6, HER3, RANK, tau, amyloid ß, huntingtin, α-synuclein, glucocerebrosidase, α-glucosidase, IL-1, IL-1R, IL-1α, IL-1β, IL-2, IL-2R, IL-4, IL-5, IL-6, IL-6R, IL-10, IL-10R, IL-17, IL-23, IL-12, p40, a member of the B7 family, c-Met, SIGLEC, MCP-1, an MHC, an MHC I, an MHC II, PD-1, or PD-L1). In some embodiments, the target molecule is over-expressed in the disease or condition and depleting the target molecule reduces the level of the target molecule, thereby treating the disease or condition. In some embodiments, the target molecule accumulates in the disease or condition and depleting the target molecule clears or reduces the accumulation, thereby treating the disease or condition. In some embodiments, the target molecule is hyper-activated or over-stimulated, and depleting the target molecule reduces a level of activity of the target molecule, thereby treating the disease or condition. Examples of diseases that may be treated using a selective depletion complex include cancers, (e.g., non-small-cell lung cancer, primary non-small-cell lung cancer, metastatic non-small-cell lung cancer, head and neck cancer, head and neck squamous cell carcinoma, glioblastoma, brain cancer, metastatic brain cancer, colorectal cancer, colon cancer, tyrosine kinase inhibitor (TKI)-resistant cancer, cetuximab-resistant cancer, necitumumab-resistant cancer, panitumumab-resistant cancer, local cancer, regionally advanced cancer, recurrent cancer, metastatic cancer, refractory cancer, KRAS wildtype cancer, KRAS mutant cancers, or exon20 mutant non-small-cell lung cancer), inflammation, inflammatory conditions, neurological conditions (e.g., neuroinflammation, neuroinflammatory disease, stroke, traumatic brain injury, Alzheimer's disease, or other tauopathies including neurofibrillary tangle dementia, chronic traumatic encephalopathy (CTE), aging-related tau astrogliopathy, frontotemporal dementia, parkinsonism, progressive supranuclear palsy, corticobasal degeneration, lytico-bodig disease, ganglioglioma, meningioangiomatosis, or subacute sclerosing panencephalitis).

Administration of a selective depletion complex of the present disclosure may be combined with an additional therapy to treat a disease or condition. For example, administration of a selective depletion complex to treat a cancer may be combined with administration of radiation therapy, chemotherapy, platinum therapy, or anti-metabolic therapy. In some embodiments, the additional therapy may comprise administering fluorouracil, FOLFIRI, irinotecan, FOLFOX, gemcitabine, or cisplatin to the subject.

### Ternary Complex Formation

Described herein are methods of forming a ternary complex between a target molecule, a receptor, and a selective depletion complex comprising a receptor-binding peptide and a target-binding peptide. The ternary complex may form through binding of the receptor-binding peptide to the receptor and binding of the target-binding peptide to the target. Ternary complex formation between the target, the receptor, and the selective depletion complex may exert a therapeutic, pharmacological, biological, or biochemical effect on a cell, tissue, or subject expressing the target and the receptor. In some embodiments, formation of a ternary complex between a receptor, a target, and a selective depletion complex may increase recycling or turnover of the target molecule, the receptor, or both. Increased recycling or turnover of the target or the receptor may alter (e.g., increase) activity of the target or the receptor, thereby exerting a therapeutic, pharmacological, biological, or biochemical effect.

Formation of the ternary complex may exert a therapeutic, pharmacological, biological, or biochemical by recruiting the target molecule to the receptor. Recruitment of the target molecule to the receptor may promote a binding interaction between the receptor and the target. In some embodiments, subsequent recycling of the receptor and the target may facilitate the therapeutic, pharmacological, biological, or biochemical effect. In some embodiments, formation of the ternary complex may stabilize the interaction between the target and the receptor.

### Physicochemical Properties of Peptides

In some embodiments, a peptide of the present disclosure (e.g., a TfR-binding peptide, a target binding peptide, or a selective depletion complex) can comprise a wide range of physicochemical properties such as molecular size and structure, pH, isoelectric point, and overall molecular net charge. These parameters can have an effect on the peptides ability to bind TfR, bind a target molecule, promote transcytosis, transport of cargo molecules across cell barrier such as the BBB, or combinations thereof.

A peptide of the present disclosure can comprise at least one amino acid residue in D configuration. In some embodiments, a peptide is about 5-100 amino acid residues long. In some embodiments, a peptide is about 10-90 amino acid residues long. In some embodiments, a peptide is about 15-80 amino acid residues long. In some embodiments, a peptide is about 15-75 amino acid residues long. In some embodiments, a peptide is about 15-70 amino acid residues long. In some embodiments, a peptide is about 20-65 amino acid residues long. In some embodiments, a peptide is about 20-60 amino acid residues long. In some embodiments, a peptide is about 25-55 amino acid residues long. In some embodiments, a peptide is about 25-50 amino acid residues long. In some embodiments, a peptide is about 25-40 amino acid residues long. In some embodiments, a peptide is about 11-35 amino acid residues long. In some embodiments, a peptide is about 10-25 amino acid residues long.

In some embodiments, a peptide is at least 5 amino acid residues long. In some embodiments, a peptide is at least 10 amino acid residues long. In some embodiments, a peptide is at least 15 amino acid residues long. In some embodiments, a peptide is at least 20 amino acid residues long. In some embodiments, a peptide is at least 25 amino acid residues long. In some embodiments, a peptide is at least 30 amino acid residues long. In some embodiments, a peptide is at least 35 amino acid residues long. In some embodiments, a peptide is at least 40 amino acid residues long. In some embodiments, a peptide is at least 45 amino acid residues long. In some embodiments, a peptide is at least 50 amino acid residues long. In some embodiments, a peptide is at least 55 amino acid residues long. In some embodiments, a peptide is at least 60 amino acid residues long. In some embodiments, a peptide is at least 65 amino acid residues long. In some embodiments, a peptide is at least 70 amino acid residues long. In some embodiments, a peptide is at least 75 amino acid residues long.

In some embodiments, an amino acid sequence of a peptide as described herein comprises at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 41, at least 42, at least 43, at least 44, at least 45, at least 46, at least 47, at least 48, at least 49, at least 50, at least 51, at least 52, at least 53, at least 54, at least 55, at least 56, at least 57, at least 58 residues, at least 59, at least 60, at least 61, at least 62, at least 63, at least 64, at least 65, at least 66, at least 67, at least 68, at least 69, at least 70, at least 71, at least 72, at least 73, at least 74, at least 75, at least 76, at least 77, at least 78, at least 79, at least 80, or at least 81 amino acid residues.

In some embodiments of the present disclosure, a three-dimensional or tertiary structure of a peptide is primarily comprised of beta-sheets and/or alpha-helix structures. In some embodiments, designed or engineered peptides (e.g., target-binding peptides, TfR-binding peptides, or selective depletion complexes) of the present disclosure are small, compact peptides or polypeptides stabilized by intra-chain disulfide bonds (e.g., mediated by cysteines) and a hydrophobic core. In some embodiments, engineered peptides have structures comprising helical bundles with at least one disulfide bridge between each of the alpha helices, thereby stabilizing the peptides. In other embodiments, the engineered TfR-binding peptides comprise structures with three alpha helices and three intra-chain disulfide bonds, one between each of the three alpha helices in the bundle of alpha helices.

At physiologic extracellular pH, peptides as described herein can have an overall molecular net charge, for example, of −5, −4, −3, −2, −1, 0, +1, +2, +3, +4, or +5. When the net charge is zero, the peptide can be uncharged or zwitterionic. In some embodiments, a peptide contains one or more disulfide bonds and has a positive net charge at physiologic extracellular pH where the net charge can be +0.5 or less than +0.5, +1 or less than +1, +1.5 or less than +1.5, +2 or less than +2, +2.5 or less than +2.5, +3 or less than +3, +3.5 or less than +3.5, +4 or less than +4, +4.5 or less than +4.5, +5 or less than +5, +5.5 or less than +5.5, +6 or less than +6, +6.5 or less than +6.5, +7 or less than +7, +7.5 or less than +7.5, +8 or less than +8, +8.5 or less than +8.5, +9 or less than +9.5, +10 or less than +10. In some embodiments, a peptide has a negative net charge at physiologic extracellular pH where the net charge can be −0.5 or less than −0.5, −1 or less than −1, −1.5 or less than −1.5, −2 or less than −2, −2.5 or less than −2.5, −3 or less than −3, −3.5 or less than −3.5, −4 or less than −4, −4.5 or less than −4.5, −5 or less than −5, −5.5 or less than −5.5, −6 or less than −6, −6.5 or less than −6.5, −7 or less than −7, −7.5 or less than −7.5, −8 or less than −8, −8.5 or less than −8.5, −9 or less than −9.5, −10 or less than −10.

In some embodiments, peptides of the present disclosure can have an isoelectric point (pI) value from 3 and 10. In other embodiments, peptides of the present disclosure can have a pI value from 4.3 and 8.9. In some embodiments, peptides of the present disclosure can have a pI value from 3-4. In some embodiments, peptides of the present disclosure can have a pI value from 3-5. In some embodiments, peptides of the present disclosure can have a pI value from 3-6. In some embodiments, peptides of the present disclosure can have a pI value from 3-7. In some embodiments, peptides of the present disclosure can have a pI value from 3-8. In some embodiments, peptides of the present disclosure can have a pI value from 3-9. In some embodiments, peptides of the present disclosure can have a pI value from 4-5. In some embodiments, peptides of the present disclosure can have a pI value from 4-6. In some embodiments, peptides of the present disclosure can have a pI value from 4-7. In some embodiments, peptides of the present disclosure can have a pI value from 4-8. In some embodiments, peptides of the present disclosure can have a pI value from 4-9. In some embodiments, peptides of the present disclosure can have a pI value from 4-10. In some embodiments, peptides of the present disclosure can have a pI value from 5-6. In some embodiments, peptides of the present disclosure can have a pI value from 5-7. In some embodiments, peptides of the present disclosure can have a pI value from 5-8. In some embodiments, peptides of the present disclosure can have a pI value from 5-9. In some embodiments, peptides of the present disclosure can have a pI value from 5-10. In some embodiments, peptides of the present disclosure can have a pI value from 6-7. In some embodiments, peptides of the present disclosure can have a pI value from 6-8. In some embodiments, peptides of the present disclosure can have a pI value from 6-9. In some embodiments, peptides of the present disclosure can have a pI value from 6-10. In some embodiments, peptides of the present disclosure can have a pI value from 7-8. In some embodiments, peptides of the present disclosure can have a pI value from 7-9. In some embodiments, peptides of the present disclosure can have a pI value from 7-10. In some embodiments, peptides of the present disclosure can have a pI value from 8-9. In some embodiments, peptides of the present disclosure can have a pI value from 8-10. In some embodiments, peptides of the present disclosure can have a pI value from 9-10.

In some cases, the engineering of one or more mutations within a peptide of the present disclosure (e.g., a TfR-binding peptide) yields a peptide with an altered isoelectric point, charge, surface charge, or rheology at physiologic extracellular pH. Such engineering of a mutation to a peptide that can be derived from a scorpion or spider complex can change the net charge of the peptide, for example, by decreasing the net charge by 1, 2, 3, 4, or 5, or by increasing the net charge by 1, 2, 3, 4, or 5. In such cases, the engineered mutation can facilitate the ability of the peptide to bind a target protein, promote transcytosis, and penetrate a cell, an endosome, or the nucleus. Suitable amino acid modifications for improving the rheology and potency of a peptide can include conservative or non-conservative mutations.

A peptide can comprise at most 1 amino acid mutation, at most 2 amino acid mutations, at most 3 amino acid mutations, at most 4 amino acid mutations, at most 5 amino acid mutations, at most 6 amino acid mutations, at most 7 amino acid mutations, at most 8 amino acid mutations, at most 9 amino acid mutations, at most 10 amino acid mutations, or another suitable number as compared to the sequence of the venom or toxin component that the peptide is derived from. In other embodiments, a peptide, or a functional fragment thereof, comprises at least 1 amino acid mutation, at least 2 amino acid mutations, at least 3 amino acid mutations, at least 4 amino acid mutations, at least 5 amino acid mutations, at least 6 amino acid mutations, at least 7 amino acid mutations, at least 8 amino acid mutations, at least 9 amino acid mutations, at least 10 amino acid mutations, or another suitable number as compared to the sequence of the venom or toxin component that the peptide is derived from. In some embodiments, mutations can be engineered within a peptide to provide a peptide that has a desired charge or stability at physiologic extracellular pH.

Generally, the nuclear magnetic resonance (NMR) solution structures, the X-ray crystal structures, as well as the primary structure sequence alignment of related structural peptide or protein homologs or in silico design can be used to generate mutational strategies that can improve the folding, stability, and/or manufacturability, while maintaining a particular biological function (e.g., TfR affinity/binding). A general strategy for producing homologs or in silico designed peptides or polypeptides can include identification of a charged surface patch or conserved residues of a protein, mutation of critical amino acid positions and loops, followed by in vitro and in vivo testing of the peptides. The overall peptide optimization process can be of iterative nature to the extent that, for example, information obtained during in vitro or in vivo testing is used for the design of the next generation of peptides. Hence, the herein disclosed methods can be used to design peptides with improved properties or to correct deleterious mutations that complicate folding and manufacturability. Key amino acid positions and loops can be retained while other residues in the peptide sequences can be mutated to improve, change, remove, or otherwise modify function, such as binding, transcytosis, or the ability to penetrate a cell, endosome, or nucleus in a cell, homing, or another activity of the peptide. These techniques can be used to predict the 3D pharmacophore of a group of structurally homologous scaffolds, as wells as to predict possible graft regions of related proteins to create chimeras with improved properties (e.g., binding properties). For example, this strategy is used to identify critical amino acid positions and loops that are used to design peptides with improved TfR receptor binding and transcytosis properties, high expression, high stability in vivo, or any combination of these properties.

The present disclosure also encompasses multimers of the various peptides described herein. Examples of multimers include dimers, trimers, tetramers, pentamers, hexamers, heptamers, and so on. A multimer can be a homomer formed from a plurality of identical subunits or a heteromer formed from a plurality of different subunits. In some embodiments, a peptide of the present disclosure is arranged in a multimeric structure with at least one other peptide, or two, three, four, five, six, seven, eight, nine, ten, or more other peptides. In certain embodiments, the peptides of a multimeric structure each have the same sequence. In other embodiments, one or more or all of the peptides of a multimeric structure have different sequences.

In some embodiments, the present disclosure provides peptide scaffolds that can be used as a starting point for generating additional, next-generation peptides with more specific or improved properties. In some embodiments, these scaffolds are derived from a variety of CDPs or knotted peptides. Some suitable peptides for scaffolds can include, but are not limited to, chlorotoxin, brazzein, circulin, stecrisp, hanatoxin, midkine, hefutoxin, potato carboxypeptidase inhibitor, bubble protein, attractin, α-GI, α-GID, μ-PIIIA, ω-MVIIA, ω-CVID, χ-MrIA, ρ-TIA, conantokin G, contulakin G, GsMTx4, margatoxin, shK, toxin K, chymotrypsin inhibitor (CTI), and EGF epiregulin core. In some embodiments, the peptide sequence is flanked by additional amino acids. One or more additional amino acids can confer a desired in vivo charge, isoelectric point, chemical conjugation site, stability, or physiologic property to a peptide.

### Pharmacokinetics of Peptides

The pharmacokinetics of any of the peptides of the present disclosure can be determined after administration of the peptide via different routes of administration. For example, the pharmacokinetic parameters of a peptide of this disclosure can be quantified after intravenous, subcutaneous, intramuscular, rectal, aerosol, parenteral, ophthalmic, pulmonary, transdermal, vaginal, optic, nasal, oral, sublingual, inhalation, dermal, intrathecal, intranasal, peritoneal, buccal, synovial, intratumoral, or topical administration. Peptides of the present disclosure can be analyzed by using tracking agents such as radiolabels or fluorophores. For example, radiolabeled peptides of this disclosure can be administered via various routes of administration. Peptide concentration or dose recovery in various biological samples such as plasma, urine, feces, any organ, skin, muscle, and other tissues can be determined using a range of methods including HPLC, fluorescence detection techniques (TECAN quantification, flow cytometry, iVIS), or liquid scintillation counting.

The methods and compositions described herein relate to pharmacokinetics of peptide administration via any route to a subject. Pharmacokinetics can be described using methods and models, for example, compartmental models or non-compartmental methods. Compartmental models include but are not limited to monocompartmental model, the two compartmental model, the multicompartmental model or the like. Models are often divided into different compartments and can be described by the corresponding scheme. For example, one scheme is the absorption, distribution, metabolism and excretion (ADME) scheme. For another example, another scheme is the liberation, absorption, distribution, metabolism and excretion (LADME) scheme. In some aspects, metabolism and excretion can be grouped into one compartment referred to as the elimination compartment. For example, liberation includes liberation of the active portion of the composition from the delivery system, absorption includes absorption of the active portion of the composition by the subject, distribution includes distribution of the composition through the blood plasma and to different tissues, metabolism, which includes metabolism or inactivation of the composition and finally excretion, which includes excretion or elimination of the composition or the products of metabolism of the composition. Compositions administered intravenously to a subject can be subject to multiphasic pharmacokinetic profiles, which can include but are not limited to aspects of tissue distribution and metabolism/excretion. As such, the decrease in plasma or serum concentration of the composition is often biphasic, including, for example an alpha phase and a beta phase, occasionally a gamma, delta or other phase is observed.

Pharmacokinetics includes determining at least one parameter associated with administration of a peptide to a subject. In some aspects, parameters include at least the dose (D), dosing interval (τ), area under curve (AUC), maximum concentration (Cₘₐₓ), minimum concentration reached before a subsequent dose is administered (Cₘᵢₙ), minimum time (Tₘᵢₙ), maximum time to reach Cₘₐₓ (Tₘₐₓ), volume of distribution (V_{d}), steady-state volume of distribution (Vₛₛ), back-extrapolated concentration at time 0 (C₀), steady state concentration (Cₛₛ), elimination rate constant (kₑ), infusion rate (kᵢₙ), clearance (CL), bioavailability (f), fluctuation (% PTF) and elimination half-life (t_{1/2}).

In certain embodiments, the peptides or peptide complexes of any of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 exhibit optimal pharmacokinetic parameters after oral administration. In other embodiments, the peptides or peptide complexes of any of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 exhibit optimal pharmacokinetic parameters after any route of administration, such as oral administration, inhalation, intranasal administration, topical administration, intravenous administration, subcutaneous administration, intra-articular administration, intramuscular administration, intraperitoneal administration, intra-synovial, or any combination thereof.

In some embodiments, any peptide or peptide complex of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 exhibits an average Tₘₐₓ of 0.5-12 hours, or 1-48 hours at which the Cₘₐₓ is reached, an average bioavailability in serum of 0.1%-10% in the subject after administering the peptide to the subject by an oral route, an average bioavailability in serum of less than 0.1% after oral administration to a subject for delivery to the GI tract, an average bioavailability in serum of 10-100% after parenteral administration, an average t_{1/2} of 0.1 hours-168 hours, or 0.25 hours-48 hours in a subject after administering the peptide to the subject, an average clearance (CL) of 0.5-100 L/hour or 0.5-50 L/hour of the peptide after administering the peptide to a subject, an average volume of distribution (V_{d}) of 200-20,000 mL in the subject after systemically administering the peptide to the subject, or optionally no systemic uptake, any combination thereof.

### Peptide Stability

A peptide of the present disclosure can be stable in various biological or physiological conditions, such as physiologic extracellular pH, endosomal or lysosomal pH, or reducing environments inside a cell, in the cytosol, in a cell nucleus, or endosome or a tumor. For example, any peptide or peptide complex comprising any of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 can exhibit resistance to reducing agents, proteases, oxidative conditions, or acidic conditions.

In some cases, biologic molecules (such as peptides and proteins) can provide therapeutic functions, but such therapeutic functions are decreased or impeded by instability caused by the in vivo environment. (Moroz et al. Adv Drug Deliv Rev 101:108-21 (2016), Mitragotri et al. Nat Rev Drug Discov 13(9):655-72 (2014), Bruno et al. Ther Deliv (11):1443-67 (2013), Sinha et al. Crit Rev Ther Drug Carrier Syst. 24(1):63-92 (2007), Hamman et al. BioDrugs 19(3):165-77 (2005)). For instance, the GI tract can contain a region of low pH (e.g. pH˜1), a reducing environment, or a protease-rich environment that can degrade peptides and proteins. Proteolytic activity in other areas of the body, such as the mouth, eye, lung, intranasal cavity, joint, skin, vaginal tract, mucous membranes, and serum, can also be an obstacle to the delivery of functionally active peptides and polypeptides. Additionally, the half-life of peptides in serum can be very short, in part due to proteases, such that the peptide can be degraded too quickly to have a lasting therapeutic effect when administering reasonable dosing regimens. Likewise, proteolytic activity in cellular compartments such as lysosomes and reduction activity in lysosomes and the cytosol can degrade peptides and proteins such that they can be unable to provide a therapeutic function on intracellular targets. Therefore, peptides that are resistant to reducing agents, proteases, and low pH can be able to provide enhanced therapeutic effects or enhance the therapeutic efficacy of co-formulated or conjugated, linked, or fused active agents in vivo.

### Methods of Manufacture

Various expression vector/host systems can be utilized for the recombinant expression of peptides described herein. Non-limiting examples of such systems include microorganisms such as bacteria transformed with recombinant bacteriophage DNA, plasmid DNA or cosmid DNA expression vectors containing a nucleic acid sequence encoding peptides, peptide complexes, or peptide fusion proteins/chimeric proteins described herein, yeast transformed with recombinant yeast expression vectors containing the aforementioned nucleic acid sequence, insect cell systems infected with recombinant virus expression vectors (e.g., baculovirus) containing the aforementioned nucleic acid sequence, plant cell systems infected with recombinant virus expression vectors (e.g., cauliflower mosaic virus (CaMV), tobacco mosaic virus (TMV)), or transformed with recombinant plasmid expression vectors (e.g., Ti plasmid) containing the aforementioned nucleic acid sequence, or animal cell systems infected with recombinant virus expression vectors (e.g., adenovirus, vaccinia virus, lentivirus) including cell lines engineered to contain multiple copies of the aforementioned nucleic acid sequence, either stably amplified (e.g., CHO/dhfr, CHO/glutamine synthetase) or unstably amplified in double-minute chromosomes (e.g., murine cell lines). Disulfide bond formation and folding of the peptide could occur during expression or after expression or both.

A host cell can be adapted to express one or more peptides described herein. The host cells can be prokaryotic, eukaryotic, or insect cells. In some cases, host cells are capable of modulating the expression of the inserted sequences or modifying and processing the gene or protein product in the specific fashion desired. For example, expression from certain promoters can be elevated in the presence of certain inducers (e.g., zinc and cadmium ions for metallothionine promoters). In some cases, modifications (e.g., phosphorylation) and processing (e.g., cleavage) of peptide products can be important for the function of the peptide. Host cells can have characteristic and specific mechanisms for the post-translational processing and modification of a peptide. In some cases, the host cells used to express the peptides secrete minimal amounts of proteolytic enzymes.

The selective depletion complexes of this disclosure can be advantageously made by a single recombinant expression system, with no need for chemical synthesis or modifications. For example, a selective depletion complex can be expressed in CHO cells, yeast, pichia, E. coli, or other organisms.

In the case of cell- or viral-based samples, organisms can be treated prior to purification to preserve and/or release a target polypeptide. In some embodiments, the cells are fixed using a fixing agent. In some embodiments, the cells are lysed. The cellular material can be treated in a manner that does not disrupt a significant proportion of cells, but which removes proteins from the surface of the cellular material, and/or from the interstices between cells. For example, cellular material can be soaked in a liquid buffer, or, in the case of plant material, can be subjected to a vacuum, in order to remove proteins located in the intercellular spaces and/or in the plant cell wall. If the cellular material is a microorganism, proteins can be extracted from the microorganism culture medium. Alternatively, the peptides can be packed in inclusion bodies. The inclusion bodies can further be separated from the cellular components in the medium. In some embodiments, the cells are not disrupted. A cellular or viral peptide that is presented by a cell or virus can be used for the attachment and/or purification of intact cells or viral particles. In addition to recombinant systems, peptides can also be synthesized in a cell-free system prior to extraction using a variety of known techniques employed in protein and peptide synthesis.

In some cases, a host cell produces a peptide that has an attachment point for a cargo molecule (e.g., a therapeutic agent). An attachment point could comprise a lysine residue, an N-terminus, a cysteine residue, a cysteine disulfide bond, a glutamic acid or aspartic acid residue, a C-terminus, or a non-natural amino acid. The peptide could also be produced synthetically, such as by solid-phase peptide synthesis, or solution-phase peptide synthesis. Peptide synthesis can be performed by fluorenylmethyloxycarbonyl (Fmoc) chemistry or by butyloxycarbonyl (Boc) chemistry. The peptide could be folded (formation of disulfide bonds) during synthesis or after synthesis or both. Peptide fragments could be produced synthetically or recombinantly. Peptide fragments can be then be joined together enzymatically or synthetically.

In other aspects, the peptides of the present disclosure can be prepared by conventional solid phase chemical synthesis techniques, for example according to the Fmoc solid phase peptide synthesis method (“Fmoc solid phase peptide synthesis, a practical approach,” edited by W. C. Chan and P. D. White, Oxford University Press, 2000).

In some embodiments, the peptides of this disclosure can be more stable during manufacturing. For example, peptides of this disclosure can be more stable during recombinant expression and purification, resulting in lower rates of degradation by proteases that are present in the manufacturing process, a higher purity of peptide, a higher yield of peptide, or any combination thereof. In some embodiments, the peptides can also be more stable to degradation at high temperatures and low temperatures during manufacturing, storage, and distribution. For example, in some embodiments peptides of this disclosure can be stable at 25° C. In other embodiments, peptides of this disclosure can be stable at 70° C. or higher than 70° C. In some embodiments, peptides of this disclosure can be stable at 100° C. or higher than 100° C.

### Pharmaceutical Compositions

A pharmaceutical composition of the disclosure can be a combination of any peptide as described herein with other chemical components, such as carriers, stabilizers, diluents, dispersing agents, suspending agents, thickening agents, antioxidants, solubilizers, buffers, osmolytes, salts, surfactants, amino acids, encapsulating agents, bulking agents, cryoprotectants, and/or excipients. The pharmaceutical composition facilitates administration of a peptide described herein to an organism. In some cases, the pharmaceutical composition comprises factors that extend half-life of the peptide and/or help the peptide to penetrate the target cells. In some embodiments, a pharmaceutical composition comprises a cell modified to express and secrete a selective depletion complex of the present disclosure.

Pharmaceutical compositions can be administered in therapeutically-effective amounts as pharmaceutical compositions by various forms and routes including, for example, intravenous, subcutaneous, intramuscular, rectal, aerosol, parenteral, ophthalmic, pulmonary, transdermal, vaginal, optic, nasal, oral, sublingual, inhalation, dermal, intrathecal, intratumoral, intranasal, and topical administration. A pharmaceutical composition can be administered in a local or systemic manner, for example, via injection of the peptide described herein directly into an organ, optionally in a depot.

Parenteral injections can be formulated for bolus injection, infusion, or continuous infusion. The pharmaceutical compositions can be in a form suitable for parenteral injection as a sterile suspension, solution or emulsion in oily or aqueous vehicles, and can contain formulatory agents such as suspending, stabilizing and/or dispersing agents. Pharmaceutical formulations for parenteral administration include aqueous solutions of a peptide described herein in water-soluble form. Suspensions of peptide-antibody complexes described herein can be prepared as oily injection suspensions. Suitable lipophilic solvents or vehicles include fatty oils such as sesame oil, or synthetic fatty acid esters, such as ethyl oleate or triglycerides, or liposomes. Aqueous injection suspensions can contain substances which increase the viscosity of the suspension, such as sodium carboxymethyl cellulose, sorbitol, or dextran. The suspension can also contain suitable stabilizers or agents which increase the solubility and/or reduce the aggregation of such peptide-antibody complexes described herein to allow for the preparation of highly concentrated solutions.

Alternatively, the peptide described herein can be lyophilized or in powder form for re-constitution with a suitable vehicle, e.g., sterile pyrogen-free water, before use. In some embodiments, a purified peptide is administered intravenously. A peptide described herein can be administered to a subject in order to home, target, migrate to, or be directed to a CNS cell, a brain cell, a cancerous cell, or a tumor. In some embodiments, a peptide can be conjugated to, linked to, or fused to another peptide that provides a targeting function to a specific target cell type in the central nervous system or across the blood brain barrier. Exemplary target cells include a CNS cell, erythrocyte, an erythrocyte precursor cell, an immune cell, a stem cell, a muscle cell, a brain cell, a thyroid cell, a parathyroid cell, an adrenal gland cell, a bone marrow cell, an appendix cell, a lymph node cell, a tonsil cell, a spleen cell, a muscle cell, a liver cell, a gallbladder cell, a pancreas cell, a cell of the gastrointestinal tract, a glandular cell, a kidney cell, a urinary bladder cell, an endothelial cell, an epithelial cell, a choroid plexus epithelial cell, a neuron, a glial cell, an astrocyte, or a cell associated with a nervous system.

A peptide of the disclosure can be applied directly to an organ, or an organ tissue or cells, such as brain or brain tissue or cells, during a surgical procedure. The recombinant peptide described herein can be administered topically and can be formulated into a variety of topically administrable compositions, such as solutions, suspensions, lotions, gels, pastes, medicated sticks, balms, creams, and ointments. Such pharmaceutical compositions can contain solubilizers, stabilizers, tonicity enhancing agents, buffers, and preservatives.

In practicing the methods of treatment or use provided herein, therapeutically effective amounts of a peptide described herein can be administered in pharmaceutical compositions to a subject suffering from a condition that affects the immune system. In some embodiments, the subject is a mammal such as a human or a primate. A therapeutically effective amount can vary widely depending on the severity of the disease, the age and relative health of the subject, the potency of the compounds used, and other factors.

In some embodiments, a peptide is cloned into a viral or non-viral expression vector. Such expression vector can be packaged in a viral particle, a virion, or a non-viral carrier or delivery mechanism, which is administered to patients in the form of gene therapy. In other embodiments, patient cells are extracted and modified to express a peptide capable of binding TfR ex vivo before the modified cells are returned back to the patient in the form of a cell-based therapy, such that the modified cells will express the peptide once transplanted back in the patient.

Pharmaceutical compositions can be formulated using one or more physiologically acceptable carriers comprising excipients and auxiliaries, which facilitate processing of the active compounds into preparations that can be used pharmaceutically. Formulation can be modified depending upon the route of administration chosen. Pharmaceutical compositions comprising a peptide described herein can be manufactured, for example, by expressing the peptide in a recombinant system, purifying the peptide, lyophilizing the peptide, mixing, dissolving, granulating, dragee-making, levigating, emulsifying, encapsulating, entrapping, or compression processes. The pharmaceutical compositions can include at least one pharmaceutically acceptable carrier, diluent, or excipient and compounds described herein as free-base or pharmaceutically acceptable salt form.

Methods for the preparation of peptide described herein comprising the compounds described herein include formulating peptide described herein with one or more inert, pharmaceutically acceptable excipients or carriers to form a solid, semi-solid, or liquid composition. Solid compositions include, for example, powders, tablets, dispersible granules, capsules, cachets, and suppositories. These compositions can also contain minor amounts of nontoxic, auxiliary substances, such as wetting or emulsifying agents, pH buffering agents, and other pharmaceutically acceptable additives.

Non-limiting examples of pharmaceutically-acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pennsylvania 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

Pharmaceutical compositions can also include permeation or absorption enhancers (Aungst et al. AAPS J. 14(1):10-8. (2012) and Moroz et al. Adv Drug Deliv Rev 101:108-21. (2016)). Permeation enhancers can facilitate uptake of molecules from the GI tract into systemic circulation. Permeation enhancers can include salts of medium chain fatty acids, sodium caprate, sodium caprylate, N-(8-[2-hydroxybenzoyl]amino)caprylic acid (SNAC), N-(5-chlorosalicyloyl)-8-aminocaprylic acid (5-CNAC), hydrophilic aromatic alcohols such as phenoxyethanol, benzyl alcohol, and phenyl alcohol, chitosan, alkyl glycosides, dodecyl-2-N,N-dimethylamino propionate (DDAIPP), chelators of divalent cations including EDTA, EGTA, and citric acid, sodium alkyl sulfate, sodium salicylate, lecithin-based, or bile salt-derived agents such as deoxycholates.

Compositions can also include protease inhibitors including soybean trypsin inhibitor, aprotinin, sodium glycocholate, camostat mesilate, vacitracin, or cyclopentadecalactone.

### Use of Peptides in Treatments

In some embodiments, a method of treating a subject using the selective depletion complexes of the present disclosure includes administering an effective amount of a peptide as described herein to a subject in need thereof.

In some embodiments, a method of treating a subject using the selective depletion complexes of the present disclosure includes modifying a cell of a subject to express and secrete a selective depletion complex of the present disclosure. In some embodiments, the cell is a cell in the subject. In some embodiments, the cell is a cell that has been removed from the subject and is re-introduced following modification. In some embodiments, the cell is modified using a viral vector (e.g., an oncolytic herpes simplex virus). In some embodiments, a gene encoding expression and secretion of a selective depletion complex is engineered into a CAR-T cell or other cellular therapy.

TfR can be expressed in various tissues such as the brain, the stomach, the liver, of the gall bladder. Hence, the peptides of the present disclosure (e.g., a selective depletion complex comprising a TfR-binding peptide) can be used in the diagnosis and treatment of disease and conditions associated with various tissues and organs. For example, drug delivery to these tissues and organs can be improved by using the herein described peptides and peptide complexes carrying a diagnostic and/or therapeutic payload.

The term “effective amount,” as used herein, refers to a sufficient amount of an agent or a compound being administered which will relieve to some extent one or more of the symptoms of the disease or condition being treated. The result can be reduction and/or alleviation of the signs, symptoms, or causes of a disease, or any other desired alteration of a biological system. Compositions containing such agents or compounds can be administered for prophylactic, enhancing, and/or therapeutic treatments. An appropriate “effective” amount in any individual case can be determined using techniques, such as a dose escalation study.

The methods, compositions, and kits of this disclosure can comprise a method to prevent, treat, arrest, reverse, or ameliorate the symptoms of a condition. The treatment can comprise treating a subject (e.g., an individual, a domestic animal, a wild animal, or a lab animal afflicted with a disease or condition) with a peptide of the disclosure. The disease can be a cancer or tumor. In treating the disease, the peptide can contact the tumor or cancerous cells. The subject can be a human. Subjects can be humans; non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. A subject can be of any age. Subjects can be, for example, elderly adults, adults, adolescents, pre-adolescents, children, toddlers, infants, and fetuses in utero.

Treatment can be provided to the subject before clinical onset of disease. Treatment can be provided to the subject after clinical onset of disease. Treatment can be provided to the subject after 1 day, 1 week, 6 months, 12 months, or 2 years or more after clinical onset of the disease. Treatment can be provided to the subject for more than 1 day, 1 week, 1 month, 6 months, 12 months, 2 years or more after clinical onset of disease. Treatment can be provided to the subject for less than 1 day, 1 week, 1 month, 6 months, 12 months, or 2 years after clinical onset of the disease. Treatment can also include treating a human in a clinical trial. A treatment can comprise administering to a subject a pharmaceutical composition, such as one or more of the pharmaceutical compositions described throughout the disclosure. A treatment can comprise a once daily dosing. A treatment can comprise delivering a peptide of the disclosure to a subject, either intravenously, subcutaneously, intramuscularly, by inhalation, dermally, topically, by intra-articular injection, orally, sublingually, intrathecally, transdermally, intranasally, via a peritoneal route, directly into a tumor e.g., injection directly into a tumor, directly into the brain, e.g., via and intracerebral ventricle route, or directly onto a joint, e.g. via topical, intra-articular injection route. A treatment can comprise administering a peptide-active agent complex to a subject, either intravenously, subcutaneously, intramuscularly, by inhalation, by intra-articular injection, dermally, topically, orally, intrathecally, transdermally, intransally, parenterally, orally, via a peritoneal route, nasally, sublingually, or directly onto cancerous tissues.

### Peptide Kits

In one aspect, peptides described herein can be provided as a kit. In another embodiment, peptide complexes described herein can be provided as a kit. In another embodiment, a kit comprises amino acids encoding a peptide described herein, a vector, a host organism, and an instruction manual. In some embodiments, a kit includes written instructions on the use or administration of the peptides.

Additional aspects and advantages of the present disclosure will become apparent to those skilled in this art from the following detailed description, wherein illustrative embodiments of the present disclosure are shown and described. As will be realized, the present disclosure is capable of other and different embodiments, and its several details are capable of modifications in various respects, all without departing from the disclosure. Accordingly, the drawings and description are to be regarded as illustrative in nature, and not as restrictive.

### EXAMPLES

The following examples are included to further describe some aspects of the present disclosure and should not be used to limit the scope of the invention.

### Example 1

### Manufacture of Peptides

This example describes the manufacture of the peptides and peptide complexes described herein (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64). Peptides derived from proteins were generated in mammalian cell culture using a published methodology. (A. D. Bandaranayke, C. Correnti, B. Y. Ryu, M. Brault, R. K. Strong, D. Rawlings. 2011. Daedalus: a robust, turnkey platform for rapid production of decigram quantities of active recombinant proteins in human cell lines using novel lentiviral vectors. Nucleic Acids Research. (39)21, e143).

The peptide sequence was reverse-translated into DNA, synthesized, and cloned in-frame with siderocalin using standard molecular biology techniques (M. R. Green, Joseph Sambrook. Molecular Cloning. 2012 Cold Spring Harbor Press). The resulting complex was packaged into a lentivirus, transduced into HEK-293 cells, expanded, isolated by immobilized metal affinity chromatography (IMAC), cleaved with tobacco etch virus (TEV) protease, and purified to homogeneity by reverse-phase chromatography. Following purification, each peptide was lyophilized and stored frozen.

### Example 2

### Peptide Expression Using a Mammalian Expression System

This example describes expression of the peptides and peptide complexes using a mammalian expression system. Peptides were expressed according to the methods described in in Bandaranayake et al., Nucleic Acids Res. 2011 November; 39(21): e143. Peptides (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) were cleaved from siderocalin using tobacco etch virus protease and were purified by reversed-phase HPLC (RP-HPLC) in a gradient of acetonitrile and water with 0.1% TFA, and then aliquoted and lyophilized for later use. Molecular weight was verified by mass spectrometry.

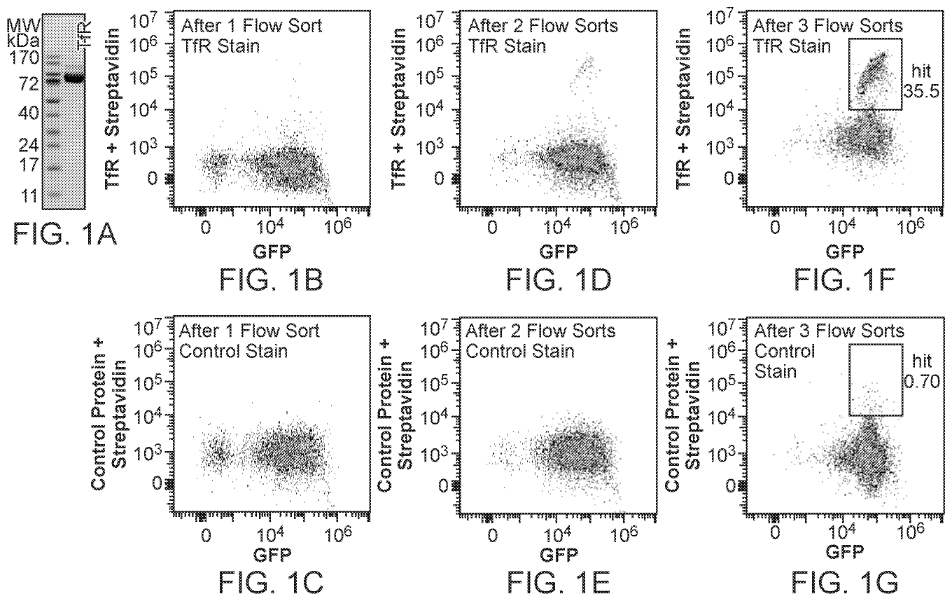

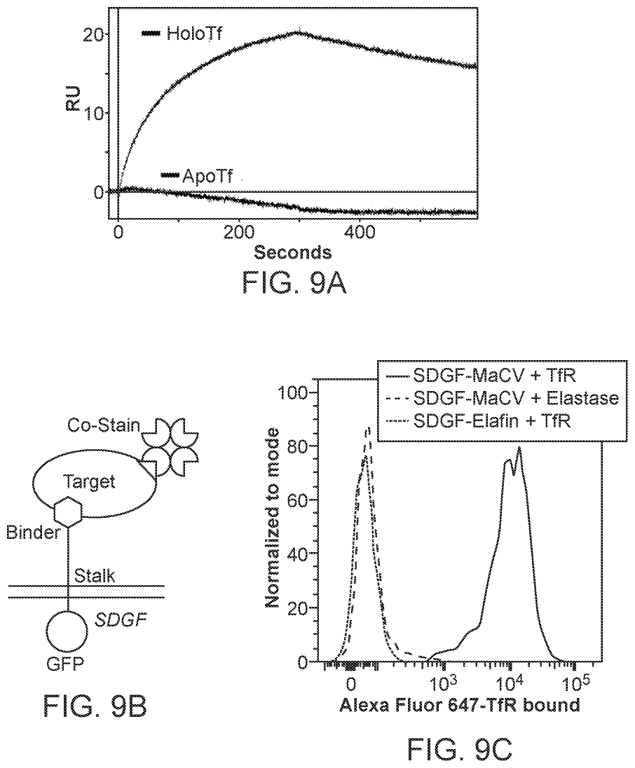

To optimize and validate the screening methodology and to identify TfR-binding peptides, transferrin receptor (TfR) ectodomain (“soluble TfR”, SEQ ID NO: 188, MRLAVGALLVCAVLGLCLADYKDEHHHHHHHGLNDIFEAQKIEWHEGGGSKTVRWCA VSEHEATKCQSFRDHMKSVIPSDGPSVACVKKASYLDCIRAIAANEADAVTLDAGLVY DAYLAPNNLKPVVAEFYGSKEDPQTFYYAVAVVKKDSGFQMNQLRGKKSCHTGLGRS AGWNIPIGLLYCDLPEPRKPLEKAVANFFSGSCAPCADGTDFPQLCQLCPGCGCSTLNQ YFGYSGAFKCLKDGAGDVAFVKHSTIFENLANKADRDQYELLCLDNTRKPVDEYKDC HLAQVPSHTVVARSMGGKEDLIWELLNQAQEHFGKDKSKEFQLFSSPHGKDLLFKDSA HGFLKVPPRMDAKMYLGYEYVTAIRNLREGTCPEAPTDECKPVKWCALSHHERLKCD EWSVNSVGKIECVSAETTEDCIAKIMNGEADAMSLDGGFVYIAGKCGLVPVLAENYDK SDNCEDTPEAGYFAVAVVKKSASDLTWDNLKGKKSCHTAVGRTAGWNIPMGLLYNKI NHCRFDEFFSEGCAPGSKKDSSLCKLCMGSGLNLCEPNNKEGYYGYTGAFRCLVEKGD VAFVKHQTVPQNTGGKNPDPWAKNLNEKDYELLCLDGTRKPVEEYANCHLARAPNH AVVTRKDKEACVHKILRQQQHLFGSDVTDCSGNFCLFRSETKDLLFRDDTVCLAKLHD RNTYEKYLGEEYVKAVGNLRKCSTSSLLEACTFRRP) was cloned into the Daedalus soluble protein production lentivector, and protein was purified from the growth media (a gel of soluble TfR is shown in FIG. 1A). The same strategy was used to produce and purify human apo-transferrin (residues 23-698, SEQ ID NO: 189, KTVRWCAVSEHEATKCQ SFRDHMKSVIPSDGPSVACVKKASYLDCIRAIAANEADAVT LDAGLVYDAYLAPNNLKPVVAEFYGSKEDPQTFYYAVAVVKKDSGFQMNQLRGKKS CHTGLGRSAGWNIPIGLLYCDLPEPRKPLEKAVANFFSGSCAPCADGTDFPQLCQLCPG CGCSTLNQYFGYSGAFKCLKDGAGDVAFVKHSTIFENLANKADRDQYELLCLDNTRKP VDEYKDCHLAQVPSHTVVARSMGGKEDLIWELLNQAQEHFGKDKSKEFQLFSSPHGK DLLFKDSAHGFLKVPPRMDAKMYLGYEYVTAIRNLREGTCPEAPTDECKPVKWCAL SH HERLKCDEWSVNSVGKIECVSAETTEDCIAKIMNGEADAMSLDGGFVYIAGKCGLVPV LAENYNKSDNCEDTPEAGYFAIAVVKKSASDLTWDNLKGKKSCHTAVGRTAGWNIPM GLLYNKINHCRFDEFFSEGCAPGSKKDSSLCKLCMGSGLNLCEPNNKEGYYGYTGAFR CLVEKGDVAFVKHQTVPQNTGGKNPDPWAKNLNEKDYELLCLDGTRKPVEEYANCHL ARAPNHAVVTRKDKEACVHKILRQQQHLFGSNVTDCSGNFCLFRSETKDLLFRDDTVC LAKLHDRNTYEKYLGEEYVKAVGNLRKCSTSSLLEACTFRRP), and some of the material was iron-loaded to produce holo-transferrin. Both apo-transferrin and holo-transferrin were tested for binding to the soluble TfR ectodomain via surface plasmon resonance (FIG. 9A), where only holo-transferrin demonstrated any interaction with the immobilized TfR.

As further validation that the soluble TfR used in the screen represents the human, endogenous protein structure, interaction with the Machupo virus glycoprotein was tested (referred to as MaCV), which uses TfR to determine tropism and mediate cell entry. For this, MaCV was cloned into a mammalian surface display vector SDGF (FIG. 9B), and transfected suspension 293 Freestyle (293F) cells with SDGF-MaCV or a control protein (SDGF-Elafin, an inhibitor of elastase known to bind some native CDPs). Transfected cells were stained with 200 nM each biotinylated TfR (all TfR used in cell binding assays is biotinylated) and Alexa Fluor 647-labeled streptavidin, and then analyzed by flow cytometry (FIG. 9C). Cells transfected with MaCV were successfully stained with TfR, while SDGF-Elafin cells were not. Meanwhile, SDGF-MaCV cells incubated with 200 nM fluorescent elastase were not stained. This validates that the soluble TfR used in the screen comprises the endogenous protein structure and demonstrates both the specificity of TfR binding to its endogenous ligand, and the utility of SDGF as a means to identify novel TfR binding partners.

### Example 3

### Mammalian Surface Display of TfR-Binding Peptides

This example describes mammalian surface display of TfR-binding peptides of the present disclosure including SEQ ID NO: 1 (SEQ ID NO: 1 is SEQ ID NO: 65 with an added N-terminal GS), SEQ ID NO: 2 (SEQ ID NO: 2 is SEQ ID NO: 66 with an added N-terminal GS), SEQ ID NO: 30 (SEQ ID NO: 30 is SEQ ID NO: 94 with an added N-terminal GS), and SEQ ID NO: 32 (SEQ ID NO: 32 is SEQ ID NO: 96 with an added N-terminal GS). Screening for TfR-binding peptides was performed by transfecting or transducing mammalian cells to display candidate peptides (FIG. 9B) followed by screening against soluble human transferrin receptor ectodomain (200 nM, FIG. 9C, FIG. 1B-FIG. 1G). Mammalian cells had improved fidelity in folding disulfide crosslinked proteins, making them a suitable cell type for display of the peptides of the present disclosure.

Mammalian cell surface display screening was carried out as follows. The screening strategy used a surface display GFP FasL (SDGF) vector. In the vector, FasL-TM is the transmembrane domain of the FasL protein. More specifically, the designed peptides were cloned as a pool into SDGF, which were then made into lentivirus. 293F cells were transduced with this library at a multiplicity of infection of ˜1, and after three days of growth, the pool of transduced cells was incubated with Alexa647-labeled TfR. For these experiments, fluorescent labeling was accomplished by co-staining TfR with fluorescent streptavidin or fluorescent anti-His antibodies. Soluble TfR contained both His tag and biotin label. Either Alexa Fluor 647 or iFluor 647 was used for the antibody/streptavidin fluorescence. A percentage of the highest-staining TfR-positive cells, from GFP and TfR double-positive cells, were sorted and expanded. At every expansion, a portion of the cells were collected, and at the end, the enriched peptides were identified by sequencing. Flow cytometry was used to assess gating criteria to identify GFP+ 293F cells expressing proteins on their surface via the SDGF peptide construct. Gating progresses using FSC-H vs SSC-H to gate out debris; FSC-H vs FSC-A to gate out doublets; FSC-H vs Pacific Blue H to gate out DAPI+ dead cells; and an optional FITC-H histogram to identify GFP+ cells. Once gated as such, Alexa Fluor 647 (a co-stain for detecting target binding) was used for sorting and analysis.

Screening was performed using a combination of magnetic sorting and flow sorting. Magnetic sorting was performed as follows: 2×10⁸ 293F cells were transduced with the SDGF CDP library at an MOI of ˜1 and expanded until 3 days post-transduction. For initial screening, magnetic cell sorting was performed. 1×10⁹ transduced cells were resuspended in a binding buffer containing 200 nM biotinylated TfR, 2 mL anti-biotin MicroBeads (UltraPure, Miltenyi 130-105-637), and 21 mL Flow Buffer (PBS+2 mM EDTA and 0.5% bovine serum albumin) in a final volume of 25 mL. Cells were incubated on ice with agitation (mild inversion every 2-5 min) for 30 mins, and were then diluted 10-fold to 250 mL with Flow Buffer, pelleted (500×g, 5 mins), and resuspended to 40 mL with High BSA Flow Buffer (PBS containing 2 mM EDTA and 3% BSA). Cells were split into four 10 mL aliquots and run through a Miltenyi autoMACS® Pro Separator using the “posseld” protocol and “quick rinses” after each sort. The running and wash buffers were High BSA Flow Buffer and PBS+2 mM EDTA, respectively. Eluted cells were pooled, pelleted, and their CDP sequences PCR amplified (Terra™ PCR Direct Polymerase Mix (Takara 639271) for 16 cycles followed by Phusion). This sub-library was cloned into SDGF as above, made into lentivirus, and transduced into a new batch of 293F cells (1×10⁷ cells, MOI˜1) for flow sorting.

Flow sorting was performed as follows: Flow sorting took place using 2.4×10⁷ cells stained in 3 mL Flow Buffer with 200 nM TfR, 200 nM streptavidin Alexa Fluor 647 conjugate (ThermoFisher S21374), and 1 μg mL⁻¹ DAPI. Cells were diluted 4-fold to 12 mL with Flow Buffer, pelleted (500×g, 5 mins), and resuspended in 3.6 mL Flow Buffer. Cells were sorted on a FACSAria II System (BD), gating based on FSC-A (medium), SSC-A (medium), DAPI-A (negative), GFP-A (positive), and APC-A (top 7% of GFP+) channels. After each flow sort, cells were cultured in FreeStyle Media starting at 0.5-1 mL in a suspension 24-well plate, shaking at 300 rpm, expanding to a final volume of 30 mL in a 125 mL baffled flask shaken at 125 RPM. At this point, cells were re-sorted as above. After the third flow sort, cells were expanded and frozen in 1.5×10⁶ cell pellets. Pellets were PCR amplified as above (Terra Direct PCR followed by InFusion), CDP inserts were subcloned into SDGF, transformed (Stellar competent cells), and colonies picked for miniprepping and sequencing of the cloned CDPs. Enriched variants were those that appeared in the sequence analysis more than once in ˜100 picked colonies. All site-saturation mutagenesis (SSM) affinity maturation screening was done as above, with the following changes. 1) Staining was sequential; first with TfR, and then with an equimolar amount of dye-labeled streptavidin. 2) TfR/streptavidin concentrations were reduced to 20 nM for the first SSM maturation and to 8 nM for the second SSM maturation screen. After each SSM screen, enriched variants were studied to assemble a compound mutant (peptide of SEQ ID NO: 2 or SEQ ID NO: 32) that showed higher TfR staining than any of the variants containing either 1 or 2 of the individual mutations.

Flow cytometry plots in FIG. 1B-FIG. 1G illustrate successive enrichment of cells that bind to TfR from pooled, high diversity library. FIG. 1A illustrates a Coomassie stained gel of transferrin receptor (TfR) protein showing successful purification of TfR. FIG. 1B illustrates a flow cytometry plot of cells displaying candidate TfR-binding peptides after one flow sort. Cells were sorted based on ability to bind to TfR labeled with a fluorescent streptavidin. Data points in the upper right region represent cells expressing a candidate peptide, quantified by GFP fluorescence, that bind TfR, quantified by fluorescence of the fluorescent TfR-streptavidin. FIG. 1C illustrates a negative control flow cytometry plot of cells displaying candidate TfR-binding peptides after one flow sort. Cells were sorted based on ability to bind to a control protein labeled with a fluorescent streptavidin. Data points in the upper right region represent cells expressing a candidate peptide, quantified by GFP fluorescence, that bind to the negative control protein, quantified by fluorescence of the fluorescent control protein-streptavidin. FIG. 1D illustrates a flow cytometry plot of cells displaying candidate TfR-binding peptides after a second flow sort, following the first cell sort illustrated in FIG. 1B. Cells were sorted based on ability to bind to TfR labeled with a fluorescent streptavidin. Data points in the upper right region represent cells expressing a candidate peptide, quantified by GFP fluorescence, that bind TfR, quantified by fluorescence of the fluorescent TfR-streptavidin. FIG. 1E illustrates a negative control flow cytometry plot of cells displaying candidate TfR-binding peptides after a second flow sort, following the first cell sort illustrated in FIG. 1C. Cells were sorted based on ability to bind to a control protein labeled with a fluorescent streptavidin. Data points in the upper right region represent cells expressing a candidate peptide, quantified by GFP fluorescence, that bind to the negative control protein, quantified by fluorescence of the fluorescent control protein-streptavidin. FIG. 1F illustrates a flow cytometry plot of cells displaying candidate TfR-binding peptides after a third flow sort, following the second cell sort illustrated in FIG. 1D. Cells were sorted based on ability to bind to TfR labeled with a fluorescent streptavidin. Data points in the upper right region represent cells expressing a candidate peptide, quantified by GFP fluorescence, that bind TfR, quantified by fluorescence of the fluorescent TfR-streptavidin. The box indicates cells expressing peptides that bind to TfR. FIG. 1G illustrates a negative control flow cytometry plot of cells displaying candidate TfR-binding peptides after a third flow sort, following the second cell sort illustrated in FIG. 1E. Cells were sorted based on ability to bind to a control protein labeled with a fluorescent streptavidin. Data points in the upper right region represent cells expressing a candidate peptide, quantified by GFP fluorescence, that bind to the negative control protein, quantified by fluorescence of the fluorescent control protein-streptavidin. The box indicates cells expressing peptides that bind to the negative control protein.

Each flow sort represents growing the library of cells to >30 million cells, staining for TfR-binding, and flow sorting the top binders. Sorted binders were allowed to grow before the next flow sort was performed.

### Example 4

### Identification of TfR-Binding Peptides

This example describes identification of TfR-binding peptides using a mammalian surface display system, as described in EXAMPLE 3.

Using the mammalian surface display system of EXAMPLE 3, a single clonal peptide was identified having a sequence of SEQ ID NO: 1 (SEQ ID NO: 1 is SEQ ID NO: 65 with an added N-terminal GS). A library of oligonucleotides encoding 10,000 CDPs was amplified and mutagenized. The CDPs were 17-50 amino acids in length, with 4, 6, 8, or 10 cysteines. While there was some weighting of the library towards annotated knottins or defensins, the library contained CDPs from every domain/kingdom of life. This library was cloned into SDGF, made into lentivirus, and transduced into suspension 293F cells. The transduced cells were subjected to staining with TfR (200 nM) and co-stain over the course of one round of magnetic cell sorting and three rounds of flow sorting, each round enriching for cells stained with TfR. Binding was validated to specifically bind TfR in the surface display assay using 200 nM of soluble AF647-TfR, which was either biotinylated or attached to a His tag. Staining was carried out using a one-step staining protocol for tetravalent target avidity. A single TfR-binding CDP, designated SEQ ID NO: 1, was identified by DNA sequencing of the final enriched cell population. It represents a randomly mutated variant of cytochrome BC1 complex subunit 6 from the marine choanoflagellate Monosiga brevicolis (Uniprot ID: A9V0D7, DOI: 10.1093/nar/gku989), is 49 amino acids in length (six cysteines) and has a predicted molecular mass of 5.6 kDa. SEQ ID NO: 65 was then subjected to affinity maturation using site saturation mutagenesis (SSM), wherein a library is created containing every possible non-cysteine single amino acid substitution (43 non-Cys amino acids×18 possible non-Cys substitutions=775 variants, including SEQ ID NO: 1).

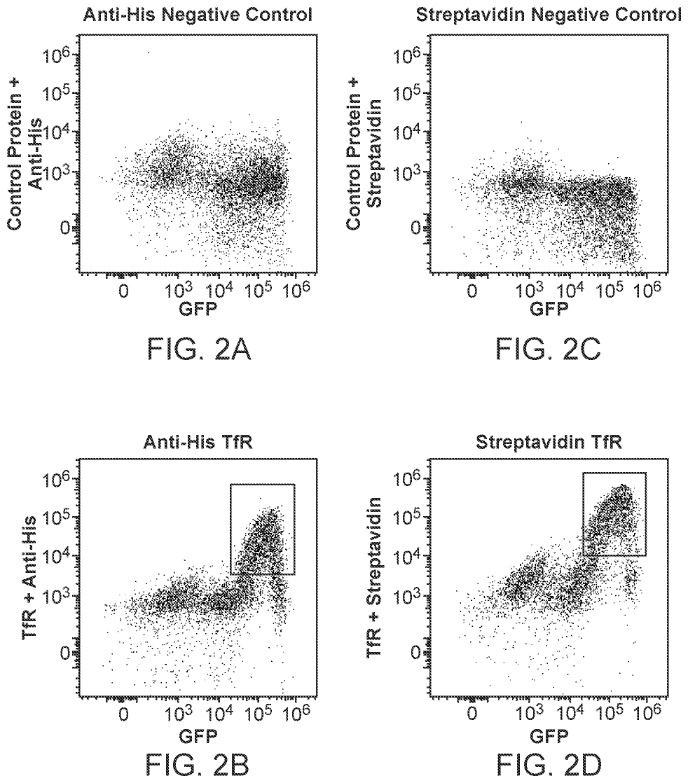

Flow cytometry plots in FIG. 2A-FIG. 2D illustrate flow cytometry of cells displaying the single clonal TfR-binding peptide and screened for binding to either TfR or a negative control protein. Flow cytometry of the single TfR-binding peptide was performed to verify that the identified TfR-binding peptide bound specifically TfR and not to the streptavidin label. The control protein used in this experiment has an amino acid sequence set forth in SEQ ID NO: 186 (MRLAVGALLVCAVLGLCLADYKDEHHHHHHGLNDIFEAQKIEWHEGGGSKTVRWCA VSEHEATKCQSFRDHMKSVIPSDGPSVACVKKASYLDCIRAIAANEADAVTLDAGLVY DAYLAPNNLKPVVAEFYGSKEDPQTFYYAVAVVKKDSGFQMNQLRGKKSCHTGLGRS AGWNIPIGLLYCDLPEPRKPLEKAVANFFSGSCAPCADGTDFPQLCQLCPGCGCSTLNQ YFGYSGAFKCLKDGAGDVAFVKHSTIFENLANKADRDQYELLCLDNTRKPVDEYKDC HLAQVPSHTVVARSMGGKEDLIWELLNQAQEHFGKDKSKEFQLFSSPHGKDLLFKDSA HGFLKVPPRMDAKMYLGYEYVTAIRNLREGTCPEAPTDECKPVKWCALSHHERLKCD EWSVNSVGKIECVSAETTEDCIAKIMNGEADAMSLDGGFVYIAGKCGLVPVLAENYDK SDNCEDTPEAGYFAVAVVKKSASDLTWDNLKGKKSCHTAVGRTAGWNIPMGLLYNKI NHCRFDEFFSEGCAPGSKKDSSLCKLCMGSGLNLCEPNNKEGYYGYTGAFRCLVEKGD VAFVKHQTVPQNTGGKNPDPWAKNLNEKDYELLCLDGTRKPVEEYANCHLARAPNH AVVTRKDKEACVHKILRQQQHLFGSDVTDCSGNFCLFRSETKDLLFRDDTVCLAKLHD RNTYEKYLGEEYVKAVGNLRKCSTSSLLEACTFRRPGSSELYENKPRRPYIL). FIG. 2A illustrates a negative control flow cytometry plot of cells expressing a TfR-binding peptide of SEQ ID NO: 1 (x-axis, GFP) screened for binding to a negative control protein labeled (y-axis, stained with a fluorescent anti-His antibody). FIG. 2B illustrates a flow cytometry plot of cells expressing a peptide of SEQ ID NO: 1 (x-axis, GFP) and TfR (y-axis, stained with a fluorescent anti-His antibody). FIG. 2C illustrates a negative control flow cytometry plot of cells expressing a TfR-binding peptide of SEQ ID NO: 1 (x-axis, GFP) screened for binding to a negative control protein labeled (y-axis, stained with a fluorescent streptavidin). FIG. 2D illustrates a flow cytometry plot of cells expressing a TfR-binding peptide of SEQ ID NO: 1 (x-axis, GFP) screened for binding to TfR (y-axis, stained with a fluorescent streptavidin).

TfR staining was observed with cells expressing the identified clone, with no staining seen with a control protein. This staining was observed when either fluorescent streptavidin or anti-His antibody was the co-stain, demonstrating that the nature of the binding is dependent only on TfR, not the co-stain. Double-positive cells (upper right quadrant) indicate peptide-expressing cells that are bound to TfR.

An alternative method was also used to identify and optimize (“mature”) TfR-binding peptides that are second and third generation binders using mammalian display screening. This library was screened with a modified staining protocol, using a lower concentration of target and co-stain (20 nM) and separate staining steps; the latter further increases stringency by eliminating the tetravalent avidity granted by streptavidin. Up to four rounds of flow sorting and enrichment were used to identify variants with improved TfR binding characteristics.

Permutation of enriched variants identified an optimal mutant (SEQ ID NO: 2 (SEQ ID NO: 2 is SEQ ID NO: 66 with an added N-terminal GS)), and this process was repeated again (8 nM TfR and co-stain; otherwise, identical protocol) to generate SEQ ID NO: 32 (SEQ ID NO: 32 is SEQ ID NO: 96 with an added N-terminal GS). This twice-matured variant contains 14 point mutations from the original library member (GSREGCASHCTKYKAELEKCEARVSSRSNTEETCVQELFDFLHCVDHCVSQ, SEQ ID NO: 191). Four point mutations from the parent sequence are found in SEQ ID NO: 1, while SEQ ID NO: 2 and SEQ ID NO: 32 contain 6 and 4 mutations, respectively, from the previous generation.

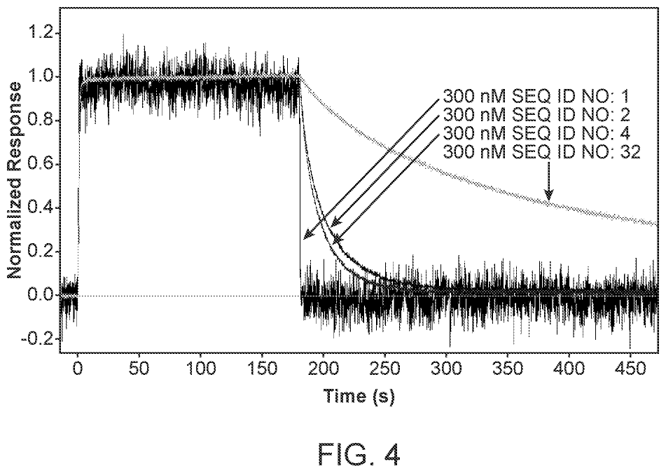

SEQ ID NO: 1 and its variants (e.g., SEQ ID NO: 2 and SEQ ID NO: 32) were produced as soluble peptides and validated by reversed-phase HPLC (RP-HPLC), SDS-PAGE, and mass spectrometry. Based on their masses of ˜5-6 kDa, their slower-than-expected mobility in SDS-PAGE was a demonstration of the interesting electrophoretic mobility characteristics that some CDPs possess. All variants showed markedly different mobility upon DTT reduction (10 mM) in both SDS-PAGE and RP-HPLC, confirming disulfide bond stabilization. Their binding to TfR was verified by surface plasmon resonance (FIG. 4), also confirming increased affinity of matured variants (SEQ ID NO: 32 [K_{D}=216±1 pM]>SEQ ID NO: 2 [K_{D}=8.7±0.4 nM]>SEQ ID NO: 1 [K_{D} not determined but available data is consistent with a K_{D}>10 μM]). All variants demonstrated full or partial resistance to cellular reducing conditions (10 mM glutathione), while the affinity matured variants showed partial resistance to pepsin (though all were vulnerable to trypsin proteolysis). The intact, non-reduced peptide of SEQ ID NO: 32 protein demonstrates substantially improved heat tolerance to that of the DTT-reduced protein, with no substantial change in circular dichroism characteristics until well above common ambient temperatures (>50° C.) and a failure to observe complete unfolding up to 95° C.

### Example 5

### Site Saturation Mutagenesis of Peptides

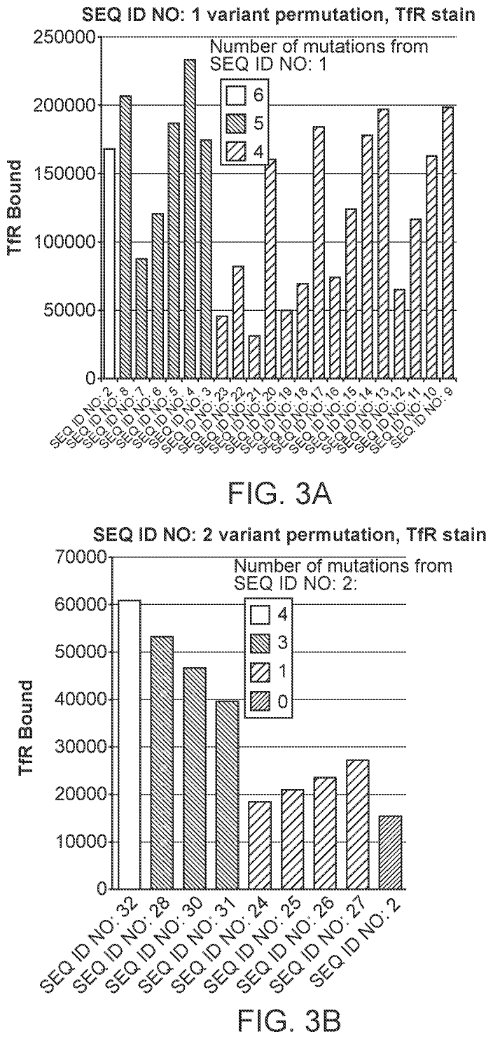

This example illustrates site saturation mutagenesis (SSM) of peptides of this disclosure to identify beneficial or deleterious mutations. Site saturation mutagenesis was performed on a peptide of SEQ ID NO: 1 (SEQ ID NO: 1 is SEQ ID NO: 65 with an added N-terminal GS, results in FIG. 3A) and a peptide of SEQ ID NO: 2 (SEQ ID NO: 2 is SEQ ID NO: 66 with an added N-terminal GS, results in FIG. 3B).

FIG. 3A and FIG. 3B show the TfR-binding capabilities of TfR-binding peptide variants identified during peptide maturation. SSM was employed for affinity maturation of the peptide having a sequence of SEQ ID NO: 1 as identified in the first mammalian surface display experiments (see e.g., FIG. 1B-FIG. 1G and FIG. 2A-FIG. 2D). During each round of maturation, a library of every possible non-cysteine point variants was constructed and screened against TfR at higher stringency than first screen. Variants with improved binding were enriched and identified by Sanger sequencing. Such enriched variant mutations were combined with one another in various permutations (shown in the data) to identify a composite, improved binder. Two rounds of SSM were completed, yielding matured peptides comprising SEQ ID NO: 2, and SEQ ID NO: 32, respectively (SEQ ID NO: 32 is SEQ ID NO: 96 with an added N-terminal GS). TfR concentration in the first round of SSM was 20 nM and SSM was carried out using one-step staining. TfR concentration in the second round of SSM was 8 nM and SSM was carried out using two-step staining.

TfR-binding of mammalian cells expressing peptides of the SSM library was performed as described in EXAMPLE 3 and EXAMPLE 4, but with a higher stringency protocol. The higher stringency protocol included a lower concentration of TfR (e.g., 20 nM).

FIG. 3A illustrates the results of a first site-saturation mutagenesis screen in SEQ ID NO: 1 (SEQ ID NO: 1 is SEQ ID NO: 65 with an added N-terminal GS), with some variants exhibiting improved binding activity to TfR such as peptides having a sequence of SEQ ID NO: 4, SEQ ID NO: 5, and SEQ ID NO: 8 (SEQ ID NO: 4, SEQ ID NO: 5, and SEQ ID NO: 8 are SEQ ID NO: 68, SEQ ID NO: 69, and SEQ ID NO: 72, respectively, with an added N-terminal GS). FIG. 3B show TfR-binding of variants identified during the second variant mutation.

The x-axis shows SEQ ID NOs of all variants and the y-axis shows the amount TfR bound in relative fluorescence units (RFUs) extrapolated from flow cytometry experiments.

### Example 6

### TfR-Binding of SSM-Generated TfR-Binding Peptide Variants

This example demonstrates TfR-binding of site saturation mutagenesis (SSM)-generated TfR-binding peptide variants, as identified during SSM as described in EXAMPLE 5

In vivo BBB penetration experiments revealed that the TfR-binding capability of a peptide does not necessarily correspond with the capability of promoting vesicular transcytosis.

The six cysteines corresponding to residues C6, C10, C20, C34, C44, and C48, with reference to SEQ ID NO: 32 (C4, C8, C18, C32, C42, and C46, with reference to SEQ ID NO: 96), participate in disulfides, and thus contribute to peptide stability.

The surface interface residues corresponding to residues G5, A7, S8, N14, L17, E18, E21, L38, L42, L45, D46, H47, S50, Q51, with reference to SEQ ID NO: 32 (G3, A5, S6, N12, L15, E16, E19, L36, L40, L43, D44, H45, S48, Q49, with reference to SEQ ID NO: 96), that are present in all three generations of TfR-binding peptides likely contribute to TfR-binding. In some embodiments, the peptide or peptide complex of the present disclosure comprises at least one or more of these corresponding residues in SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. Such peptides can accordingly be engineered with enhanced binding to TfR.

Hydrophilic surface-distal residues such as D, E, H, K, R, N, Q, S, or T likely contribute to peptide solubility corresponding to the following amino acid residues R3, E4, R9, K12, D14, E15, K19, R23, S26, S28, N29, T30, E31, E32, D33, E35, Q36, E37, E39, and D40, with reference to SEQ ID NO: 32 (R1, E3, R7, K10, D12, E13, K17, R21, S24, S26, N27, T28, E29, E30, D31, E33, Q34, E35, E37, and D38, with reference to SEQ ID NO: 96). In some embodiments, the peptide or peptide complex of the present disclosure comprises at least one or more of these corresponding residues in SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. Such peptides can accordingly be engineered with enhanced solubility.

Higher binding affinity is associated with the presence of hydrophilic residues such as D, E, H, K, R, N, Q, S, or T as shown by improved binding from a mutation away from a nonpolar or hydrophobic residue such as A, M, I, L, V, F, W, or Y at the residues corresponding to D15, E35, E39, and H49, with reference to SEQ ID NO: 32 (D13, E33, E37, and H47, with reference to SEQ ID NO: 96). In some embodiments, the peptide or peptide complex of the present disclosure comprises at least one or more of these corresponding residues in SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. Such peptides can accordingly be engineered with modified binding affinity.

Higher binding affinity to TfR is associated with nonpolar or hydrophobic residues such as A, M, I, L, V, F, W, or Y as shown by improved binding from a mutation away from a hydrophilic residue such as D, E, H, K, R, N, Q, S, or T at the amino acid residues corresponding to M11, M25, and M27, with reference to SEQ ID NO: 32 (M9, M23, and M25, with reference to SEQ ID NO: 96). In some embodiments, the peptide or peptide complex of the present disclosure comprises at least one or more of these corresponding residues in SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64. Such peptides can accordingly be engineered with modified binding affinity.

A higher TfR-binding affinity is associated with aliphatic residues such as A, M, I, L, or V as shown by improved binding from a mutation away from large, aromatic residues such as F, W, or Y at the amino acid residue corresponding to L45 with reference to SEQ ID NO: 32 (L43 with reference to SEQ ID NO: 96). Substitutions of any one or more F, W, or Y in a peptide of the present disclosure to an aliphatic residue comprising A, M, I, L, or V can be used to enhance the binding affinity of the peptide to TfR.

Any of peptides or peptide complexes of the present disclosure (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) can be modified at one or more of the corresponding residues described herein, to generate peptide variants with improved properties including enhanced stability and increased (or decreased) binding properties or modified TfR-binding affinity and increased (or decreased) transcytosis properties, including modified kₐ (association) and k_{d} (dissociation) rate constants.

Sequence alignments of certain TfR-binding peptides are shown in TABLE 9. Certain residues involved in the interaction with TfR are shown in bold. Surface interacting residues include but are not limited to those indicated.

TABLE 9
Corresponding Residues in TfR-Binding Peptides

| SEQ ID NO | Sequence |
|---|---|
| SEQ ID NO: 1 | GSREGCASRCTKYNAELEKCEARVSSMSNTEETCVQELFDLLHCVDHCVSQ |
| SEQ ID NO: 2 | GSREGCASRCTKYNAELEKCEARVMSMSNTEEDCEQELEDLLHCLDHCHSQ |
| SEQ ID NO: 30 | GSREGCASRCMKYNAELEKCEARMMSMSNTEEDCEQELEDLLYCLDHCHSQ |
| SEQ ID NO: 32 | GSREGCASRCMKYNDELEKCEARMMSMSNTEEDCEQELEDLLYCLDHCHSQ |
| SEQ ID NO: 65 | REGCASRCTKYNAELEKCEARVSSMSNTEETCVQELFDLLHCVDHCVSQ |
| SEQ ID NO: 66 | REGCASRCTKYNAELEKCEARVMSMSNTEEDCEQELEDLLHCLDHCHSQ |
| SEQ ID NO: 94 | REGCASRCMKYNAELEKCEARMMSMSNTEEDCEQELEDLLYCLDHCHSQ |
| SEQ ID NO: 96 | REGCASRCMKYNDELEKCEARMMSMSNTEEDCEQELEDLLYCLDHCHSQ |
| SEQ ID NO: 33 | GSREGCASRCTRYNAELERCEARVSSMSNTEETCVQELFDLLHCVDHCVSQ |
| SEQ ID NO: 34 | GSREGCASRCTRYNAELERCEARVMSMSNTEEDCEQELEDLLHCLDHCHSQ |
| SEQ ID NO: 62 | GSREGCASRCMRYNAELERCEARMMSMSNTEEDCEQELEDLLYCLDHCHSQ |
| SEQ ID NO: 64 | GSREGCASRCMRYNDELERCEARMMSMSNTEEDCEQELEDLLYCLDHCHSQ |
| SEQ ID NO: 97 | REGCASRCTRYNAELERCEARVSSMSNTEETCVQELFDLLHCVDHCVSQ |
| SEQ ID NO: 98 | REGCASRCTRYNAELERCEARVMSMSNTEEDCEQELEDLLHCLDHCHSQ |
| SEQ ID NO: 126 | REGCASRCMRYNAELERCEARMMSMSNTEEDCEQELEDLLYCLDHCHSQ |
| SEQ ID NO: 128 | REGCASRCMRYNDELERCEARMMSMSNTEEDCEQELEDLLYCLDHCHSQ |

### Example 7

### Surface Plasmon Resonance (SPR) Analysis of Peptide Binding Interactions

This example illustrates surface plasmon resonance (SPR) analysis of peptide binding interactions with TfR.

Various peptides of the present disclosure were analyzed for binding affinity to TfR. Briefly, binding affinity was analyzed by SPR experiments using captured biotinylated TfR, and which were performed at 25° C. on a Biacore T100 instrument (GE Healthcare) with Series S SA chips. HBS-EP+ (10 mM HEPES, pH 7.4, 150 mM NaCl, 3 mM EDTA, 0.05% surfactant P20) was used as a running buffer in the experiments with 0.1 mg/mL bovine serum albumin (BSA). Soluble TfR-binding peptides were evaluated for binding by incubation of a dilution series, in which the concentration range was varied depending on the TfR-binding peptide being tested with 2 μg/ml TfR, capturing ˜300 resonance units (RUs) of protein for SPR experiments.

First, an allelic series of TfR-binding peptides with varying affinities was confirmed by SPR as shown in FIG. 4. Peptides having a sequence of SEQ ID NO: 1, SEQ ID NO: 2, SEQ ID NO: 4, and SEQ ID NO: 32 (SEQ ID NO: 1, SEQ ID NO: 2, SEQ ID NO: 4, and SEQ ID NO: 32 are SEQ ID NO: 65, SEQ ID NO: 66, SEQ ID NO: 68, and SEQ ID NO: 96, respectively, with an added N-terminal GS) were tested at a concentration of 300 nM. Data was normalized to the maximum response of each trace. The results confirmed that peptide variants from later rounds of affinity maturation exhibited different binding affinities for TfR. That is, the peptide from the latest round of affinity maturation having a sequence of SEQ ID NO: 32 showed the highest binding affinity for TfR, whereas SEQ ID NO: 1 showed the lowest binding affinity for human TfR (hTfR).

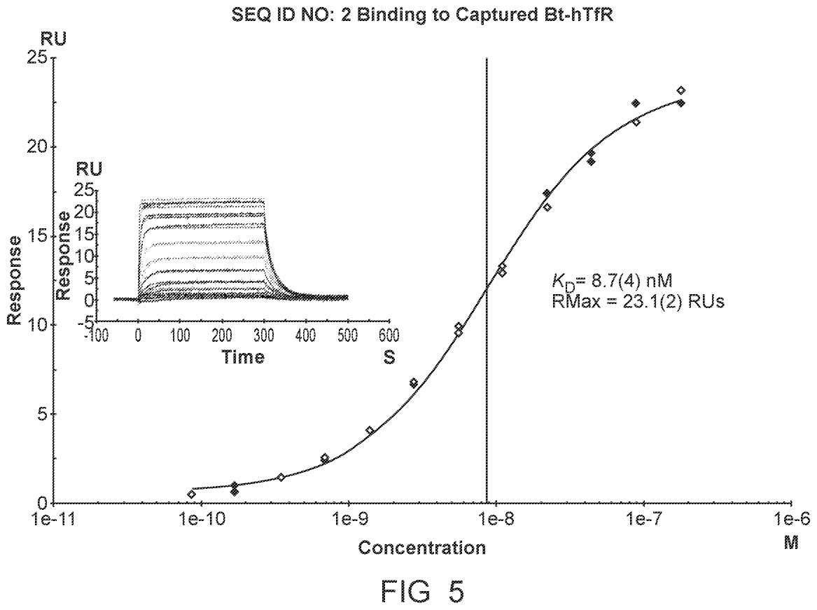

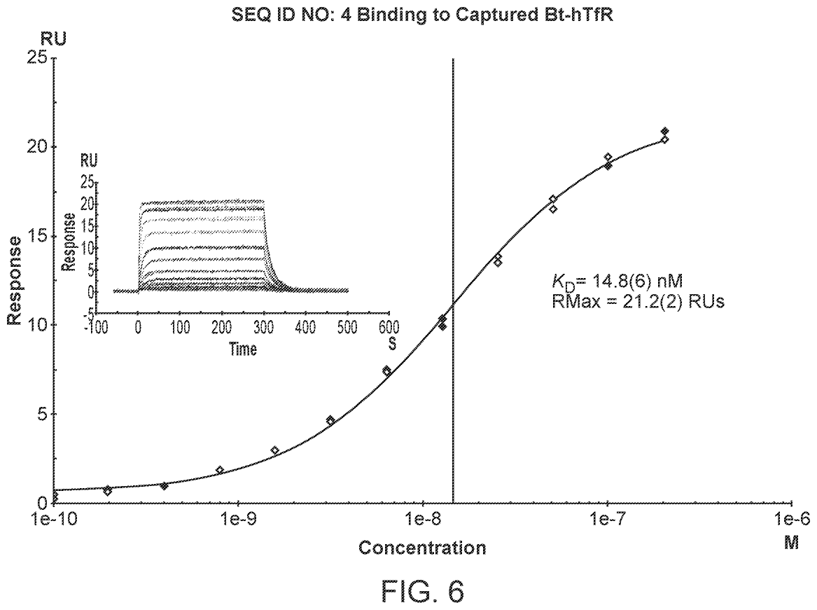

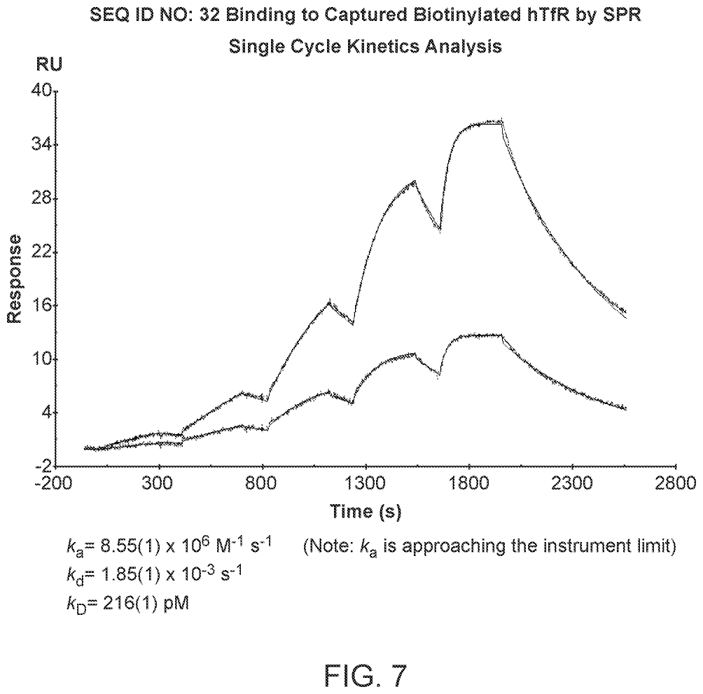

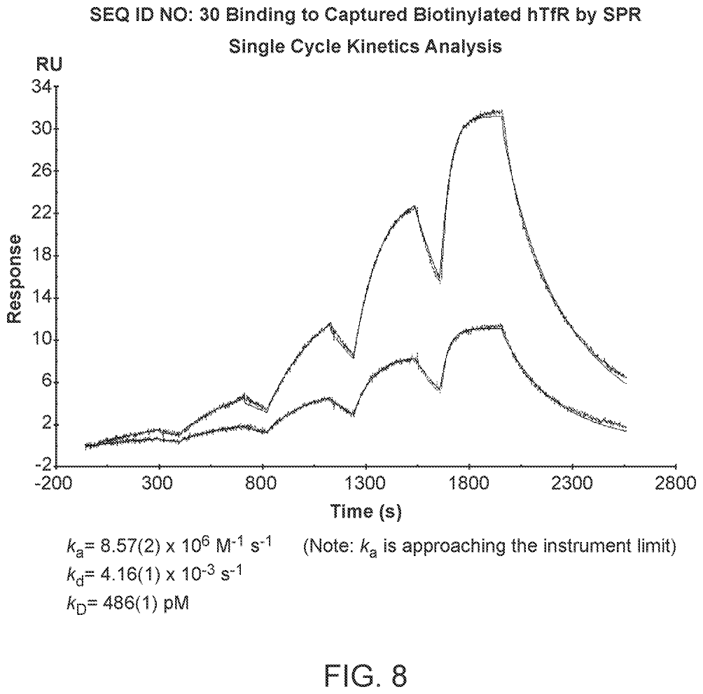

Next, the binding of four peptides having a sequence of SEQ ID NO: 2, SEQ ID NO: 4, SEQ ID NO: 30, and SEQ ID NO: 32, respectively, (SEQ ID NO: 2, SEQ ID NO: 4, SEQ ID NO: 30, and SEQ ID NO: 32 are SEQ ID NO: 66, SEQ ID NO: 68, SEQ ID NO: 94, and SEQ ID NO: 96, respectively, with an added N-terminal GS) to captured and biotinylated hTfR was measured. FIG. 5 illustrates a surface plasmon resonance (SPR) trace showing TfR-binding for varying concentrations of the peptide from 100 μM to 200 nM having a sequence of SEQ ID NO: 2. FIG. 6 illustrates a surface plasmon resonance (SPR) trace showing TfR-binding for varying concentrations of the peptide from 100 μM to 200 nM having a sequence of SEQ ID NO: 4. FIG. 7 illustrates binding and single cycle kinetics data of SEQ ID NO: 32 binding to captured biotinylated (Bt) hTfR by SPR. 5 concentrations of a peptide having a sequence of SEQ ID NO: 32 (0.037 nM, 0.11 nM, 0.33 nM, 1 nM, 3 nM) were injected over 2 densities of captured Bt-hTfR and analyzed globally. FIG. 8 illustrates binding and single cycle kinetics data of SEQ ID NO: 30 binding to captured biotinylated hTfR by SPR. 5 concentrations of a peptide having a sequence of SEQ ID NO: 30 (0.037 nM, 0.11 nM, 0.33 nM, 1 nM, 3 nM) were injected over 2 densities of captured Bt-hTfR and analyzed globally.

The binding SEQ ID NO: 2 and SEQ ID NO: 4 was measured at serial dilutions between 100 μM and 200 nM, while SEQ ID NO: 30 and SEQ ID NO: 32 were tested at serial dilutions of 37 μM to 3 nM, to yield kinetic data. For the peptides of SEQ ID NO: 30 and SEQ ID NO: 32, the kinetic testing was performed by injecting the peptides over two densities (shown in FIG. 7 and FIG. 8) of captured and biotinylated hTfR and data was analyzed globally.

TABLE 10 below summarizes the data obtained from the analysis of the graphs shown in FIG. 5-FIG. 8. For the peptide having a sequence of SEQ ID NO: 2 a K_{D} of 8.7±4 nM and an Rₘₐₓ of 23.1±2 RUs was determined. For the peptide having a sequence of SEQ ID NO: 4 a K_{D} of 14.8±6 nM and an Rₘₐₓ of 21.2±2 RUs was determined. For the peptide having a sequence of SEQ ID NO: 32 a K_{D} of 216±1 μM was determined. For the peptide having a sequence of SEQ ID NO: 30 a K_{D} of 468±1 μM was determined. Lower K_{D} values indicate a higher binding affinity. Rₘₐₓ represents the maximum binding capacity of the peptide to hTfR. As shown in TABLE 10 below, SEQ ID NO: 32 had the lowest K_{D}, indicating that it displayed the strongest binding to hTfR. An increased TfR-binding affinity can correspond to an improved transcytosis function. In some cases, an increased TfR-binding affinity can correspond to a reduced transcytosis function, wherein in some cases, an increased TfR-binding affinity does not correspond to a change in transcytosis function compared to the reference peptide. Without being bound to any theory, it is assumed that the ratio of Kₐ/K_{d} can affect the transcytosis function of a peptide, and thus modulation of Kₐ and/or K_{d} can be used to generate TfR-binding peptides with optimal TfR binding affinity and transcytosis function.

TABLE 10
SPR Analysis Results

| SEQ ID NO | kₐ (M⁻¹ s⁻¹) | k_{d} (s⁻¹) | K_{D} | | Analysis Method |
|---|---|---|---|---|---|
| SEQ ID NO: 1 | | | + | | |
| SEQ ID NO: 2 | — | — | 8.7(4) | nM | Steady State |
| SEQ ID NO: 4 | — | — | 14.8(6) | nM | Steady State |
| SEQ ID NO: 32 | 8.55(1) × 10⁶ | 1.85(1) × 10⁻³ | 216(1) | pM | Single Cycle |
| | | | | | Kinetics |
| SEQ ID NO: 30 | 8.57(2) × 10⁶ | 4.16(1) × 10⁻³ | 486(1) | pM | Single Cycle |

Kinetics
* reported kₐ is approaching the limits that can be measured by the instrument

### Example 8

### pH-Independent Binding of a Transferrin Receptor-Binding Peptide

This example describes pH-independent binding of a transferrin receptor-binding peptide. A CDP that binds to transferrin receptor (TfR) and has a sequence of SEQ ID NO: 32 (corresponding to SEQ ID NO: 96 with an added N-terminal GS) was identified using site saturation mutagenesis as described in EXAMPLE 5. The pH-dependence of the binding affinity of the TfR-binding peptide for TfR was then compared at an exemplary extracellular pH of 7.4 and at an exemplary endosomal pH of 5.5.

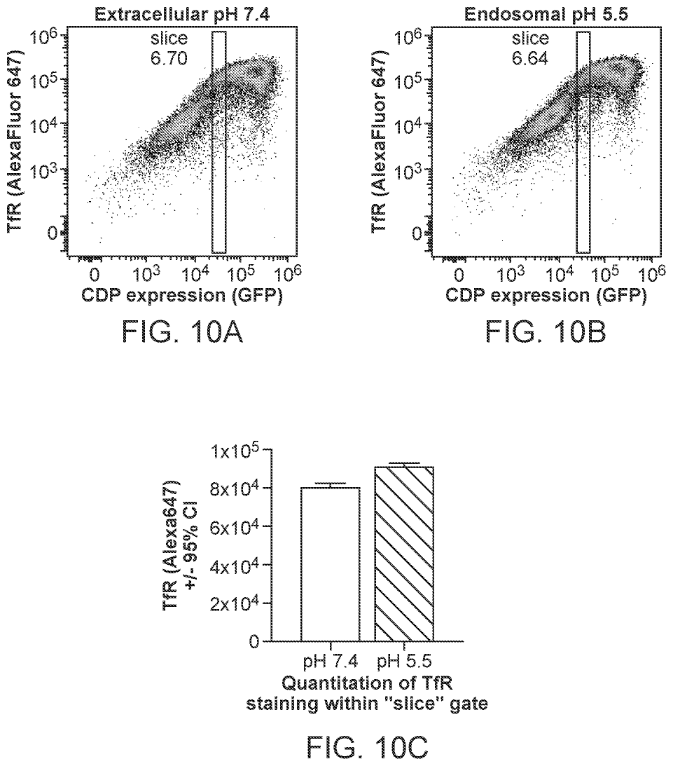

Cells expressing the peptide of SEQ ID NO: 32 were stained with 10 nM of biotinylated TfR labeled with streptavidin-AlexaFluor 647. Staining was performed in either a buffer at an exemplary extracellular pH (pH 7.4, FIG. 10A) or in a buffer at an exemplary endosomal pH (pH 5.5, FIG. 10B). TfR fluorescence was measured as a function of expression of SEQ ID NO: 32. A slice gate corresponding to a desired peptide expression level was selected for comparison. The level TfR fluorescence within the selected slice gate was indicative of the affinity of the peptide for TfR at the tested pH. The results showed that the TfR-binding peptide bound to TfR with slightly higher affinity at endosomal pH (pH 5.5) than at physiologic extracellular pH (pH 7.4, FIG. 10C), with a slightly higher affinity at pH 5.5.

The results show that the TfR binding peptide of SEQ ID NO: 32 can bind TfR at a range of pHs including extracellular and endosomal pHs, and that it has a relatively pH-independent affinity for binding TfR. This demonstrates the suitability of the TfR-binding peptide for use in a method recruiting target molecules to endosomes while remaining bound to TfR inside the endosome. These results suggest that the TfR-binding peptide of SEQ ID NO: 32, and similar TfR binding CDPs of this disclosure, along with peptides linked to the TfR-binding peptide, can be recycled back to the cell surface along with TfR following TfR-mediated endocytosis.

### Example 9

### PD-L1-Binding Peptides for pH-Dependent Endosomal Delivery of PD-L1

This example describes development and in vitro testing of PD-L1-binding peptides capable of pH-dependent dissociation from PD-L1, for example, at endosomal pH (e.g., pH 5.5).

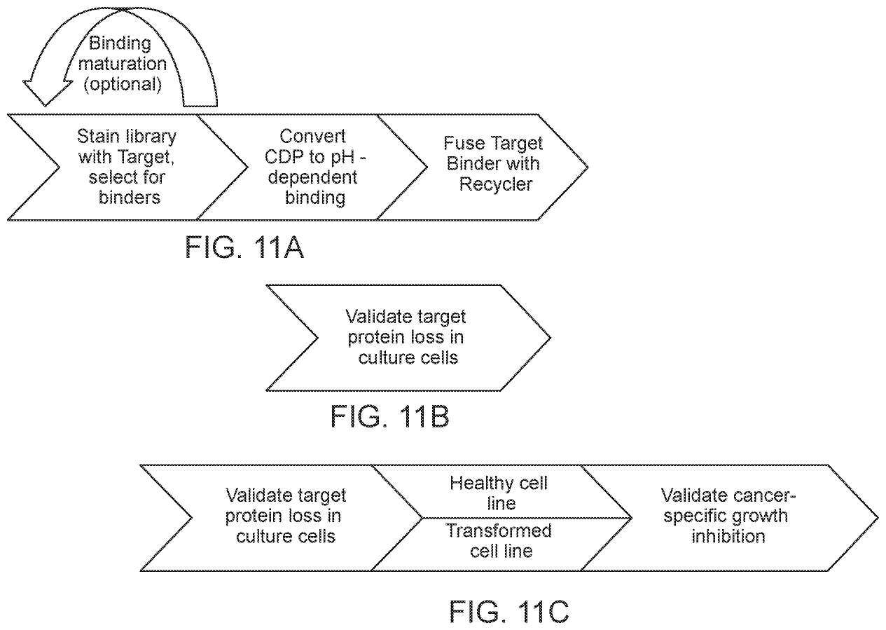

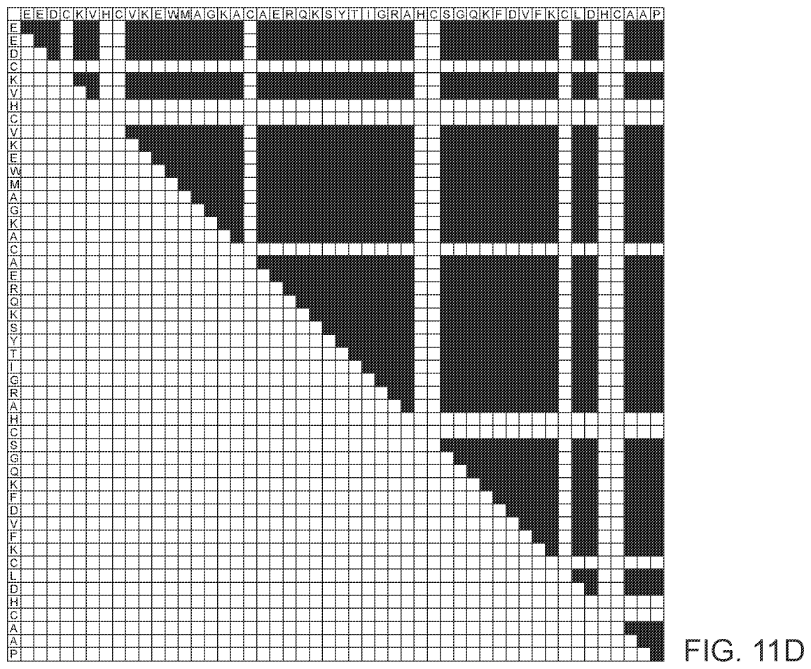

Imparting pH-dependent binding to a target-engaging domain (CDP or otherwise) can done in a variety of ways, an example of which is provided here. Here, a library of variants was designed containing histidine substitutions. Histidine residues were introduced because, of all of the natural amino acids, His is the only one with a side chain whose charge changes significantly between neutral (e.g., pH 7.4) and acidic (e.g., pH<6) endosomal conditions. This change of charge can alter binding, either directly (introducing a positive charge at low pH that could result in charge repulsion of nearby cationic groups) or indirectly (the change in charge imparts a subtle change in the binder's structure, disrupting a protein-protein interface) as the pH changes, for example from a physiologic extracellular environment to an endosomal environment as the endosome acidifies. In its simplest form, this could be executed by generating double-His doped libraries, where, for a CDP, every non-Cys, non-His residue could be substituted with a His one- or two-at-a-time. FIG. 11D shows a high-affinity PD-L1-binding CDP sequence (SEQ ID NO: 187, EEDCKVHCVKEWMAGKACAERQKSYTIGRAHCSGQKFDVFKCLDHCAAP) above and to the side of a His substitution matrix. Each black box represents a first and second site in which His could be substituted. Those purely along the top-left to bottom-right diagonal represent single His substitutions. Each black box represents a variant with one or two native-to-His substitutions, representing 821 peptide variants to be screened. A variant library containing the parental sequence and variants with one or two native-to-His substitutions was generated and tested.

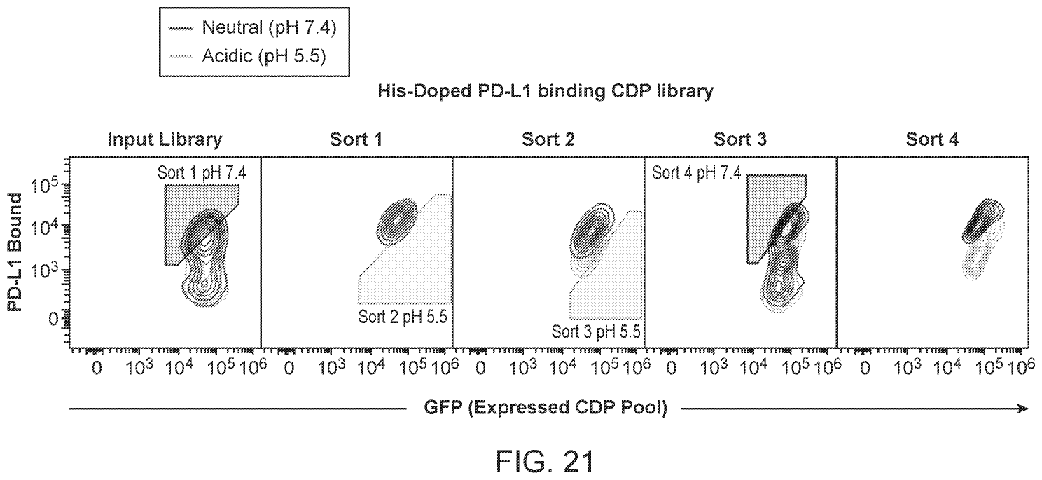

The resulting histidine-enriched PD-L1-binding peptides were evaluated for their PD-L1 binding in comparative binding experiments at various pH levels or ranges. A variant library of PD-L1-binding peptides was expressed via mammalian surface display, with each variant containing zero, one or two His substitutions. These variants were tested for maintenance of binding under extracellular pH (such as pH 7.4), and for reduced binding under endosomal pH (such as pH 5.5). Sequential screening was performed, as shown in FIG. 21. The input library was initially screened for PD-L1 binding at pH 7.4, and strong binders were selected (shaded area). The second and third rounds of screening (“Sort 1” and “Sort 2,” respectively) were performed at pH 5.5 to mimic endosomal pH, and the weak binders were collected (shaded area). The final round of screening (“Sort 3”) was performed at pH 7.4, and strong binders were selected. Differential binding at pH 7.4 and pH 5.5 was observed following screening (“Sort 4”).

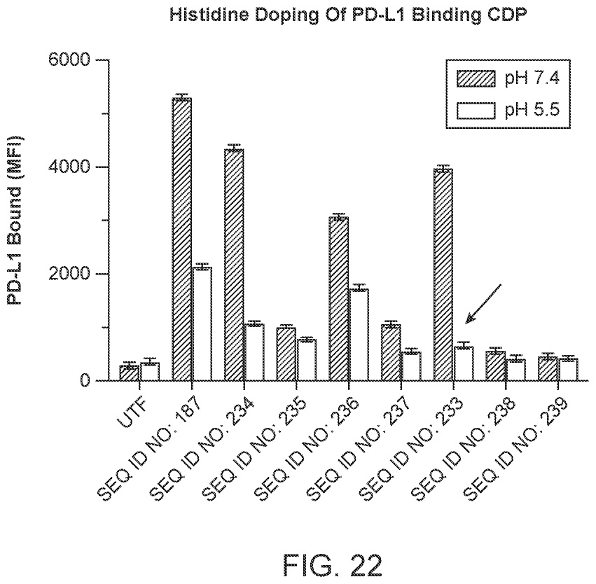

Variants of SEQ ID NO: 187 containing histidine substitutions at one, two, or three of E2H, M13H, and K16H amino acid positions were identified in the pooled screen as pH-dependent binders of PD-L1. pH-dependent binding was validated by measuring PD-L1 binding at pH 7.4 and pH 5.5 to cells surface expressing single variants, as shown in FIG. 22. Peptides containing substitutions at E2H (EHDCKVHCVKEWMAGKACAERQKSYTIGRAHCSGQKFDVFKCLDHCAAP, SEQ ID NO: 234), M13H (EEDCKVHCVKEWHAGKACAERQKSYTIGRAHCSGQKFDVFKCLDHCAAP, SEQ ID NO: 235), K16H (EEDCKVHCVKEWMAGHACAERQKSYTIGRAHCSGQKFDVFKCLDHCAAP, SEQ ID NO: 236), E2H and M13H (EHDCKVHCVKEWHAGKACAERQKSYTIGRAHCSGQKFDVFKCLDHCAAP, SEQ ID NO: 237), E2H and K16H (EHDCKVHCVKEWMAGHACAERQKSYTIGRAHCSGQKFDVFKCLDHCAAP, SEQ ID NO: 233), M13H and K16H (EEDCKVHCVKEWHAGHACAERQKSYTIGRAHCSGQKFDVFKCLDHCAAP, SEQ ID NO: 238), or E2H, M13H, and K16H (EHDCKVHCVKEWHAGHACAERQKSYTIGRAHCSGQKFDVFKCLDHCAAP, SEQ ID NO: 239) were compared to SEQ ID NO: 187. The variant corresponding to SEQ ID NO: 233, containing substitutions at E2H and K16H, showed strong binding to PD-L1 at pH 7.4 and substantial loss of binding at pH 5.5 (black arrow). The other variants and the parent peptide showed varying levels of PD-L1 binding at pH 7.4 and at pH 5.5, with varying degrees of pH dependence to the binding.

### Example 10

### Development of Selective Depletion Complexes Containing pH-Dependent PD-L1-Binding Peptides for Selective Depletion of PD-L1

This example describes development of selective depletion complexes containing pH-dependent PD-L1-binding peptides for selective depletion of PD-L1. Peptides with high PD-L1 binding affinity at physiologic extracellular pH but a significantly reduced binding affinity at lower pH levels such as endosomal pH of 5.5 are selected for cellular binding, uptake, and intra-endosomal or intra-vesicular release as described in EXAMPLE 9. PD-L1-binding peptides with high endosomal delivery capabilities are identified and characterized. PD-L1 binding peptides with high PD-L1 binding affinity at physiologic extracellular pH (e.g., pH 7.4) and reduced binding affinity at endosomal pH (e.g., pH 5.5) are fused recombinantly, chemically synthesized as a single fusion, separately recombinantly expressed and conjugated, or separately chemically synthesized and conjugated to a TfR-binding peptide with a TfR-binding affinity that is substantially the same at a physiologic extracellular pH and at endosomal pH (e.g., a TfR-binding peptide of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64), optionally with any linker or no linker in between the PD-L1 binding peptide and the TfR binding peptide.

A sample screening pipeline showing progression from screening for target binding CDPs, to modifying such CDPs for pH-dependent binding, to incorporation into compositions for selective depletion is shown in FIG. 11A. A peptide library of CDPs is screened for the ability to bind to a target molecule. Target-binding peptides from the library are distinguished by accumulation of signal from bound target molecules. Optionally, identified target-binding peptides are selected and further matured for binding, for example using point mutation screens. Identified target binding peptides are converted to pH-dependent binders, for example by performing histidine point mutation scans as illustrated in FIG. 11D and described in EXAMPLE 9. The pH-dependent target-binding peptide is fused or linked to a recycler peptide (e.g., a TfR-binding peptide of any of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64), to form a selective depletion complex. Optionally, the selective depletion complexes are validated by testing target depletion in cells expressing the selective depletion complexes, as shown in FIG. 11B. Complexes can be further tested in healthy cells and in transformed cell lines to measure disease-specific functionalities of the selective depletion complexes, as shown in FIG. 11C. Specificity of the complexes is measured by testing for changes in a target-specific cellular function, such as cancer-specific growth inhibition upon depletion of an apoptosis inhibitor. Target-specific cellular functions can depend on extrinsic or intrinsic factors, or a combination of extrinsic and intrinsic factors. Degradation of the target and selective impairment of cancer cells suggests that a therapeutic window exists in patients.

Cancer-specific growth inhibition by a selective depletion complex comprising a PD-L1-binding peptide may be tested using cells co-cultured with T cells. By removing some PD-L1 from cancer cell surfaces, the checkpoint inhibition signaling can be reduced, and the tumor cells can be more likely to be recognized by and attacked by the immune system, leading to reduced tumor growth, reduced metastasis, or increase of other beneficial tumor responses.

### Example 11

### Selective Depletion of a Soluble Target Molecule Via TfR-Mediated Endocytosis

This example describes selective depletion of a soluble target molecule via TfR-mediated endocytosis. A composition containing a TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) conjugated to a target-binding peptide is contacted to cells expressing TfR. The TfR-binding peptide binds TfR with high affinity at both physiologic extracellular pH (such as pH 7.4) and at endosomal pH (such as pH 5.5), and the target-binding peptide binds to a soluble target molecule with higher affinity at physiologic extracellular pH and with lower affinity at endosomal pH. Upon contact, the TfR-binding peptide binds to TfR on the cell surface, and the target-binding peptide binds to the soluble target molecule in solution (FIG. 12A, (1)). The composition containing the TfR-binding peptide and the target-binding peptide is endocytosed via TfR-mediated endocytosis along with the TfR and the bound target molecule (FIG. 12A, (2)). As the endosomal compartment acidifies, the target molecule is released from the target-binding peptide (FIG. 12A, (3)). The target molecule is then degraded in a lysosomal compartment (FIG. 12A, (4)), and the complex is recycled to the cell surface along with the TfR (FIG. 12A, (5)).

### Example 12

### Selective Depletion of a Surface Target Molecule Via TfR-Mediated Endocytosis

This example describes selective depletion of a surface target molecule via TfR-mediated endocytosis. A composition containing a TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) conjugated to a target-binding peptide is contacted to cells expressing TfR. The TfR-binding peptide binds TfR with high affinity at both physiologic extracellular pH (such as at pH 7.4) and at endosomal pH (such as at pH 5.5), and the target-binding peptide binds to a surface target molecule with higher affinity at physiologic extracellular pH and with lower affinity at endosomal pH. Upon contact, the TfR-binding peptide binds to TfR on the cell surface, and the target-binding peptide binds to the surface target molecule on the cell surface (FIG. 12B, (1)). The composition containing the TfR-binding peptide and the target-binding peptide is endocytosed via TfR-mediated endocytosis along with the TfR and the bound target molecule (FIG. 12B, (2)). As the endosomal compartment acidifies, the target molecule is released from the target-binding peptide (FIG. 12B, (3)). The target molecule is then degraded in a lysosomal compartment (FIG. 12B, (4)), and the complex is recycled to the cell surface along with the TfR (FIG. 12B, (5)).

### Example 13

### Extension of Peptide Plasma Half-Life Using Serum Albumin-Binding Peptide Complexes

This example demonstrates a method of extending the serum or plasma half-life of a peptide using serum albumin-binding peptide complexes as disclosed herein. A peptide or peptide complex having a sequence of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64 is modified in order to increase its plasma half-life. The peptide and the serum half-life extending moiety are fused recombinantly, chemically synthesized as a single fusion, separately recombinantly expressed and conjugated, or separately chemically synthesized and conjugated. Fusing the peptide to a serum albumin-binding peptide extends the serum half-life of the peptide complex. The peptide or peptide complex is conjugated to a serum albumin-binding peptide, such as SA21 (SEQ ID NO: 178). Optionally, the peptide fused to SA21 has a sequence of any one of SEQ ID NO: 181 or SEQ ID NO: 184. Optionally, the peptide fused to SA21 is linked to SA21 via a peptide linker having a sequence of SEQ ID NO: 179. The linker having a sequence corresponding to SEQ ID NO: 179 links two separately functional CDPs to incorporate serum half-life extension function into the peptide or peptide complex. The linker having a sequence corresponding to SEQ ID NO: 179 enables SA21 to cyclize without steric impediment from either member of the peptide complex. Alternatively, conjugation of the peptide to an albumin binder, such as Albu-tag or a fatty acid, such as a C₁₄-C₁₈ fatty acid or palmitic acid, is used to extend plasma half-life. Plasma half-life is also optionally extended as a result of reduced immunogenicity by using minimal non-human protein sequences.

### Example 14

### Purification of a TfR-Binding Serum Albumin-Binding Peptide Fusion

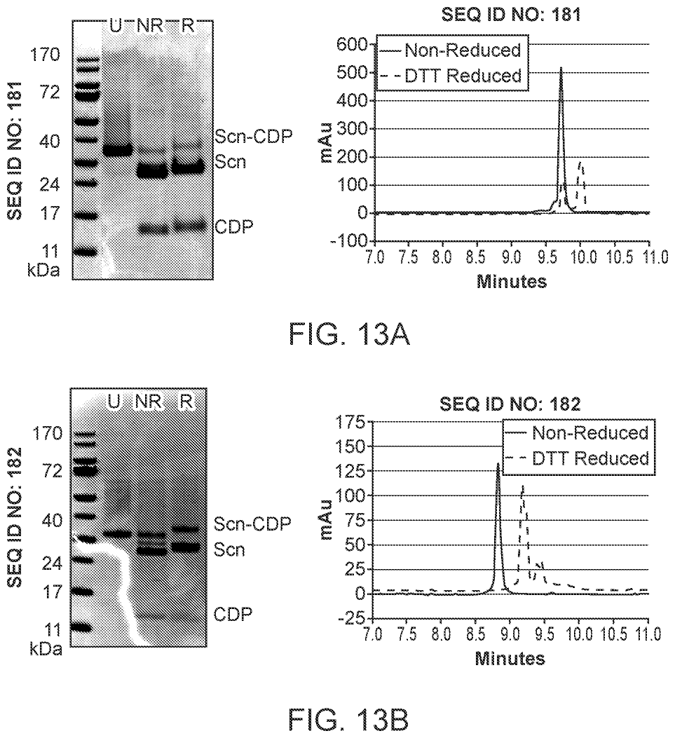

This example describes the purification of a TfR-binding peptide fused to the serum albumin-binding peptide SA21. FIG. 13A and FIG. 13B illustrate the purification of SA21 fusion peptides. SA21 was recombinantly expressed as a fusion peptide with a CDP and purified by HPLC. Peptides were purified fused to siderocalin (“Scn-CDP”) and cleaved to produce the cleaved SA21 fusion peptide (“CDP”) and siderocalin (“Scn”). FIG. 13A shows purification of a peptide TfR-binding peptide fused to a serum albumin-binding peptide (SA21) corresponding to SEQ ID NO: 181. Purity was verified by SDS-PAGE (left) and RP-HPLC (right) under DTT reducing (“R”) or non-reducing (“NR”) conditions. SDS-PAGE was also run on the uncleaved (“U”) siderocalin-CDP fusion peptide. In the SDS-PAGE, distinct bands were seen corresponding to the siderocalin-CDP fusion (“Scn-CDP”) in the uncleaved (“U”) sample, as well as bands corresponding to the cleaved SA21 fusion (“CDP”), siderocalin alone (“Scn”), and the uncleaved fusion (“Scn-CDP”) in the reduced (“R”) and non-reduced (“NR”) samples. The presence of a single peak in the unreduced RP-HPLC trace was indicative of a pure, undegraded sample. FIG. 13B shows purification of a peptide fused to SA21 corresponding to SEQ ID NO: 182 (GSRLIEDICLPRWGCLWEDDGGGGSGGGGSVRIPVSCKHSGQCLKPCKDAGMRF GKCMNGKCDCTPK). Purity was verified by SDS-PAGE (left) and RP-HPLC (right) under DTT reducing (“R”) or non-reducing (“NR”) conditions. SDS-PAGE was also run on the uncleaved (“U”) siderocalin-CDP fusion peptide. In the SDS-PAGE, distinct bands were seen corresponding to the siderocalin-CDP fusion (“Scn-CDP”) in the uncleaved (“U”) sample, as well as bands corresponding to the cleaved SA21 fusion (“CDP”), siderocalin alone (“Scn”), and the uncleaved fusion (“Scn-CDP”) in the reduced (“R”) and non-reduced (“NR”) samples. The presence of a single peak in the unreduced RP-HPLC trace was indicative of a pure, undegraded sample.

### Example 15

### Linkers for Conjugation and Half-Life Extension of Target-Binding Peptides and TfR-Binding Peptides

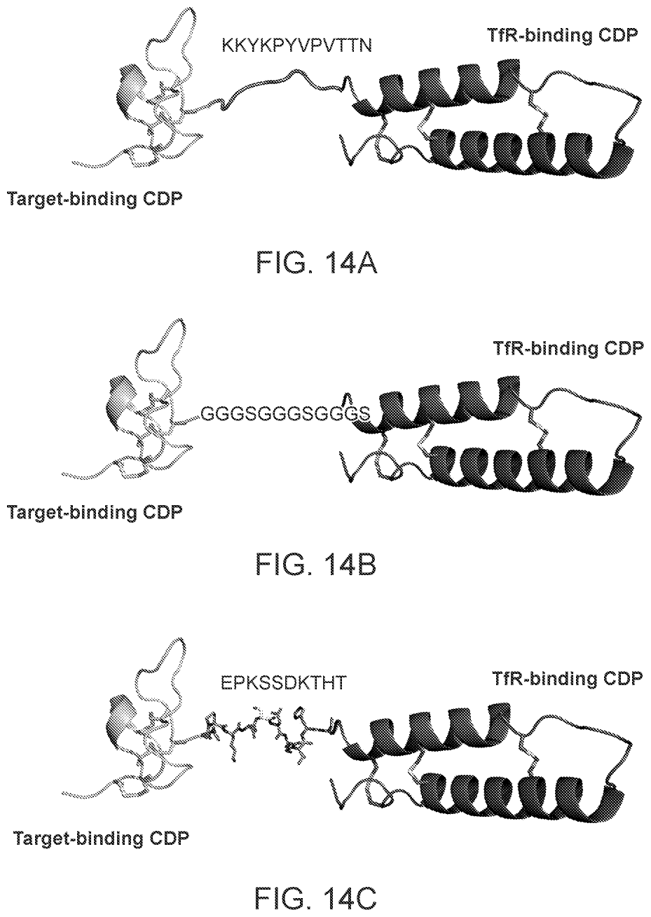

This example describes linkers for conjugation and optionally for half-life extension of target-binding peptides and TfR-binding peptides. A TfR-binding peptide (e.g., any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64) is conjugated to a target-binding peptide (e.g., a target-binding CDP selected for pH-dependent binding as described in EXAMPLE 9) via a linker. The target-binding peptide can be fused to the TfR-binding peptide via a DkTx peptide (SEQ ID NO: 139, KKYKPYVPVTTN) from native CDP dimer, as shown in FIG. 14A. The DkTx peptide linker is from a native knottin-knottin dimer from the Tau-theraphotoxin-Hs1a, also known as DkTx (double-knot toxin), in Haplopelma schmidti. Natively, the DkTx linker separates two independently folding CDP domains and is well-suited for maintaining the function of the two dimerizing CDPs. The target-binding peptide can be fused to the TfR-binding peptide via a poly-GlySer linker such as (SEQ ID NO: 138, GGGSGGGSGGGS), containing varying lengths of glycines interspaced by serines for solubility, as shown in FIG. 14B. The target-binding peptide can be fused to the TfR-binding peptide via a human IgG linker with a Cys-to-Ser mutation at position 5 (SEQ ID NO: 140, EPKSSDKTHT) to prevent crosslinking during secretion, as shown in FIG. 14C. A peptide linker for dimerizing two peptides optionally has the following properties: 1) the linker does not disturb the independent folding of the TfR- and target-binding domains, 2) the linker provides sufficient length to the mature molecule so as to facilitate contact between the target molecule and the TfR via the TfR-binding peptide target-biding peptide dimer, 3) the linker does not negatively impact manufacturability (synthetic or recombinant) of the TfR-binding peptide target-biding peptide dimer, and 4) the linker does not impair any required post-synthesis chemical alteration of the TfR-binding peptide target-biding peptide dimer (e.g., conjugation of a fluorophore or albumin-binding chemical group).

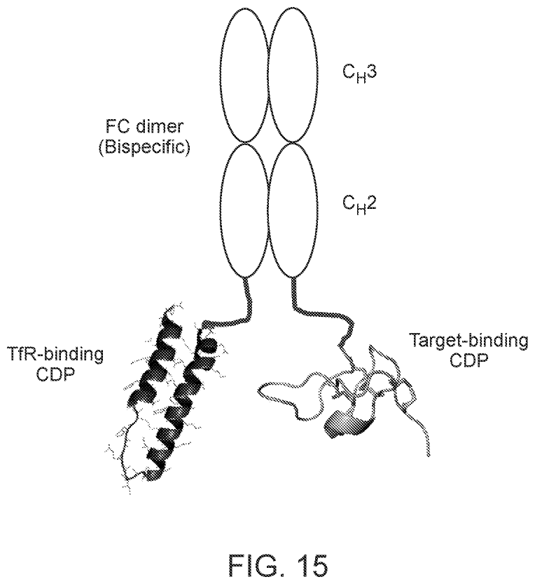

CDPs (or other protein-based target-engaging modalities) can also be dimerized using immunoglobulin heavy chain Fc domains. These are commonly used in modern molecular medicine to dimerize functional domains, either based on antibodies or other otherwise soluble functional domains. The target-binding peptide can be non-covalently linked to the TfR-binding peptide via an IgG-based Fc domain, as shown in FIG. 15. An Fc domain can be used to homo- or hetero-dimerize functional domains and to impart serum half-life extension via a domain that interacts with the recycling Fc receptor (FcRn). Dimerization can be homodimeric if the Fc sequences are native, but if one wants to drive heterodimer formation, the Fc can be mutated into “knob-in-hole” format, where one Fc CH3 contains novel residues (knob) designed to fit into a cavity (hole) on the other Fc CH3 domain. Through this process, knob+knob dimers are highly energetically unfavorable. Hole+hole dimers can be formed, but if a purification tag is added specifically to the “knob” side, hole+hole dimers can be excluded, ensuring that only knob+hole dimers are purified. Fc domains can separately be used as a recycling receptor-engaging domain, so use of Fc for dimerization can enhance peptide complex recycling or selective degradation complex.

TfR-binding and target-binding CDPs can be further functionalized and multimerized by adding a third (or more) functional domain. In this example, an albumin-binding domain from a Finegoldia magna peptostreptococcal albumin-binding protein (SEQ ID NO: 192) is shown, as it is a simple three-helical structure that would be unlikely to disturb the independent folding of the other CDP domains. Such added functional domains could be included in any orientation relative to the TfR- and target-binding domains, as shown in FIG. 16A-FIG. 16C. Example peptides are shown with a poly-GlySer linker, but any of a number of linkers (e.g., any one of SEQ ID NO: 129-SEQ ID NO: 141 or SEQ ID NO: 195-SEQ ID NO: 218) could be used. An albumin binding domain (e.g., a peptide of SEQ ID NO: 178 or SEQ ID NO: 192) can be fused to the TfR-binding peptide, the target-binding peptide, or both. The albumin binding domain can include a peptide linker (e.g., any one of SEQ ID NO: 129-SEQ ID NO: 141 or SEQ ID NO: 195-SEQ ID NO: 218). The albumin binding domain can be linked to the target-binding peptide and the TfR-binding peptide, as shown in FIG. 16A. The albumin binding domain can be linked to the target-binding peptide, as shown in FIG. 16B. The albumin binding domain can be linked to the TfR-binding peptide, as shown in FIG. 16C. Addition of the albumin binding domain can increase the serum half-life of a composition containing the TfR-binding peptide and the target-binding peptide.

Similar methods and designs can be used for selective depletion complexes containing a PD-L1-binding domain (e.g., a PD-L1-binding peptide) rather than a TfR-binding domain the as the recycling receptor, in methods where PD-L1 is used as the recycling receptor.

### Example 16

### Functional Binding of CDP-CDP Dimers Containing a TfR-Binding Peptide and a Target-Binding Peptide

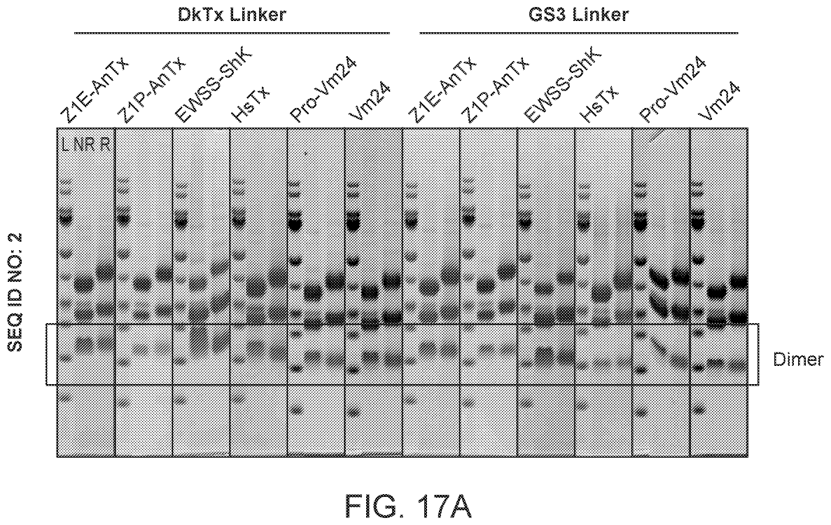

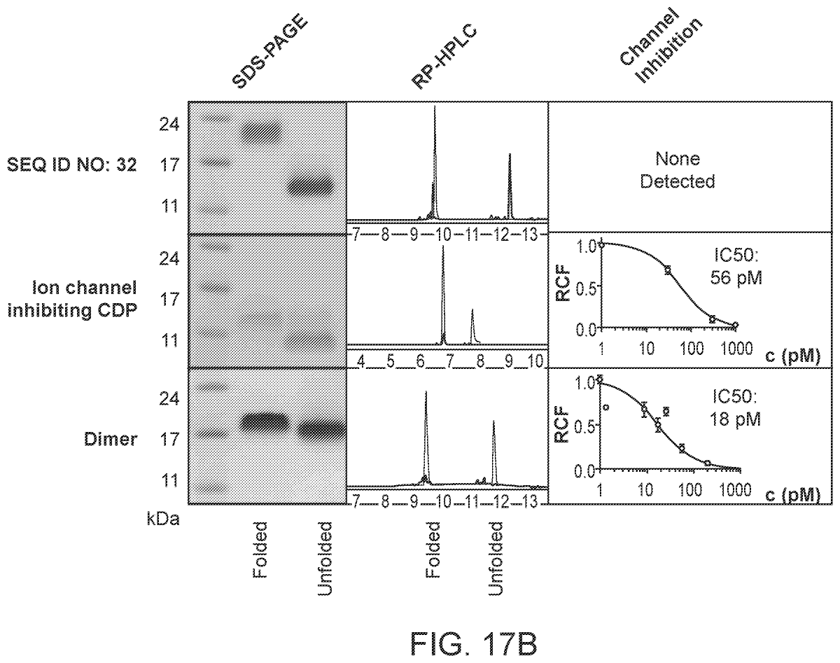

This example describes functional binding of CDP-CDP dimers containing a TfR-binding peptide and a target-binding peptide. CDP-CDP dimers containing a TfR-binding peptide of SEQ ID NO: 2 and an ion-channel inhibitory CDP. The TfR-binding peptide was linked to a peptide inhibitor of the Kv1.3 voltage-gated potassium channel (Z1E-AnTx, Z1P-AnTx, EWSS-ShK, HsTx, Pro-Vm24, or Vm24) by either a DkTx linker (SEQ ID NO: 139) or a GS3 linker (SEQ ID NO: 141). The CDP-CDP dimer peptides were expressed as a fusion with a siderocalin carrier peptide (SEQ ID NO: 147), which was cleaved off with a TEV protease. The purified peptides were run on an SDS-PAGE gel to verify that the peptide fusions were intact (FIG. 17A). Each gel contained, from left to right, a molecular weight latter (“L”), the peptide sample under non-reducing conditions (“NR”), and the peptide sample under reducing conditions (“R”). The easily distinguishable bands in the peptide sample lanes corresponded to, from top to bottom, uncleaved CDP-CDP dimer with siderocalin, cleaved siderocalin, and cleaved CDP-CDP dimer. All of the CDP-CDP dimer complexes expressed well, as indicated by the band intensity, and appeared folded, as indicated by the shift upon reduction with DTT.

In a second assay, a different TfR-binding CDP corresponding to SEQ ID NO: 32 was fused to the Vm24 ion channel inhibitor via a polyGly-Ser linker (SEQ ID NO: 138). The resulting CDP-CDP dimer was purified and run on an SDS-PAGE gel (FIG. 17B, left bottom). The TfR-binding peptide (SEQ ID NO: 32) and the Vm24 ion channel-inhibiting CDP were also purified individually (FIG. 17B, left top and left middle). Purity of the TfR-binding peptide, the ion channel-inhibiting CDP, and the CDP-CDP dimer was compared using reverse phase high pressure liquid chromatography (RP-HPLC, FIG. 17B, center). The ability of each complex to inhibit a Kv1.3 ion channel was then tested (FIG. 17B, right). The CDP-CDP dimer retained its ability to inhibit the ion channel compared to the ion channel-inhibiting CDP alone. As expected, the TfR-binding peptide alone did not inhibit Kv1.3.

### Example 17

### Cross Reactivity of TfR-Binding Peptides to Murine TfR

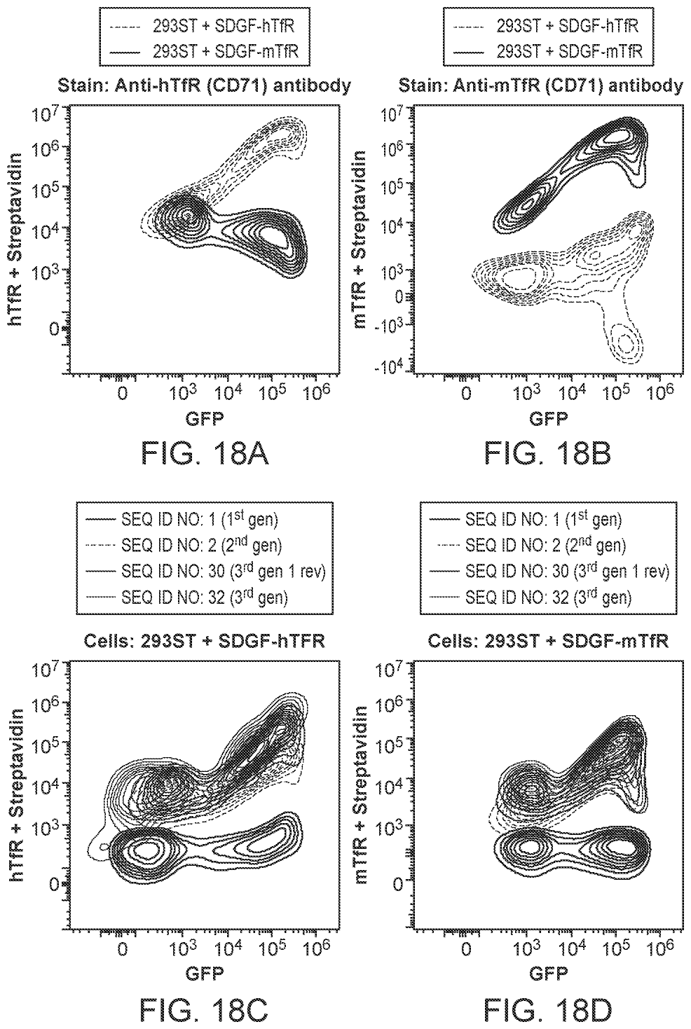

This example illustrates cross reactivity of TfR-binding peptides of the present disclosure to murine TfR in cell surface binding assays. 293F cells expressing either human or mouse TfR on their surface were stained with soluble TfR-binding peptides that were directly labeled with AlexaFluor 647 dye via NHS-ester conjugation. FIG. 18A and FIG. 18B show flow cytometry plots that verify human versus mouse TfR expression using species-specific antibodies. FIG. 18C and FIG. 18D demonstrate that the peptides effectively bind to both homologs. In a similar experiment, flow cytometry was used to demonstrate effective binding of SEQ ID NO: 1, SEQ ID NO: 2, SEQ ID NO: 32, and Anti-Tf antibodies (positive controls).

### Example 18

### Activation of Neuronal CRE Reporter Mice

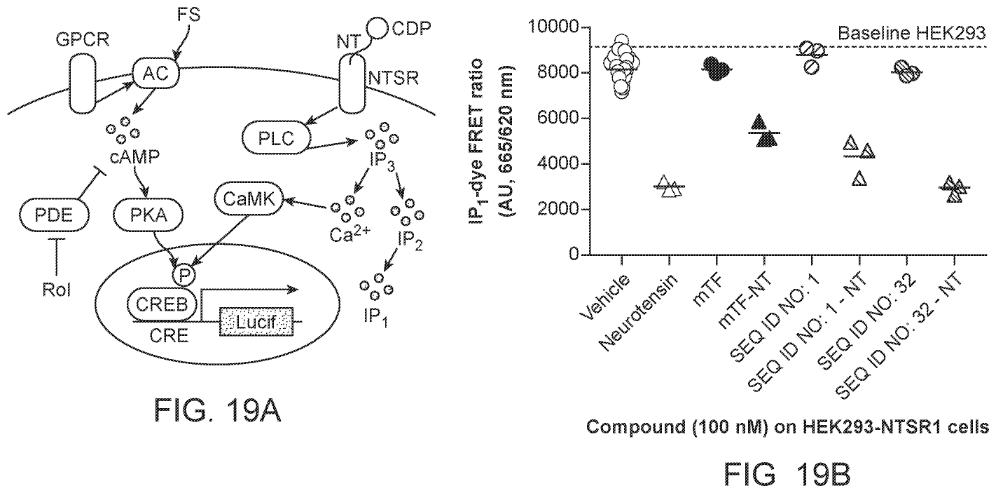

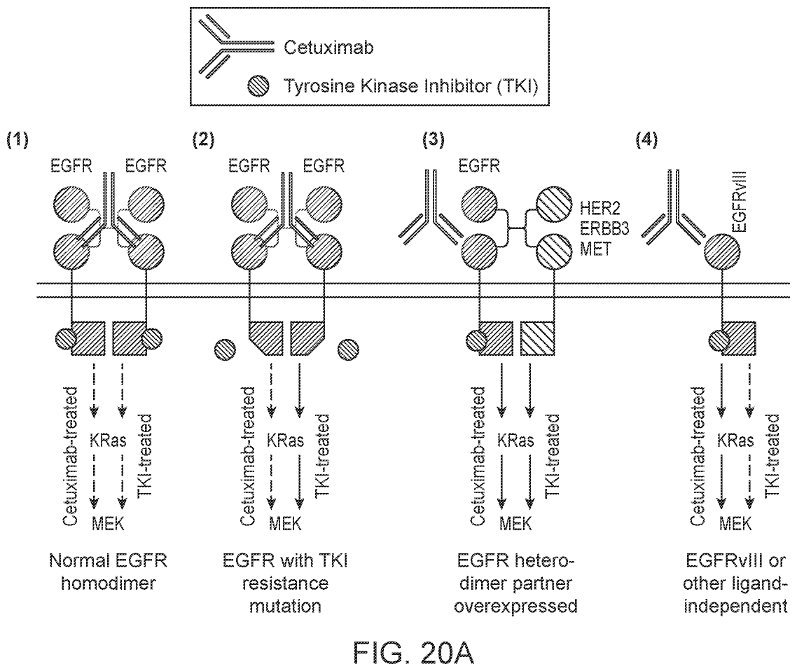

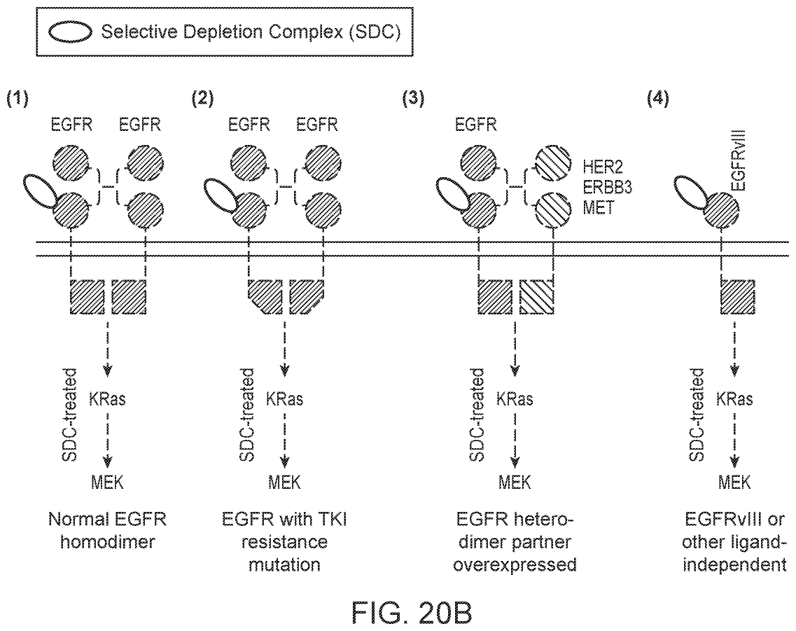

This example describes activation of neuronal CRE transporter mice using peptide complexes comprising one or more TfR-binding peptides as described herein. In this case, a fusion peptide comprising TfR-binding peptides and a neurotensin peptide was used. Peptides corresponding to SEQ ID NO: 1, SEQ ID NO: 2, and SEQ ID NO: 32 (SEQ ID NO: 1, SEQ ID NO: 2, and SEQ ID NO: 32 are SEQ ID NO: 65, SEQ ID NO: 66, and SEQ ID NO: 96, respectively, with an added N-terminal GS) were fused with neurotensin at the C-terminus of each peptide to produce the peptide-NT complexes. The downstream activity of neurotensin involves intracellular Ca²⁺ regulation and cAMP response element (CRE) driven transcriptional programs (FIG. 19A), and its modulation has been explored for suppression of chronic pain. Peptide-NT were expressed recombinantly in 293F cells and purified. Molecular weights of the purified peptides were verified using mass spectrometry.

Binding to the neurotensin receptor was demonstrated with a HEK-293 cell line expressing NTSR1. To demonstrate that the neurotensin extension on various proteins was functional, NTSR activity in HEK293 cells, or HEK293 cells transduced with a lentivector delivering human NTSR1 (HEK293-NTSR1), was measured using the IP-One-Gq kit (CisBio 62IPAPEB, FIG. 19B). Cells were grown in DMEM+10% fetal bovine serum, removed from the plates with Accutase, pelleted, and suspended in Hanks Buffered Salt Solution at a density of 1.5×10⁶ cells per mL. HTFR reactions were set up in HTFR 96 well low volume plates (CisBio #66PL96025) according to the manufacturer's instructions. 10,000 cells (7 μL) were used per 25 μL reaction. The plate was incubated for 60 mins at 37° C. Then 3 μL IP1-d2 working solution was then added, followed by 3 μL Anti IP1-Cryptate working solution. After incubating for 1 hour at room temperature, the plate was scanned in a Perkin Elmer 2104 EnVision Multilabel Reader for fluorescence emission after excitement at 665 nm and 620 nm wavelengths. FRET ratio was calculated as 10,000× (Signal 665 nm/Signal 620 nm). In mammalian HEK-293 cells neurotensin (NT) receptor engagement showed IP₁ accumulation only in response to NT or NT peptide complexes (SEQ ID NO: 1 conjugated to NT and SEQ ID NO: 32 conjugated to NT), as well as mTf-NT and NT, but not for SEQ ID NO: 1 or SEQ ID NO: 32, vehicle, or mTf), N=3 wells for all except vehicle, which had N=36 (FIG. 19B). Horizontal bar indicates sample mean.

### Example 19

### Development of a High Affinity and pH-Dependent EGFR Binding Nanobody

This example describes development of a high affinity and pH-dependent EGFR binding nanobody. A nanobody that binds EGFR (QVKLEESGGGSVQTGGSLRLTCAASGRTSRSYGMGWFRQAPGKEREFVSGISWRGDS TGYADSVKGRFTISRDNAKNTVDLQMNSLKPEDTAIYYCAAAAGSAWYGTLYEYDY WGQGTQVTVSS, SEQ ID NO: 219) was modified for higher affinity and for pH-dependent binding to EGFR. A peptide library containing histidine point mutations at each residue in the two complementarity-determining regions shown by crystal structure to interact with EGFR (underlined in SEQ ID NO: 219 showing CDR1 and CDR3, respectively) was generated. Because CDR1 contains 10 non-histidine residues, one can generate up to 56 variants with 0, 1, or 2 histidines. Because CDR3 contains 17 non-histidine residues, one can generate up to 154 variants with 0, 1, or 2 histidines. Histidine mutants were tested individually and then hits combined into a single VHH variant, or they were genetically recombined and tested as a variant library of 56×154=8,624 members. Hits from this library were identified by screening for retaining high cell staining/affinity at pH ˜7.4 and demonstrating low cell staining/affinity at pH˜6 or lower.

Two EGFR-binding nanobodies were identified using this screen. A first EGFR-binding nanobody (SEQ ID NO: 242) was identified as a high-affinity EGFR-binding peptide that bound EGFR with higher affinity than SEQ ID NO: 219. A second EGFR-binding nanobody (SEQ ID NO: 243) was identified as a pH-dependent EGFR-binding nanobody that bound to EGFR with high affinity at ˜7.4 and showed a decrease in binding affinity at ˜6 or lower.

### Example 20

### Site Saturation Mutagenesis to Identify pH-Dependent Target Binding Peptides

This example describes site saturation mutagenesis to identify pH-dependent target binding peptides. A peptide (e.g., a nanobody) that binds to a target molecule (e.g., PD-L1, VEGF, PD-1, EGFR, CD38, GD2, SLAMF7, CTLA-4, CCR4, CD20, PDGFRα, VEGFR2, HER2, CD33, CD30, CD22, CD79B, Nectin-4, or TROP2) is modified for pH-dependent binding by performing site saturation mutagenesis as described with respect to a TfR-binding peptide in EXAMPLE 5. The site saturation mutant library is screened for binding to the target molecule at physiologic extracellular pH (e.g., pH 7.4) and at endosomal pH (e.g., pH 5.5). Mutants that show a higher binding affinity at physiologic extracellular pH and reduced binding affinity at endosomal pH are selected and further screened. Subsequent rounds of site saturation mutagenesis are performed on the hits to further improve pH-dependent binding.

### Example 21

### Delivery of Selective Depletion Complexes Using Gene Therapy or Cell Therapy

This example describes delivery of selective depletion complexes using an oncolytic herpes simplex virus. A gene encoding expression and secretion of a selective depletion complex is introduced to a target cell using an oncolytic herpes simplex virus (oHSV) vector. For gene therapy, the target cell is a cell within a patient. For cell therapy, a target cell is a patient cell that has been collected and is re-introduced into the patient after modification with the viral vector. The oHSV infects cancer cells, and cancer cells that are not killed by the virus express and secrete the selective depletion complex. The remaining cells modify the tumor microenvironment to suppress immune activity against the cancer cells. Selective depletion complexes are secreted from the tumor cells in situ and act on the cancers are directed against immunosuppressive factors on T cells or in the tumor microenvironment.

Alternatively, selective depletion complexes that modify tumor or T-cell activity could are engineered into CAR-T cells or other cellular therapies. CAR-T cells are already being specialized through genetic modification to target tumor tissue, killing tumor cells that carry cell surface markers targeted by the expressed chimeric antigen receptors (CAR). If supplemental activity is desired, such as suppression of regulatory, immunosuppressive signaling present in these tumors, the CAR-T cell is engineered to also secrete selective depletion complexes that suppress regulatory, immunosuppressive signaling.

### Example 22

### Ternary Complex Formation Between Selective Depletion Complexes, Target Molecules, and Receptors

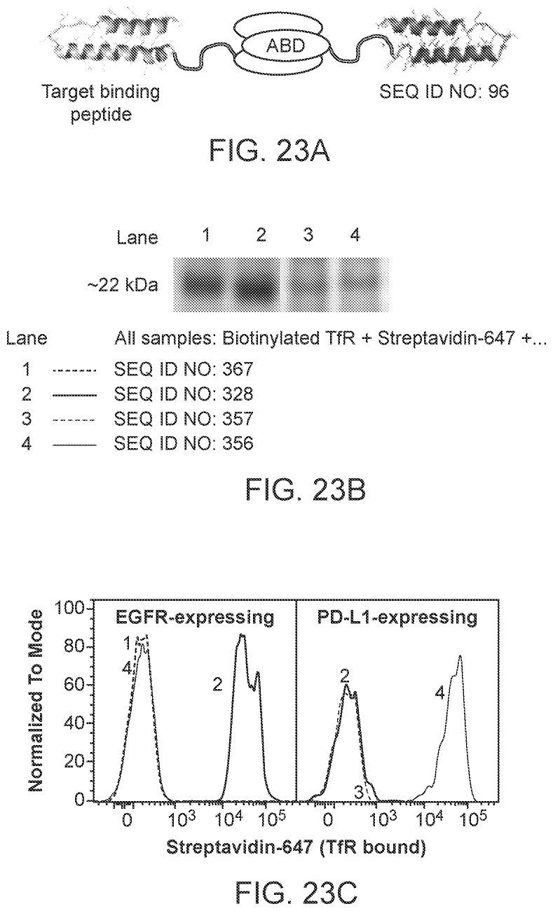

This example describes ternary complex formation between selective depletion complexes, target molecules, and receptors while on the surface of a cell. Selective depletion complexes (SDCs) containing a target-binding peptide, a first peptide linker (GGGGS×4, SEQ ID NO: 224), an albumin binding peptide (SEQ ID NO: 227), a second peptide linker (GGGGS×4, SEQ ID NO: 224), and a TfR-binding peptide were designed to bind a target molecule and a transferrin receptor, as illustrated in FIG. 23A. SDCs can contain one binding end that binds in a pH dependent fashion. Preferably, the pH dependent binding has a significant differential in binding at endosomal pH (e.g., pH 5.5, 6.0, 6.5, 5.0, 4.5) versus binding at extracellular pH (e.g., pH 7.4, 7.0); however milder differences in endosomal versus extracellular pH binding may also be effective. Peptide complexes containing: a target-binding peptide that binds EGFR with mild pH dependence (SEQ ID NO: 244) and a low affinity TfR-binding peptide with a sequence of REGCASHCTKYKAELEKCEARVSSRSNTEETCVQELFDFLHCVDHCVSQ corresponding to SEQ ID NO: 232 (peptide 1, SEQ ID NO: 367, NSDSECPLSHDGYCLHGGVCMYIKAVDRYACNCVVGYIGERCQYRDLTWWGPRGTG GGGSGGGGSGGGGSGGGGSLKEAKEKAIEELKKAGITSDYYFDLINKAKAVEGVNALK DEILKAGGGGSGGGGSGGGGSGGGGSREGCASHCTKYKAELEKCEARVSSRSNTEETC VQELFDFLHCVDHCVSQ); a target-binding peptide that binds EGFR (SEQ ID NO: 244) and a high affinity TfR-binding peptide corresponding to SEQ ID NO: 96 (peptide 2, SEQ ID NO: 328); a target-binding peptide that binds PD-L1 with moderate pH dependence (SEQ ID NO: 187) and a low affinity TfR-binding peptide corresponding to SEQ ID NO: 232 (peptide 3, SEQ ID NO: 357, EEDCKVHCVKEWMAGKACAERQKSYTIGRAHCSGQKFDVFKCLDHCAAPGTGGGGS GGGGSGGGGSGGGGSLKEAKEKAIEELKKAGITSDYYFDLINKAKAVEGVNALKDEIL KAGGGGSGGGGSGGGGSGGGGSREGCASHCTKYKAELEKCEARVSSRSNTEETCVQE LFDFLHCVDHCVSQ); or a target-binding peptide that binds PD-L1 with moderate pH dependence (SEQ ID NO: 187) and a high affinity TfR-binding peptide corresponding to SEQ ID NO: 96 (peptide 4, SEQ ID NO: 356; EEDCKVHCVKEWMAGKACAERQKSYTIGRAHCSGQKFDVFKCLDHCAAPGTGGGGS GGGGSGGGGSGGGGSLKEAKEKAIEELKKAGITSDYYFDLINKAKAVEGVNALKDEIL KAGGGGSGGGGSGGGGSGGGGSREGCASRCMKYNDELEKCEARMMSMSNTEEDCEQ ELEDLLYCLDHCHSQ) were expressed and purified, as shown in FIG. 23B. FIG. 23B shows SDS-PAGE analysis of the four peptide complexes, confirming successful expression and purification of the molecules. The four peptide complexes were screened for their ability to form ternary complexes by binding to both a cell surface-expressed target molecule (EGFR or PD-L1) and a receptor molecule (soluble TfR ectodomain fluorescently labeled with streptavidin-647).

Cells expressing either EGFR or PD-L1 were co-stained with the four peptide complexes, corresponding to SEQ ID NO: 367 (1), SEQ ID NO: 328 (2), SEQ ID NO: 357 (3), and SEQ ID NO: 356 (4) and labeled with soluble TfR ectodomain fluorescently labeled with streptavidin-647, and the binding of the molecules to the cell surfaces was assessed by flow cytometry as shown in FIG. 23C. The selective depletion complex containing an EGFR-binding peptide and a high-affinity TfR-binding peptide (peptide 2, corresponding to SEQ ID NO: 328) formed ternary complexes with cells expressing EGFR (left) but not with cells expressing PD-L1 (right). Conversely, the selective depletion complex containing a PD-L1-binding peptide and a high-affinity TfR-binding peptide (peptide 4, corresponding to SEQ ID NO: 356) formed ternary complexes with cells expressing PD-L1 (right) but not with cells expressing EGFR (left). Comparator peptide complexes with low affinity binding to TfR (peptides 1 and 3, corresponding to SEQ ID NO: 367 and SEQ ID NO: 357, respectively) did not form ternary complexes, as seen by a lack of fluorescent labeling in the right and left panels of FIG. 23C. Together, this data demonstrates that selective depletion molecules containing a target-binding peptide and a high-affinity receptor-binding peptide form ternary complexes with the target (e.g., EGFR or PD-L1) and the receptor (e.g., TfR) on a cell surface.

Additional selective complexes and complex components use as comparators are provided in TABLE 11.

TABLE 11
Comparator Peptide Complexes and Complex Components

| SEQ ID | |
|---|---|
| NO | Sequence |
| SEQ ID | QVKLEESGGGSVQTGGSLRLTCAASGHTSRSYGMHWFRQAPGKEREFV |
| NO: 314 | SGISWRGDSTGYADSVKGRFTISRDNAKNTVDLQMNSLKPEDTAIYYCA |
| | AAAGSHWYGTHYEYDYWGQGTQVTVSSGGGGSGGGGSGGGGSEPKSS |
| | DKTHTCPPCPAPEAAGGPSVFLFPPKPKDTLMISRTPEVTCVVVDVSHED |
| | PEVKFNWYVDGVEVHNAKTKPREEQYNSTYRVVSVLTVLHQDWLNGK |
| | EYKCKVSNKALGAPIEKTISKAKGQPREPQVYTLPPSREEMTKNQVSLTC |
| | LVKGFYPSDIAVEWESNGQPENNYKTTPPVLDSDGSFFLYSKLTVDKSR |
| | WQQGNVFSCSVMHEALHNHYTQKSLSLSPGKGGGGSGGGGSGGGGSRE |
| | GCASHCTKYKAELEKCEARVSSRSNTEETCVQELFDFLHCVDHCVSQ |
| SEQ ID | REGCASRCMKYNDELEKCEARMMSMSNTEEDCEQELEDLLYCLDHCHS |
| NO: 349 | QGGGGSGGGGSGGGGSQVKLEESGGGSVQTGGSLRLTCAASGHTSRSY |
| | GMGWFRQAPGKEREFVSGISWRGDSTGYADSVKGRFTISRDNAKNTVD |
| | LQMNSLKPEDTAIYYCAAAAGSAWYHTLYEYDYWGQGTQVTVSS |
| SEQ ID | QVQLQESGGGVVQPGRSLRLSCAASRFTFNNYAMHWVRQAPGKGLEW |
| NO: 350 | VAVISYDGSNKYYADSVKGRFTISRDNSKNTLYLQMNSLRAEDTAVYYC |
| | ARDLSGYGDYPDYWGQGTLVTVSSGGGGSGGGGSGGGGSSELTQDPAV |
| | SVALGQTVRITCQGDSLRSYYASWYQQKPGQAPVLVMYGRNERPSGVP |
| | DRFSGSKSGTSASLAISGLQPEDEANYYCAGWDDSLTGPVFGGGTKLTV |
| | LGGGGGSGGGGSGGGGSQVKLEESGGGSVQTGGSLRLTCAASGHTSRS |
| | YGMGWFRQAPGKEREFVSGISWRGDSTGYADSVKGRFTISRDNAKNTV |
| | DLQMNSLKPEDTAIYYCAAAAGSAWYHTLYEYDYWGQGTQVTVSS |
| SEQ ID | QVKLEESGGGSVQTGGSLRLTCAASGHTSRSYGMGWFRQAPGKEREFV |
| NO: 353 | SGISWRGDSTGYADSVKGRFTISRDNAKNTVDLQMNSLKPEDTAIYYCA |
| | AAAGSAWYHTLYEYDYWGQGTQVTVSSGGGGSGGGGSGGGGSEPKSS |
| | DKTHTCPPCPAPEAAGGPSVFLFPPKPKDTLMISRTPEVTCVVVDVSHED |
| | PEVKFNWYVDGVEVHNAKTKPREEQYNSTYRVVSVLTVLHQDWLNGK |
| | EYKCKVSNKALGAPIEKTISKAKGQPREPQVYTLPPSREEMTKNQVSLTC |
| | LVKGFYPSDIAVEWESNGQPENNYKTTPPVLDSDGSFFLYSKLTVDKSR |
| | WQQGNVFSCSVMHEALHNHYTQKSLSLSPGKGGGGSGGGGSGGGGSRE |
| | GCASRCMKYNDELEKCEARMMSMSNTEEDCEQELEDLLYCLDHCHSQ |
| SEQ ID | QVQLVESGGALVQPGGSLRLSCAASGFPVNRYSMRWYRQAPGKEREWV |
| NO: 354 | AGMSSAGDRSSYEDSVKGRFTISRDDARNTVYLQMNSLKPEDTAVYYC |
| | NVNVGFEYWGQGTQVTVSSGGGGSGGGGSGGGGSEPKSSDKTHTCPPC |
| | PAPEAAGGPSVFLFPPKPKDTLMISRTPEVTCVVVDVSHEDPEVKFNWYV |
| | DGVEVHNAKTKPREEQYNSTYRVVSVLTVLHQDWLNGKEYKCKVSNK |
| | ALGAPIEKTISKAKGQPREPQVYTLPPSREEMTKNQVSLTCLVKGFYPSDI |
| | AVEWESNGQPENNYKTTPPVLDSDGSFFLYSKLTVDKSRWQQGNVFSCS |
| | VMHEALHNHYTQKSLSLSPGKGGGGGGGGSGGGGSREGCASRCMKY |
| | NDELEKCEARMMSMSNTEEDCEQELEDLLYCLDHCHSQ |
| SEQ ID | EEDCKVHCVKEWMAGKACAERQKSYTIGRAHCSGQKFDVFKCLDHCA |
| NO: 357 | APGTGGGGSGGGGSGGGGSGGGGSLKEAKEKAIEELKKAGITSDYYFDL |
| | INKAKAVEGVNALKDEILKAGGGGSGGGGSGGGGSGGGGSREGCASHC |
| | TKYKAELEKCEARVSSRSNTEETCVQELFDFLHCVDHCVSQ |
| SEQ ID | EHDCKVHCVKEWMAGHACAERQKSYTIGRAHCSGQKFDVFKCLDHCA |
| NO: 366 | APGTGGGGSGGGGSGGGGSGGGGSLKEAKEKAIEELKKAGITSDYYFDL |
| | INKAKAVEGVNALKDEILKAGGGGSGGGGSGGGGSGGGGSREGCASHC |
| | TKYKAELEKCEARVSSRSNTEETCVQELFDFLHCVDHCVSQ |
| SEQ ID | NSDSECPLSHDGYCLHGGVCMYIKAVDRYACNCVVGYIGERCQYRDLT |
| NO: 367 | WWGPRGTGGGGSGGGGSGGGGSGGGGSLKEAKEKAIEELKKAGITSDY |
| | YFDLINKAKAVEGVNALKDEILKAGGGGSGGGGSGGGGSGGGGSREGC |
| | ASHCTKYKAELEKCEARVSSRSNTEETCVQELFDFLHCVDHCVSQ |
| SEQ ID | QVKLEESGGGSVQTGGSLRLTCAASGRTSRSYGMGWFRQAPGKEREFVS |
| NO: 368 | GISWRGDSTGYADSVKGRFTISRDNAKNTVDLQMNSLKPEDTAIYYCAA |
| | AAGSAWYGTLYEYDYWGQGTQVTVSSGTGGGGSGGGGSGGGGSGGG |
| | GSLKEAKEKAIEELKKAGITSDYYFDLINKAKTVEGVNALKDEILKAGGG |
| | GSGGGGSGGGGSGGGGSREGCASRCMKYNDELEKCEARMMSMSNTEE |
| | DCEQELEDLLYCLDHCHSQ |
| SEQ ID | QVKLEESGGGSVQTGGSLRLTCAASGRTSRSYGMGWFRQAPGKEREFVS |
| NO: 369 | GISWRGDSTGYADSVKGRFTISRDNAKNTVDLQMNSLKPEDTAIYYCAA |
| | AAGSAWYGTLYEYDYWGQGTQVTVSSGTGGGGSGGGGSGGGGSGGG |
| | GSLKEAKEKAIEELKKAGITSDYYFDLINKAKAVEGVNALKDEILKAGG |
| | GGSGGGGSGGGGSGGGGSREGCASRCMKYNDELEKCEARMMSMSNTE |
| | EDCEQELEDLLYCLDHCHSQ |
| SEQ ID | QVKLEESGGGSVQTGGSLRLTCAASGRTSRSYGMGWFRQAPGKEREFVS |
| NO: 370 | GISWRGDSTGYADSVKGRFTISRDNAKNTVDLQMNSLKPEDTAIYYCAA |
| | AAGSAWYGTLYEYDYWGQGTQVTVSSGTGGGGSGGGGSGGGGSGGG |
| | GSLKEAKEKAIEELKKAGITSDYYFDLINKAKAVEGVNALKDEILKAGG |
| | GGSGGGGSGGGGSGGGGSREGCASHCTKYKAELEKCEARVSSRSNTEET |
| | CVQELFDFLHCVDHCVSQ |
| SEQ ID | QVKLEESGGGSVQTGGSLRLTCAASGRTSRSYGMGWFRQAPGKEREFVS |
| NO: 372 | GISWRGDSTGYADSVKGRFTISRDNAKNTVDLQMNSLKPEDTAIYYCAA |
| | AAGSAWYGTLYEYDYWGQGTQVTVSSGTGGGGSGGGGSGGGGSGGG |
| | GSEPKSCDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLHQD |
| | WLNGKEYKCKVSNKALGAPIEKTISKAKGQPREPQVYTLPPSRDELTKN |
| | QVSLSCAVKGFYPSDIAVEWESNGQPENNYKTTPPVLDSDGSFFLVSKLT |
| | VDKSRWQQGNVFSCSVMHEALHNHYTQKSLSLSPGKGGGGSGGGGSG |
| | GGGSREGCASRCMKYNDELEKCEARMMSMSNTEEDCEQELEDLLYCLD |
| | HCHSQ |
| SEQ ID | QVKLEESGGGSVQTGGSLRLTCAASGRTSRSYGMGWFRQAPGKEREFVS |
| NO: 374 | GISWRGDSTGYADSVKGRFTISRDNAKNTVDLQMNSLKPEDTAIYYCAA |
| | AAGSAWYGTLYEYDYWGQGTQVTVSSGTGSPVPSTPPTPSPSTPPTPSPS |
| | ASLKEAKEKAIEELKKAGITSDYYFDLINKAKTVEGVNALKDEILKAGSP |
| | VPSTPPTPSPSTPPTPSPSASREGCASRCMKYNDELEKCEARMMSMSNTE |
| | EDCEQELEDLLYCLDHCHSQ |
| SEQ ID | QVKLEESGGGSVQTGGSLRLTCAASGRTSRSYGMGWFRQAPGKEREFVS |
| NO: 375 | GISWRGDSTGYADSVKGRFTISRDNAKNTVDLQMNSLKPEDTAIYYCAA |
| | AAGSAWYGTLYEYDYWGQGTQVTVSSGTGSPVPSTPPTPSPSTPPTPSPS |
| | ASLKEAKEKAIEELKKAGITSDYYFDLINKAKAVEGVNALKDEILKAGSP |
| | VPSTPPTPSPSTPPTPSPSASREGCASRCMKYNDELEKCEARMMSMSNTE |
| | EDCEQELEDLLYCLDHCHSQ |
| SEQ ID | QVKLEESGGGSVQTGGSLRLTCAASGRTSRSYGMGWFRQAPGKEREFVS |
| NO: 377 | GISWRGDSTGYADSVKGRFTISRDNAKNTVDLQMNSLKPEDTAIYYCAA |
| | AAGSAWYGTLYEYDYWGQGTQVTVSSGTGSPVPSTPPTPSPSTPPTPSPS |
| | ASEPKSCDKTHTCPPCPAPEAAGGPSVFLFPPKPKDTLMISRTPEVTCVVV |
| | DVSHEDPEVKFNWYVDGVEVHNAKTKPREEQYNSTYRVVSVLTVLHQD |
| | WLNGKEYKCKVSNKALGAPIEKTISKAKGQPREPQVYTLPPSRDELTKN |
| | QVSLSCAVKGFYPSDIAVEWESNGQPENNYKTTPPVLDSDGSFFLVSKLT |
| | VDKSRWQQGNVFSCSVMHEALHNHYTQKSLSLSPGKGGGGSGGGGSG |
| | GGGSREGCASRCMKYNDELEKCEARMMSMSNTEEDCEQELEDLLYCLD |
| | HCHSQ |
| SEQ ID | QVKLEESGGGSVQTGGSLRLTCAASGHTSRSYGMGWFRQAPGKEREFV |
| NO: 379 | SGISWRGDSTGYADSVKGRFTISRDNAKNTVDLQMNSLKPEDTAIYYCA |
| | AAAGSAWYHTLYEYDYWGQGTQVTVSSGTGGGGSGGGGSGGGGSGG |
| | GGSLKEAKEKAIEELKKAGITSDYYFDLINKAKTVEGVNALKDEILKAGG |
| | GGSGGGGSGGGGSGGGGSREGCASRCMKYNDELEKCEARMMSMSNTE |
| | EDCEQELEDLLYCLDHCHSQ |
| SEQ ID | QVKLEESGGGSVQTGGSLRLTCAASGHTSRSYGMGWFRQAPGKEREFV |
| NO: 380 | SGISWRGDSTGYADSVKGRFTISRDNAKNTVDLQMNSLKPEDTAIYYCA |
| | AAAGSAWYHTLYEYDYWGQGTQVTVSSGTGGGGSGGGGSGGGGSGG |
| | GGSLKEAKEKAIEELKKAGITSDYYFDLINKAKAVEGVNALKDEILKAG |
| | GGGSGGGGSGGGGSGGGGSREGCASRCMKYNDELEKCEARMMSMSNT |
| | EEDCEQELEDLLYCLDHCHSQ |
| SEQ ID | QVKLEESGGGSVQTGGSLRLTCAASGHTSRSYGMGWFRQAPGKEREFV |
| NO: 381 | SGISWRGDSTGYADSVKGRFTISRDNAKNTVDLQMNSLKPEDTAIYYCA |
| | AAAGSAWYHTLYEYDYWGQGTQVTVSSGTGGGGSGGGGSGGGGSGG |
| | GGSLKEAKEKAIEELKKAGITSDYYFDLINKAKAVEGVNALKDEILKAG |
| | GGGSGGGGSGGGGSGGGGSREGCASHCTKYKAELEKCEARVSSRSNTE |
| | ETCVQELFDFLHCVDHCVSQ |
| SEQ ID | QVKLEESGGGSVQTGGSLRLTCAASGHTSRSYGMGWFRQAPGKEREFV |
| NO: 383 | SGISWRGDSTGYADSVKGRFTISRDNAKNTVDLQMNSLKPEDTAIYYCA |
| | AAAGSAWYHTLYEYDYWGQGTQVTVSSGTGGGGSGGGGSGGGGSGG |
| | GGSEPKSCDKTHTCPPCPAPEAAGGPSVFLFPPKPKDTLMISRTPEVTCVV |
| | VDVSHEDPEVKFNWYVDGVEVHNAKTKPREEQYNSTYRVVSVLTVLHQ |
| | DWLNGKEYKCKVSNKALGAPIEKTISKAKGQPREPQVYTLPPSRDELTK |
| | NQVSLSCAVKGFYPSDIAVEWESNGQPENNYKTTPPVLDSDGSFFLVSKL |
| | TVDKSRWQQGNVFSCSVMHEALHNHYTQKSLSLSPGKGGGGSGGGGSG |
| | GGGSREGCASRCMKYNDELEKCEARMMSMSNTEEDCEQELEDLLYCLD |
| | HCHSQ |
| SEQ ID | QVKLEESGGGSVQTGGSLRLTCAASGHTSRSYGMGWFRQAPGKEREFV |
| NO: 385 | SGISWRGDSTGYADSVKGRFTISRDNAKNTVDLQMNSLKPEDTAIYYCA |
| | AAAGSAWYHTLYEYDYWGQGTQVTVSSGTGSPVPSTPPTPSPSTPPTPSP |
| | SASLKEAKEKAIEELKKAGITSDYYFDLINKAKTVEGVNALKDEILKAGS |
| | PVPSTPPTPSPSTPPTPSPSASREGCASRCMKYNDELEKCEARMMSMSNT |
| | EEDCEQELEDLLYCLDHCHSQ |
| SEQ ID | QVKLEESGGGSVQTGGSLRLTCAASGHTSRSYGMGWFRQAPGKEREFV |
| NO: 386 | SGISWRGDSTGYADSVKGRFTISRDNAKNTVDLQMNSLKPEDTAIYYCA |
| | AAAGSAWYHTLYEYDYWGQGTQVTVSSGTGSPVPSTPPTPSPSTPPTPSP |
| | SASLKEAKEKAIEELKKAGITSDYYFDLINKAKAVEGVNALKDEILKAGS |
| | PVPSTPPTPSPSTPPTPSPSASREGCASRCMKYNDELEKCEARMMSMSNT |
| | EEDCEQELEDLLYCLDHCHSQ |
| SEQ ID | QVKLEESGGGSVQTGGSLRLTCAASGHTSRSYGMGWFRQAPGKEREFV |
| NO: 388 | SGISWRGDSTGYADSVKGRFTISRDNAKNTVDLQMNSLKPEDTAIYYCA |
| | AAAGSAWYHTLYEYDYWGQGTQVTVSSGTGSPVPSTPPTPSPSTPPTPSP |
| | SASEPKSCDKTHTCPPCPAPEAAGGPSVFLFPPKPKDTLMISRTPEVTCVV |
| | VDVSHEDPEVKFNWYVDGVEVHNAKTKPREEQYNSTYRVVSVLTVLHQ |
| | DWLNGKEYKCKVSNKALGAPIEKTISKAKGQPREPQVYTLPPSRDELTK |
| | NQVSLSCAVKGFYPSDIAVEWESNGQPENNYKTTPPVLDSDGSFFLVSKL |
| | TVDKSRWQQGNVFSCSVMHEALHNHYTQKSLSLSPGKGGGGSGGGGSG |
| | GGGSREGCASRCMKYNDELEKCEARMMSMSNTEEDCEQELEDLLYCLD |
| | HCHSQ |
| SEQ ID | QVKLEESGGGSVQTGGSLRLTCAASGHTSRSYGMHWFRQAPGKEREFV |
| NO: 390 | SGISWRGDSTGYADSVKGRFTISRDNAKNTVDLQMNSLKPEDTAIYYCA |
| | AAAGSHWYGTHYEYDYWGQGTQVTVSSGTGGGGSGGGGSGGGGSGG |
| | GGSLKEAKEKAIEELKKAGITSDYYFDLINKAKAVEGVNALKDEILKAG |
| | GGGSGGGGSGGGGSGGGGSREGCASHCTKYKAELEKCEARVSSRSNTE |
| | ETCVQELFDFLHCVDHCVSQ |

### Example 23

### Cooperative Binding of Selective Depletion Complexes for Cell-Specific Targeting

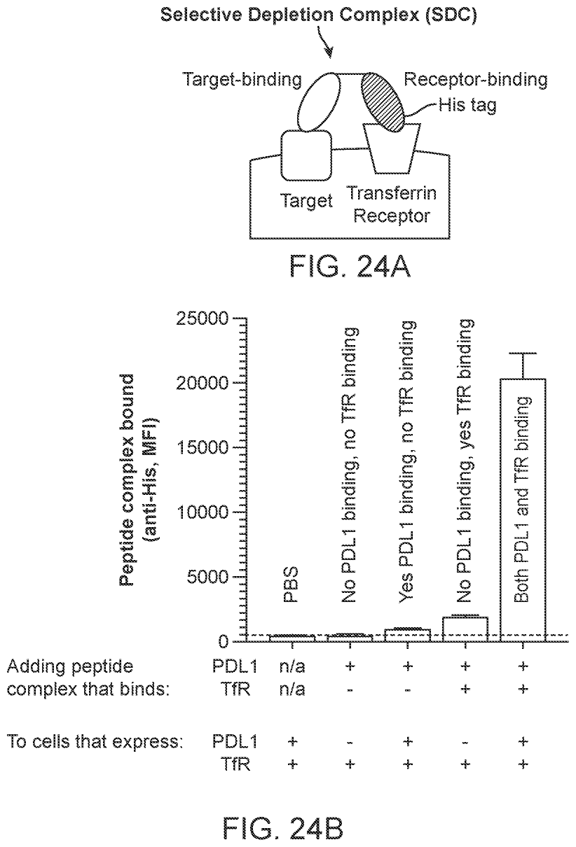

This example describes cooperative binding of selective depletion complexes for cell-specific targeting. Selective depletion complexes (SDCs) containing a target-binding peptide and a receptor-binding peptide and labeled with a His tag (SEQ ID NO: 228), as illustrated in FIG. 24A, as well as a control peptide that does not contain a high-affinity target-binding moiety but that does contain receptor-binding moiety and a His-tag, were tested for the ability to cooperatively bind to a target molecule and a receptor on a cell surface. Cells expressing TfR (PDL1 −, TfR +) or both PD-L1 and TfR (PDL1 +, TfR +) were incubated with peptide complexes capable of binding PD-L1 (PDL1 +, TfR −) both PD-L1 and TfR (PDL1 +, TfR +), or no peptide (PBS; PDL1 −, TfR −) and labeled with a fluorescent anti-His antibody. Fluorescence was used as a readout to measure binding of peptide complexes to cells. The SDC capable of binding both PD-L1 and TfR (SEQ ID NO: 356) cooperatively bound to cells expressing both PD-L1 and TfR, as indicated by high fluorescence shown in FIG. 24B. The same SDC showed significant but substantially lower binding to cells that expressed TfR but not PD-L1, as indicates by moderate fluorescence shown in FIG. 24B. Peptide complexes lacking the ability to bind TfR with high affinity but containing a PD-L1-binding domain (SEQ ID NO: 357) showed low binding to cells expressing TfR and PD-L1. Together, this data demonstrates that selective depletion complexes containing both a functional target-binding domain (e.g., a PD-L1-binding peptide) and a functional receptor-binding domain (e.g., a TfR-binding peptide) cooperatively bind to cells expressing both the target molecule and the receptor. The data also indicates that SDCs that contain a functional receptor-binding domain (e.g., a TfR-binding peptide), and a domain that binds a target that is not expressed on the cell surface (such as a soluble target), binds to cells that express the receptor.

### Example 24

### Designing Selective Depletion Complexes

This example describes designing selective depletion complexes to bind to and deplete a target molecule. Selective depletion complexes containing a target-binding peptide and a receptor-binding peptide are designed deplete a target molecule by binding to a receptor that is recycled via the endocytic pathway (e.g., TfR or PD-L1) and also binding the target molecule (e.g., PD-L1 or EGFR). One of the binding peptides in the SDC exhibits pH dependent binding (that is, higher binding to the target or receptor at extracellular pH than at endosomal/lysosomal pH). If the receptor is bound pH-independently (such as with similar binding at extracellular pH as at endosomal or lysosomal pH) and the target is bound with pH dependence, the SDC may be catalytic. If the receptor is bound with pH dependence and the target is bound with pH independence, the SDC may be non-catalytic. The receptor-binding peptide is complexed with the target-binding peptide by direct fusion through a linker or by dimerization through a dimerization domain. Examples of selective depletion complexes and comparator molecules are shown in FIG. 25A and FIG. 25B. The selective depletion complexes and complex components are assembled by connecting a target-binding peptide to a receptor-binding peptide through a linker or a dimerization domain. Examples of receptor-binding peptides include a TfR-binding CDP (SEQ ID NO: 96, “T”) or a TfR-binding single chain antibody (SEQ ID NO: 221, “N5”; or SEQ ID NO: 222, “M16”). SEQ ID NO: 96 and SEQ ID NO: 221 can bind TfR with pH independence and SEQ ID NO: 222 can bind TfR with pH dependence. Examples of target-binding peptides include an EGFR-binding nanobody (SEQ ID NO: 242, “G2”) with limited pH dependence, a pH-dependent EGFR-binding nanobody (SEQ ID NO: 243; “P”), a PD-L1-binding CDP with moderate pH dependence (SEQ ID NO: 187; solid dark circle), or a PD-L1-binding CDP with extreme pH dependence (SEQ ID NO: 233; solid light circle). Peptide linkers (e.g., SEQ ID NO: 129-SEQ ID NO: 141, SEQ ID NO: 194-SEQ ID NO: 218, SEQ ID NO: 223-SEQ ID NO: 227, or SEQ ID NO: 391) or dimerization domains (e.g., SEQ ID NO: 245-SEQ ID NO: 287) are used to link the target-binding peptide to the receptor-binding peptide in a single polypeptide chain, as seen in the first row of complexes, or to link the target-binding peptide or the receptor-binding peptide to a dimerization domain, as seen in the second row of complexes in FIG. 25A.

The dimerization domain can be an Fc homodimerization domain (e.g., any of SEQ ID NO: 245-SEQ ID NO: 259) or a knob-in-hole (KIH) Fc heterodimerization domain (e.g., SEQ ID NO: 260-SEQ ID NO: 287). To form heterodimers, SEQ ID NO: 260 dimerizes with SEQ ID NO: 261; SEQ ID NO: 262 dimerizes with SEQ ID NO: 263; SEQ ID NO: 264 dimerizes with SEQ ID NO: 265; SEQ ID NO: 266 dimerizes with SEQ ID NO: 267; SEQ ID NO: 268 dimerizes with SEQ ID NO: 269; SEQ ID NO: 270 dimerizes with SEQ ID NO: 271; SEQ ID NO: 272 dimerizes with SEQ ID NO: 273; SEQ ID NO: 274 dimerizes with SEQ ID NO: 275; SEQ ID NO: 276 dimerizes with SEQ ID NO: 277; SEQ ID NO: 278 dimerizes with SEQ ID NO: 279; SEQ ID NO: 280 dimerizes with SEQ ID NO: 281; SEQ ID NO: 282 dimerizes with SEQ ID NO: 283; SEQ ID NO: 284 dimerizes with SEQ ID NO: 285; and SEQ ID NO: 286 dimerizes with SEQ ID NO: 287. Components are mixed and matched to produce the preferred target-binding, receptor-binding, and valency properties.

Examples of monovalent selective depletion complexes are shown in FIG. 25A. Monovalent selective depletion complexes are designed as single polypeptide chains containing a target-binding peptide (e.g., SEQ ID NO: 242, “G2”; SEQ ID NO: 243, “P”; SEQ ID NO: 187, solid dark circle; or SEQ ID NO: 233, solid light circle) linked to a receptor-binding peptide (e.g., SEQ ID NO: 96, “T”; SEQ ID NO: 221, “N5”; or SEQ ID NO: 222, “M16”) via a linker (e.g., SEQ ID NO: 129-SEQ ID NO: 141, SEQ ID NO: 194-SEQ ID NO: 218, SEQ ID NO: 223-SEQ ID NO: 227, or SEQ ID NO: 391). Alternatively, monovalent selective depletion complexes are designed as a polypeptide containing a target-binding peptide heterodimerized with a polypeptide containing a receptor-binding polypeptide via complementary heterodimerization domains (e.g., a KIH heterodimerization pair selected from SEQ ID NO: 260-SEQ ID NO: 287).

The target:SDC:receptor complex is trafficked to the endosome, where the pH is progressively lowered (such as in an early endosome, late endosome, and lysosome). At the lower pH, a pH-dependent binding end of the SDC may no longer bind to the target or receptor. Representative catalytic active molecules may bind to TfR in a pH-independent fashion (e.g., using SEQ ID NO: 96 or SEQ ID NO: 221) and to the target in a pH-dependent fashion (e.g., using SEQ ID NO: 243, SEQ ID NO: 187, SEQ ID NO: 233, or SEQ ID NO: 234) and will therefore remain bound to TfR but release target in the low-pH endosome. The target may be trafficked to a lysosome and degraded. TfR is recycled back to the cell surface, bringing the catalytic active SDC molecule with it. Non-catalytic active molecules may bind to TfR in a pH-dependent fashion (e.g., using SEQ ID NO: 222) and to the target in a pH-independent fashion (e.g., using SEQ ID NO: 242) and will therefore release from TfR and remain bound to the target in low pH conditions. The target and the SDC may then both be subject to endosomal/lysosomal degradation. Control molecules may be designed whose binding to both TfR (e.g., using SEQ ID NO: 96 or SEQ ID NO: 221) and to target (e.g., using SEQ ID NO: 242) is pH-independent, and these molecules may not release target in the low pH endosome and therefore may not facilitate target degradation.

Examples of bivalent selective depletion complexes are shown in FIG. 25B. These examples only show one example TfR-binding moiety (SEQ ID NO: 96) and one example target-binding moiety (SEQ ID NO: 243), but the concept may be applied to any TfR-binding moiety or any target-binding moiety and may be subject to the same expectations for catalytic activity, non-catalytic activity, or comparator complex behavior based on pH-dependence as demonstrated in FIG. 25A. A bivalent selective depletion complex contains one or two target-binding peptides and one or two receptor-binding peptides. Bivalent selective depletion complexes are designed as homodimers or heterodimers containing one or more target-binding peptides (e.g., SEQ ID NO: 243, “P”; or SEQ ID NO: 242, SEQ ID NO: 187, SEQ ID NO: 233, or SEQ ID NO: 234 (not shown)) linked to one or more receptor-binding peptide (e.g., SEQ ID NO: 96, “T”; or SEQ ID NO: 221, “or SEQ ID NO: 222, (not shown)) through one or more linkers (e.g., SEQ ID NO: 129-SEQ ID NO: 141, SEQ ID NO: 194-SEQ ID NO: 218, SEQ ID NO: 223-SEQ ID NO: 227, or SEQ ID NO: 391) and/or a homodimerization domain (e.g., any of SEQ ID NO: 245-SEQ ID NO: 259) or a heterodimerization domain pair (e.g., a KIH heterodimerization pair selected from SEQ ID NO: 260-SEQ ID NO: 287). Alternatively, bivalent selective depletion complexes are designed as a single polypeptide chain containing one or two target-binding peptides and one or two receptor-binding peptides connected via a linker. Higher valence SDCs can also be designed. SDCs that are bivalent or multivalent may exhibit increased binding due to cooperativity, which may increase the potency or function of the molecule for target protein degradation. For example, if the receptor-binding peptide has a fairly rapid off rate, an SDC that is bivalent for binding the receptor may increase the ability of the SDC to bind the cell and may also increase the ability of the SDC to remain bound to the receptor during the trafficking of the receptor back to the cell surface. An SDC can have 1, 2, 3, 4, 5 or more target-binding peptides and 1, 2, 3, 4, 5 or more receptor-binding peptides. An IgM, polymer, or dendritic scaffold may be used to multimerize the SDC.

While selective depletion complexes in FIG. 25A and FIG. 25B are shown with the receptor-binding peptide positioned toward the N-terminus of the complex and the target-binding peptide positioned toward the C-terminus, complexes may be arranged with the target-binding peptide toward the N-terminus and the receptor-binding peptide toward the C-terminus. Additionally, selective depletion complexes may be designed as multivalent complexes containing three or more target-binding peptides and/or three or more receptor-binding peptides.

### Example 25

### Selective Depletion of PD-L1 Using a Selective Depletion Complex

This example describes selective depletion of PD-L1 using a selective depletion complex. A selective depletion complex containing a pH-dependent PD-L1-binding peptide of SEQ ID NO: 233 or SEQ ID NO: 234 and a TfR-binding peptide of SEQ ID NO: 96 or SEQ ID NO: 221 is contacted to a cell expressing TfR and PD-L1. The selective depletion complex cooperatively binds to TfR via the TfR-binding peptide and to PD-L1 via the PD-L1-binding peptide, forming a ternary complex on the cell surface. The TfR is endocytosed along with the bound selective depletion complex and PD-L1. Upon acidification during endosomal/lysosomal maturation, the selective depletion complex releases the PD-L1 and remains bound to the TfR. The PD-L1 is degraded in the lysosome, thereby selectively depleting the PD-L1. The TfR and selective depletion complex are recycled to the cell surface.

The selective depletion complex is SEQ ID NO: 290, SEQ ID NO: 291, SEQ ID NO: 308, SEQ ID NO: 317, SEQ ID NO: 318, SEQ ID NO: 322, SEQ ID NO: 323; or the selective depletion complex is SEQ ID NO: 292, SEQ ID NO: 294, SEQ ID NO: 315, SEQ ID NO: 316, heterodimerized with SEQ ID NO: 304, SEQ ID NO: 306, SEQ ID NO: 319, SEQ ID NO: 320, SEQ ID NO: 321, SEQ ID NO: 324, or SEQ ID NO: 325; or the selective depletion complex is SEQ ID NO: 295 or SEQ ID NO: 297, heterodimerized with SEQ ID NO: 304, SEQ ID NO: 319, SEQ ID NO: 321, or SEQ ID NO: 324; or the selective depletion complex is SEQ ID NO: 298 or SEQ ID NO: 300, heterodimerized with SEQ ID NO: 303; or the selective depletion complex is SEQ ID NO: 326, heterodimerized with SEQ ID NO: 306, SEQ ID NO: 311, SEQ ID NO: 320 or SEQ ID NO: 325.

### Example 26

### Treatment of Cancer by Selectively Depleting PD-L1

This example describes treatment of cancer by selectively depleting PD-L1. A selective depletion complex containing a pH-dependent PD-L1-binding peptide of SEQ ID NO: 233 or SEQ ID NO: 234 and a TfR-binding peptide, such as that of SEQ ID NO: 96 or SEQ ID NO: 221, is administered to a subject having a PD-L1 positive cancer. The selective depletion complex binds to PD-L1 and TfR on the surface of a cancer cell, and the ternary complex of the selective depletion complex, PD-L1, and TfR is endocytosed. The PD-L1 is released upon acidification in the endosome and degraded, thereby depleting PD-L1. The TfR and selective depletion complex are recycled to the cell surface. Depletion of PD-L1 inhibits evasion of the host immune response by the cancer cell and increases apoptosis of the cancer cell, thereby treating the cancer.

### Example 27

### Selective Depletion of EGFR Using a Selective Depletion Complex

This example describes selective depletion of EGFR using a selective depletion complex. A selective depletion complex containing a pH-dependent EGFR-binding peptide of SEQ ID NO: 242, SEQ ID NO: 243 or SEQ ID NO: 244 and a TfR-binding peptide of SEQ ID NO: 96, SEQ ID NO: 221, or SEQ ID NO: 222 in any combination of TfR-binding valence (e.g. monovalent, bivalent, or greater) and EGFR-binding valence (e.g. monovalent, bivalent, or greater) is contacted to a cell expressing TfR and EGFR. The selective depletion complex cooperatively binds to TfR via the TfR-binding peptide and to EGFR via the EGFR-binding peptide, forming a ternary complex on the cell surface. The TfR is endocytosed along with the bound selective depletion complex and EGFR. Upon acidification in the endosome, the selective depletion complex releases the EGFR and remains bound to the TfR. The EGFR is degraded in the endosome/lysosome, thereby selectively depleting the EGFR. The TfR and selective depletion complex are recycled to the cell surface. Activation of EGFR and downstream pathways such as KRAS and MEK may be reduced. Binding to the cell surface, endosomal uptake, trafficking, degradation, and pathway activation can be detected using flow cytometry, fluorescent microscopy, Western blotting, ELISA, histology, IHC, or other methods. These can be monitored after in vitro or in vivo exposure of EGFR-expressing cells.

The selective depletion complex is SEQ ID NO: 288, SEQ ID NO: 289, SEQ ID NO: 307, SEQ ID NO: 313, SEQ ID NO: 327, SEQ ID NO: 328, SEQ ID NO: 332, SEQ ID NO: 333, SEQ ID NO: 337, SEQ ID NO: 338, SEQ ID NO: 342, or SEQ ID NO: 343; or the selective depletion complex is SEQ ID NO: 292, SEQ ID NO: 293, SEQ ID NO: 310, SEQ ID NO: 315, SEQ ID NO: 316 heterodimerized with SEQ ID NO: 302; SEQ ID NO: 305, SEQ ID NO: 339, SEQ ID NO: 340; SEQ ID NO: 344; SEQ ID NO: 345; or the selective depletion complex is SEQ ID NO: 296 heterodimerized with SEQ ID NO: 302, SEQ ID NO: 339, or SEQ ID NO: 344; or the selective depletion complex is SEQ ID NO: 298 or SEQ ID NO: 299 heterodimerized with SEQ ID NO: 301; or the selective depletion complex is SEQ ID NO: 331 or SEQ ID NO: 336 heterodimerized with SEQ ID NO: 330 or SEQ ID NO: 335; or the selective depletion complex is SEQ ID NO: 292, SEQ ID NO: 315, or SEQ ID NO: 316 heterodimerized with SEQ ID NO: 329, SEQ ID NO: 330, SEQ ID NO: 334, or SEQ ID NO: 335.

Similar methods and designs can be used for selective depletion complexes containing a PD-L1-binding domain (e.g., a PD-L1-binding peptide) rather than a TfR-binding domain the as the recycling receptor, in methods where PD-L1 is used as the recycling receptor. For Example, EGFR can similarly be depleted as described by use of a selective depletion complex that comprises a PD-L1-binding moiety and an EGFR-binding moiety where at least one moiety binds with a lower affinity at endosomal pH than at extracellular pH.

### Example 28

### Treatment of Cancer by Selectively Depleting EGFR

This example describes treatment of cancer by selectively depleting EGFR. A selective depletion complex containing a pH-dependent EGFR-binding peptide, such as that of SEQ ID NO: 243 or SEQ ID NO: 244, and a TfR-binding peptide, such as that of SEQ ID NO: 96, is administered to a subject having an EGFR positive cancer. The subject may be a human, a non-human primate, a mouse, a rat, or another species. A selective depletion complex may be administered subcutaneously, intravenously, intramuscularly, interperitoneally, or by another route. It may be administered 1, 2, 3, 4, 5, 10, or more times and at a frequency of 1, 2, 3, 4, 5, 6, or 7 times per week or every other week or every third week, or monthly or less frequently. The EGFR positive cancer is non-small-cell lung cancer, head and neck cancer, glioblastoma, metastatic brain cancer, colorectal cancer, TKI-resistant cancer, cetuximab-resistant cancer, necitumumab-resistant cancer, or panitumumab-resistant cancer. The selective depletion complex binds to EGFR and TfR on the surface of an EGFR positive cancer cell, and the ternary complex of the selective depletion complex, EGFR, and TfR is endocytosed. The EGFR is released upon acidification in the endosome and degraded, thereby depleting EGFR. The TfR and selective depletion complex are recycled to the cell surface. Depletion of EGFR reduces pro-growth signaling in the cancer cell, slowing cancer growth or metastases, thereby treating the cancer. Optionally, because the selective depletion complex targets cells that express both EGFR and TfR, the skin toxicity caused by the selective complex is less than the skin toxicity caused by anti-EGFR antibody or tyrosine kinase inhibitor therapy, which inhibit EGFR without TfR tissue targeting.

Cancers may also be similarly treated by using a selective depletion complex that binds both PD-L1 and EGFR.

### Example 29

### Selective Depletion of TNFα Using a Selective Depletion Complex

This example describes selective depletion of TNFα using a selective depletion complex. A selective depletion complex containing a pH-dependent TNFα-binding peptide of and a TfR-binding peptide, such as that of SEQ ID NO: 96, is contacted to a cell expressing TfR where there is TNFα present, such as in the extracellular fluid, serum, on the cell surface, or in the cell culture media. The selective depletion complex cooperatively binds to TfR via the TfR-binding peptide and to TNFα via the TNFα-binding peptide, forming a ternary complex on the cell surface. The TfR is endocytosed along with the bound selective depletion complex and TNFα. Upon acidification in the endosome, the selective depletion complex releases the TNFα and remains bound to the TfR. The TNFα is degraded in the endosome/lysosome, thereby selectively depleting the TNFα. The TfR and selective depletion complex are recycled to the cell surface.

### Example 30

### Treatment of a CNS Inflammatory Disorder by Selectively Depleting TNFα

This example describes treatment of a CNS inflammatory disorder by selectively depleting TNFα. A selective depletion complex containing a pH-dependent TNFα-binding peptide and a TfR-binding peptide of SEQ ID NO: 96 is administered to a subject having a disorder in the CNS that involves inflammation. The CNS inflammatory disorder is optionally neuroinflammation, stroke, traumatic brain injury, Alzheimer's disease, or a tauopathy. The SDC crosses the BBB, thereby contacting cells and molecules within the subjects CNS. The SDC may be transported across the BBB via binding transferrin and undergoing transcytosis. The selective depletion complex binds to TfR on the surface of a cell and also to TNFα, and the ternary complex of the selective depletion complex, TNFα, and TfR is endocytosed. The TNFα is released upon acidification in the endosome and degraded, thereby depleting TNFα. The TfR and selective depletion complex are recycled to the cell surface. Depletion of TNFα reduces cytokine signaling in the CNS, reducing neuroinflammation, thereby treating the CNS inflammatory disorder.

### Example 31

### Selective Depletion of CD47 Using a Selective Depletion Complex

This example describes selective depletion of CD47 using a selective depletion complex. A selective depletion complex containing a TfR-binding peptide and a CD47-binding peptide, where one of the binding peptides is pH-dependent in its binding and the other binding peptide is pH-independent in its binding, is contacted to a cell expressing TfR and CD47. The selective depletion complex cooperatively binds to TfR via the TfR-binding peptide and to CD47 via the CD47-binding peptide, forming a ternary complex on the cell surface. The TfR is endocytosed along with the bound selective depletion complex and CD47. Upon acidification in the endosome, the selective depletion complex releases the CD47 and remains bound to the TfR or the SDC released the TfR and remains bound to the CD47. The CD47 is degraded in the endosome/lysosome, thereby selectively depleting the CD47.

### Example 32

### Treatment of Cancer by Selectively Depleting CD47

This example describes treatment of cancer by selectively depleting CD47. A selective depletion complex containing a TfR-binding peptide and a CD47-binding peptide, such as that described in EXAMPLE 31, is administered to a subject having a CD47 positive cancer. The selective depletion complex binds at low or no amount to mature red blood cells because mature red blood cells do not express TfR, resulting in preferential binding to cancer cells as compared to red blood cells. The selective depletion complex binds to CD47 and TfR on the surface of a cancer cell, and the ternary complex formed from the selective depletion complex, CD47, and TfR is endocytosed. The CD47 is trafficked to the endosome/lysosome and degraded, thereby depleting CD47. The cell is depleted of CD47, eliminating an immuno-suppressive or anti-apoptotic signal from the cell. Depletion of CD47 inhibits evasion of the host immune response by the cancer cell and allows response to various pro-apoptotic signals which increase immune cell attack of or apoptosis of the cancer cell, thereby treating the cancer.

Treatment of a cancer in a first subject with a selective depletion complex that targets and depletes CD47 is compared to treatment of a cancer in a second subject by administering an antibody that binds CD47. The antibody binds to all cells that expressed CD47, including red blood cells. Aging red blood cells in the second subject treated with the anti-CD47 antibody also display pro-apoptotic signals, thus once the CD47 is depleted from the aging red blood cell surface, the aging red blood cells are targeted and removed by the immune system, and the second subject is depleted of red blood cells and becomes anemic. Since the selective depletion complex does not bind to red blood cells, the CD47 is not reduced on the red blood cells of the first subject. Thus, the first subject that is treated with the selective depletion complex of this example does not develop anemia.

### Example 33

### Treatment of Cancer by Selectively Depleting CD39

This example describes treatment of cancer by selectively depleting CD39. CD39 is a cell surface ectoenzyme that degrades ATP to AMP; CD73 then processes AMP to adenosine, which is immunosuppressive, whereas ATP can activate macrophages to secrete IL-1ß and IL-18 which activate T cells. A selective depletion complex (SDC) containing a TfR-binding peptide and a CD39-binding peptide is administered to a subject having a CD39 positive cancer. The SDC causes removal of CD39 from the cell surface. The cell is depleted of CD39, thereby inhibiting conversion of ATP to AMP. The resulting tumor microenvironment contains more ATP and less adenosine than the tumor microenvironment prior to treatment with the SDC. The tumor microenvironment becomes more inflammatory and less immunosuppressed, leading to enhanced targeting of the cancer cells by the immune system and for apoptosis, thereby treating the cancer. The SDC causes the CD39 to be removed from the cell surface at a rate much faster than the rate of regeneration of CD39, leading to extended depletion of CD39 and sustained reduction of ATP processing to adenosine.

Treatment of a cancer in a first subject with a selective depletion complex that targets and depletes CD39 is compared to treatment of a cancer in a second subject by administering an antibody that binds CD39. The concentration of antibody in the second subject's circulation varies over the dosing intervals such that CD39 is not fully occupied by the antibody at all times. Low occupancy of CD39 by the anti-CD39 antibody in the second subject results in less adenosine depletion in the second subject compared to the first subject treated with the SDC due to constant activity of the CD39 enzyme in the second subject. The antibody also binds to CD39 on the red blood cells of the second subject, causing anemia. The SDC does not deplete CD39 from red blood cells in the first subject because the mature red blood cells do not express TfR. As a result, the CD39 is not reduced on the red blood cells of the first subject. Thus, the first subject that is treated with the selective depletion complex of this example does not develop anemia.

### Example 34

### Selective Depletion of a Soluble Target Molecule Via PD-L1-Mediated Endocytosis

This example describes selective depletion of a soluble target molecule via PD-L1-mediated endocytosis. A selective depletion complex (SDC) containing a PD-L1-binding peptide (e.g., any one of SEQ ID NO: 187, SEQ ID NO: 236, SEQ ID NO: 400, or SEQ ID NO: 401) with PD-L1-binding at endosomal pH conjugated to a target-binding peptide is contacted to cells expressing PD-L1. The PD-L1-binding peptide binds PD-L1 at both physiologic extracellular pH (such as pH 7.4) and at endosomal pH (such as pH 5.5), and the target-binding peptide binds to a soluble target molecule with higher affinity at physiologic extracellular pH and with lower affinity at endosomal pH. Upon contact, the PD-L1-binding peptide binds to PD-L1 on the cell surface, and the target-binding peptide binds to the soluble target molecule in solution. The PD-L1-binding SDC undergoes the same recycling process illustrated in FIG. 12A, where “TfR-binding CDP (pH-independent)” and “TfR (Recycling)” are substituted with “PD-L1-binding CDP (pH-independent)” and “PD-L1 (Recycling),” respectively. The SDC binds to the soluble target molecule, as illustrated in FIG. 12A (1). The complex formed from the SDC, PD-L1, and the target molecule is endocytosed via PD-L1-mediated endocytosis as illustrated in FIG. 12A (2). As the endosomal compartment acidifies, the target molecule is released from the target-binding peptide, as illustrated in FIG. 12A (3). The target molecule is then degraded in a lysosomal compartment, as illustrated in FIG. 12A (4), and the complex is recycled to the cell surface along with the PD-L1, as illustrated in FIG. 12A (5).

### Example 35

### Selective Depletion of a Surface Target Molecule Via PD-L1-Mediated Endocytosis

This example describes selective depletion of a surface target molecule via PD-L1-mediated endocytosis. A selective depletion complex (SDC) containing a PD-L1-binding peptide (e.g., any one of any one of SEQ ID NO: 187, SEQ ID NO: 235-SEQ ID NO: 239, SEQ ID NO: 400, or SEQ ID NO: 401) with PD-L1-binding at endosomal pH conjugated to a target-binding peptide is contacted to cells expressing PD-L1. The PD-L1-binding peptide binds PD-L1 at both physiologic extracellular pH (such as at pH 7.4) and at endosomal pH (such as at pH 5.5), and the target-binding peptide binds to a surface target molecule with higher affinity at physiologic extracellular pH and with lower affinity at endosomal pH. Upon contact, the PD-L1-binding peptide binds to PD-L1 on the cell surface, and the target-binding peptide binds to the target molecule on the cell surface. The PD-L1-binding SDC undergoes the same recycling process illustrated in FIG. 12B, where “TfR-binding CDP (pH-independent)” and “TfR (Recycling)” are substituted with “PD-L1-binding CDP (pH-independent)” and “PD-L1 (Recycling),” respectively. The SDC binds to the cell surface target molecule, as illustrated in FIG. 12B (1). The complex formed from the SDC, PD-L1, and the target molecule is endocytosed via PD-L1-mediated endocytosis as illustrated in FIG. 12B (2). As the endosomal compartment acidifies, the target molecule is released from the target-binding peptide, as illustrated in FIG. 12B (3). The target molecule is then degraded in a lysosomal compartment, as illustrated in FIG. 12B (4), and the complex is recycled to the cell surface along with the PD-L1, as illustrated in FIG. 12B (5).

### Example 36

### Selective Depletion of HLA-G Using a PD-L1-Binding Selective Depletion Complex

This example describes selective depletion of HLA-G using a selective depletion complex that binds PD-L1. A selective depletion complex (SDC) containing a PD-L1-binding peptide and an HLA-G-binding peptide, is constructed. Optionally, PD-L1 binding peptide, such as a PD-L1-binding peptide of SEQ ID NO: 187, SEQ ID NO: 236, SEQ ID NO: 400, or SEQ ID NO: 401, binds PD-L1 at both extracellular and endosomal pH, and the HLA-G-binding peptide binds HLA-G with high affinity at extracellular pH and lower affinity at endosomal pH. Alternatively, the PD-L1 binding peptide, such as a PD-L1-binding peptide of SEQ ID NO: 233 or SEQ ID NO: 234, binds PD-L1 at extracellular pH and lower affinity at endosomal pH, and the HLA-G-binding peptide binds HLA-G with high affinity at both extracellular and endosomal pH. The SDC of this example is contacted to a cancer cell. The SDC binds PD-L1 and HLA-G expressed on the surface of the cancer cell, forming a ternary complex. The SDC is endocytosed along with the PD-L1 and the HLA-G. If the HLA-G-binding peptide binds HLA-G with high affinity at extracellular pH and lower affinity at endosomal pH, the SDC releases the HLA-G upon acidification of the endosome and the HLA-G is targeted to the endosomal/lysosomal system for degradation. If the PD-L1-binding peptide binds PD-L1 with high affinity at both extracellular and endosomal pH, the SDC is recycled back to the cell surface, where it may bind another HLA-G for degradation. If the PD-L1 binding peptide binds PD-L1 with high affinity at extracellular pH and lower affinity at endosomal pH, the SDC releases the PD-L1 upon acidification of the endosome, and the HLA-G and the SDC may be trafficked to the endosomal/lysosomal system. The PD-L1 may be recycled back to the cell surface.

### Example 37

### Structure of a High-Affinity PD-L1-Binding Cystine Dense Peptide

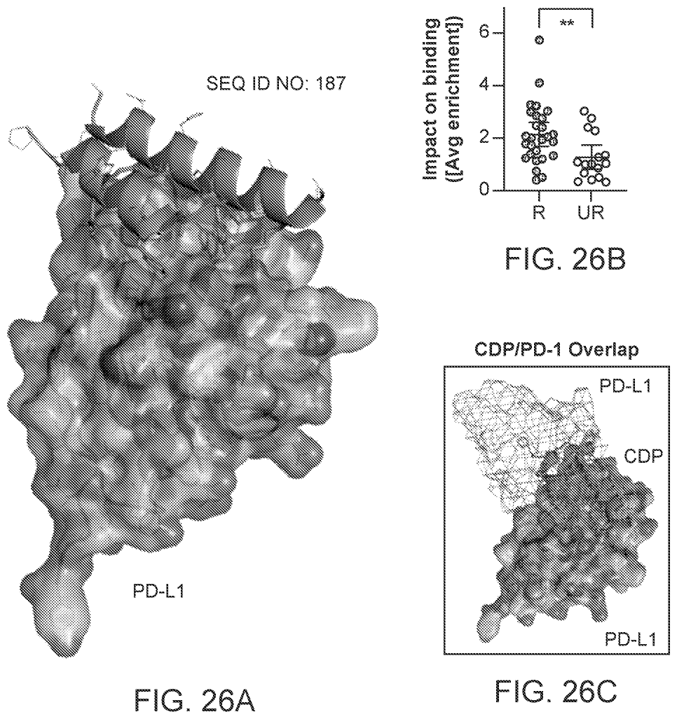

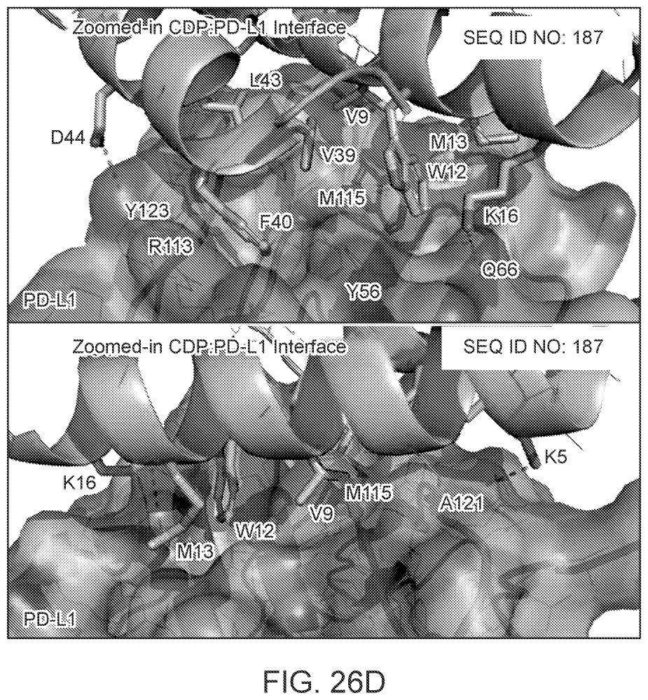

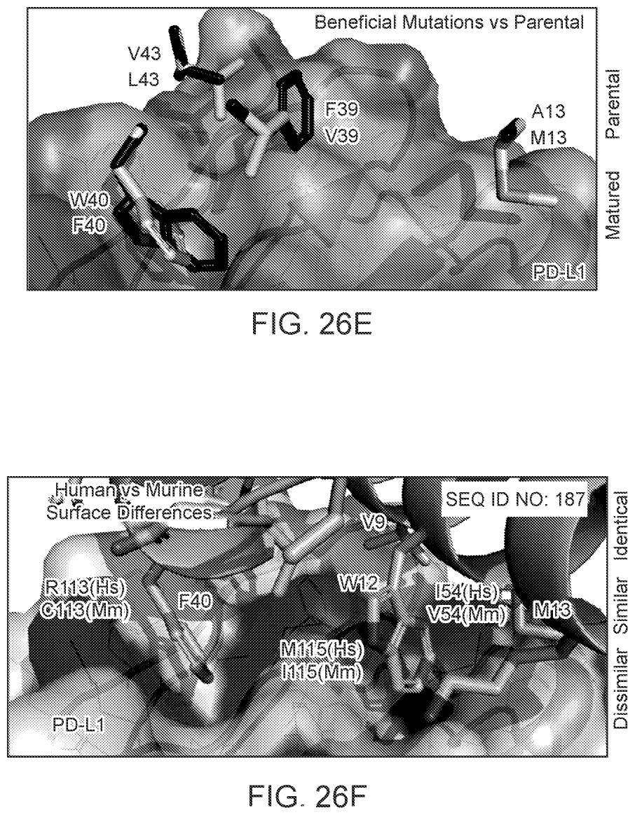

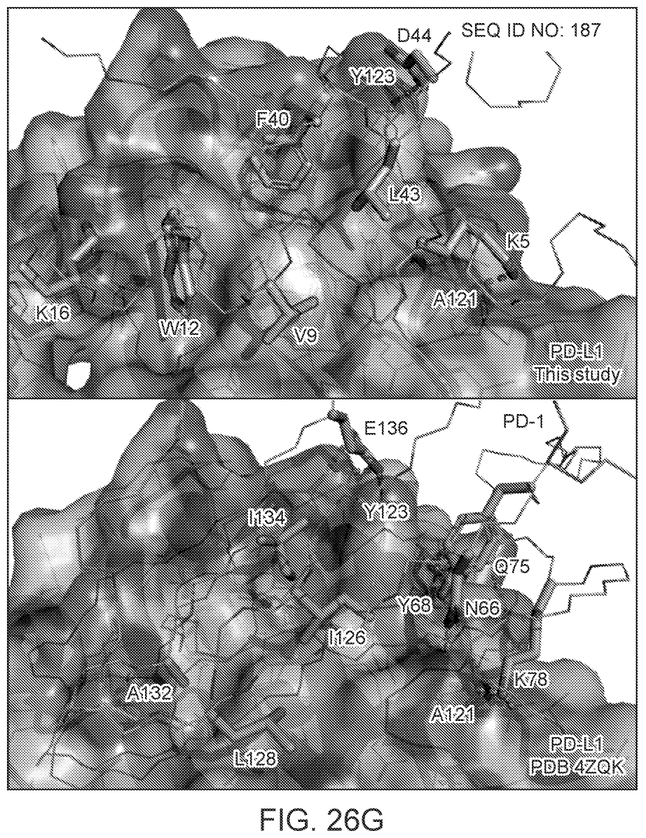

This example describes the structure of a high-affinity PD-L1-binding cystine dense peptide. A PD-L1-binding CDP (SEQ ID NO: 187) was co-crystalized with PD-L1 to confirm the CDP binding site and visualize the surface interactions with PD-L1, as shown in FIG. 26A. SEQ ID NO: 187, a variant that eliminated a canonical N-linked glycosite acquired during affinity maturation, was produced as a soluble molecule as described in EXAMPLE 1 and was co-crystalized with PD-L1. A portion of the CDP, from A19 through Q35, was unresolved in the 2.0 Å structure. Enrichment analysis was performed to determine the impact of amino acid substitutions at residues that were resolved in the crystal structure relative to residues that were unresolved in the crystal structure. The average SSM enrichment scores of unresolved residues were less extreme (deviated less from 0) than seen with resolved residues, as shown in FIG. 26B, showing the specific side chain identities of unresolved residues were less important to high affinity binding. The portion that did resolve matched the model from E1 through C18 and from D38 through A48. K36 and F37 resolved but were not part of the D38-A48 helix.

The resolved portion had an interface surface area of 620 Å² as assessed by PISA (PDBe PISA v1.52), which was similar to the observed interface surface area of PD-L1 with PD-1 (622 Å², PDB 4ZQK). The CDP's location on PD-L1 fell squarely within both the PD-1 occupancy space, as shown in FIG. 26C, showing the in silico low-resolution docking enrichment was predictive of the interface of this hit. A closer look at the interface, shown in FIG. 26D and in FIG. 26G, revealed that the CDP makes use of many of the same interaction sites as PD-1. Both K5 of SEQ ID NO: 187 and K78 of PD-1 made a salt bridge with the A121 backbone oxygen of PD-L1, while both D44 of SEQ ID NO: 187 and E136 of PD-1 similarly formed a salt bridge with Y123 of PD-L1. F40 of SEQ ID NO: 187 sat in pocket formed by Y56, R113, M115, and Y123 of PD-L1, making hydrophobic contacts (M115), herringbone ring stacking interactions (two Y's), and a cation-pi interaction (R113). This pocket was also occupied by I134 of PD-1. Furthermore, V9, W12, and L43 of SEQ ID NO: 187 also shared sites of hydrophobic interactions used by L128, A132, and I126 of PD-1, respectively. The interface-adjacent mutations that differentiated SEQ ID NO: 187 from its parental scaffold would be expected to disrupt binding when reverted to the parental side chains, as illustrated in FIG. 26E. The hydrophobic interactions of both M13 and L43 with the surface of PD-L1 would be lost in the parental A13 and V43; the pocket occupied by F40 would have to distort to accommodate the parental W40, altering the interface elsewhere; and parental F39 does not neatly fit against the surface as V39 does. Finally, analysis of the human/mouse and human/cynomolgus monkey (cyno) homology on the PD-L1 surface revealed that the interaction site contained several non-homologous side chains between human and mice, as shown in FIG. 26F.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

1-164. (canceled)

165. A peptide complex comprising:
a transferrin receptor-binding peptide; and
a target-binding peptide complexed with the transferrin receptor-binding peptide, wherein the target-binding peptide has a target-binding affinity for a target that is lower in an endosome than in an extracellular environment.

166. The peptide complex of claim 165, wherein the target is an extracellular protein, a secreted peptide, a secreted protein, a circulating protein, a soluble protein, a cell surface protein, or a transmembrane protein.

167. The peptide complex of claim 165, wherein the transferrin receptor-binding peptide, the target-binding peptide, or both comprises a miniprotein, a nanobody, an antibody, an antibody fragment, an scFv, a DARPin, or an affibody.

168. The peptide complex of claim 167, wherein the miniprotein comprises a cystine-dense peptide, an affitin, an adnectin, an avimer, a Kunitz domain, a nanofittin, a fynomer, a bicyclic peptide, a beta-hairpin, or a stapled peptide.

169. The peptide complex of claim 165, wherein the transferrin receptor-binding peptide comprises a cystine-dense peptide comprising at least three disulfide bonds.

170. The peptide complex of claim 165, wherein the transferrin receptor-binding peptide comprises a sequence of SEQ ID NO: 170.

171. The peptide complex of claim 165, wherein the transferrin receptor-binding peptide comprises a sequence having at least 90% sequence identity to any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64, or wherein the transferrin receptor-binding peptide comprises a sequence having at least 90% sequence identity to a fragment of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64.

172. The peptide complex of claim 165, wherein the transferrin receptor-binding peptide comprises a sequence of SEQ ID NO: 96, SEQ ID NO: 66, SEQ ID NO: 220, SEQ ID NO: 221, or SEQ ID NO: 222.

173. The peptide complex of claim 165, wherein the target-binding affinity of the target-binding peptide is at least 2-fold greater at pH 7.4 than at pH 5.5.

174. The peptide complex of claim 165, wherein the target-binding affinity of the target-binding peptide at pH 5.5 is lower than a transferrin receptor-binding affinity of the transferrin receptor-binding peptide for a transferrin receptor at pH 5.5.

175. The peptide complex of claim 165, wherein the target is a growth factor receptor, a receptor tyrosine kinase, a cell signaling molecule, an extracellular matrix macromolecule, a neurotransmitter, a cytokine, a growth factor, a tumor associated antigen, a tumor specific antigen, a hormone, a checkpoint inhibitor, an immune checkpoint inhibitor, an inhibitory immune receptor, a ligand of an inhibitory immune receptor, a macrophage surface protein, a lipopolysaccharide, an antibody, an inhibitory immune receptor, a tumor associated antigen, a tumor specific antigen, or an autoantibody.

176. The peptide complex of claim 165, wherein the target is collagen, elastin, a microfibrillar protein, a proteoglycan, CD200R, CD300a, CD300f, CEACAM1, FcgRiib, ILT-2, ILT-3, ILT-4, ILT-5, LAIR-1, PECAM-1, PILR-alpha, SIRL-1, and SIRP-alpha, CLEC4A, Ly49Q, MIC, CD3, CD47, CD28, CD137, CD89, CD14, CD16, CD29, CD44, CD71, CD73, CD90, CD105, CD166, CD27, CD39, CD24, CD25, CD74, CD40L, MUC1, MUC16, MUC2, MUCSAC, MUC4, OX40, 4-1BB, HLA-G, LAG3, Tim3, TIGIT, GITR, TCR, TNF-α, EGFR, EGFRvIII, TKI-resistant EGFR, HER2, ERBB3, PDGFR, FGF, VEGF, VEGFR, IGFR1, CTLA4, STRO1, complement factor C4, complement factor C1q, complement factor C1s, complement factor C1r, complement factor C3, complement factor C3a, complement factor C3b, complement factor C5, complement factor C5a, TGFβ, PCSK9, P2Y6, HER3, RANK, tau, amyloid β, huntingtin, α-synuclein, glucocerebrosidase, α-glucosidase, apolipoprotein E4, IL-1, IL-1R, IL-1α, IL-1β, IL-2, IL-2R, IL-4, IL-5, IL-6, IL-6R, IL-10, IL-10R, IL-17, IL-23, IL-12, p40, a member of the B7 family, c-Met, SIGLEC, MCP-1, an MHC, an MHC I, an MHC II, PD-1, or PD-L1.

177. The peptide complex of claim 165, wherein the target is EGFR, EGFRvIII, PD-L1, IL-1R, IL-1β, c-Met, FGFR-1, ERBB3, HER2, AXL receptor, CD44, IGF1R, MUC1, PDGFR, CD47, CD73, CD30, VEGFR, VEGFR2, complement factor C4, tau, amyloid ß, α-synuclein, PCSK9, or TNFα.

178. The peptide complex of claim 165, wherein the target-binding peptide comprises a sequence having at least 90% sequence identity to SEQ ID NO: 187, SEQ ID NO: 233, SEQ ID NO: 242, SEQ ID NO: 243, SEQ ID NO: 219, SEQ ID NO: 244, SEQ ID NO: 234, SEQ ID NO: 235-SEQ ID NO: 239, SEQ ID NO: 400-SEQ ID NO: 456, or SEQ ID NO: 240.

179. The peptide complex of claim 165, wherein the target-binding peptide comprises one or more histidine residues at a target-binding interface.

180. The peptide complex of claim 165, wherein the transferrin receptor-binding peptide and the target-binding peptide form a single polypeptide chain.

181. The peptide complex of claim 165, wherein the peptide complex comprises a dimer dimerized via a dimerization domain.

182. The peptide complex of claim 181, wherein the dimerization domain comprises an Fc domain, a homodimerization domain, a first heterodimerization domain, a second heterodimerization domain, or combinations thereof.

183. The peptide complex of claim 182, wherein the homodimerization domain comprises a sequence that has at least 90% sequence identity with any one of SEQ ID NO: 245-SEQ ID NO: 259.

184. The peptide complex of claim 182, wherein the first heterodimerization domain comprises a sequence that has at least 90% sequence identity with any one of SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, or SEQ ID NO: 286.

185. The peptide complex of claim 182, wherein the second heterodimerization domain comprises a sequence that has at least 90% sequence identity with any one of SEQ ID NO: 261, SEQ ID NO: 263, SEQ ID NO: 265, SEQ ID NO: 267, SEQ ID NO: 269, SEQ ID NO: 271, SEQ ID NO: 273, SEQ ID NO: 275, SEQ ID NO: 277, SEQ ID NO: 279, SEQ ID NO: 281, SEQ ID NO: 283, SEQ ID NO: 285, or SEQ ID NO: 287.

186. The peptide complex of claim 165, comprising a sequence that has at least 90% sequence identity with any one of SEQ ID NO: 288-SEQ ID NO: 313 or SEQ ID NO: 315-SEQ ID NO: 346.

187. A method of delivering a target molecule to an endosome, the method comprising:
contacting a peptide complex to a cell expressing a transferrin receptor, wherein the peptide complex comprises:
a transferrin receptor-binding peptide, and
a target-binding peptide complexed with the transferrin receptor-binding peptide;
binding the target-binding peptide to the target molecule under extracellular conditions;
binding the transferrin receptor-binding peptide to the transferrin receptor under extracellular conditions;
endocytosing the peptide complex, the target molecule, and the transferrin receptor; and
unbinding the target-binding peptide from the target molecule under endosomal conditions, thereby delivering the target molecule to the endosome.

188. The method of claim 187, wherein the target molecule is an extracellular protein, a secreted peptide, a secreted protein, a circulating protein, a soluble protein, a cell surface protein, or a transmembrane protein.

189. The method of claim 187, further comprising degrading the target molecule in an endocytic or lysosomal compartment.

190. The method of claim 187, further comprising recycling the peptide complex and the transferrin receptor.

191. The method of claim 189, further comprising depleting the target molecule.

192. A method of treating a disease or condition in a subject, the method comprising:
administering to the subject a peptide complex comprising a transferrin receptor-binding peptide and a target-binding peptide complexed with the transferrin receptor-binding peptide;
binding the target-binding peptide under extracellular conditions to a target molecule associated with the disease or condition on a cell of the subject expressing the target molecule and a transferrin receptor;
binding the transferrin receptor-binding peptide under extracellular conditions to the transferrin receptor on the cell of the subject; and
endocytosing the peptide complex, the target molecule, and the transferrin receptor;
unbinding the target-binding peptide from the target molecule, the transferrin receptor-binding peptide from the transferrin receptor, or both under endosomal conditions, thereby treating the disease or condition.

193. The method of claim 192, wherein the target molecule is an extracellular protein, a secreted peptide, a secreted protein, a circulating protein, a soluble protein, a cell surface protein, or a transmembrane protein.

194. The method of claim 192, further comprising degrading the target molecule in an endocytic or lysosomal compartment.

195. The method of claim 194, further comprising depleting the target molecule.

196. The method of claim 192, wherein the disease or condition is a cancer, a neurodegenerative disease, a lysosomal storage disease, an inflammatory disease, an autoimmune disease, a neuroinflammatory disease, an immune disease, or pain.

197. The method of claim 196, wherein the cancer is breast cancer, liver cancer, colon cancer, brain cancer, leukemia, lymphoma, non-Hodgkin lymphoma, myeloma, blood-cell-derived cancer, lung cancer, sarcoma, stomach cancer, a gastrointestinal cancer, glioblastoma, head and neck cancer, non-small-cell lung cancer, squamous non-small cell lung cancer, pancreatic cancer, ovarian cancer, blood cancer, skin cancer, liver cancer, kidney cancer, endometrial cancer, melanoma, bladder cancer, osteosarcoma, prostate cancer, myeloma, spleen cancer, bone marrow cell cancer, or colorectal cancer.

198. The method of claim 196, wherein the cancer is TKI-resistant, cetuximab-resistant, necitumumab-resistant, panitumumab-resistant, an advanced cancer, a metastatic cancer, a metastatic cancer in the central nervous system, metastatic breast cancer, metastatic skin cancer, a refractory cancer, a KRAS wild type cancer, a KRAS mutant cancer, or an exon20 mutant non-small-cell lung cancer.

199. The method of claim 196, wherein the neurodegenerative disease is Alzheimer's disease, amyotrophic lateral sclerosis, Friedreich's ataxia, Huntington's disease, schizophrenia, Parkinson's disease, or spinal muscular atrophy.

200. The method of claim 196, wherein the inflammatory disease is rheumatoid arthritis, psoriasis, multiple sclerosis, glomerulonephritis, lupus, inflammatory bowel disease, ulcerative colitis, Crohn's disease, cutaneous vasculitis, neuroinflammatory disease, inflammation-associated neurodegeneration, Alzheimer's disease, stroke, traumatic brain injury, Sjogren's disease, or cystic fibrosis.

201. A peptide complex comprising:
a transferrin receptor-binding peptide; and
a target-binding peptide complexed with the transferrin receptor-binding peptide, wherein the target-binding peptide has a binding affinity for a target.

202. The peptide complex of claim 201, wherein the binding affinity of the target-binding peptide for the target is pH-independent.

203. The peptide complex of claim 201, wherein the binding affinity of the target-binding peptide for the target is substantially the same the binding affinity at pH 7.4 and pH 5.5.

204. The peptide complex of claim 201, wherein the target is an extracellular protein, a secreted peptide, a secreted protein, a circulating protein, a soluble protein, a cell surface protein, or a transmembrane protein.

205. The peptide complex of claim 201, wherein the target is a growth factor receptor, a receptor tyrosine kinase, a cell signaling molecule, an extracellular matrix macromolecule, a neurotransmitter, a cytokine, a growth factor, a tumor associated antigen, a tumor specific antigen, a hormone, a checkpoint inhibitor, an immune checkpoint inhibitor, an inhibitory immune receptor, a ligand of an inhibitory immune receptor, a macrophage surface protein, a lipopolysaccharide, an antibody, an inhibitory immune receptor, a tumor associated antigen, a tumor specific antigen, or an autoantibody.

206. The peptide complex of claim 201, wherein the target is collagen, elastin, a microfibrillar protein, a proteoglycan, CD200R, CD300a, CD300f, CEACAM1, FcgRiib, ILT-2, ILT-3, ILT-4, ILT-5, LAIR-1, PECAM-1, PILR-alpha, SIRL-1, and SIRP-alpha, CLEC4A, Ly49Q, MIC, CD3, CD47, CD28, CD137, CD89, CD14, CD16, CD29, CD44, CD71, CD73, CD90, CD105, CD166, CD27, CD39, CD24, CD25, CD74, CD40L, MUC1, MUC16, MUC2, MUC5AC, MUC4, OX40, 4-1BB, HLA-G, LAG3, Tim3, TIGIT, GITR, TCR, TNF-α, EGFR, EGFRvIII, TKI-resistant EGFR, HER2, ERBB3, PDGFR, FGF, VEGF, VEGFR, IGFR1, CTLA4, STRO1, complement factor C4, complement factor C1q, complement factor C1s, complement factor C1r, complement factor C3, complement factor C3a, complement factor C3b, complement factor C5, complement factor C5a, TGFβ, PCSK9, P2Y6, HER3, RANK, tau, amyloid ß, huntingtin, α-synuclein, glucocerebrosidase, α-glucosidase, apolipoprotein E4, IL-1, IL-1R, IL-1α, IL-1β, IL-2, IL-2R, IL-4, IL-5, IL-6, IL-6R, IL-10, IL-10R, IL-17, IL-23, IL-12, p40, a member of the B7 family, c-Met, SIGLEC, MCP-1, an MHC, an MHC I, an MHC II, PD-1, or PD-L1.

207. The peptide complex of claim 201, wherein the target is EGFR, EGFRvIII, PD-L1, IL-1R, IL-1β, c-Met, FGFR-1, ERBB3, HER2, AXL receptor, CD44, IGF1R, MUC1, PDGFR, CD47, CD73, CD30, VEGFR, VEGFR2, complement factor C4, tau, amyloid ß, α-synuclein, PCSK9, or TNFα.

208. The peptide complex of claim 201, wherein the transferrin receptor-binding peptide, the target-binding peptide, or both comprises a miniprotein, a nanobody, an antibody, an antibody fragment, an scFv, a DARPin, or an affibody.

209. The peptide complex of claim 201, wherein the transferrin receptor-binding peptide comprises a cystine-dense peptide comprising at least three disulfide bonds.

210. The peptide complex of claim 201, wherein the transferrin receptor-binding peptide comprises a sequence of SEQ ID NO: 170.

211. The peptide complex of claim 201, wherein the transferrin receptor-binding peptide comprises a sequence having at least 90% sequence identity to any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64, or wherein the transferrin receptor-binding peptide comprises a sequence having at least 90% sequence identity to a fragment of any one of SEQ ID NO: 96, SEQ ID NO: 65-SEQ ID NO: 95, SEQ ID NO: 97-SEQ ID NO: 128, SEQ ID NO: 220-SEQ ID NO: 222, or SEQ ID NO: 1-SEQ ID NO: 64.

212. The peptide complex of claim 201, wherein the transferrin receptor-binding peptide comprises a sequence of SEQ ID NO: 96, SEQ ID NO: 66, SEQ ID NO: 220, SEQ ID NO: 221, or SEQ ID NO: 222.

213. The peptide complex of claim 201, wherein the target-binding peptide comprises a sequence that has at least 90% sequence identity with SEQ ID NO: 219 or SEQ ID NO: 242.

214. The peptide complex of claim 201, wherein the target-binding peptide comprises a sequence that has at least 90% sequence identity with SEQ ID NO: 242.

215. The peptide complex of claim 201, wherein the target-binding peptide comprises a sequence that has at least 90% sequence identity with SEQ ID NO: 219.

216. The peptide complex of claim 201, wherein the target-binding peptide comprises one or more histidine residues at a target-binding interface.

217. The peptide complex of claim 201, wherein the peptide complex comprises a dimer dimerized via a dimerization domain, wherein the dimerization domain comprises an Fc domain, a homodimerization domain, a first heterodimerization domain, a second heterodimerization domain, or combinations thereof.

218. The peptide complex of claim 217, wherein the first heterodimerization domain comprises a sequence that has at least 90% sequence identity with any one of SEQ ID NO: 260, SEQ ID NO: 262, SEQ ID NO: 264, SEQ ID NO: 266, SEQ ID NO: 268, SEQ ID NO: 270, SEQ ID NO: 272, SEQ ID NO: 274, SEQ ID NO: 276, SEQ ID NO: 278, SEQ ID NO: 280, SEQ ID NO: 282, SEQ ID NO: 284, or SEQ ID NO: 286.

219. The peptide complex of claim 201, comprising a sequence that has at least 90% sequence identity with any one of SEQ ID NO: 292, SEQ ID NO: 295, SEQ ID NO: 298, SEQ ID NO: 299, SEQ ID NO: 301, SEQ ID NO: 311, SEQ ID NO: 315, SEQ ID NO: 316, SEQ ID NO: 347, SEQ ID NO: 349, SEQ ID NO: 350, SEQ ID NO: 353, SEQ ID NO: 368, SEQ ID NO: 369, SEQ ID NO: 371-SEQ ID NO: 380, SEQ ID NO: 382-SEQ ID NO: 389.

220. A method of depleting a target molecule from a cellular surface or an extracellular space, the method comprising:
contacting a peptide complex to a cell expressing a transferrin receptor, wherein the peptide complex comprises:
a transferrin receptor-binding peptide, and
a target-binding peptide complexed with the transferrin receptor-binding peptide;
binding the target-binding peptide to the target molecule under extracellular conditions;
binding the transferrin receptor-binding peptide to the transferrin receptor under extracellular conditions; and
endocytosing the peptide complex, the target molecule, and the transferrin receptor; and
thereby depleting the target molecule from the cellular surface or the extracellular space.

221. The method of claim 220, wherein the target molecule is an extracellular protein, a secreted peptide, a secreted protein, a circulating protein, a soluble protein, a cell surface protein, or a transmembrane protein.
